SEQUENCE LISTING (33 sequences)

SEQ ID NO: 1 - DNA, 1616 bp, Homo sapiens
gccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 2 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 3 - DNA, 2289 bp, Artificial Sequence
  Description of Artificial Sequence Synthetic polynucleotide
atgctactagtaaatcagtcacaccaaggcttcaataaggaacacacaagcaagatggtaagcgctattgttttatatgtgcttttggcggcggcggcgcattctgcctttgcggcggatccgcatcatcatcatcatcacagctccggaatcgagggacgtggtaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgctgagactttgagtgaagtagcctaa

SEQ ID NO: 4 - protein, 719 aa, Artificial Sequence
  Description of Artificial Sequence Synthetic polypeptide
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSKHVIYAPSSHNKYAGESFPGIYDALFDIESKVDPSKAWGEVKRQIYVAAFTVQAAAETLSEVA

SEQ ID NO: 5 - DNA, 528 bp, Homo sapiens
gtcgacatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgtcgac

SEQ ID NO: 6 - protein, 172 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 7 - DNA, 678 bp, Artificial Sequence
  Description of Artificial Sequence Synthetic polynucleotide
ctcgagggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgag

SEQ ID NO: 8 - protein, 222 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSG

SEQ ID NO: 9 - DNA, 888 bp, Artificial Sequence
  Description of Artificial Sequence Synthetic polynucleotide
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccc

SEQ ID NO: 10 - protein, 296 aa, Artificial Sequence
  Description of Artificial Sequence Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 11 - DNA, 2217 bp, Homo sapiens
atgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagagaaaccatttactgttgagatctcccctggaccccggattgctgctcagattggagactcagtcatgttgacatgtagtgtcatgggctgtgaatccccatctttctcctggagaacccagatagacagccctctgagcgggaaggtgaggagtgaggggaccaattccacgctgaccctgagccctgtgagttttgagaacgaacactcttatctgtgcacagtgacttgtggacataagaaactggaaaagggaatccaggtggagctctactcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacacttgatgttcaaggaagagaaaacaacaaagactatttttctcctgagcttctcgtgctctattttgcatcctccttaataatacctgccattggaatgataatttactttgcaagaaaagccaacatgaaggggtcatatagtcttgtagaagcacagaaatcaaaagtg

SEQ ID NO: 12 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSEQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 13 - DNA, 636 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatg

SEQ ID NO: 14 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 15 - DNA, 1404 bp, Homo sapiens
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccaga

SEQ ID NO: 16 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 17 - protein, 16 aa, Homo sapiens
MGSNKSKPKDASQRRR

SEQ ID NO: 18 - protein, 5 aa, Artificial Sequence
  Description of Artificial Sequence Synthetic peptide
MGCXC

SEQ ID NO: 19 - protein, 9 aa, Unknown
  Description of Unknown Melanoma-associated antigen 3 peptide
FLWGPRALV

SEQ ID NO: 20 - protein, 9 aa, Influenza virus
GILGFVFTL

SEQ ID NO: 21 - protein, 9 aa, Human immunodeficiency virus type 1
SLYNTVATL

SEQ ID NO: 22 - protein, 21 aa, Clostridium tetani
FNNFTVSFWLRVPKVSASHLE

SEQ ID NO: 23 - DNA, 35 bp, Artificial Sequence
  Description of Artificial Sequence Synthetic primer
atatactcgagaaaaaggtggccaagaagccaacc

SEQ ID NO: 24 - DNA, 36 bp, Artificial Sequence
  Description of Artificial Sequence Synthetic primer
atatagtcgactcactgtctctcctgcactgagatg

SEQ ID NO: 25 - DNA, 33 bp, Artificial Sequence
  Description of Artificial Sequence Synthetic primer
acatcaactcgagatggctgcaggaggtcccgg

SEQ ID NO: 26 - DNA, 34 bp, Artificial Sequence
  Description of Artificial Sequence Synthetic primer
actcatagtcgaccagggacaaggccttggcaag

SEQ ID NO: 27 - DNA, 45 bp, Artificial Sequence
  Description of Artificial Sequence Synthetic oligonucleotide
ggaggcggaggcagcggaggtggcggttccggaggcggaggttct

SEQ ID NO: 28 - protein, 15 aa, Artificial Sequence
  Description of Artificial Sequence Synthetic peptide
GGGGSGGGGSGGGGS

SEQ ID NO: 29 - protein, 8 aa, Artificial Sequence
  Description of Artificial Sequence Synthetic peptide
SIINFEKL

SEQ ID NO: 30 - protein, 17 aa, Artificial Sequence
  Description of Artificial Sequence Synthetic peptide
ISQAVHAAHAEINEAGR

SEQ ID NO: 31 - protein, 9 aa, Artificial Sequence
  Description of Artificial Sequence Synthetic peptide
SVYDFFVWL

SEQ ID NO: 32 - protein, 7 aa, Artificial Sequence
  Description of Artificial Sequence Synthetic peptide
SINFEKL

SEQ ID NO: 33 - protein, 6 aa, Artificial Sequence
  Description of Artificial Sequence Synthetic 6xHis tag
HHHHHH

(19) United States
(12) Patent Application Publication
(10) Pub. No.: US 2014/0023647 A1
(43) Pub. Date: Jan. 23, 2014

(54) METHOD FOR TREATING SOLID TUMORS

(71) Applicant: BELLICUM PHARMACEUTICALS, INC., Houston, TX (US)

(72) Inventors: Kevin SLAWIN, Houston, TX (US); David Spencer, Houston, TX (US); Natalia Lapteva, Houston, TX (US)

(73) Assignee: BELLICUM PHARMACEUTICALS, INC., Houston, TX (US)

(21) Appl. No.: 13/786,652

(22) Filed: Mar. 6, 2013

Related U.S. Application Data
(62) Division of application No. 13/087,329, filed on Apr. 14, 2011.
(60) Provisional application No. 61/325,127, filed on Apr. 16, 2010.
(60) Provisional application No. 61/351,760, filed on Jun. 4, 2010.
(60) Provisional application No. 61/442,582, filed on Feb. 14, 2011.

(51) Int. Cl. A61K 39/00; A61K 31/337; C07K 14/705

(52) U.S. Cl. CPC A61K 39/0011; C07K 14/705; A61K 31/337; C07K 14/70578
USPC 424/134.1; 424/192.1; 424/185.1

(57) ABSTRACT

Provided herein are methods for treating a solid tumor in a subject in need thereof by activating an immune response against a tumor antigen. Also provided are methods for treating a solid tumor in a subject in need thereof by activating antigen-presenting cells and eliciting an immune response against a tumor antigen. Also provided herein are optimized therapeutic treatments of solid tumors, which comprise determining the presence, absence or amount of a biomarker after the therapy has been administered, and determining whether a subsequent dose of the therapy should be maintained, increased, or decreased based on the biomarker assessment.

### RELATED PATENT APPLICATIONS

This patent application is a divisional application of U.S. patent application Ser. No. 13/087,329, filed Apr. 14, 2011, and entitled METHOD FOR TREATING SOLID TUMORS, naming Kevin Slawin, David M. Spencer, and Natalia Lapteva as inventors, which is a non-provisional patent application claiming priority to U.S. Provisional Patent Application Ser. No. 61/442,582, filed Feb. 14, 2011, and entitled “Method for Treating Solid Tumors;” to U.S. Provisional Patent Application Ser. No. 61/351,760, filed Jun. 4, 2010, and entitled “Method for Treating Solid Tumors;” and to U.S. Provisional Patent Application Ser. No. 61/325,127, filed Apr. 16, 2010, and entitled “Method for Treating Solid Tumors;” which are all referred to and all incorporated by reference herein in their entirety. This application incorporates by reference the computer readable “Sequence Listing” that was filed on Aug. 11, 2011, in U.S. patent application Ser. No. 13/087,329, filed Apr. 14, 2011.

### FIELD

The technology relates generally to the field of immunology and relates in part to methods for treating a solid tumor in a subject in need thereof by inducing an immune response. The technology further relates in part to optimized therapeutic treatments of solid tumors.

### BACKGROUND

Antigen-presenting cells present foreign antigens to naïve T cells, inducing a cytotoxic T lymphocyte response. Dendritic cells are effective antigen presenting cells, and activation of the cells often results in a high level expression of costimulatory and cytokine molecules. In order to have effective immunotherapy against cancer cells, such as tumor cells, any immune response against the cells needs to have a long enough life span to be able to continually activate T cells. For use as a vaccine against cancer cells, the antigen presenting cells need to be sufficiently activated, have sufficient migration to the lymph node, and have a lifespan that is long enough to activate T cells in the lymph node.

Dendritic cells and other vaccines acting through antigen presenting cells have been tested for use as vaccines against prostate cancer, including, for example, Sipuleucel-T and Prostvac, but no statistically significant benefit in time to disease progression was found in treated subjects in randomized clinical trials evaluating either agent. (Drugs R & D (2006) 7:197-201; Kantoff, P., et al., (2010) New Eng. J. Med. 363:411-422; Kantoff, P., et al. (2010) J. Clin. One. 28:1099-1105).

### SUMMARY

An inducible CD40 (iCD40) system has been applied to human dendritic cells, and used to reduce tumor size in cancer patients. These features form the basis of cancer immunotherapies for treating or preventing such cancers as advanced, hormone-refractory prostate cancer, for example. Accordingly, it has been found that inducing CD40 in antigen presenting cells, and activating an antigenic response against a prostate cancer antigen, for example, a prostate specific membrane antigen (PSMA) provides an anti-tumor effect against not only prostate cancer associated tumors, but also other solid tumors by both direct effects and by targeting tumor vasculature. By inducing an immune response against prostate specific protein antigen, for example, a PSMA polypeptide, the size or growth of solid tumors may be reduced. The therapeutic course of treatment may be monitored by determining the size and vascularity of tumors by various imaging modalities (e.g. CT, bonescan, MRI, PET scans, Trofex scans), by various standard blood biomarkers (e.g. PSA, Circulating Tumor Cells), or by serum levels of various inflammatory, hypoxic cytokines, or other factors in the treated patient.

Thus featured in some embodiments are methods of treating or preventing prostate cancer in a subject, comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a prostate cancer antigen, such as, for example, a prostate specific protein antigen, for example, a prostate specific membrane antigen; and administering a multimeric ligand that binds to the multimeric ligand binding region, whereby the antigen presenting cell and ligand are administered in an amount effective to treat or prevent the prostate cancer in the subject.

Thus also featured in some embodiments are methods of inducing an immune response against a tumor antigen, such as, for example, a prostate cancer antigen, a prostate specific protein antigen, or a prostate specific membrane antigen, in a subject, comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen or a prostate specific membrane antigen; and administering an FK506 dimer or a dimeric FK506 analog ligand. whereby the antigen presenting cell and ligand are administered in an amount effective to induce an immune response in the subject. In some embodiments, the immune response is a cytotoxic T-lymphocyte immune response.

Also featured in some embodiments are methods of reducing tumor size or inhibiting tumor growth in a subject, comprising inducing an immune response against a tumor antigen, for example, a prostate cancer antigen, a prostate specific protein antigen, or a prostate specific membrane antigen in the subject. In some embodiments, the immune response is a cytotoxic T-lymphocyte immune response. In some embodiments, the method comprises administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with an antigen, for example, a prostate specific membrane antigen; and administering a multimeric ligand that binds to the multimeric ligand binding region, whereby the antigen presenting cell and ligand are administered in an amount effective to treat reduce tumor size or inhibit tumor growth in the subject. In some embodiments, the subject has prostate cancer. In some embodiments, the tumor is in the prostate. In some embodiments, the tumor is in a lung, bone, liver, prostate, brain, breast, ovary, bowel, testes, colon, pancreas, kidney, bladder, neuroendocrine system, lymphatic system, or is a soft tissue sarcoma, glioblastoma, or malignant myeloma. In some embodiments, the transduced or transfected antigen presenting cell is loaded with an antigen, for example, a prostate specific membrane antigen by contacting the cell with a tumor antigen, such as, for example, a prostate cancer antigen, a prostate specific protein antigen, or a prostate specific membrane antigen. In some embodiments, the transduced or transfected antigen presenting cell is loaded with an antigen, for example, a prostate specific membrane antigen by transducing or transfecting the antigen presenting cell with a nucleic acid coding for a tumor antigen, such as, for example, a prostate cancer antigen, a prostate specific protein antigen, or a prostate specific membrane antigen. In some embodiments, the tumor is in the prostate, in some embodiments the subject has prostate cancer. In some embodiments, wherein the tumor is in the lung; in some embodiments, the subject has lung cancer. In some embodiments, the tumor is in the lung, lymph node, bone, or liver.

Also featured in some embodiments are methods of reducing tumor vascularization or inhibiting tumor vascularization in a subject, comprising inducing an immune response against a tumor antigen, such as, for example, a prostate cancer antigen, a prostate specific protein antigen, or a prostate specific membrane antigen in the subject. In some embodiments, the immune response is a cytotoxic T-lymphocyte immune response. In some embodiments, the method comprises administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with an antigen, for example, a prostate specific membrane antigen; and administering a multimeric ligand that binds to the multimeric ligand binding region, whereby the antigen presenting cell and ligand are administered in an amount effective to treat reduce tumor vascularization or inhibit tumor vascularization in the subject. In some embodiments, the subject has prostate cancer. In some embodiments, the tumor is in the prostate. In some embodiments, the tumor is in a lung, bone, liver, prostate, brain, breast, ovary, bowel, testes, colon, pancreas, kidney, bladder, neuroendocrine system, lymphatic system, or is a soft tissue sarcoma, glioblastoma, or malignant myeloma. In some embodiments, the transduced or transfected antigen presenting cell is loaded with an antigen, for example, a prostate specific membrane antigen by contacting the cell with an antigen, for example, a prostate specific membrane antigen. In some embodiments, the transduced or transfected antigen presenting cell is loaded with an antigen, for example, a prostate specific membrane antigen by transducing or transfecting the antigen presenting cell with a nucleic acid coding for the antigen, for example, a prostate specific membrane antigen. In some embodiments, the level of vascularization is determined by molecular imaging. In some embodiments, wherein the molecular imaging comprises administration of an iodine 123-labelled PSA, for example, PSMA inhibitor. In some embodiments, the inhibitor is TROFEX™/MIP-1072/1095.

Also featured in some embodiments are methods of reducing or slowing tumor vascularization in a subject, comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, for example, a prostate cancer antigen, a prostate specific protein antigen, or a prostate specific membrane antigen; and administering a multimeric ligand that binds to the multimeric ligand binding region, whereby the antigen presenting cell and ligand are administered in an amount effective to reduce or slow tumor vascularization in the subject.

In some embodiments, the tumor vascularization is reduced in the prostate. In some embodiments, the subject has prostate cancer. In some embodiments, the tumor is in the lung, liver, lymph node, or bone.

In some embodiments, the membrane targeting region is selected from the group consisting of a myristoylation region, palmitoylation region, prenylation region, and transmembrane sequences of receptors. In some embodiments, the membrane targeting region is a myristoylation region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the ligand is AP1903. In some embodiments, the antigen presenting cell is administered to the subject by intravenous, intradermal, subcutaneous, intratumor, intraprotatic, or intraperitoneal administration. In some embodiments, the prostate cancer is selected from the group consisting of metastatic, metastatic castration resistant, metastatic castration sensitive, regionally advanced, and localized prostate cancer. In some embodiments, at least two doses of the antigen presenting cell and the ligand are administered to the subject. In some embodiments, the antigen presenting cell is a dendritic cell. In some embodiments, the CD40 cytoplasmic polypeptide region is encoded by a polynucleotide sequence in SEQ ID NO: 1. In some embodiments, the prostate specific membrane antigen comprises the amino acid sequence of SEQ ID NO: 4, or a fragment thereof, or is encoded by the nucleotide sequence of SEQ ID NO: 3, or a fragment thereof. In some embodiments, the antigen presenting cell is transfected with a vector, for example, a virus vector, for example, an adenovirus vector. In some embodiments, the antigen presenting cell is transfected with an Ad5f35 vector. In some embodiments, the FKB12 region is an FKB12v36 region.

In some embodiments, the method further comprises determining the level of IL-6 in the subject after the administration of the antigen presenting cell and the ligand. In some embodiments, the method further comprises determining whether to administer an additional dose or additional doses of the antigen presenting cell and the ligand to the subject, wherein the determination is based upon the level of IL-6 in the subject after administration of at least one dose. In some embodiments, an additional dose is administered where the IL-6 level is above normal. In some embodiments, the IL-6 is from serum.

In some embodiments, the methods further comprise determining the level of VCAM-1 in the subject after the administration of the antigen presenting cell and the ligand. In some embodiments, the method further comprises determining whether to administer an additional dose or additional doses of the antigen presenting cell and the ligand to the subject, wherein the determination is based upon the level of VCAM-1 in the subject after administration of at least one dose. In some embodiments, an additional dose is administered where the VCAM-1 level is above normal. In some embodiments, the VCAM-1 is from serum.

In some embodiments, the progression of prostate cancer is prevented or progression of prostate cancer is delayed in the subject. In some embodiments, the transduced or transfected antigen presenting cell is loaded with a prostate cancer antigen, for example, a prostate specific protein antigen or a prostate specific membrane antigen by contacting the cell with a prostate cancer antigen, for example, a prostate specific membrane antigen. In some embodiments, the transduced or transfected antigen presenting cell is loaded with a prostate cancer antigen, for example, a prostate specific membrane antigen by transducing or transfecting the antigen presenting cell with a nucleic acid coding for a prostate cancer antigen, for example, a prostate specific membrane antigen. In some embodiments, the nucleic acid coding for the prostate cancer antigen, for example, a prostate specific membrane antigen is DNA. In some embodiments, the nucleic acid coding for the prostate cancer antigen, for example, a prostate specific membrane antigen is RNA. In some embodiments, the antigen presenting cell is a B cell. In some embodiments, the chimeric protein further comprises a MyD88 polypeptide or a truncated MyD88 polypeptide lacking the TIR domain. In some embodiments, the truncated MyD88 polypeptide has the peptide sequence of SEQ ID NO: 6, or a fragment thereof, or is encoded by the nucleotide sequence of SEQ ID NO: 5, or a fragment thereof. In some embodiments, the prostate cancer antigen, for example, a prostate specific membrane antigen is a prostate specific membrane antigen polypeptide.

Also featured in some embodiments are methods of treating or preventing prostate cancer in a subject, comprising administering a composition comprising a nucleotide sequence that encodes a chimeric protein and a nucleotide sequence encoding a prostate cancer antigen, for example, a prostate specific protein antigen or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region; whereby the composition and ligand are administered in an amount effective to treat or prevent the prostate cancer in the subject. Also featured in some embodiments are methods of treating or preventing prostate cancer in a subject, comprising administering a nucleotide sequence that encodes a chimeric protein, and a nucleotide sequence encoding a prostate cancer antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding a prostate cancer antigen, for example, a prostate specific membrane antigen are delivered using a vector, for example, a virus vector, for example, an adenovirus vector; and administering a multimeric ligand that binds to the multimeric ligand binding region; whereby the composition and ligand are administered in an amount effective to treat or prevent the prostate cancer in the subject.

In some embodiments, progression of prostate cancer is prevented or delayed at least 6 months. In some embodiments, progression of prostate cancer is prevented or delayed at least 12 months. In some embodiments, the prostate cancer has a Gleason score of 7, 8, 9, 10, or greater. In some embodiments, the subject has a partial or complete response by 3 months after administration of the multimeric ligand. In some embodiments, the subject has a partial or complete response by 6 months after administration of the multimeric ligand. In some embodiments, the subject has a partial or complete response by 9 months after administration of the multimeric ligand. In some embodiments, the level of serum PSA in the subject is reduced 20%, 30%, 40%. 50%, 60%, 70%, 80% 90% or 95% by 6 weeks after administration of the multimeric ligand. In some embodiments, the level of serum PSA in the subject is reduced by 3 months 20%, 30%, 40%. 50%, 60%, 70%, 80% 90% or 95% after administration of the multimeric ligand. In some embodiments, the level of serum PSA in the subject is reduced 20%, 30%, 40%. 50%, 60%, 70%, 80% 90% or 95% by 6 months after administration of the multimeric ligand. In some embodiments, the level of serum PSA in the subject is reduced 20%, 30%, 40%. 50%, 60%, 70%, 80% 90% or 95% by 9 months after administration of the multimeric ligand. In some embodiments, the size of the prostate cancer tumor is reduced 30%, 40%. 50%, 60%, 70%, 80% 90% or 95% by 3 months after administration of the multimeric ligand. In some embodiments, the size of the prostate cancer tumor is reduced 30%, 40%. 50%, 60%, 70%, 80% 90% or 95% by 6 months after administration of the multimeric ligand. In some embodiments, the size of the prostate cancer tumor is reduced 30%, 40%. 50%, 60%, 70%, 80% 90% or 95% by 9 months after administration of the multimeric ligand. In some embodiments, the vascularization of the prostate cancer tumor is reduced 30%, 40%. 50%, 60%, 70%, 80% 90% or 95% by 3 months after administration of the multimeric ligand. In some embodiments, the vascularization of the prostate cancer tumor is reduced 30%, 40%. 50%, 60%, 70%, 80% 90% or 95% by 6 months after administration of the multimeric ligand. In some embodiments, the vascularization of the prostate cancer tumor is reduced 30%, 40%. 50%, 60%, 70%, 80% 90% or 95% by 9 months after administration of the multimeric ligand. In some embodiments, a T_{H}1 or T_{H}2 antigen-specific immune response is detected in the subject after administration of the multimeric ligand.

Also featured in some embodiments are methods of inducing an immune response against a tumor antigen, for example, a prostate cancer antigen, a prostate specific protein antigen, or a prostate specific membrane antigen in a subject, comprising administering a composition comprising a nucleotide sequence that encodes a chimeric protein and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region. In some embodiments, the composition and the ligand are administered in an amount effective to induce an immune response in the subject. Also featured in some embodiments are methods of inducing an immune response against a tumor antigen, for example, a prostate cancer antigen, a prostate specific protein antigen, or a prostate specific membrane antigen, in a subject, comprising administering a nucleotide sequence that encodes a chimeric protein, and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding the antigen, for example, a prostate specific membrane antigen are delivered using a vector, for example, a virus vector, for example, an adenovirus vector; and administering a multimeric ligand that binds to the multimeric ligand binding region. In some embodiments, the nucleotide sequences and the ligand are administered in an amount effective to induce an immune response in the subject. In some embodiments, the immune response is a cytotoxic T-lymphocyte immune response.

Also featured in some embodiments are methods of reducing tumor size or inhibiting tumor growth in a subject, comprising inducing an immune response against a tumor antigen, for example, a prostate cancer antigen, a prostate specific protein antigen, or a prostate specific membrane antigen, in the subject. In some embodiments, the method comprises administering a composition comprising a nucleotide sequence that encodes a chimeric protein and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region. In some embodiments, the method comprises administering a nucleotide sequence that encodes a chimeric protein, and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding the antigen, for example, a prostate specific membrane antigen are delivered using a vector, for example, a virus vector, for example, an adenovirus vector; and administering a multimeric ligand that binds to the multimeric ligand binding region. In some embodiments, the composition or nucleotide sequences and the ligand are administered in an amount effective to reduce tumor size or inhibit tumor growth in the subject. In some embodiments, the subject has prostate cancer. In some embodiments, the tumor is in the prostate. In some embodiments, the tumor is in a lung, bone, liver, prostate, brain, breast, ovary, bowel, testes, colon, pancreas, kidney, bladder, neuroendocrine system, lymphatic system, or is a soft tissue sarcoma, glioblastoma, or malignant myeloma. In some embodiments, the tumor is in the lung, liver, lymph node, or bone.

Also featured in some embodiments are methods of reducing tumor vascularization or inhibiting tumor vascularization in a subject, comprising inducing an immune response against a tumor antigen, for example a prostate cancer antigen, a prostate specific protein antigen, or a prostate specific membrane antigen in the subject. In some embodiments, the method comprises administering a composition comprising a nucleotide sequence that encodes a chimeric protein and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region. In some embodiments, the method comprises administering a nucleotide sequence that encodes a chimeric protein, and a nucleotide sequence encoding an antigen, for example, a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding the antigen, for example, a prostate specific membrane antigen are delivered using a vector, for example, a virus vector, for example, an adenovirus vector; and administering a multimeric ligand that binds to the multimeric ligand binding region. In some embodiments, the composition or nucleotide sequences and the ligand are administered in an amount effective to reduce tumor vascularization or inhibit tumor vascularization in the subject. In some embodiments, the subject has prostate cancer. In some embodiments, the tumor is in the prostate. In some embodiments, the tumor is in a lung, bone, liver, prostate, brain, breast, ovary, bowel, testes, colon, pancreas, kidney, bladder, neuroendocrine system, lymphatic system, or is a soft tissue sarcoma, glioblastoma, or malignant myeloma. In some embodiments, the tumor is in a bone, lung, liver, or lymph node. In some embodiments, the level of vascularization is determined by molecular imaging. In some embodiments, the molecular imaging comprises administration of an iodine 123-labelled PSA, for example, PSMA inhibitor. In some embodiments, the inhibitor is TROFEX™/MIP-1072/1095.

Thus featured in some embodiments are methods comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, for example, a prostate cancer antigen, a prostate specific protein antigen, or a prostate specific membrane antigen, administering a multimeric ligand that binds to the multimeric ligand binding region; identifying the presence, absence or amount of a biomarker in the subject, wherein the biomarker is IL-6 or VCAM-1, or a portion of the foregoing; and maintaining a subsequent dosage of the cells or ligand or adjusting a subsequent dosage of the cells or ligand to the subject based on the presence, absence or amount of the biomarker identified in the subject.

Also featured in some embodiments are methods comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, for example, a prostate cancer antigen, a prostate specific protein antigen, or a prostate specific membrane antigen; administering a multimeric ligand that binds to the multimeric ligand binding region; identifying the presence, absence or amount of a biomarker in the subject, wherein the biomarker is IL-6 or VCAM-1, or a portion of the foregoing; and determining whether the dosage of the cells or ligand subsequently administered to the subject is adjusted based on the presence, absence or amount of the biomarker identified in the subject.

Thus featured in some embodiments are methods comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the presence, absence or amount of a biomarker in the subject, wherein the biomarker is uPAR, HGF, EGF, or VEGF, or a portion of the foregoing; and maintaining a subsequent dosage of the cells or ligand or adjusting a subsequent dosage of the cells or ligand to the subject based on the presence, absence or amount of the biomarker identified in the subject.

Also featured in some embodiments are methods comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a, prostate specific membrane antigen; administering a multimeric ligand that binds to the multimeric ligand binding region; identifying the presence, absence or amount of a biomarker in the subject, wherein the biomarker is uPAR, HGF, EGF, or VEGF, or a portion of the foregoing; and determining whether the dosage of the cells or ligand subsequently administered to the subject is adjusted based on the presence, absence or amount of the biomarker identified in the subject.

In some embodiments, at least two doses of the antigen presenting cells and the ligand are administered to the subject with 10 to 18 days between each dose. In some embodiments, six doses of the antigen presenting cell and the ligand are administered to the subject with 10 to 18 days between each dose. In some embodiments, three doses of the antigen presenting cell and the ligand are administered to the subject, with 24-32 days between each dose. In some embodiments, six doses of the antigen presenting cell and the ligand are administered to the subject, with two weeks between each dose. In some embodiments, three doses of the antigen presenting cell and the ligand are administered to the subject, with four weeks between each dose. In some embodiments, each dose of antigen presenting cells comprises about 4×10⁶ cells. In some embodiments, each dose of antigen presenting cells comprises about 12.5×10⁶ cells. In some embodiments, each dose of antigen presenting cells comprises about 25×10⁶ cells.

In some embodiments, the methods further comprise administering a chemotherapeutic agent. In some embodiments, whereby the composition, ligand, and the chemotherapeutic agent are administered in an amount effective to treat the prostate cancer in the subject. In some embodiments, the composition or the nucleotide sequences, the ligand, and the chemotherapeutic agent are administered in an amount effective to treat the prostate cancer in the subject. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the antigen presenting cell or the ligand. In other embodiments, the chemotherapeutic agent is administered after the administration of the ligand. In other embodiments, the chemotherapeutic agent is administered from 1 to 4 weeks or from 1 week to 1 month, 1 week to 2 months, or 1 week to 3 months after the administration of the ligand. In other embodiments, the methods further comprise administering the chemotherapeutic agent from 1 to 4 weeks, or from 1 week to 1 month, 1 week to 2 months, or 1 week to 3 months before the administration of the antigen presenting cell. In some embodiments, the chemotherapeutic agent is administered at least 2 weeks before administering the antigen presenting cell. In some embodiments, the chemotherapeutic agent is administered at least 1 month before administering the antigen presenting cell. In some embodiments, the chemotherapeutic agent is administered after administering the multimeric ligand. In some embodiments, the chemotherapeutic agent is administered at least 2 weeks after administering the multimeric ligand. In some embodiments, wherein the chemotherapeutic agent is administered at least 1 month after administering the multimeric ligand.

In some embodiments, the methods further comprise administering two or more chemotherapeutic agents. In some embodiments, the chemotherapeutic agents are selected from the group consisting of carboplatin, Estramustine phosphate, and thalidomide. In some embodiments, at least one chemotherapeutic agent is a taxane. The taxane may be, for example, selected from the group consisting of docetaxel, paclitaxel, and cabazitaxel. In some embodiments, the taxane is docetaxel. In some embodiments, the chemotherapeutic agents are administered at the same time or within one week after the administration of the antigen presenting cell or the ligand. In other embodiments, the chemotherapeutic agents are administered after the administration of the ligand. In other embodiments, the chemotherapeutic agents are administered from 1 to 4 weeks or from 1 week to 1 month, 1 week to 2 months, or 1 week to 3 months after the administration of the ligand. In other embodiments, the methods further comprise administering the chemotherapeutic agents from 1 to 4 weeks or from 1 week to 1 month, 1 week to 2 months, or 1 week to 3 months before the administration of the antigen presenting cell.

Also featured in some embodiments are methods of increasing the chemosensitivity of a tumor, comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a prostate specific membrane antigen; and administering a multimeric ligand that binds to the multimeric ligand binding region, whereby the antigen presenting cell and ligand are administered in an amount effective to increase the chemosensitivity of the tumor in the subject. The tumor may become more chemo-sensitive to any chemotherapeutic, such as, for example, a taxane, such as, for example, docetaxel or cabazitaxel.

By increasing the chemo-sensitivity of a tumor is meant, for example, increasing the sensitivity of a tumor to any chemotherapeutic, as measured by any method such as, for example, tumor size, growth rate, appearance, or vascularity. By increasing the chemo-sensitivity of a tumor is meant that the tumor is more sensitive to the chemotherapeutic than before vaccine therapy by, for example, at least 5%, 10%, 20%, 30%, 40%, 50%, 60%, 70%, 80%, 90%, or 100%.

Also featured in some embodiments are methods comprising: identifying the presence, absence or amount of a biomarker in a subject to whom a prostate membrane protein antigen-loaded antigen presenting cell and a multimeric ligand have been administered, the antigen presenting cell having been transduced or transfected with a nucleic acid including a nucleotide sequence that encodes a chimeric protein, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and maintaining a subsequent dosage of the cells or ligand or adjusting a subsequent dosage of the cells or ligand administered to the subject based on the presence, absence or amount of the biomarker identified in the subject.

Also featured in some embodiments are methods comprising: identifying the presence, absence or amount of a biomarker in a subject to whom a prostate membrane protein antigen-loaded antigen presenting cell and a multimeric ligand have been administered, the antigen presenting cell having been transduced or transfected with a nucleic acid including a nucleotide sequence that encodes a chimeric protein, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and determining whether the dosage of the cells or ligand subsequently administered to the subject is adjusted based on the presence, absence or amount of the biomarker identified in the subject.

Also featured in some embodiments are methods comprising: receiving information comprising the presence, absence or amount of a biomarker in a subject to whom a prostate membrane protein antigen-loaded antigen presenting cell and a multimeric ligand have been administered, the antigen presenting cell having been transduced or transfected with a nucleic acid including a nucleotide sequence that encodes a chimeric protein, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and maintaining a subsequent dosage of the cells or ligand or adjusting a subsequent dosage of the cells or ligand to the subject based on the presence, absence or amount of the biomarker identified in the subject.

Also featured in some embodiments are methods comprising: identifying the presence, absence or amount of a biomarker in a subject to whom a prostate membrane protein antigen peptide-loaded antigen presenting cell and a multimeric ligand have been administered, the antigen presenting cell having been transduced or transfected with a nucleic acid including a nucleotide sequence that encodes a chimeric protein, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and transmitting the presence, absence or amount of the biomarker to a decision maker who maintains a subsequent dosage of the cells or ligand or adjusts a subsequent dosage of the cells or ligand administered to the subject based on the presence, absence or amount of the biomarker identified in the subject.

Also featured in some embodiments are methods comprising: identifying the presence, absence or amount of a biomarker in a subject to whom a prostate membrane protein antigen peptide-loaded antigen presenting cell and a multimeric ligand have been administered, the antigen presenting cell having been transduced or transfected with a nucleic acid including a nucleotide sequence that encodes a chimeric protein, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and transmitting an indication to maintain a subsequent dosage of the cells or ligand or adjust a subsequent dosage of the cells or ligand administered to the subject based on the presence, absence or amount of the biomarker identified in the subject.

Also featured in some embodiments are methods for optimizing therapeutic efficacy, comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the presence, absence or amount of a biomarker in the subject, wherein the biomarker is IL-6 or VCAM-1, or the biomarker is uPAR, HGF, EGF, or VEGF, or a portion of the foregoing; and maintaining a subsequent dosage of the cells or ligand or adjusting a subsequent dosage of the cells or ligand to the subject based on the presence, absence or amount of the biomarker identified in the subject.

Also featured in some embodiments are methods for reducing toxicity of a treatment, comprising: administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the presence, absence or amount of a biomarker in the subject, wherein the biomarker is IL-6 or VCAM-1, or the biomarker is uPAR, HGF, EGF, or VEGF, or a portion of the foregoing; and maintaining a subsequent dosage of the cells or ligand or adjusting a subsequent dosage of the cells or ligand to the subject based on the presence, absence or amount of the biomarker identified in the subject.

Also featured in some embodiments are methods for administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the amount of IL-6 polypeptide or portion thereof in the subject; and maintaining a subsequent dosage of the cells or ligand or adjusting a subsequent dosage of the cells or ligand administered to the subject based on the amount of the IL-6 polypeptide or portion thereof identified in the subject. In some embodiments, the subject has a level of IL-6 polypeptide or portion thereof that is elevated relative to healthy subjects prior to administration of the cells.

Also featured in some embodiments are methods comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the amount of VCAM-1 polypeptide or portion thereof in the subject; and maintaining a subsequent dosage of the cells or ligand or adjusting a subsequent dosage of the cells or ligand administered to the subject based on the amount of the VCAM-1 polypeptide or portion thereof identified in the subject. In some embodiments, method of embodiment 111, wherein the subject has a level of VCAM-1 polypeptide or portion thereof that is elevated relative to healthy subjects prior to administration of the cells.

Also featured in some embodiments are methods comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the amount of uPAR, HGF, EGF, or VEGF, polypeptide or portion thereof in the subject; and maintaining a subsequent dosage of the cells or ligand or adjusting a subsequent dosage of the cells or ligand administered to the subject based on the amount of the VCAM-1 polypeptide or portion thereof identified in the subject. In some embodiments, method of embodiment I11, wherein the subject has a level of uPAR, HGF, EGF, or VEGF polypeptide or portion thereof that is elevated relative to healthy subjects prior to administration of the cells.

Also featured in some embodiments are methods comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; identifying the amount of an individual secreted factor, or a panel of secreted factors, in the subject wherein the secreted factors are selected from the group consisting of GM-CSF, MIP-1alpha, MIP-1beta, MCP-1, IFN-gamma, RANTES, EGF and HGF; and maintaining a subsequent dosage of the cells or ligand or adjusting a subsequent dosage of the cells or ligand administered to the subject based on the amount or a change in the amount of the individual serum factor or panel of serum factors identified in the subject.

Also featured in some embodiments are methods of reducing or slowing tumor vascularization in a subject, comprising administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region. Also featured in some embodiments are methods of reducing or slowing tumor vascularization in a subject, comprising administering a nucleotide sequence that encodes a chimeric protein, and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding a tumor antigen, such as, for example, a prostate cancer antigen, a prostate specific protein antigen, or a prostate specific membrane antigen are delivered using a vector, for example, a virus vector, for example, an adenovirus vector; and administering a multimeric ligand that binds to the multimeric ligand binding region.

In some embodiments, the nucleotide sequence encoding the tumor antigen, such as, for example, a prostate cancer antigen, a prostate specific protein antigen, or a prostate specific membrane antigen, and the nucleotide sequence encoding the chimeric protein are on different nucleic acids or on the same nucleic acid. In some embodiments, the nucleotide sequence encoding the tumor antigen, such as, for example, a prostate cancer antigen, a prostate specific protein antigen, or a prostate specific membrane antigen and the nucleotide sequence encoding the chimeric protein are on different adenovirus vectors or on the same adenovirus vector. In some embodiments, the membrane targeting region is selected from the group consisting of a myristoylation region, palmitoylation region, prenylation region, and transmembrane sequences of receptors. In some embodiments, the membrane targeting region is a myristoylation region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the prostate tumor antigen, for example, is a prostate specific membrane antigen polypeptide. In some embodiments, the composition further comprises particles, and the composition is administered by a propelling force. In some embodiments, the particles are gold particles or nanoparticles. In some embodiments, the ligand is AP1903. In some embodiments, the prostate cancer is selected from the group consisting of metastatic, metastatic castration resistant, metastatic castration sensitive, regionally advanced, and localized prostate cancer. In some embodiments, at least two doses of the composition and the ligand are administered to the subject. In some embodiments, at least two doses of the adenovirus vector or vectors and the ligand are administered to the subject. In some embodiments, the CD40 cytoplasmic polypeptide region is encoded by a polynucleotide sequence in SEQ ID NO: 1. In some embodiments, the prostate specific membrane antigen comprises the amino acid sequence of SEQ ID NO: 4 or a fragment thereof, or is encoded by the nucleotide sequence of SEQ ID NO: 3 or a fragment thereof. In some embodiments, the FKB12 region is an FKB12v36 region.

In some embodiments, the methods further comprise determining the level of IL-6 in the subject after the administration of the composition or adenovirus vectors and the ligand. In some embodiments, the method further comprises determining whether to administer an additional dose or additional doses to the subject, wherein the determination is based upon the level of IL-6 in the subject after administration of at least one dose. In some embodiments, the method further comprises administering an additional dose where the IL-6 level is above normal. In some embodiments, the IL-6 is from serum.

In some embodiments, the methods further comprise determining the level of VCAM-1 in the subject after the administration of the composition or adenovirus vectors and the ligand. In some embodiments, the method further comprises determining whether to administer an additional dose or additional doses to the subject, wherein the determination is based upon the level of VCAM-1 in the subject after administration of at least one dose. In some embodiments, the method further comprises administering an additional dose is where the VCAM-1 level is above normal. In some embodiments, the VCAM-1 is from serum.

In some embodiments, the methods further comprise determining the level of uPAR, HGF, EGF, or VEGF in the subject after the administration of the composition or adenovirus vectors and the ligand. In some embodiments, the method further comprises determining whether to administer an additional dose or additional doses to the subject, wherein the determination is based upon the level of uPAR, HGF, EGF, or VEGF in the subject after administration of at least one dose. In some embodiments, the method further comprises administering an additional dose is where the VCAM-1 level is above normal. In some embodiments, the uPAR, HGF, EGF, or VEGF is from serum.

In some embodiments, the progression of prostate cancer is prevented or progression of prostate cancer is delayed in the subject. In some embodiments, the transduced or transfected antigen presenting cell is loaded with a tumor antigen, such as, for example, a prostate cancer antigen, a prostate specific protein antigen, or a prostate specific membrane antigen by contacting the cell with a tumor antigen, such as, for example, a prostate cancer antigen, a prostate specific protein antigen, or a prostate specific membrane antigen. In some embodiments, the transduced or transfected antigen presenting cell is loaded with tumor antigen, such as, for example, a prostate cancer antigen, a prostate specific protein antigen, or a prostate specific membrane antigen by transducing or transfecting the antigen presenting cell with a nucleic acid coding for a tumor antigen, such as, for example, a prostate cancer antigen, a prostate specific protein antigen, or a prostate specific membrane antigen. In some embodiments, the nucleic acid coding for the tumor antigen, such as, for example, a prostate cancer antigen, a prostate specific protein antigen, or a prostate specific membrane antigen is DNA. In some embodiments, the nucleic acid coding for the tumor antigen, such as, for example, a prostate cancer antigen, a prostate specific protein antigen, or a prostate specific membrane antigen is RNA. In some embodiments, the antigen presenting cell is a B cell. In some embodiments, the chimeric protein further comprises a MyD88 polypeptide or a truncated MyD88 polypeptide lacking the TIR domain. In some embodiments, the truncated MyD88 polypeptide has the peptide sequence of SEQ ID NO: 6, or a fragment thereof, or is encoded by the nucleotide sequence of SEQ ID NO: 5, or a fragment thereof. In some embodiments, the tumor antigen, such as, for example, a prostate cancer antigen, a prostate specific protein antigen, or a prostate specific membrane antigen is a prostate specific membrane antigen polypeptide.

Also featured in some embodiments, are methods comprising administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the presence, absence or amount of a biomarker in the subject, wherein the biomarker is IL-6 or VCAM-1, or a portion of the foregoing; and maintaining a subsequent dosage of the composition or ligand or adjusting a subsequent dosage of the composition or ligand to the subject based on the presence, absence or amount of the biomarker identified in the subject.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the presence, absence or amount of a biomarker in the subject, wherein the biomarker is IL-6 or VCAM-1, or a portion of the foregoing; and determining whether the dosage of the composition or ligand subsequently administered to the subject is adjusted based on the presence, absence or amount of the biomarker identified in the subject.

Also featured in some embodiments, are methods comprising administering a composition comprising a nucleotide sequence that encodes a chimeric protein and a nucleotide sequence encoding tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the presence, absence or amount of a biomarker in the subject, wherein the biomarker is uPAR, HGF, EGF, or VEGF, or a portion of the foregoing; and maintaining a subsequent dosage of the composition or ligand or adjusting a subsequent dosage of the composition or ligand to the subject based on the presence, absence or amount of the biomarker identified in the subject.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the presence, absence or amount of a biomarker in the subject, wherein the biomarker is uPAR, HGF, EGF, or VEGF, or a portion of the foregoing; and determining whether the dosage of the composition or ligand subsequently administered to the subject is adjusted based on the presence, absence or amount of the biomarker identified in the subject.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; maintaining a subsequent dosage of the composition or ligand or adjusting a subsequent dosage of the composition or ligand administered to the subject based on the presence, absence or amount of the biomarker identified in the subject.

Also featured in some embodiments are methods comprising: identifying the presence, absence or amount of a biomarker in a subject to whom a composition and a multimeric ligand have been administered, the composition comprising a nucleotide sequence that encodes a chimeric protein and a nucleotide sequence encoding a tumor antigen, such as, for example, a prostate cancer antigen, a prostate specific protein antigen, or a prostate specific membrane antigen, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and determining whether the dosage of the composition or ligand subsequently administered to the subject is adjusted based on the presence, absence or amount of the biomarker identified in the subject.

Also featured in some embodiments are methods comprising: receiving information comprising the presence, absence or amount of a biomarker in a subject to whom a composition and a multimeric ligand have been administered, the composition comprising a nucleotide sequence that encodes a chimeric protein and a nucleotide sequence encoding a tumor antigen, such as, for example, a prostate cancer antigen, a prostate specific protein antigen, or a prostate specific membrane antigen w herein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and wherein the multimeric ligand binds to the multimeric ligand binding region; and maintaining a subsequent dosage of the composition or adjusting a subsequent dosage of the composition administered to the subject based on the presence, absence or amount of the biomarker identified in the subject.

Also featured in some embodiments are methods comprising: identifying the presence, absence or amount of a biomarker in a subject to whom a composition and a multimeric ligand have been administered, the composition comprising a nucleotide sequence that encodes a chimeric protein and a nucleotide sequence encoding tumor antigen, such as, for example, a prostate cancer antigen, a prostate specific protein antigen, or a prostate specific membrane antigen, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and wherein the multimeric ligand binds to the multimeric ligand binding region; and transmitting the presence, absence or amount of the biomarker to a decision maker who maintains a subsequent dosage of the composition or ligand or adjusts a subsequent dosage of the composition or ligand administered to the subject based on the presence, absence or amount of the biomarker identified in the subject.

Also featured in some embodiments are methods comprising: identifying the presence, absence or amount of a biomarker in a subject to whom a composition and a multimeric ligand have been administered, the composition comprising a nucleotide sequence that encodes a chimeric protein and a nucleotide sequence encoding a tumor antigen, such as, for example, a prostate cancer antigen, a prostate specific protein antigen, or a prostate specific membrane antigen, wherein the chimeric protein comprises a membrane targeting region, a multimeric ligand binding region and a CD40 cytoplasmic polypeptide region lacking the CD40 extracellular domain, and wherein the multimeric ligand binds to the multimeric ligand binding region; and wherein the multimeric ligand binds to the multimeric ligand binding region; and transmitting an indication to maintain a subsequent dosage of the composition or ligand or adjust a subsequent dosage of the composition or ligand administered to the subject based on the presence, absence or amount of the biomarker identified in the subject.

Also featured in some embodiments are methods for optimizing therapeutic efficacy, comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain;

administering a multimeric ligand that binds to the multimeric ligand binding region; identifying the presence, absence or amount of a biomarker in the subject, wherein the biomarker is IL-6 or VCAM-1, or a portion of the foregoing; and maintaining a subsequent dosage of the composition or ligand or adjusting a subsequent dosage of the composition or ligand to the subject based on the presence, absence or amount of the biomarker identified in the subject.

Also featured in some embodiments are methods for reducing toxicity of a treatment, comprising: administering a composition comprising a nucleotide sequence that encodes a chimeric protein and a nucleotide sequence encoding tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the presence, absence or amount of a biomarker in the subject, wherein the biomarker is IL-6 or VCAM-1, or uPAR, HGF, EGF, or VEGF, or a portion of the foregoing; and maintaining a subsequent dosage of the composition or ligand or adjusting a subsequent dosage of the composition or ligand to the subject based on the presence, absence or amount of the biomarker identified in the subject.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the amount of IL-6 polypeptide or portion thereof in the subject; and maintaining a subsequent dosage of the composition or ligand or adjusting a subsequent dosage of the composition or ligand administered to the subject based on the amount of the IL-6 polypeptide or portion thereof identified in the subject. In some embodiments, the subject has a level of IL-6 polypeptide or portion thereof that is elevated relative to healthy subjects prior to administration of the composition.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the amount of VCAM-1 polypeptide or portion thereof in the subject; and maintaining a subsequent dosage of the composition or ligand or adjusting a subsequent dosage of the composition or ligand administered to the subject based on the amount of the VCAM-1 polypeptide or portion thereof identified in the subject. In some embodiments, the subject has a level of VCAM-1 polypeptide or portion thereof that is elevated relative to healthy subjects prior to administration of the composition.

Also featured in some embodiments are methods comprising: administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the amount of uPAR, HGF, EGF, or VEGF polypeptide or portion thereof in the subject; and maintaining a subsequent dosage of the composition or ligand or adjusting a subsequent dosage of the composition or ligand administered to the subject based on the amount of the uPAR, HGF, EGF, or VEGF polypeptide or portion thereof identified in the subject. In some embodiments, the subject has a level of uPAR, HGF, EGF, or VEGF polypeptide or portion thereof that is elevated relative to healthy subjects prior to administration of the composition.

Also featured in some embodiments are methods comprising administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; identifying the amount of an individual secreted factor, or a panel of secreted factors, in the subject wherein the secreted factors are selected from the group consisting of GM-CSF, MIP-1 alpha, MIP-1 beta, MCP-1, IFN-gamma, RANTES, EGF and HGF, and maintaining a subsequent dosage of the cells or ligand or adjusting a subsequent dosage of the cells or ligand administered to the subject based on the amount or a change in the amount of the individual serum factor or panel of serum factors identified in the subject.

In some embodiments, the subject has prostate cancer, in some embodiments, the subject has a solid tumor, in some embodiments, an immune response against a tumor antigen, such as, for example, a prostate cancer antigen, a prostate specific protein antigen, or a prostate specific membrane antigen is induced by administration of the cells or composition and the ligand. In some embodiments, a cytotoxic T lymphocyte response is induced. In some embodiments, tumor vascularization is decreased or inhibited by administration of the cells or composition and the ligand. In some embodiments, the subject is in need of preventing prostate cancer. In some embodiments, the chimeric protein further comprises a MyD88 polypeptide or a truncated MyD88 polypeptide lacking the TIR domain.

In some embodiments, the presence, absence or amount of the biomarker is determined from a biological sample from the subject. In some embodiments, the sample contains blood or a blood fraction.

In some embodiments, the biomarker is the IL-6 polypeptide or portion thereof. In some embodiments, the presence, absence or amount of the IL-6 polypeptide or portion thereof is determined by a method that comprises contacting the IL-6 polypeptide or portion thereof with an antibody that specifically binds to the IL-6 polypeptide or portion thereof. In some embodiments, the presence, absence or amount of the IL-6 polypeptide or portion thereof is determined by a method that comprises analyzing the IL-6 polypeptide or portion thereof by high performance liquid chromatography. In some embodiments, the presence, absence or amount of the IL-6 polypeptide or portion thereof is determined by a method that comprises analyzing the IL-6 polypeptide or portion thereof by mass spectrometry.

In some embodiments, the biomarker is the VCAM-1 polypeptide or portion thereof. In some embodiments, the presence, absence or amount of the VCAM-1 polypeptide or portion thereof is determined by a method that comprises contacting the VCAM-1 polypeptide or portion thereof with an antibody that specifically binds to the VCAM-1 polypeptide or portion thereof. In some embodiments, the presence, absence or amount of the VCAM-1 polypeptide or portion thereof is determined by a method that comprises analyzing the VCAM-1 polypeptide or portion thereof by high performance liquid chromatography. In some embodiments, the presence, absence or amount of the VCAM-1 polypeptide or portion thereof is determined by a method that comprises analyzing the VCAM-1 polypeptide or portion thereof by mass spectrometry.

Also featured in some embodiments are methods for treating a solid tumor in a subject, comprising administering a pharmaceutical composition in an amount effective to reduce the amount of IL-6 or the amount of VCAM-1, or both, in the subject. In some embodiments, the method further comprises comprising administering an antibody to the subject. In some embodiments, the method further comprises administering a steroid agent to the subject. In some embodiments, the method further comprises administering a chemotherapy agent to the subject. In some embodiments, the pharmaceutical composition comprises a nucleic acid composition. In some embodiments, the solid tumor is classified as a prostate cancer tumor.

In some embodiments, the biomarker is the uPAR, HGF, EGF, or VEGF polypeptide or portion thereof. In some embodiments, the presence, absence or amount of the uPAR, HGF, EGF, or VEGF polypeptide or portion thereof is determined by a method that comprises contacting the uPAR, HGF, EGF, or VEGF polypeptide or portion thereof with an antibody that specifically binds to the uPAR, HGF, EGF, or VEGF polypeptide or portion thereof. In some embodiments, the presence, absence or amount of the uPAR, HGF, EGF, or VEGF polypeptide or portion thereof is determined by a method that comprises analyzing the uPAR, HGF, EGF, or VEGF polypeptide or portion thereof by high performance liquid chromatography. In some embodiments, the presence, absence or amount of the uPAR, HGF, EGF, or VEGF polypeptide or portion thereof is determined by a method that comprises analyzing the uPAR, HGF, EGF, or VEGF polypeptide or portion thereof by mass spectrometry.

Also featured in some embodiments are methods for improving quality of life in a subject, comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; and administering a multimeric ligand that binds to the multimeric ligand binding region; whereby the antigen presenting cell, and the ligand are administered in an amount effective to improve quality of life in the subject. In some embodiments, the subject has cancer, for example, end stage cancer. In some embodiments, the subject has prostate cancer, for example, end stage prostate cancer. In some embodiments, one or more symptoms of cachexia, fatigue, or anemia is alleviated. In some embodiments, two or more symptoms of cachexia, fatigue, or anemia are alleviated.

Also featured in some embodiments are methods for improving quality of life in a subject, comprising administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen, to a subject in need thereof, wherein the chimeric protein comprises a membrane targeting region, a multimeric ligand binding region and a CD40 cytoplasmic polypeptide region lacking the CD40 extracellular domain; and administering a multimeric ligand that binds to the multimeric ligand binding region; whereby the antigen compound, and the ligand are administered in an amount effective to improve quality of life in the subject. Also featured in some embodiments are methods for improving quality of life in a subject, comprising administering a nucleotide sequence that encodes a chimeric protein, and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding a tumor antigen, such as, for example, a prostate cancer antigen, a prostate specific protein antigen, or a prostate specific membrane antigen are delivered using a vector, for example, a virus vector, for example, an adenovirus vector; and administering a multimeric ligand that binds to the multimeric ligand binding region; whereby the nucleotide sequences and ligand are administered in an amount effective to improve quality of life in the subject. In some embodiments, the subject has cancer, for example, end stage cancer. In some embodiments, the subject has prostate cancer, for example, end stage prostate cancer. In some embodiments, one or more symptoms of cachexia, fatigue, or anemia is alleviated. In some embodiments, two or more symptoms of cachexia, fatigue, or anemia are alleviated.

Also featured in some embodiments are methods comprising administering a transduced or transfected antigen presenting cell to a subject in need thereof, wherein: the antigen presenting cell is transduced or transfected with a nucleic acid including a nucleotide sequence that encodes a chimeric protein, the chimeric protein comprises a membrane targeting region, a multimeric ligand binding region and a CD40 cytoplasmic polypeptide region lacking the CD40 extracellular domain, the transduced or transfected antigen presenting cell is loaded with a tumor antigen, such as, for example, a prostate cancer antigen, a prostate specific protein antigen, or a prostate specific membrane antigen; administering a multimeric ligand that binds to the multimeric ligand binding region; and measuring one or more quality of life indicators in the subject. In some embodiments, the subject has cancer, for example end stage cancer. In some embodiments, the subject has prostate cancer, for example, end stage prostate cancer. In some embodiments, one or more symptoms of cachexia, fatigue, or anemia is measured. In some embodiments, two or more symptoms of cachexia, fatigue, or anemia are measured.

Also featured in some embodiments are methods comprising administering a composition comprising a nucleotide sequence that encodes a chimeric protein and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain; administering a multimeric ligand that binds to the multimeric ligand binding region; and measuring one or more quality of life indicators in the subject. Also featured in some embodiments are methods comprising administering a nucleotide sequence that encodes a chimeric protein, and a nucleotide sequence encoding a tumor antigen, such as, for example, a prostate cancer antigen, a prostate specific protein antigen, or a prostate specific membrane antigen to a subject in need thereof, wherein the chimeric protein comprises a membrane targeting region, a multimeric ligand binding region and a CD40 cytoplasmic polypeptide region lacking the CD40 extracellular domain, wherein the nucleotide sequence encoding the chimeric protein and the nucleotide sequence encoding a tumor antigen, such as, for example, a prostate cancer antigen, a prostate specific protein antigen, or a prostate specific membrane antigen are delivered using a vector, for example, a virus vector, for example, an adenovirus vector; administering a multimeric ligand that binds to the multimeric ligand binding region; and measuring one or more quality of life indicators in the subject. In some embodiments, the subject has cancer, for example end stage cancer. In some embodiments, the subject has prostate cancer, for example, end stage prostate cancer. In some embodiments, one or more symptoms of cachexia, fatigue, or anemia is measured. In some embodiments, two or more symptoms of cachexia, fatigue, or anemia are measured.

Also featured in some embodiments are methods of the embodiments herein wherein a nucleotide sequence that encodes a chimeric protein and a tumor antigen, such as, for example, a prostate cancer antigen, a prostate specific protein antigen, or a prostate specific membrane antigen, are delivered to a subject, wherein the chimeric protein comprises a membrane targeting region, a multimeric ligand binding region and a CD40 cytoplasmic polypeptide region lacking the CD40 extracellular domain, and administering a multimeric ligand that binds to the multimeric ligand binding region, Thus, in the embodiments here wherein a nucleotide sequences encoding the chimeric protein and the tumor antigen are employed in the methods, in this embodiment, a prostate specific membrane antigen polypeptide is administered to the subject rather than a nucleotide sequence encoding a prostate specific membrane antigen polypeptide.

In some embodiments, the subject is a mammal. In some embodiments, the subject is a human.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1. Schematic diagram of iCD40 and expression in human DCs. A. The human CD40 cytoplasmic domain can be subcloned downstream of a myristoylation-targeting domain (M) and two tandem domains (Fv)(Clackson T, Yang W, Rozamus L W, et al., Proc Natl Acad Sci USA. 1998; 95:10437-10442). The expression of M-Fv-Fv-CD40 chimeric protein, referred to here as inducible CD40 (iCD40) can be under cytomegalovirus (CMV) promoter control. B. The expression of endogenous (eCD40) and recombinant inducible (iCD40) forms of CD40 assessed by Western blot. Lane 1, wild type DCs (endogenous CD40 control); lane 2, DCs stimulated with 1 microgram/ml of LPS; lanes 3 and 4, DCs transduced with 10,000 VP/cell (MOI˜160) of Ad5/f35-iCD40 (iCD40-DCs) with and without AP20187 dimerizer drug respectively; lane 5, iCD40-DCs stimulated with LPS and AP20187; lane 6, DCs stimulated with CD40L (CD40 ligand, a protein a TNF family member) and LPS; lane 7, DCs transduced with Ad5/f35-GFP (GFP-DCs) at MOI 160 and stimulated with AP20187 and LPS; lane 8, GFP-DCs stimulated with AP20187; lane 9, 293 T cells transduced with Ad5/f35-iCD40 (positive control for inducible form of CD40). The expression levels of alpha-tubulin served as internal control.

FIG. 2. iRIG-1 and iMyD88 in RAW264.7 cells. RAW 264.7 cells were cotransfected transiently with 3 micrograms expression plasmids for iRIG-1 and 1 microgram IFNgamma-dependent SEAP reporter plasmid; and 3 micrograms iMyD88 with 1 microgram NF-kappaB-dependent SEAP reporter plasmid.

FIG. 3 is a schematic of inducible CD40 and MyD88 receptors and induction of NF-kappa B activity.

FIG. 4 is a schematic of inducible chimeric CD40/MyD88 receptors and induction of NF-kappaB activity.

FIG. 5 is a graph of NF-kappa B activation in 293 cells by inducible MyD88 and chimeric MyD88-CD40 receptors. CD40T indicates “turbo” CD40, wherein the receptor includes 3 copies of the FKBP12v₃₆ domain (Fv′).

FIG. 6 is a graph of NF-kappa B activity by inducible truncated MyD88 (MyD88L) and chimeric inducible truncated MyD88/CD40 after 3 hours of incubation with substrate.

FIG. 7 is a graph of NF-kappa B activity by inducible truncated MyD88 (MyD88L) and chimeric inducible truncated MyD88/CD40 after 22 hours of incubation with substrate. Some assay saturation is present in this assay.

FIG. 8 is a Western blot of HA protein, following adenovirus-MyD88L transduction of 293T cells.

FIG. 9 is a Western blot of HA protein, following adenovirus-MyD88L-CD40 transduction of 293T cells.

FIG. 10 is a graph of an ELISA assay after adenovirus infection of bone marrow derived DCs with the indicated inducible CD40 and MyD88 constructs.

FIG. 11 is a graph of the results of an ELISA assay similar to that in FIG. 10.

FIG. 12 is a graph of the results of an ELISA assay similar to that in FIGS. 10 and 11, after infection with a higher amount of adenovirus.

FIG. 13 is a construct map of pShuttleX-iMyD88.

FIG. 14 is a construct map of pShuttleX-CD4-TLR4L3-E.

FIG. 15 is a construct map of pShuttleX-iMyD88E-CD40.

FIG. 16 is a bar graph depicting the results of a dose-dependent induction of IL-12p70 expression in human monocyte-derived dendritic cells (moDCs) transduced with different multiplicity of infections of adenovirus expressing an inducible MyD88.CD40 composite construct.

FIG. 17 is a bar graph depicting of the results of a drug-dependent induction of IL-12p70 expression in human monocyte-derived dendritic cells (moDCs) transduced with adenoviruses expressing different inducible constructs.

FIG. 18 is a bar graph depicting the IL-12p70 levels in transduced dendritic cells prior to vaccination.

FIG. 19(a) is a graph of EG.7-OVA tumor growth inhibition in mice vaccinated with transduced dendritic cells; FIG. 19(b) presents photos of representative vaccinated mice; FIG. 19(c) is the graph of 19(a), including error bars.

FIG. 20(a) is a scatter plot, and 20(b) is a bar graph, showing the enhanced frequency of Ag-specific CD8+ T cells induced by transduced dendritic cells.

FIG. 21 is a bar graph showing the enhanced frequency of Ag-Specific IFN gamma+CD8+ T cells and CD4+ TH1 cells induced by transduced dendritic cells.

FIG. 22 presents a schematic and the results of an in vivo cytotoxic lymphocyte assay. FIG. 22 discloses “SIINFEKL” as SEQ ID NO: 29.

FIG. 23 is a bar graph summarizing the data from an enhanced in vivo CTL activity induced by dendritic cells.

FIG. 24 presents representative results of a CTL assay in mice induced by transduced dendritic cells.

FIG. 25 presents the results of intracellular staining for IL-4 producing TH2 cells in mice inoculated by transduced dendritic cells.

FIGS. 26A-26C present the results of a tumor growth inhibition assay in mice treated with Ad5-iCD40.MyD88 transduced cells: FIG. 26A is a line graph of tumor volume and days after tumor inoculation. FIG. 26B is a line graph of tumor volume and days after tumor inoculation. FIG. 26C is a bar graph of IL-12p70.

FIGS. 27A-27F present a tumor specific T cell assay in mice treated with Ad5-iCD40.MyD88 transduced cells: FIG. 27A presents % CD8+ SINFEKL-Ter cells. FIG. 27B presents counts and CFSE FITC-A. FIG. 27C presents counts and CFSE FITC-A. FIG. 27D is a bar graph of % specific lysis. FIG. 27E is a bar graph of numbers of spots/10⁶ cells. FIG. 27F is a bar graph of numbers of spots/10⁶ cells.

FIG. 28 presents the results of a natural killer cell assay using splenocytes from the treated mice as effectors.

FIG. 29 presents the results of a cytotoxic lymphocyte assay using splenocytes from the treated mice as effectors.

FIG. 30 presents the results of an IFN-gamma ELISPot assay using T cells co-cultured with dendritic cells transduced with the indicated vector.

FIG. 31 presents the results of a CCR7 upregulation assay using dendritic cells transformed with the indicated vector, with or without LPS as an adjuvant.

FIG. 32 presents the results of a CCR7 upregulation assay, with the data from multiple animals included in one graph.

FIG. 33 is a plasmid map of Ad5f35ihCD40.

FIG. 34 is a chart presenting exploratory efficacy assessments.

FIG. 35 is a chart of the 12 week immunological and clinical response summary for subjects 1001-1006.

FIG. 36 presents waterfall plots presenting the analysis of a 12 week change from baseline for measurable metastatic disease, vascularity, and PSA levels.

FIG. 37 is a graph of cytokine levels in Subject 1008 following treatment.

FIG. 38 is a graph of the results of VCAM-1 serum analysis.

FIG. 39 is a waterfall plot of PSA levels at 12 weeks.

FIG. 40 presents the results of CT scans of patient 1003 at 7, 12, and 52 weeks.

FIG. 41 presents a graph of a soft tissue partial response of Subject 1003.

FIG. 42 presents a graph of various serum markers showing a potential anti-vasculature effect.

FIG. 43 presents PSA levels measured in Subject 1003.

FIG. 44 presents a map of an inducible CD40 transgene.

FIG. 45 is a graph of serum marker analysis of patient 1001.

FIG. 46 is a graph of serum marker analysis of patient 1002.

FIG. 47 is a graph of serum marker analysis of patient 1003.

FIG. 48 is a graph of serum marker analysis of patient 1004.

FIG. 49 is a graph of serum marker analysis of patient 1005.

FIG. 50 is a graph of serum marker analysis of patient 1006.

FIG. 51 is a bar graph of a PSMA specific injection site immune response in patient 1006.

FIG. 52 presents graphs of KPS and CTC assessments.

FIG. 53 presents a graph of PSA levels serum concentration for subject 1006 over the course of treatment.

FIG. 54 presents a graph of uPAR, HGF, EGF, and VEGF concentrations for subject 1003 over the course of treatment.

FIG. 55 is a Safety and Response Summary table for subjects 1001 through 1006.

FIG. 56 is a Safety and Response Summary table for subjects 1007 through 1012.

FIG. 57 is a Patient Demographics table for subjects 1001 through 1012.

FIG. 58 is a timeline presenting the clinical trial status for subjects 1001 through 1012.

FIG. 59 presents photos showing lung tumor shrinkage following treatment of Subject 1008.

FIG. 60 is a graph of PSA levels for Subject 1011.

FIG. 61 is a graph of PSA levels for Subject 1010.

FIG. 62 presents photographs of bone scans of subject 1010.

FIG. 63 is a chart of subject responses to combination treatment with taxane-based chemotherapy and vaccine therapy.

FIG. 64 presents photos showing tumor shrinkage in Subject 1006.

### DETAILED DESCRIPTION

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The term “allogeneic” as used herein, refers to HLA or MHC loci that are antigenically distinct.

Thus, cells or tissue transferred from the same species can be antigenically distinct. Syngeneic mice can differ at one or more loci (congenics) and allogeneic mice can have the same background.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. Exemplary organisms include but are not limited to, Helicobacters, Campylobacters, Clostridia, Corynebacterium diphtheriae, Bordetella pertussis, influenza virus, parainfluenza viruses, respiratory syncytial virus, Borrelia burgdorfei, Plasmodium, herpes simplex viruses, human immunodeficiency virus, papillomavirus, Vibrio cholera, E. coli, measles virus, rotavirus, shigella, Salmonella typhi, Neisseria gonorrhea. Therefore, any macromolecules, including virtually all proteins or peptides, can serve as antigens. Furthermore, antigens can be derived from recombinant or genomic DNA. Any DNA that contains nucleotide sequences or partial nucleotide sequences of a pathogenic genome or a gene or a fragment of a gene for a protein that elicits an immune response results in synthesis of an antigen. Furthermore, the present methods are not limited to the use of the entire nucleic acid sequence of a gene or genome. It is readily inherent that the present invention includes, but is not limited to, the use of partial nucleic acid sequences of more than one gene or genome and that these nucleic acid sequences are arranged in various combinations to elicit the desired immune response.

The term “antigen-presenting cell” is any of a variety of cells capable of displaying, acquiring, or presenting at least one antigen or antigenic fragment on (or at) its cell surface. In general, the term “antigen-presenting cell” can be any cell that accomplishes the goal of aiding the enhancement of an immune response (i.e., from the T-cell or —B-cell arms of the immune system) against an antigen or antigenic composition. As discussed in Kuby, 2000, Immunology, 4.sup.th edition, W.H. Freeman and company, for example, (incorporated herein by reference), and used herein in certain embodiments, a cell that displays or presents an antigen normally or with a class II major histocompatibility molecule or complex to an immune cell is an “antigen-presenting cell.” In certain aspects, a cell (e.g., an APC cell) may be fused with another cell, such as a recombinant cell or a tumor cell that expresses the desired antigen. Methods for preparing a fusion of two or more cells are discussed in, for example, Goding, J. W., Monoclonal Antibodies: Principles and Practice, pp. 65-66, 71-74 (Academic Press, 1986); Campbell, in: Monoclonal Antibody Technology, Laboratory Techniques in Biochemistry and Molecular Biology, Vol. 13, Burden & Von Knippenberg, Amsterdam, Elseview, pp. 75-83, 1984; Kohler & Milstein, Nature, 256:495-497, 1975; Kohler & Milstein, Eur. J. Immunol., 6:511-519, 1976, Gefter et al., Somatic Cell Genet., 3:231-236, 1977, each incorporated herein by reference. In some cases, the immune cell to which an antigen-presenting cell displays or presents an antigen to is a CD4+ TH cell. Additional molecules expressed on the APC or other immune cells may aid or improve the enhancement of an immune response. Secreted or soluble molecules, such as for example, cytokines and adjuvants, may also aid or enhance the immune response against an antigen. Various examples are discussed herein.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “iCD40 molecule” is defined as an inducible CD40. This iCD40 can bypass mechanisms that extinguish endogenous CD40 signaling. The term “iCD40” embraces “iCD40 nucleic acids,” “iCD40 polypeptides” and/or iCD40 expression vectors.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

The term “dendritic cell” (DC) is an antigen-presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

As used herein, the term “ex vivo” refers to “outside” the body. The terms “ex vivo” and “in vitro” can be used interchangeably herein.

As used herein, the term “functionally equivalent,” as it relates to CD40, for example, refers to a CD40 nucleic acid fragment, variant, or analog, refers to a nucleic acid that codes for a CD40 polypeptide, or a CD40 polypeptide, that stimulates an immune response to destroy tumors or hyperproliferative disease. “Functionally equivalent” refers, for example, to a CD40 polypeptide that is lacking the extracellular domain, but is capable of amplifying the T cell-mediated tumor killing response by upregulating dendritic cell expression of antigen presentation molecules. When the term “functionally equivalent” is applied to other nucleic acids or polypeptides, such as, for example, PSA peptide, PSMA peptide, MyD88, or truncated MyD88, it refers to fragments, variants, and the like that have the same or similar activity as the reference polypeptides of the methods herein.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

As used herein, the term “gene” is defined as a functional protein, polypeptide, or peptide-encoding unit. As will be understood, this functional term includes genomic sequences, cDNA sequences, and smaller engineered gene segments that express, or are adapted to express, proteins, polypeptides, domains, peptides, fusion proteins, and mutants.

The term “immunogenic composition” or “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunocompromised” as used herein is defined as a subject that has reduced or weakened immune system. The immunocompromised condition may be due to a defect or dysfunction of the immune system or to other factors that heighten susceptibility to infection and/or disease. Although such a categorization allows a conceptual basis for evaluation, immunocompromised individuals often do not fit completely into one group or the other. More than one defect in the body's defense mechanisms may be affected. For example, individuals with a specific T-lymphocyte defect caused by HIV may also have neutropenia caused by drugs used for antiviral therapy or be immunocompromised because of a breach of the integrity of the skin and mucous membranes. An immunocompromised state can result from indwelling central lines or other types of impairment due to intravenous drug abuse; or be caused by secondary malignancy, malnutrition, or having been infected with other infectious agents such as tuberculosis or sexually transmitted diseases, e.g., syphilis or hepatitis.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “regulate an immune response” or “modulate an immune response” refers to the ability to modify the immune response. For example, the composition is capable of enhancing and/or activating the immune response. Still further, the composition is also capable of inhibiting the immune response. The form of regulation is determined by the ligand that is used with the composition. For example, a dimeric analog of the chemical results in dimerization of the co-stimulatory polypeptide leading to activation of the DCs, however, a monomeric analog of the chemical does not result in dimerization of the co-stimulatory polypeptide, which would not activate the DCs.

The term “transfection” and “transduction” are interchangeable and refer to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like.

As used herein, the term “syngeneic” refers to cells, tissues or animals that have genotypes that are identical or closely related enough to allow tissue transplant, or are immunologically compatible. For example, identical twins or animals of the same inbred strain. Syngeneic and isogeneic can be used interchangeably.

The term “subject” as used herein includes, but is not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

As used herein, the term “under transcriptional control” or “operatively linked” is defined as the promoter is in the correct location and orientation in relation to the nucleic acid to control RNA polymerase initiation and expression of the gene.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

As used herein, the term “vaccine” refers to a formulation which contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

In some embodiments, the nucleic acid is contained within a viral vector. In certain embodiments, the viral vector is an adenoviral vector. It is understood that in some embodiments, the antigen-presenting cell is contacted with the viral vector ex vivo, and in some embodiments, the antigen-presenting cell is contacted with the viral vector in vivo.

In some embodiments, the antigen-presenting cell is a dendritic cell, for example, a mammalian dendritic cell. Often, the antigen-presenting cell is a human dendritic cell.

In certain embodiments, the antigen-presenting cell is also contacted with an antigen. Often, the antigen-presenting cell is contacted with the antigen ex vivo. Sometimes, the antigen-presenting cell is contacted with the antigen in vivo. In some embodiments, the antigen-presenting cell is in a subject and an immune response is generated against the antigen. Sometimes, the immune response is a cytotoxic T-lymphocyte (CTL) immune response. Sometimes, the immune response is generated against a tumor antigen. In certain embodiments, the antigen-presenting cell is activated without the addition of an adjuvant.

In some embodiments, the antigen-presenting cell is transduced with the nucleic acid ex vivo and administered to the subject by intradermal administration. In some embodiments, the antigen-presenting cell is transduced with the nucleic acid ex vivo and administered to the subject by subcutaneous administration. Sometimes, the antigen-presenting cell is transduced with the nucleic acid ex vivo. Sometimes, the antigen-presenting cell is transduced with the nucleic acid in vivo.

By MyD88 is meant the myeloid differentiation primary response gene 88, for example, but not limited to the human version, cited as ncbi Gene ID 4615. By “truncated,” is meant that the protein is not full length and may lack, for example, a domain. For example, a truncated MyD88 is not full length and may, for example, be missing the TIR domain. One example of a truncated MyD88 is indicated as MyD88L herein, and is also presented as SEQ ID NOS: 5 (nucleic acid sequence) and 6 (peptide sequence). SEQ ID NO: 5 includes the linkers added during subcloning. By a nucleic acid sequence coding for “truncated MyD88” is meant the nucleic acid sequence coding for the truncated MyD88 peptide, the term may also refer to the nucleic acid sequence including the portion coding for any amino acids added as an artifact of cloning, including any amino acids coded for by the linkers.

In the methods herein, the inducible CD40 portion of the peptide may be located either upstream or downstream from the inducible MyD88 or truncated MyD88 polypeptide portion. Also, the inducible CD40 portion and the inducible MyD88 or truncated MyD88 adapter protein portions may be transfected or transduced into the cells either on the same vector, in cis, or on separate vectors, in trans.

The antigen-presenting cell in some embodiments is contacted with an antigen, sometimes ex vivo. In certain embodiments the antigen-presenting cell is in a subject and an immune response is generated against the antigen, such as a cytotoxic T-lymphocyte (CTL) immune response. In certain embodiments, an immune response is generated against a tumor antigen (e.g., PSMA). In some embodiments, the nucleic acid is prepared ex vivo and administered to the subject by intradermal administration or by subcutaneous administration, for example. Sometimes the antigen-presenting cell is transduced or transfected with the nucleic acid ex vivo or in vivo. In some embodiments, the nucleic acid comprises a promoter sequence operably linked to the polynucleotide sequence. Alternatively, the nucleic acid comprises an ex vivo-transcribed RNA, containing the protein-coding region of the chimeric protein.

By “reducing tumor size” or “inhibiting tumor growth” of a solid tumor is meant a response to treatment, or stabilization of disease, according to standard guidelines, such as, for example, the Response Evaluation Criteria in Solid Tumors (RECIST) criteria. For example, this may include a reduction in the diameter of a solid tumor of about 5%, 10%, 20%, 30%, 40%, 50%, 60%, 70%, 80%, 90%, or 100%, or the reduction in the number of tumors, circulating tumor cells, or tumor markers, of about 5%, 10%, 20%, 30%, 40%, 50%, 60%, 70%, 80%, 90%, or 100%. The size of tumors may be analyzed by any method, including, for example, CT scan, MRI, for example, CT-MRI, chest X-ray (for tumors of the lung), or molecular imaging, for example, PET scan, such as, for example, a PET scan after administering an iodine 123-labelled PSA, for example, PSMA ligand, such as, for example, where the inhibitor is TROFEX™/MIP-1072/1095, or molecular imaging, for example, SPECT, or a PET scan using PSA, for example, PSMA antibody, such as, for example, capromad pendetide (Prostascint), a 111-iridium labeled PSMA antibody.

By “reducing, slowing, or inhibiting tumor vascularization is meant a reduction in tumor vascularization of about 5%, 10%, 20%, 30%, 40%, 50%, 60%, 70%, 80%, 90%, or 100%, or a reduction in the appearance of new vasculature of about 5%, 10%, 20%, 30%, 40%, 50%, 60%, 70%, 80%, 90%, or 100%, when compared to the amount of tumor vascularization before treatment. The reduction may refer to one tumor, or may be a sum or an average of the vascularization in more than one tumor. Methods of measuring tumor vascularization include, for example, CAT scan, MRI, for example, CT-MRI, or molecular imaging, for example, SPECT, or a PET scan, such as, for example, a PET scan after administering an iodine 123-labelled PSA, for example, PSMA ligand, such as, for example, where the inhibitor is TROFEX™/MIP-1072/1095, or a PET scan using PSA, for example, PSMA antibody, such as, for example, capromad pendetide (Prostascint), a 111-iridium labeled PSMA antibody.

A tumor is classified as a prostate cancer tumor when, for example, the tumor is present in the prostate gland, or has derived from or metastasized from a tumor in the prostate gland, or produces PSA. A tumor has metastasized from a tumor in the prostate gland, when, for example, it is determined that the tumor has chromosomal breakpoints that are the same as, or similar to, a tumor in the prostate gland of the subject.

### Prostate Cancer

In the United States, prostate cancer is the most common solid tumor malignancy in men. It was expected to account for an estimated 186,320 new cases of prostate cancer in 2008 and 28,660 deaths. Jemal A, et al., Cancer statistics, 2008. CA Cancer J Clin. 58: 71-96, 2008. Approximately 70% of patients who experience PSA-progression after primary therapy will have metastases at some time during the course of their disease. Gittes R F, N Engl J Med. 324: 236-45, 1991. Androgen deprivation therapy (ADT) is the standard therapy for metastatic prostate cancer and achieves temporary tumor control or regression in 80-85% of patients. Crawford E D, et al., N Engl J Med. 321: 419-24, 1989; Schellhammer P F, et al., J Urol. 157: 1731-5, 1997; Scher H I and Kelly W K, J Clin Oncol. 11: 1566-72, 1993; Small E J and Srinivas S, Cancer. 76: 1428-34, 1995. Duration of response to hormone therapy, as well as survival after the initiation of hormone therapy, has been shown to be dependent on a number of factors, including the Gleason Sum of the original tumor, the ability to achieve an undetectable nadir PSA after initiation of ADT, and the PSA doubling time prior to initiation of ADT. Despite hormonal therapy, virtually all patients with metastatic prostate cancer ultimately develop progressive disease. Kelly W K and Slovin S F, Curr Oncol Rep. 2: 394-401, 2000; Scher H I, et al., J Natl Cancer Inst. 88: 1623-34, 1996; Small E J and Vogelzang N J, J Clin Oncol. 15: 382-8, 1997. The Gleason Sum of the original tumor, or the Gleason score, is used to grade levels of prostate cancer in men, based on the microscopic evaluation of the tumor. A higher Gleason score denotes a cancer that has a worse prognosis as it is more aggressive, and is more likely to spread. An example of the grading system is discussed in Gleason D F., The Veteran's Administration Cooperative Urologic Research Group: histologic grading and clinical staging of prostatic carcinoma. In Tannenbaum M (ed.) Urologic Pathology: The Prostate. Lea and Febiger, Philadelphia, 1977; 171-198.

Most patients with prostate cancer who have been started on ADT are treated for a rising PSA after failure of primary therapy (e.g. radical prostatectomy, brachytherapy, external beam radiation therapy, cryo-ablation, etc.). In the absence of clinical metastases, these patients experience a relatively long disease-free interval in the range of 7-11 years; however, the majority of these patients eventually develop hormone-resistant disease as evidenced by the return of a rising PSA level in the face of castrate levels of serum testosterone. These patients, too, have a poor prognosis, with the majority developing clinical metastases within 9 months and a median survival of 24 months. Bianco F J, et al., Cancer Symposium: Abstract 278, 2005. The term “prostate cancer” includes different forms or stages, including, for example, metastatic, metastatic castration resistant, metastatic castration sensitive, regionally advanced, and localized prostate cancer.

### Antigen Presenting Cells

Antigen presenting cells (APCs) are cells that can prime T-cells against a foreign antigen by displaying the foreign antigen with major histocompatibility complex (MHC) molecules on their surface. There are two types of APCs, professional and non-professional. The professional APCs express both MHC class I molecules and MHC class II molecules, the non-professional APCs do not constitutively express MHC class II molecules. In particular embodiments, professional APCs are used in the methods herein. Professional APCs include, for example, B-cells, macrophages, and dendritic cells.

An antigen-presenting cell is “activated,” when one or more activities associated with activated antigen-presenting cells may be observed and/or measured. For example, an antigen-presenting cell is activated when following contact with an expression vector presented herein, an activity associated with activation may be measured in the expression vector-contacted cell as compared to an antigen-presenting cell that has either not been contacted with the expression vector, or has been contacted with a negative control vector. In one example, the increased activity may be at a level of two, three, four, five, six, seven, eight, nine, or ten fold, or more, than that of the non-contacted cell, or the cell contacted with the negative control. For example, one of the following activities may be enhanced in an antigen-presenting cell that has been contacted with the expression vector: co-stimulatory molecule expression on the antigen-presenting cell, nuclear translocation of NF-kappaB in antigen-presenting cells, DC maturation marker expression, such as, for example, toll-like receptor expression or CCR7 expression, specific cytotoxic T lymphocyte responses, such as, for example, specific lytic activity directed against tumor cells, or cytokine (for example, IL-2) or chemokine expression.

An amount of a composition that activates antigen-presenting cells or that “enhances” an immune response refers to an amount in which an immune response is observed that is greater or intensified or deviated in any way with the addition of the composition when compared to the same immune response measured without the addition of the composition. For example, the lytic activity of cytotoxic T cells can be measured, for example, using a ⁵¹Cr release assay, with and without the composition. The amount of the substance at which the CTL lytic activity is enhanced as compared to the CTL lytic activity without the composition is said to be an amount sufficient to enhance the immune response of the animal to the antigen. For example, the immune response may be enhanced by a factor of at least about 2, or, for example, by a factor of about 3 or more. The amount of cytokines secreted may also be altered.

The enhanced immune response may be an active or a passive immune response. Alternatively, the response may be part of an adaptive immunotherapy approach in which antigen-presenting cells are obtained with from a subject (e.g., a patient), then transduced or transfected with a composition comprising the expression vector or construct presented herein. The antigen-presenting cells may be obtained from, for example, the blood of the subject or bone marrow of the subject. The antigen-presenting cells may then be administered to the same or different animal, or same or different subject (e.g., same or different donors). In certain embodiments the subject (for example, a patient) has or is suspected of having a cancer, such as for example, prostate cancer, or has or is suspected of having an infectious disease. In other embodiments the method of enhancing the immune response is practiced in conjunction with a known cancer therapy or any known therapy to treat the infectious disease.

### Dendritic Cells

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism (Janeway et al. (1989) Cold Spring Harb. Symp. Quant. Biol., 54: 1-13; Medzhitov et al., Nature, 388:394-397, 1997).

The receptors of the innate immune system that recognize PAMPs are called Pattern Recognition Receptors (PRRs) (Janeway et al., 1989; Medzhitov et al., 1997). These receptors vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly (Medzhitov et al., 1997; Fearon et al. (1996) Science 272: 50-3).

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, as discussed below. This latter function allows efficient mobilization of effector forces to combat the invaders.

The primary function of dendritic cells (DCs) is to acquire antigen in the peripheral tissues, travel to secondary lymphoid tissue, and present antigen to effector T cells of the immune system (Banchereau, J., et al., Annu Rev Immunol, 2000, 18: p. 767-811; Banchereau, J., & Steinman, R. M., Nature 392, 245-252 (1998)). As DCs carry out their crucial role in the immune response, they undergo maturational changes allowing them to perform the appropriate function for each environment (Termeer, C. C., et al., J Immunol, 2000, Aug. 15, 165: p. 1863-70). During DC maturation, antigen uptake potential is lost, the surface density of major histocompatibility complex (MHC) class I and class II molecules increases by 10-100 fold, and CD40, costimulatory and adhesion molecule expression also greatly increases (Lanzavecchia, A. and F. Sallusto, Science, 2000. 290: p. 92-96). In addition, other genetic alterations permit the DCs to home to the T cell-rich paracortex of draining lymph nodes and to express T-cell chemokines that attract naïve and memory T cells and prime antigen-specific naïve TH0 cells (Adema, G. J., et al., Nature, 1997, Jun. 12. 387: p. 713-7). During this stage, mature DCs present antigen via their MHC II molecules to CD4+ T helper cells, inducing the upregulation of T cell CD40 ligand (CD40L) that, in turn, engages the DC CD40 receptor. This DC:T cell interaction induces rapid expression of additional DC molecules that are crucial for the initiation of a potent CD8+ cytotoxic T lymphocyte (CTL) response, including further upregulation of MHC I and II molecules, adhesion molecules, costimulatory molecules (e.g., B7.1, B7.2), cytokines (e.g., IL-12) and anti-apoptotic proteins (e.g., Bcl-2) (Anderson, D. M., et al., Nature, 1997, Nov. 13, 390: p. 175-9; Ohshima, Y., et al., J Immunol, 1997, Oct. 15, 159: p. 3838-48; Sallusto, F., et al., Eur J Immunol, 1998, Sep. 28: p. 2760-9; Caux, C. Adv Exp Med Biol. 1997, 417:21-5;). CD8+ T cells exit lymph nodes, reenter circulation and home to the original site of inflammation to destroy pathogens or malignant cells.

One key parameter influencing the function of DCs is the CD40 receptor, serving as the “on switch” for DCs (Bennett, S. R., et al., Nature, 1998, Jun. 4, 393: p. 478-80; Clarke, S. R., J Leukoc Biol, 2000, May. 67: p. 607-14; Fernandez, N. C., et al., Nat Med, 1999, Apr. 5: p. 405-11; Ridge, J. P., D. R. F, and P. Nature, 1998, Jun. 4, 393: p. 474-8; Schoenberger, S. P., et al., Nature, 1998, Jun. 4. 393: p. 480-3). CD40 is a 48-kDa transmembrane member of the TNF receptor superfamily (McWhirter, S. M., et al., Proc Natl Acad Sci USA, 1999, Jul. 20, 96: p. 8408-13). CD40-CD40L interaction induces CD40 trimerization, necessary for initiating signaling cascades involving TNF receptor associated factors (TRAFs) (Ni, C., et al., PNAS, 2000, 97(19): 10395-10399; Pullen, S. S., et al., J Biol Chem, 1999, May 14.274: p. 14246-54). CD40 uses these signaling molecules to activate several transcription factors in DCs, including NF-kappa B, AP-1, STAT3, and p38MAPK (McWhirter, S. M., et al., 1999).

Due to their unique method of processing and presenting antigens and the potential for high-level expression of costimulatory and cytokine molecules, dendritic cells (DC) are effective antigen-presenting cells (APCs) for priming and activating naïve T cells (Banchereau J, et al., Ann N Y Acad Sci. 2003; 987:180-187). This property has led to their widespread use as a cellular platform for vaccination in a number of clinical trials with encouraging results (O'Neill D W, et al., Blood. 2004; 104:2235-2246; Rosenberg S A, Immunity. 1999; 10:281-287). However, the clinical efficacy of DC vaccines in cancer patients has been unsatisfactory, probably due to a number of key deficiencies, including suboptimal activation, limited migration to draining lymph nodes, and an insufficient life span for optimal T cell activation in the lymph node environment.

A parameter in the optimization of DC-based cancer vaccines is the interaction of DCs with immune effector cells, such as CD4+, CD8+ T cells and T regulatory (Treg) cells. In these interactions, the maturation state of the DCs is a key factor in determining the resulting effector functions (Steinman R M, Annu Rev Immunol. 2003; 21:685-711). To maximize CD4+ and CD8+ T cell priming while minimizing Treg expansion, DCs need to be fully mature, expressing high levels of co-stimulatory molecules, (like CD40, CD80, and CD86), and pro-inflammatory cytokines, like IL-12p70 and IL-6. Equally important, the DCs must be able to migrate efficiently from the site of vaccination to draining lymph nodes to initiate T cell interactions (Vieweg J, et al., Springer Semin Immunopathol. 2005; 26:329-341).

For the ex vivo maturation of monocyte-derived immature DCs, the majority of DC-based trials have used a standard maturation cytokine cocktail (MC), comprised of TNF-alpha, IL-1beta, IL-6, and PGE2. The principal function of prostaglandin E2 (PGE2) in the standard maturation cocktail is to sensitize the CC chemokine receptor 7 (CCR7) to its ligands, CC chemokine ligand 19 (CCL19) and CCL21 and thereby enhance the migratory capacity of DCs to the draining lymph nodes (Scandella E, et al., Blood. 2002; 100:1354-1361; Luft T, et al., Blood. 2002; 100:1362-1372). However, PGE2 has also been reported to have numerous properties that are potentially deleterious to the stimulation of an immune response, including suppression of T-cell proliferation, (Goodwin J S, et al., J Exp Med. 1977; 146:1719-1734; Goodwin J S, Curr Opin Immunol. 1989; 2:264-268) inhibition of pro-inflammatory cytokine production (e.g., IL-12p70 and TNF-alpha (Kalinski P, Blood. 2001; 97:3466-3469; van der Pouw Kraan T C, et al., J Exp Med. 1995; 181:775-779)), and down-regulation of major histocompatibility complex (MHC) II surface expression (Snyder D S, Nature. 1982; 299:163-165). Therefore, maturation protocols that can avoid PGE2 while promoting migration are likely to improve the therapeutic efficacy of DC-based vaccines.

A DC activation system based on targeted temporal control of the CD40 signaling pathway has been developed to extend the pro-stimulatory state of DCs within lymphoid tissues. DC functionality was improved by increasing both the amplitude and the duration of CD40 signaling (Hanks B A, et al., Nat. Med. 2005; 11:130-137). To accomplish this, the CD40 receptor was re-engineered so that the cytoplasmic domain of CD40 was fused to synthetic ligand-binding domains along with a membrane-targeting sequence. Administration of a lipid-permeable, dimerizing drug, AP20187 (AP), called a chemical inducer of dimerization (CID) (Spencer D M, et al., Science. 1993; 262:1019-1024), led to the in vivo induction of CD40-dependent signaling cascades in murine DCs. This induction strategy significantly enhanced the immunogenicity against both defined antigens and tumors in vivo beyond that achieved with other activation modalities (Hanks B A, et al., Nat. Med. 2005; 11:130-137).

Pattern recognition receptor (PRR) signaling, an example of which is Toll-like receptor (TLR) signaling also plays a critical role in the induction of DC maturation and activation; human DCs express, multiple distinct TLRs (Kadowaki N, et al., J Exp Med. 2001; 194:863-869). The eleven mammalian TLRs respond to various pathogen-derived macromolecules, contributing to the activation of innate immune responses along with initiation of adaptive immunity. Lipopolysaccharide (LPS) and a clinically relevant derivative, monophosphoryl lipid A (MPL), bind to cell surface TLR-4 complexes (Kadowaki N, et al., J Exp Med. 2001; 194:863-869), leading to various signaling pathways that culminate in the induction of transcription factors, such as NF-kappaB and IRF3, along with mitogen-activated protein kinases (MAPK) p38 and c-Jun kinase (JNK) (Ardeshna K M, et al., Blood. 2000; 96:1039-1046; Ismaili J, et al., J. Immunol. 2002; 168:926-932). During this process DCs mature, and partially upregulate pro-inflammatory cytokines, like IL-6, IL-12, and Type I interferons (Rescigno M, et al., J Exp Med. 1998; 188:2175-2180). LPS-induced maturation has been shown to enhance the ability of DCs to stimulate antigen-specific T cell responses in vitro and in vivo (Lapointe R, et al., Eur J Immunol. 2000; 30:3291-3298). Methods for activating an antigen-presenting cell, comprising transducing the cell with a nucleic acid coding for a CD40 peptide have been discussed in U.S. Pat. No. 7,404,950, and methods for activating an antigen-presenting cell, comprising transfecting the cell with a nucleic acid coding for a chimeric protein including an inducible CD40 peptide and a Pattern Recognition Receptor, or other downstream proteins in the pathway have been discussed in International Patent Application No. PCT/US2007/081963, filed Oct. 19, 2007, published as WO 2008/049113, which are hereby incorporated by reference herein.

An inducible CD40 (iCD40) system has been applied to human dendritic cells (DCs) and it has been demonstrated that combining iCD40 signaling with Pattern recognition receptor (PRR) adapter ligation causes persistent and robust activation of human DCs. (Spencer, et al., U.S. Ser. No. 12/563,991, filed Sep. 21, 2009, related international application published on Mar. 25, 2010 as WO 2010/033949, hereby incorporated by reference herein).

### Engineering Expression Constructs

Expression constructs encode a co-stimulatory polypeptide and a ligand-binding domain, all operatively linked. In general, the term “operably linked” is meant to indicate that the promoter sequence is functionally linked to a second sequence, wherein the promoter sequence initiates and mediates transcription of the DNA corresponding to the second sequence. More particularly, more than one ligand-binding domain is used in the expression construct. Yet further, the expression construct contains a membrane-targeting sequence. Appropriate expression constructs may include a co-stimulatory polypeptide element on either side of the above FKBP ligand-binding elements. The expression construct may be inserted into a vector, for example a viral vector or plasmid. The steps of the methods provided may be performed using any suitable method, these methods include, without limitation, methods of transducing, transforming, or otherwise providing nucleic acid to the antigen-presenting cell, presented herein. In some embodiments, the truncated MyD88 peptide is encoded by the nucleotide sequence of SEQ ID NO: 5 (with or without DNA linkers or has the amino acid sequence of SEQ ID NO: 6). In some embodiments, the CD40 cytoplasmic polypeptide region is encoded by a polynucleotide sequence in SEQ ID NO: 1.

Co-Stimulatory Polypeptides

Co-stimulatory polypeptide molecules are capable of amplifying the T-cell-mediated response by upregulating dendritic cell expression of antigen presentation molecules. Co-stimulatory proteins that are contemplated include, for example, but are not limited, to the members of tumor necrosis factor (TNF) family (i.e., CD40, RANK/TRANCE-R, OX40, 4-1B), Toll-like receptors, C-reactive protein receptors, Pattern Recognition Receptors, and HSP receptors.

Co-stimulatory polypeptides include any molecule or polypeptide that activates the NF-kappaB pathway, Akt pathway, and/or p38 pathway. The DC activation system is based upon utilizing a recombinant signaling molecule fused to a ligand-binding domains (i.e., a small molecule binding domain) in which the co-stimulatory polypeptide is activated and/or regulated with a ligand resulting in oligomerization (i.e., a lipid-permeable, organic, dimerizing drug). Other systems that may be used to crosslink or for oligomerization of co-stimulatory polypeptides include antibodies, natural ligands, and/or artificial cross-reacting or synthetic ligands. Yet further, other dimerization systems contemplated include the coumermycin/DNA gyrase B system.

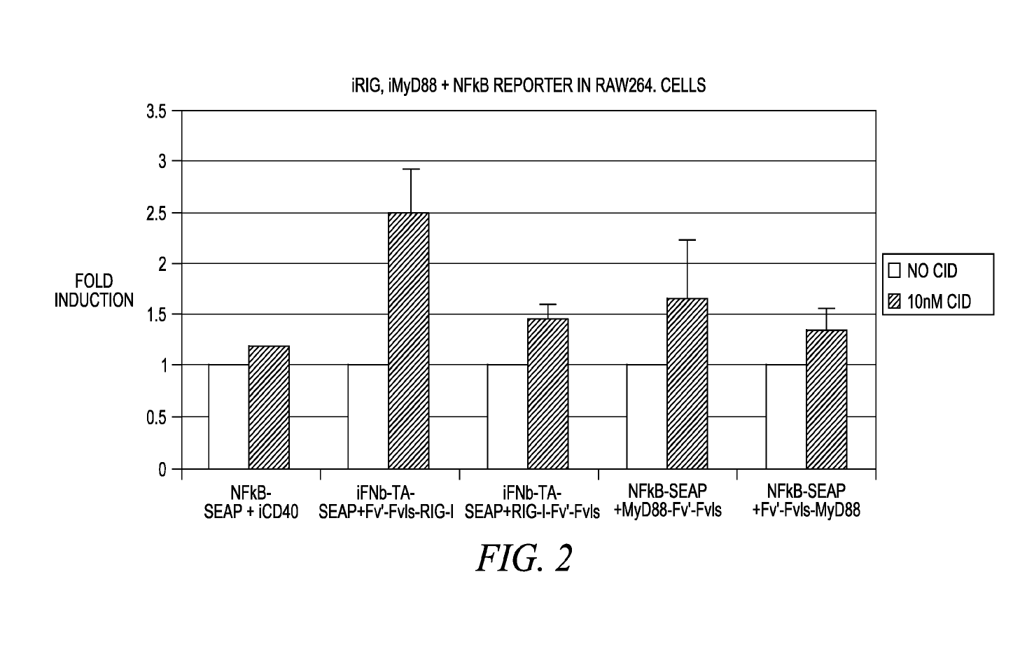

Co-stimulatory polypeptides that can be used include those that activate NF-kappaB and other variable signaling cascades for example the p38 pathway and/or Akt pathway. Such co-stimulatory polypeptides include, but are not limited to Pattern Recognition Receptors, C-reactive protein receptors (i.e., Nod1, Nod2, PtX3-R), TNF receptors (i.e., CD40, RANK/TRANCE-R, OX40, 4-1 BB), and HSP receptors (Lox-1 and CD-91). Pattern Recognition Receptors include, but are not limited to endocytic pattern-recognition receptors (i.e., mannose receptors, scavenger receptors (i.e., Mac-1, LRP, peptidoglycan, techoic acids, toxins, CD11c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), RIG1, and PRRs shown in FIG. 2. Pattern Recognition Receptors suitable for the present methods and composition, also include, for example, those discussed in, for example, Werts C., et al., Cell Death and Differentiation (2006) 13:798-815; Meylan, E., et al., Nature (2006) 442:39-44; and Strober, W., et al., Nature Reviews (2006) 6:9-20.

In specific embodiments, the co-stimulatory polypeptide molecule is CD40. The CD40 molecule comprises a nucleic acid molecule which: (1) hybridizes under stringent conditions to a nucleic acid having the sequence of a known CD40 gene and (2) codes for a CD40 polypeptide. The CD40 polypeptide may, in certain examples, lack the extracellular domain. Exemplary polynucleotide sequences that encode CD40 polypeptides include, but are not limited to SEQ. ID. NO: 1 and CD40 isoforms from other species. It is contemplated that other normal or mutant variants of CD40 can be used in the present methods and compositions. Thus, a CD40 region can have an amino acid sequence that differs from the native sequence by one or more amino acid substitutions, deletions and/or insertions. For example, one or more TNF receptor associated factor (TRAF) binding regions may be eliminated or effectively eliminated (e.g., a CD40 amino acid sequence is deleted or altered such that a TRAF protein does not bind or binds with lower affinity than it binds to the native CD40 sequence). In particular embodiments, a TRAF 3 binding region is deleted or altered such that it is eliminated or effectively eliminated (e.g., amino acids 250-254 may be altered or deleted; Hauer et al., PNAS 102(8): 2874-2879 (2005)).

In certain embodiments, the present methods involve the manipulation of genetic material to produce expression constructs that encode an inducible form of CD40 (iCD40). Such methods involve the generation of expression constructs containing, for example, a heterologous nucleic acid sequence encoding CD40 cytoplasmic domain and a means for its expression. The vector can be replicated in an appropriate helper cell, viral particles may be produced therefrom, and cells infected with the recombinant virus particles.

Thus, the CD40 molecule presented herein may, for example, lack the extracellular domain. In specific embodiments, the extracellular domain is truncated or removed. It is also contemplated that the extracellular domain can be mutated using standard mutagenesis, insertions, deletions, or substitutions to produce a CD40 molecule that does not have a functional extracellular domain. A CD40 nucleic acid may have the nucleic acid sequence of SEQ. ID. NO: 1. The CD40 nucleic acids also include homologs and alleles of a nucleic acid having the sequence of SEQ. ID. NO: 1, as well as, functionally equivalent fragments, variants, and analogs of the foregoing nucleic acids. Methods of constructing an inducible CD40 vector are discussed in, for example, U.S. Pat. No. 7,404,950, issued Jul. 29, 2008.

In the context of gene therapy, the gene will be a heterologous polynucleotide sequence derived from a source other than the viral genome, which provides the backbone of the vector. The gene is derived from a prokaryotic or eukaryotic source such as a bacterium, a virus, yeast, a parasite, a plant, or even an animal. The heterologous DNA also is derived from more than one source, i.e., a multigene construct or a fusion protein. The heterologous DNA also may include a regulatory sequence, which is derived from one source and the gene from a different source.

Ligand-Binding Regions

The ligand-binding (“dimerization”) domain of the expression construct can be any convenient domain that will allow for induction using a natural or unnatural ligand, for example, an unnatural synthetic ligand. The ligand-binding domain can be internal or external to the cellular membrane, depending upon the nature of the construct and the choice of ligand. A wide variety of ligand-binding proteins, including receptors, are known, including ligand-binding proteins associated with the cytoplasmic regions indicated above. As used herein the term “ligand-binding domain can be interchangeable with the term “receptor”. Of particular interest are ligand-binding proteins for which ligands (for example, small organic ligands) are known or may be readily produced. These ligand-binding domains or receptors include the FKBPs and cyclophilin receptors, the steroid receptors, the tetracycline receptor, the other receptors indicated above, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In certain embodiments, the ligand-binding region is selected from the group consisting of FKBP ligand-binding region, cyclophilin receptor ligand-binding region, steroid receptor ligand-binding region, cyclophilin receptors ligand-binding region, and tetracycline receptor ligand-binding region. Often, the ligand-binding region comprises an Fv′Fvls sequence. Sometimes, the Fv′Fvls sequence further comprises an additional Fv′ sequence. Examples include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology From Small Molecules to Systems Biology and Drug Design (Schreiber, s., et al., eds., Wiley, 2007)).

For the most part, the ligand-binding domains or receptor domains will be at least about 50 amino acids, and fewer than about 350 amino acids, usually fewer than 200 amino acids, either as the natural domain or truncated active portion thereof. The binding domain may, for example, be small (<25 kDa, to allow efficient transfection in viral vectors), monomeric, nonimmunogenic, have synthetically accessible, cell permeable, nontoxic ligands that can be configured for dimerization.

The receptor domain can be intracellular or extracellular depending upon the design of the expression construct and the availability of an appropriate ligand. For hydrophobic ligands, the binding domain can be on either side of the membrane, but for hydrophilic ligands, particularly protein ligands, the binding domain will usually be external to the cell membrane, unless there is a transport system for internalizing the ligand in a form in which it is available for binding. For an intracellular receptor, the construct can encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

The portion of the expression construct encoding the receptor can be subjected to mutagenesis for a variety of reasons. The mutagenized protein can provide for higher binding affinity, allow for discrimination by the ligand of the naturally occurring receptor and the mutagenized receptor, provide opportunities to design a receptor-ligand pair, or the like. The change in the receptor can involve changes in amino acids known to be at the binding site, random mutagenesis using combinatorial techniques, where the codons for the amino acids associated with the binding site or other amino acids associated with conformational changes can be subject to mutagenesis by changing the codon(s) for the particular amino acid, either with known changes or randomly, expressing the resulting proteins in an appropriate prokaryotic host and then screening the resulting proteins for binding.

Antibodies and antibody subunits, e.g., heavy or light chain, particularly fragments, more particularly all or part of the variable region, or fusions of heavy and light chain to create high-affinity binding, can be used as the binding domain. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen). Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding domain is known and there is a useful ligand for binding.

Oligomerization

The transduced signal will normally result from ligand-mediated oligomerization of the chimeric protein molecules, i.e., as a result of oligomerization following ligand-binding, although other binding events, for example allosteric activation, can be employed to initiate a signal. The construct of the chimeric protein will vary as to the order of the various domains and the number of repeats of an individual domain.

For multimerizing the receptor, the ligand for the ligand-binding domains/receptor domains of the chimeric surface membrane proteins will usually be multimeric in the sense that it will have at least two binding sites, with each of the binding sites capable of binding to the ligand receptor domain. Desirably, the subject ligands will be a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively higher orders of the ligands, e.g., trimeric can be used. For embodiments involving unnatural receptors, e.g., antibody subunits, modified antibody subunits, single chain antibodies comprised of heavy and light chain variable regions in tandem, separated by a flexible linker domain, or modified receptors, and mutated sequences thereof, and the like, any of a large variety of compounds can be used. A significant characteristic of these ligand units is that each binding site is able to bind the receptor with high affinity and they are able to be dimerized chemically. Also, methods are available to balance the hydrophobicity/hydrophilicity of the ligands so that they are able to dissolve in serum at functional levels, yet diffuse across plasma membranes for most applications.

In certain embodiments, the present methods utilize the technique of chemically induced dimerization (CID) to produce a conditionally controlled protein or polypeptide. In addition to this technique being inducible, it also is reversible, due to the degradation of the labile dimerizing agent or administration of a monomeric competitive inhibitor.

The CID system uses synthetic bivalent ligands to rapidly crosslink signaling molecules that are fused to ligand-binding domains. This system has been used to trigger the oligomerization and activation of cell surface (Spencer, D. M., et al., Science, 1993. 262: p. 1019-1024; Spencer D. M. et al., Curr Biol 1996, 6:839-847; Blau, C. A. et al., Proc Natl Acad. Sci. USA 1997, 94:3076-3081), or cytosolic proteins (Luo, Z. et al., Nature 1996, 383:181-185; MacCorkle, R. A. et al., Proc Natl Acad Sci USA 1998, 95:3655-3660), the recruitment of transcription factors to DNA elements to modulate transcription (Ho, S, N. et al., Nature 1996, 382:822-826; Rivera, V. M. et al., Nat. Med. 1996, 2:1028-1032) or the recruitment of signaling molecules to the plasma membrane to stimulate signaling (Spencer D. M. et al., Proc. Natl. Acad. Sci. USA 1995, 92:9805-9809; Holsinger, L. J. et al., Proc. Natl. Acad. Sci. USA 1995, 95:9810-9814).

The CID system is based upon the notion that surface receptor aggregation effectively activates downstream signaling cascades. In the simplest embodiment, the CID system uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12 permits specific activation of the recombinant receptor in vivo without the induction of non-specific side effects through endogenous FKBP12. FKBP12 variants having amino acid substitutions and deletions, such as FKBP12V₃₆, that bind to a dimerizer drug, may also be used. In addition, the synthetic ligands are resistant to protease degradation, making them more efficient at activating receptors in vivo than most delivered protein agents.

The ligands used are capable of binding to two or more of the ligand-binding domains. The chimeric proteins may be able to bind to more than one ligand when they contain more than one ligand-binding domain. The ligand is typically a non-protein or a chemical. Exemplary ligands include, but are not limited to dimeric FK506 (e.g., FK1012).

In some embodiments, the ligand is a small molecule. The appropriate ligand for the selected ligand-binding region may be selected. Often, the ligand is dimeric, sometimes, the ligand is a dimeric FK506 or a dimeric FK506 analog. In certain embodiments, the ligand is AP1903 (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9Cl) CAS Registry Number: 195514-63-7; Molecular Formula: C78H98N4O20 Molecular Weight: 1411.65). In certain embodiments, the ligand is AP20187.

In such methods, the multimeric molecule can be an antibody that binds to an epitope in the CD40 extracellular domain (e.g., humanized anti-CD40 antibody; Tai et al., Cancer Research 64, 2846-2852 (2004)), can be a CD40 ligand (e.g., U.S. Pat. No. 6,497,876 (Maraskovsky et al.)) or may be another co-stimulatory molecule (e.g., B7/CD28). It is understood that conservative variations in sequence, that do not affect the function, as assayed herein, are within the scope of the present claims.

Since the mechanism of CD40 activation is fundamentally based on trimerization, this receptor is particularly amenable to the CID system. CID regulation provides the system with 1) temporal control, 2) reversibility by addition of a non-active monomer upon signs of an autoimmune reaction, and 3) limited potential for non-specific side effects. In addition, inducible in vivo DC CD40 activation would circumvent the requirement of a second “danger” signal normally required for complete induction of CD40 signaling and would potentially promote DC survival in situ allowing for enhanced T cell priming. Thus, engineering DC vaccines to express iCD40 amplifies the T cell-mediated killing response by upregulating DC expression of antigen presentation molecules, adhesion molecules, TH1 promoting cytokines, and pro-survival factors.

Other dimerization systems contemplated include the coumermycin/DNA gyrase B system. Coumermycin-induced dimerization activates a modified Raf protein and stimulates the MAP kinase cascade. See Farrar et al., 1996.

Membrane-Targeting

A membrane-targeting sequence provides for transport of the chimeric protein to the cell surface membrane, where the same or other sequences can encode binding of the chimeric protein to the cell surface membrane. Molecules in association with cell membranes contain certain regions that facilitate the membrane association, and such regions can be incorporated into a chimeric protein molecule to generate membrane-targeted molecules. For example, some proteins contain sequences at the N-terminus or C-terminus that are acylated, and these acyl moieties facilitate membrane association. Such sequences are recognized by acyltransferases and often conform to a particular sequence motif. Certain acylation motifs are capable of being modified with a single acyl moiety (often followed by several positively charged residues (e.g. human c-Src: M-G-S-N-K-S-K-P-K-D-A-S-Q-R-R-R) (SEQ ID NO: 17) to improve association with anionic lipid head groups) and others are capable of being modified with multiple acyl moieties. For example the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys (SEQ ID NO: 18), where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can modified with C15 or C10 isoprenyl moieties, from the C-terminus of G-protein gamma subunits and other proteins (e.g., World Wide Web address ebi.ac.uk/interpro/DisplaylproEntry?ac=IPRO01230) also can be utilized. These and other acylation motifs include, for example, those discussed in Gauthier-Campbell et al., Molecular Biology of the Cell 15: 2205-2217 (2004); Glabati et al., Biochem. J. 303: 697-700 (1994) and Zlakine et al., J. Cell Science 110: 673-679 (1997), and can be incorporated in chimeric molecules to induce membrane localization. In certain embodiments, a native sequence from a protein containing an acylation motif is incorporated into a chimeric protein. For example, in some embodiments, an N-terminal portion of Lck, Fyn or Yes or a G-protein alpha subunit, such as the first twenty-five N-terminal amino acids or fewer from such proteins (e.g., about 5 to about 20 amino acids, about 10 to about 19 amino acids, or about 15 to about 19 amino acids of the native sequence with optional mutations), may be incorporated within the N-terminus of a chimeric protein. In certain embodiments, a C-terminal sequence of about 25 amino acids or less from a G-protein gamma subunit containing a CAAX box motif sequence (e.g., about 5 to about 20 amino acids, about 10 to about 18 amino acids, or about 15 to about 18 amino acids of the native sequence with optional mutations) can be linked to the C-terminus of a chimeric protein. In some embodiments, an acyl moiety has a log p value of +1 to +6, and sometimes has a log p value of +3 to +4.5. Log p values are a measure of hydrophobicity and often are derived from octanol/water partitioning studies, in which molecules with higher hydrophobicity partition into octanol with higher frequency and are characterized as having a higher log p value. Log p values are published for a number of lipophilic molecules and log p values can be calculated using known partitioning processes (e.g., Chemical Reviews, Vol. 71, Issue 6, page 599, where entry 4493 shows lauric acid having a log p value of 4.2). Any acyl moiety can be linked to a peptide composition discussed above and tested for antimicrobial activity using known methods and those discussed hereafter. The acyl moiety sometimes is a C1-C20 alkyl, C2-C20 alkenyl, C2-C20 alkynyl, C3-C6 cycloalkyl, C1-C4 haloalkyl, C4-C12 cyclalkylalkyl, aryl, substituted aryl, or aryl (C1-C4) alkyl, for example. Any acyl-containing moiety sometimes is a fatty acid, and examples of fatty acid moieties are propyl (C3), butyl (C4), pentyl (C5), hexyl (C6), heptyl (C7), octyl (C8), nonyl (C9), decyl (C10), undecyl (C11), lauryl (C12), myristyl (C14), palmityl (C16), stearyl (C18), arachidyl (C20), behenyl (C22) and lignoceryl moieties (C24), and each moiety can contain 0, 1, 2, 3, 4, 5, 6, 7 or 8 unsaturations (i.e., double bonds). An acyl moiety sometimes is a lipid molecule, such as a phosphatidyl lipid (e.g., phosphatidyl serine, phosphatidyl inositol, phosphatidyl ethanolamine, phosphatidyl choline), sphingolipid (e.g., shingomyelin, sphingosine, ceramide, ganglioside, cerebroside), or modified versions thereof. In certain embodiments, one, two, three, four or five or more acyl moieties are linked to a membrane association region.

A chimeric protein herein also may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFbeta, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

Any membrane-targeting sequence can be employed that is functional in the host and may, or may not, be associated with one of the other domains of the chimeric protein. In some embodiments, such sequences include, but are not limited to myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster J P et al, Biology of the Cell (2007) 99, 1-12, Vincent, S., et al., Nature Biotechnology 21:936-40, 1098 (2003).

Additional protein domains exist that can increase protein retention at various membranes. For example, an ˜120 amino acid pleckstrin homology (PH) domain is found in over 200 human proteins that are typically involved in intracellular signaling. PH domains can bind various phosphatidylinositol (PI) lipids within membranes (e.g. PI (3,4,5)-P3, PI (3,4)-P2, PI (4,5)-P2) and thus play a key role in recruiting proteins to different membrane or cellular compartments. Often the phosphorylation state of PI lipids is regulated, such as by PI-3 kinase or PTEN, and thus, interaction of membranes with PH domains is not as stable as by acyl lipids.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as EGFP, beta-gal or chloramphenicol acetyltransferase (CAT).

Control Regions

1. Promoters

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene products are toxic (add in more inducible promoters).

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity. (Bojak, A., et al., 2002. Vaccine. 20:1975-79; Cazeaux., N., et al., 2002. Vaccine 20:3322-31). For example, tissue specific promoters such as the PSA associated promoter or prostate-specific glandular kallikrein, or the muscle creatine kinase gene may be used where appropriate.

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8, 42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

2. Enhancers

Enhancers are genetic elements that increase transcription from a promoter located at a distant position on the same molecule of DNA. Early examples include the enhancers associated with immunoglobulin and T cell receptors that both flank the coding sequence and occur within several introns. Many viral promoters, such as CMV, SV40, and retroviral LTRs are closely associated with enhancer activity and are often treated like single elements. Enhancers are organized much like promoters. That is, they are composed of many individual elements, each of which binds to one or more transcriptional proteins. The basic distinction between enhancers and promoters is operational. An enhancer region as a whole stimulates transcription at a distance and often independent of orientation; this need not be true of a promoter region or its component elements. On the other hand, a promoter has one or more elements that direct initiation of RNA synthesis at a particular site and in a particular orientation, whereas enhancers lack these specificities. Promoters and enhancers are often overlapping and contiguous, often seeming to have a very similar modular organization. A subset of enhancers are locus-control regions (LCRs) that can not only increase transcriptional activity, but (along with insulator elements) can also help to insulate the transcriptional element from adjacent sequences when integrated into the genome.

Any promoter/enhancer combination (as per the Eukaryotic Promoter Data Base EPDB) can be used to drive expression of the gene, although many will restrict expression to a particular tissue type or subset of tissues. (reviewed in, for example, Kutzler, M. A., and Weiner, D. B., 2008. Nature Reviews Genetics 9:776-88). Examples include, but are not limited to, enhancers from the human actin, myosin, hemoglobin, muscle creatine kinase, sequences, and from viruses CMV, RSV, and EBV. Appropriate enhancers may be selected for particular applications. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the present methods, and any such sequence is employed such as human or bovine growth hormone and SV40 polyadenylation signals and LTR polyadenylation signals. One non-limiting example is the SV40 polyadenylation signal present in the pCEP3 plasmid (Invitrogen, Carlsbad, Calif.). Also contemplated as an element of the expression cassette is a terminator. These elements can serve to enhance message levels and to minimize read through from the cassette into other sequences. Termination or poly(A) signal sequences may be, for example, positioned about 11-30 nucleotides downstream from a conserved sequence (AAUAAA) at the 3′ end of the mRNA. (Montgomery, D. L., et al., 1993. DNA Cell Biol. 12:777-83; Kutzler, M. A., and Weiner, D. B., 2008. Nature Rev. Gen. 9:776-88).

4. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. The initiation codon is placed in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements is used to create multigene, or polycistronic messages. IRES elements are able to bypass the ribosome-scanning model of 5′ methylated cap-dependent translation and begin translation at internal sites (Pelletier and Sonenberg, Nature, 334:320-325, 1988). IRES elements from two members of the picornavirus family (polio and encephalomyocarditis) have been discussed (Pelletier and Sonenberg, 1988), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Sequence Optimization

Protein production may also be increased by optimizing the codons in the transgene. Species specific codon changes may be used to increase protein production. Also, codons may be optimized to produce an optimized RNA, which may result in more efficient translation. By optimizing the codons to be incorporated in the RNA, elements such as those that result in a secondary structure that causes instability, secondary mRNA structures that can, for example, inhibit ribosomal binding, or cryptic sequences that can inhibit nuclear export of mRNA can be removed. (Kutzler, M. A., and Weiner, D. B., 2008. Nature Rev. Gen. 9:776-88; Yan., J. et al., 2007. Mol. Ther. 15:411-21; Cheung, Y. K., et al., 2004. Vaccine 23:629-38; Narum., D. L., et al., 2001. 69:7250-55; Yadava, A., and Ockenhouse, C. F., 2003. Infect. Immun. 71:4962-69; Smith., J. M., et al., 2004. AIDS Res. Hum. Retroviruses 20:1335-47; Zhou, W., et al., 2002. Vet. Microbiol. 88:127-51; Wu, X., et al., 2004. Biochem. Biophys. Res. Commun. 313:89-96; Zhang, W., et al., 2006. Biochem. Biophys. Res. Commun. 349:69-78; Deml, L. A., et al., 2001. J. Virol. 75:1099-11001; Schneider, R. M., et al., 1997. J. Virol. 71:4892-4903; Wang, S. D., et al., 2006. Vaccine 24:4531-40; zur Megede, J., et al., 2000. J. Virol. 74:2628-2635).

Leader Sequences

Leader sequences may be added to enhance the stability of mRNA and result in more efficient translation. The leader sequence is usually involved in targeting the mRNA to the endoplasmic reticulum. Examples include, the signal sequence for the HIV-1 envelope glycoprotein (Env), which delays its own cleavage, and the IgE gene leader sequence (Kutzler, M. A., and Weiner, D. B., 2008. Nature Rev. Gen. 9:776-88; L1, V., et al., 2000. Virology 272:417-28; Xu, Z. L., et al. 2001. Gene 272:149-56; Malin, A. S., et al., 2000. Microbes Infect. 2:1677-85; Kutzler, M. A., et al., 2005. J. Immunol. 175:112-125; Yang., J. S., et al., 2002. Emerg. Infect. Dis. 8:1379-84; Kumar., S., et al., 2006. DNA Cell Biol. 25:383-92; Wang, S., et al., 2006. Vaccine 24:4531-40). The IgE leader may be used to enhance insertion into the endoplasmic reticulum (Tepler, I, et al. (1989) J. Biol. Chem. 264:5912).

Expression of the transgenes may be optimized and/or controlled by the selection of appropriate methods for optimizing expression. These methods include, for example, optimizing promoters, delivery methods, and gene sequences, (for example, as presented in Laddy, D. J., et al., 2008. PLoS.ONE 3 e2517; Kutzler, M. A., and Weiner, D. B., 2008. Nature Rev. Gen. 9:776-88).

### Nucleic Acids

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides.

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are often used for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. The low or high stringency conditions may be further modified to suit a particular application.

### Nucleic Acid Modification

Any of the modifications discussed below may be applied to a nucleic acid. Examples of modifications include alterations to the RNA or DNA backbone, sugar or base, and various combinations thereof. Any suitable number of backbone linkages, sugars and/or bases in a nucleic acid can be modified (e.g., independently about 5%, 10%, 15%, 20%, 25%, 30%, 35%, 40%, 45%, 50%, 55%, 60%, 65%, 70%, 75%, 80%, 85%, 90%, 95%, up to 100%). An unmodified nucleoside is any one of the bases adenine, cytosine, guanine, thymine, or uracil joined to the 1′ carbon of beta-D-ribo-furanose.

A modified base is a nucleotide base other than adenine, guanine, cytosine and uracil at a 1′ position. Non-limiting examples of modified bases include inosine, purine, pyridin-4-one, pyridin-2-one, phenyl, pSEQdouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and the like. Other non-limiting examples of modified bases include nitropyrrolyl (e.g., 3-nitropyrrolyl), nitroindolyl (e.g., 4-, 5-, 6-nitroindolyl), hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, difluorotolyl, 4-fluoro-6-methylbenzimidazole, 4-methylbenzimidazole, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl and the like.

In some embodiments, for example, a nucleid acid may comprise modified nucleic acid molecules, with phosphate backbone modifications. Non-limiting examples of backbone modifications include phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl modifications. In certain instances, a ribose sugar moiety that naturally occurs in a nucleoside is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. The hexose may be a D-hexose, glucose, or mannose. In certain instances, the polycyclic heteroalkyl group may be a bicyclic ring containing one oxygen atom in the ring. In certain instances, the polycyclic heteroalkyl group is a bicyclo[2.2.1]heptane, a bicyclo[3.2.1]octane, or a bicyclo[3.3.1]nonane.

Nitropyrrolyl and nitroindolyl nucleobases are members of a class of compounds known as universal bases. Universal bases are those compounds that can replace any of the four naturally occurring bases without substantially affecting the melting behavior or activity of the oligonucleotide duplex. In contrast to the stabilizing, hydrogen-bonding interactions associated with naturally occurring nucleobases, oligonucleotide duplexes containing 3-nitropyrrolyl nucleobases may be stabilized solely by stacking interactions. The absence of significant hydrogen-bonding interactions with nitropyrrolyl nucleobases obviates the specificity for a specific complementary base. In addition, 4-, 5- and 6-nitroindolyl display very little specificity for the four natural bases. Procedures for the preparation of 1-(2′-O-methyl-beta.-D-ribofuranosyl)-5-nitroindole are discussed in Gaubert, G.; Wengel, J. Tetrahedron Letters 2004, 45, 5629. Other universal bases include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof.

Difluorotolyl is a non-natural nucleobase that functions as a universal base. Difluorotolyl is an isostere of the natural nucleobase thymine. But unlike thymine, difluorotolyl shows no appreciable selectivity for any of the natural bases. Other aromatic compounds that function as universal bases are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above, see: Schweitzer et al., J. Org. Chem., 59:7238-7242 (1994);

In addition, chemical substituents, for example cross-linking agents, may be used to add further stability or irreversibility to the reaction. Non-limiting examples of cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

A nucleotide analog may also include a “locked” nucleic acid. Certain compositions can be used to essentially “anchor” or “lock” an endogenous nucleic acid into a particular structure. Anchoring sequences serve to prevent disassociation of a nucleic acid complex, and thus not only can prevent copying but may also enable labeling, modification, and/or cloning of the endogeneous sequence. The locked structure may regulate gene expression (i.e. inhibit or enhance transcription or replication), or can be used as a stable structure that can be used to label or otherwise modify the endogenous nucleic acid sequence, or can be used to isolate the endogenous sequence, i.e. for cloning.

Nucleic acid molecules need not be limited to those molecules containing only RNA or DNA, but further encompass chemically-modified nucleotides and non-nucleotides. The percent of non-nucleotides or modified nucleotides may be from 1% to 100% (e.g., about 5, 10, 15, 20, 25, 30, 35, 40, 45, 50, 55, 60, 65, 70, 75, 80, 85, 90 or 95%).

### Nucleic Acid Preparation

In some embodiments, a nucleic acid is provided for use as a control or standard in an assay, or therapeutic, for example. A nucleic acid may be made by any technique known in the art, such as for example, chemical synthesis, enzymatic production or biological production. Nucleic acids may be recovered or isolated from a biological sample. The nucleic acid may be recombinant or it may be natural or endogenous to the cell (produced from the cell's genome). It is contemplated that a biological sample may be treated in a way so as to enhance the recovery of small nucleic acid molecules. Generally, methods may involve lysing cells with a solution having guanidinium and a detergent.

Nucleic acid synthesis may also be performed according to standard methods. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques or via deoxynucleoside H-phosphonate intermediates. Various different mechanisms of oligonucleotide synthesis have been disclosed elsewhere.

Nucleic acids may be isolated using known techniques. In particular embodiments, methods for isolating small nucleic acid molecules, and/or isolating RNA molecules can be employed. Chromatography is a process used to separate or isolate nucleic acids from protein or from other nucleic acids. Such methods can involve electrophoresis with a gel matrix, filter columns, alcohol precipitation, and/or other chromatography. If a nucleic acid from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

Methods may involve the use of organic solvents and/or alcohol to isolate nucleic acids. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column is effective for such isolation procedures.

A nucleic acid isolation processes may sometimes include: a) lysing cells in the sample with a lysing solution comprising guanidinium, where a lysate with a concentration of at least about 1 M guanidinium is produced; b) extracting nucleic acid molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the nucleic acid molecules from the solid support with an ionic solution; and, f) capturing the nucleic acid molecules. The sample may be dried down and resuspended in a liquid and volume appropriate for subsequent manipulation.

### Methods of Gene Transfer

In order to mediate the effect of the transgene expression in a cell, it will be necessary to transfer the expression constructs into a cell. Such transfer may employ viral or non-viral methods of gene transfer. This section provides a discussion of methods and compositions of gene transfer. A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

A host cell can, and has been, used as a recipient for vectors. Host cells may be derived from prokaryotes or eukaryotes, depending upon whether the desired result is replication of the vector or expression of part or all of the vector-encoded polynucleotide sequences. Numerous cell lines and cultures are available for use as a host cell, and they can be obtained through the American Type Culture Collection (ATCC), which is an organization that serves as an archive for living cultures and genetic materials. In specific embodiments, the host cell is a dendritic cell, which is an antigen-presenting cell.

An appropriate host may be determined. Generally this is based on the vector backbone and the desired result. A plasmid or cosmid, for example, can be introduced into a prokaryote host cell for replication of many vectors. Bacterial cells used as host cells for vector replication and/or expression include DH5alpha, JM109, and KC8, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Eukaryotic cells that can be used as host cells include, but are not limited to yeast, insects and mammals. Examples of mammalian eukaryotic host cells for replication and/or expression of a vector include, but are not limited to, HeLa, NIH3T3, Jurkat, 293, COS, CHO, Saos, and PC12. Examples of yeast strains include, but are not limited to, YPH499, YPH500 and YPH501.

Nucleic acid vaccines may include, for example, non-viral DNA vectors, “naked” DNA and RNA, and viral vectors. Methods of transforming cells with these vaccines, and for optimizing the expression of genes included in these vaccines are known and are also discussed herein.

Examples of Methods of Nucleic Acid or Viral Vector Transfer

Any appropriate method may be used to transfect or transform the antigen presenting cells, or to administer the nucleotide sequences or compositions of the present methods. Certain examples are presented herein, and further include methods such as delivery using cationic polymers, lipid like molecules, and certain commercial products such as, for example, IN-VIVO-JET PEI.

1. Ex Vivo Transformation

Various methods are available for transfecting vascular cells and tissues removed from an organism in an ex vivo setting. For example, canine endothelial cells have been genetically altered by retroviral gene transfer in vitro and transplanted into a canine (Wilson et al., Science, 244:1344-1346, 1989). In another example, Yucatan minipig endothelial cells were transfected by retrovirus in vitro and transplanted into an artery using a double-balloon catheter (Nabel et al., Science, 244(4910):1342-1344, 1989). Thus, it is contemplated that cells or tissues may be removed and transfected ex vivo using the polynucleotides presented herein. In particular aspects, the transplanted cells or tissues may be placed into an organism. For example, dendritic cells from an animal, transfect the cells with the expression vector and then administer the transfected or transformed cells back to the animal.

2. Injection

In certain embodiments, an antigen presenting cell or a nucleic acid or viral vector may be delivered to an organelle, a cell, a tissue or an organism via one or more injections (i.e., a needle injection), such as, for example, subcutaneous, intradermal, intramuscular, intravenous, intraprotatic, intratumor, intraperitoneal, etc. Methods of injection include, foe example, injection of a composition comprising a saline solution. Further embodiments include the introduction of a polynucleotide by direct microinjection. The amount of the expression vector used may vary upon the nature of the antigen as well as the organelle, cell, tissue or organism used. Intradermal, intranodal, or intralymphatic injections are some of the more commonly used methods of DC administration. Intradermal injection is characterized by a low rate of absorption into the bloodstream but rapid uptake into the lymphatic system. The presence of large numbers of Langerhans dendritic cells in the dermis will transport intact as well as processed antigen to draining lymph nodes. Proper site preparation is necessary to perform this correctly (i.e., hair is clipped in order to observe proper needle placement). Intranodal injection allows for direct delivery of antigen to lymphoid tissues. Intralymphatic injection allows direct administration of DCs.

3. Electroporation

In certain embodiments, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

Transfection of eukaryotic cells using electroporation has been quite successful. Mouse pre-B lymphocytes have been transfected with human kappa-immunoglobulin genes (Potter et al., (1984) Proc. Nat'l Acad. Sci. USA, 81, 7161-7165), and rat hepatocytes have been transfected with the chloramphenicol acetyltransferase gene (Tur-Kaspa et al., (1986) Mol. Cell Biol., 6, 716-718) in this manner.

4. Calcium Phosphate

In other embodiments, a polynucleotide is introduced to the cells using calcium phosphate precipitation. Human KB cells have been transfected with adenovirus 5 DNA (Graham and van der Eb, (1973) Virology, 52, 456-467) using this technique. Also in this manner, mouse L(A9), mouse C127, CHO, CV-1, BHK, NIH3T3 and HeLa cells were transfected with a neomycin marker gene (Chen and Okayama, Mol. Cell Biol., 7(8):2745-2752, 1987), and rat hepatocytes were transfected with a variety of marker genes (Rippe et al., Mol. Cell Biol., 10:689-695, 1990).

5. DEAE-Dextran

In another embodiment, a polynucleotide is delivered into a cell using DEAE-dextran followed by polyethylene glycol. In this manner, reporter plasmids were introduced into mouse myeloma and erythroleukemia cells (Gopal, T. V., Mol Cell Biol. 1985 May; 5(5):1188-90).

6. Sonication Loading

Additional embodiments include the introduction of a polynucleotide by direct sonic loading. LTK-fibroblasts have been transfected with the thymidine kinase gene by sonication loading (Fechheimer et al., (1987) Proc. Nat'l Acad. Sci. USA, 84, 8463-8467).

7. Liposome-Mediated Transfection

In a further embodiment, a polynucleotide may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine (Gibco BRL) or Superfect (Qiagen).

8. Receptor Mediated Transfection

Still further, a polynucleotide may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity.

Certain receptor-mediated gene targeting vehicles comprise a cell receptor-specific ligand and a polynucleotide-binding agent. Others comprise a cell receptor-specific ligand to which the polynucleotide to be delivered has been operatively attached. Several ligands have been used for receptor-mediated gene transfer (Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085), which establishes the operability of the technique. Specific delivery in the context of another mammalian cell type has been discussed (Wu and Wu, Adv. Drug Delivery Rev., 12:159-167, 1993; incorporated herein by reference). In certain aspects, a ligand is chosen to correspond to a receptor specifically expressed on the target cell population. In other embodiments, a polynucleotide delivery vehicle component of a cell-specific polynucleotide-targeting vehicle may comprise a specific binding ligand in combination with a liposome. The polynucleotide(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a polynucleotide to cells that exhibit upregulation of the EGF receptor.

In still further embodiments, the polynucleotide delivery vehicle component of a targeted delivery vehicle may be a liposome itself, which may, for example, comprise one or more lipids or glycoproteins that direct cell-specific binding. For example, lactosyl-ceramide, a galactose-terminal asialoganglioside, have been incorporated into liposomes and observed an increase in the uptake of the insulin gene by hepatocytes (Nicolau et al., (1987) Methods Enzymol., 149, 157-176). It is contemplated that the tissue-specific transforming constructs may be specifically delivered into a target cell in a similar manner.

9. Microprojectile Bombardment

Microprojectile bombardment techniques can be used to introduce a polynucleotide into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., (1987) Nature, 327, 70-73). There are a wide variety of microprojectile bombardment techniques known in the art, many of which are applicable to the present methods. In this microprojectile bombardment, one or more particles may be coated with at least one polynucleotide and delivered into cells by a propelling force. Several devices for accelerating small particles have been developed. One such device relies on a high voltage discharge to generate an electrical current, which in turn provides the motive force (Yang et al., (1990) Proc. Nat'l Acad. Sci. USA, 87, 9568-9572). The microprojectiles used have consisted of biologically inert substances such as tungsten or gold particles or beads. Exemplary particles include those comprised of tungsten, platinum, and, in certain examples, gold, including, for example, nanoparticles. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

Examples of Methods of Viral Vector-Mediated Transfer

Any viral vector suitable for administering nucleotide sequences, or compositions comprising nucleotide sequences, to a cell or to a subject, such that the cell or cells in the subject may express the genes encoded by the nucleotide sequences may be employed in the present methods. In certain embodiments, a transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present methods, it is possible to achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol Biol. 1984 Jun. 5; 175(4):493-510). Therefore, inclusion of these elements in an adenoviral vector may permits replication.

In addition, the packaging signal for viral encapsulation is localized between 194-385 by (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element derives from the packaging function of adenovirus.

It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pSEQdotyped” virus, Ad5f35, has been the basis for several clinically developed viral isolates. Moreover, various biochemical methods exist to modify the fiber to allow re-targeting of the virus to target cells, such as dendritic cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

2. Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248). An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pSEQdorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

4. Other Viral Vectors

Other viral vectors are employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

Enhancement of an Immune Response

In certain embodiments, a DC activation strategy is contemplated, that incorporates the manipulation of signaling co-stimulatory polypeptides that activate biological pathways, for example, immunological pathways, such as, for example, NF-kappaB pathways, Akt pathways, and/or p38 pathways. This DC activation system can be used in conjunction with or without standard vaccines to enhance the immune response since it replaces the requirement for CD4+ T cell help during APC activation (Bennett, S. R., et al., Nature, 1998, Jun. 4, 393: p. 478-80; Ridge, J. P., D. R. F, and P. Nature, 1998, Jun. 4, 393: p. 474-8; Schoenberger, S. P., et al., Nature, 1998, Jun. 4, 393: p. 480-3). Thus, the DC activation system presented herein enhances immune responses by circumventing the need for the generation of MHC class II-specific peptides.

In specific embodiments, the DC activation is via CD40 activation. Thus, DC activation via endogenous CD40/CD40L interactions may be subject to downregulation due to negative feedback, leading rapidly to the “IL-12 burn-out effect”. Within 7 to 10 hours after CD40 activation, an alternatively spliced isoform of CD40 (type II) is produced as a secretable factor (Tone, M., et al., Proc Natl Acad Sci USA, 2001. 98(4): p. 1751-1756). Type II CD40 may act as a dominant negative receptor, downregulating signaling through CD40L and potentially limiting the potency of the immune response generated. Therefore, the present methods co-opt the natural regulation of CD40 by creating an inducible form of CD40 (iCD40), lacking the extracellular domain and activated instead by synthetic dimerizing ligands (Spencer, D. M., et al., Science, 1993. 262: p. 1019-1024) through a technology termed chemically induced dimerization (CID).

Included are methods of enhancing the immune response in an subject comprising the step of administering the expression vector, expression construct or transduced antigen-presenting cells to the subject. The expression vector encodes a co-stimulatory polypeptide, such as iCD40.

In certain embodiments the antigen-presenting cells are in an animal, such as human, non-human primate, cow, horse, pig, sheep, goat, dog, cat, or rodent. The subject may be, for example, an animal, such as a mammal, for example, a human, non-human primate, cow, horse, pig, sheep, goat, dog, cat, or rodent. The subject may be, for example, human, for example, a patient suffering from an infectious disease, and/or a subject that is immunocompromised, or is suffering from a hyperproliferative disease.

In further embodiments, the expression construct and/or expression vector can be utilized as a composition or substance that activates antigen-presenting cells. Such a composition that “activates antigen-presenting cells” or “enhances the activity antigen-presenting cells” refers to the ability to stimulate one or more activities associated with antigen-presenting cells. For example, a composition, such as the expression construct or vector of the present methods, can stimulate upregulation of co-stimulatory molecules on antigen-presenting cells, induce nuclear translocation of NF-kappaB in antigen-presenting cells, activate toll-like receptors in antigen-presenting cells, or other activities involving cytokines or chemokines.

The expression construct, expression vector and/or transduced antigen-presenting cells can enhance or contribute to the effectiveness of a vaccine by, for example, enhancing the immunogenicity of weaker antigens such as highly purified or recombinant antigens, reducing the amount of antigen required for an immune response, reducing the frequency of immunization required to provide protective immunity, improving the efficacy of vaccines in subjects with reduced or weakened immune responses, such as newborns, the aged, and immunocompromised individuals, and enhancing the immunity at a target tissue, such as mucosal immunity, or promote cell-mediated or humoral immunity by eliciting a particular cytokine profile.

In certain embodiments, the antigen-presenting cell is also contacted with an antigen. Often, the antigen-presenting cell is contacted with the antigen ex vivo. Sometimes, the antigen-presenting cell is contacted with the antigen in vivo. In some embodiments, the antigen-presenting cell is in a subject and an immune response is generated against the antigen. Sometimes, the immune response is a cytotoxic T-lymphocyte (CTL) immune response. Sometimes, the immune response is generated against a tumor antigen. In certain embodiments, the antigen-presenting cell is activated without the addition of an adjuvant.

In some embodiments, the antigen-presenting cell is transduced with the nucleic acid ex vivo and administered to the subject by intradermal administration. In some embodiments, the antigen-presenting cell is transduced with the nucleic acid ex vivo and administered to the subject by subcutaneous administration. Sometimes, the antigen-presenting cell is transduced with the nucleic acid ex vivo. Sometimes, the antigen-presenting cell is transduced with the nucleic acid in vivo.

In certain embodiments, the antigen-presenting cell can be transduced ex vivo or in vivo with a nucleic acid that encodes the chimeric protein. The antigen-presenting cell may be sensitized to the antigen at the same time the antigen-presenting cell is contacted with the multimeric ligand, or the antigen-presenting cell can be pre-sensitized to the antigen before the antigen-presenting cell is contacted with the multimerization ligand. In some embodiments, the antigen-presenting cell is contacted with the antigen ex vivo. In certain embodiments the antigen-presenting cell is transduced with the nucleic acid ex vivo and administered to the subject by intradermal administration, and sometimes the antigen-presenting cell is transduced with the nucleic acid ex vivo and administered to the subject by subcutaneous administration. The antigen may be a tumor antigen, and the CTL immune response can induced by migration of the antigen-presenting cell to a draining lymph node. A tumor antigen is any antigen such as, for example, a peptide or polypeptide, that triggers an immune response in a host. The tumor antigen may be a tumor-associated antigen, that is associated with a neoplastic tumor cell.

In some embodiments, an immunocompromised individual or subject is a subject that has a reduced or weakened immune response. Such individuals may also include a subject that has undergone chemotherapy or any other therapy resulting in a weakened immune system, a transplant recipient, a subject currently taking immunosuppressants, an aging individual, or any individual that has a reduced and/or impaired CD4 T helper cells. It is contemplated that the present methods can be utilized to enhance the amount and/or activity of CD4 T helper cells in an immunocompromised subject.

Challenge with Target Antigens

In specific embodiments, prior to administering the transduced antigen-presenting cell, the cells are challenged with antigens (also referred herein as “target antigens”). After challenge, the transduced, loaded antigen-presenting cells are administered to the subject parenterally, intradermally, intranodally, or intralymphatically. Additional parenteral routes include, but are not limited to subcutaneous, intramuscular, intraperitoneal, intravenous, intraarterial, intramyocardial, transendocardial, transepicardial, intrathecal, intraprotatic, intratumor, and infusion techniques. The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like.

Target antigen may be derived or isolated from, for example, pathogenic yeast including Aspergillus, invasive Candida (U.S. Pat. No. 5,645,992), Nocardia, Histoplasmosis, Cryptosporidia and the like.

Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii. Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBOJ, 11:527-535, 1992), TAG72, KSA, CA-125, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each. An antigen may be expressed more frequently in cancer cells than in non-cancer cells. The antigen may result from contacting the modified dendritic cell with a prostate specific membrane antigen, for example, a prostate specific membrane antigen (PSMA) or fragment thereof.

Prostate antigen (PA001) is a recombinant protein consisting of the extracellular portion of PSMA antigen. PSMA is a ˜100 kDa (84 kDa before glycosylation, ˜180 kDa as dimer) type II membrane protein with neuropeptidase and folate hydrolase activities, but the true function of PSMA is currently unclear. Carter R E, et al., Proc Natl Acad Sci USA. 93: 749-53, 1996; Israeli R S, et al., Cancer Res. 53: 227-30, 1993; Pinto J T, et al., Clin Cancer Res. 2: 1445-51, 1996.

Expression is largely, but not exclusively, prostate-specific and is maintained in advanced and hormone refractory disease. Israeli R S, et al., Cancer Res. 54: 1807-11, 1994. Weak non-prostatic detection in normal tissues has also been seen in the salivary gland, brain, small intestines, duodenal mucosa, proximal renal tubules and neuroendocrine cells in colonic crypts. Silver D A, et al., Clin Cancer Res. 3: 81-5, 1997; Troyer J K, et al., Int J. Cancer. 62: 552-8, 1995. Moreover, PSMA is up-regulated following androgen deprivation therapy (ADT). Wright G L, Jr., et al., Urology. 48: 326-34, 1996. While most PSMA is expressed as a cytoplasmic protein, the alternatively-spliced transmembrane form is the predominate form on the apical surface of neoplastic prostate cells. Su S L, et al., Cancer Res. 55: 1441-3, 1995; Israeli R S, et al., Cancer Res. 54: 6306-10, 1994.

Moreover, PSMA is internalized following cross-linking and has been used to internalize bound antibody or ligand complexed with radionucleotides or viruses and other complex macromolecules. Liu H, et al., Cancer Res. 58: 4055-60, 1998; Freeman L M, et al., Q J Nucl Med. 46: 131-7, 2002; Kraaij R, et al., Prostate. 62: 253-9, 2005. Bander and colleagues demonstrated that pretreatment of tumors with microtubule inhibitors increases aberrant basal surface targeting and antibody-mediated internalization of PSMA. Christiansen J J, et al., Mol Cancer Ther. 4: 704-14, 2005. Tumor targeting may be facilitated by the observation of ectopic expression of PSMA in tumor vascular endothelium of not only prostate, but also renal and other tumors. Liu H, et al., Cancer Res. 57: 3629-34, 1997; Chang S S, et al., Urology. 57: 801-5, 2001; Chang S S, et al., Clin Cancer Res. 5: 2674-81, 1999.

PSMA is not found in the vascular endothelial cells of corresponding benign tissue. de la Taille A, et al., Cancer Detect Prey. 24: 579-88, 2000. Although one early histological study of metastatic prostate disease suggested that only ˜50% (8 of 18) of bone metastases (with 7 of 8 lymph node metastases) expressed PSMA, the more sensitive reagent, 177Lu-radiolabeled MoAb J591, targeted to the ectodomain of PSMA, could target all known sites of bone and soft tissue metastasis in 30 of 30 patients, suggesting near universal expression in advanced prostate disease. Bander N H, et al., J Clin Oncol. 23: 4591-601, 2005.

A prostate specific antigen, or PSA, is meant to include any antigen that can induce an immune response, such as, for example, a cytotoxic T lymphocyte response, against a PSA, for example, a PSMA, and may be specifically recognized by any anti-PSA antibody. PSAs used in the present method are capable of being used to load the antigen presenting cell, as assayed using conventional methods. Thus, “prostate specific antigen” or “PSA” may, for example, refer to a protein having the wild type amino acid sequence of a PSA, or a polypeptide that includes a portion of the a PSA protein,

A prostate specific membrane antigen, or PSMA, is meant to include any antigen that can induce an immune response, such as, for example, a cytotoxic T lymphocyte response, against PSMA, and may be specifically recognized by an anti-PSMA antibody. PSMAs used in the present method are capable of being used to load the antigen presenting cell, as assayed using conventional methods. Thus, “prostate specific membrane antigen” or “PSMA” may, for example, refer to a protein having the wild type amino acid sequence of PSMA, or a polypeptide that includes a portion of the PSMA protein, such as one encoded by SEQ ID NO: 3, or a portion of the nucleotide sequence of SEQ ID NO:3, or having the polypeptide of SEQ ID NO: 4, or a portion thereof. The term may also refer to, for example, a peptide having an amino acid sequence of a portion of SEQ ID NO: 4, or any peptide that may induce an immune response against PSMA. Also included are variants of any of the foregoing, including, for example, those having substitutions and deletions. Proteins, polypeptides, and peptides having differential post-translational processing, such as differences in glycosylation, from the wild type PSMA, may also be used in the present methods. Further, various sugar molecules that are capable of inducing an immune response against PSMA, are also contemplated.

A PSA, for example, a PSMA, polypeptide may be used to load the modified antigen presenting cell. In certain embodiments, the modified antigen presenting cell is contacted with a PSMA polypeptide fragment having the amino acid sequence of SEQ ID NO: 4 (e.g., encoded by the nucleotide sequence of SEQ ID NO: 3), or a fragment thereof. In some embodiments, the PSA, for example, PSMA polypeptide fragment does not include the signal peptide sequence. In other embodiments, the modified antigen presenting cell is contacted with a PSA, for example, PSMA polypeptide fragment comprising substitutions or deletions of amino acids in the polypeptide, and the fragment is sufficient to load antigen presenting cells.

A prostate specific protein antigen, or s PSPA, also referred to in this specification as a prostate specific antigen, or a PSA, is meant to include any antigen that can induce an immune response, such as, for example, a cytotoxic T lymphocyte response, against a prostate specific protein antigen. This includes, for example, a prostate specific protein antigen or Prostate Specific

Antigen. PSPAs used in the present method are capable of being used to load the antigen presenting cell, as assayed using conventional methods. Prostate Specific Antigen, or PSA, may, for example, refer to a protein having the wild type amino acid sequence of a PSA, or a polypeptide that includes a portion of the PSA protein,

A prostate specific membrane antigen, or PSMA, is meant to include any antigen that can induce an immune response, such as, for example, a cytotoxic T lymphocyte response, against PSMA, and may be specifically recognized by an anti-PSMA antibody. PSMAs used in the present method are capable of being used to load the antigen presenting cell, as assayed using conventional methods. Thus, “prostate specific membrane antigen” or “PSMA” may, for example, refer to a protein having the wild type amino acid sequence of PSMA, or a polypeptide that includes a portion of the PSMA protein, such as one encoded by SEQ ID NO: 3, or a portion of the nucleotide sequence of SEQ ID NO:3, or having the polypeptide of SEQ ID NO: 4, or a portion thereof. The term may also refer to, for example, a peptide having an amino acid sequence of a portion of SEQ ID NO: 4, or any peptide that may induce an immune response against PSMA. Also included are variants of any of the foregoing, including, for example, those having substitutions and deletions. Proteins, polypeptides, and peptides having differential post-translational processing, such as differences in glycosylation, from the wild type PSMA, may also be used in the present methods. Further, various sugar molecules that are capable of inducing an immune response against PSMA, are also contemplated.

A PSPA, for example, a PSMA, polypeptide may be used to load the modified antigen presenting cell. In certain embodiments, the modified antigen presenting cell is contacted with a PSMA polypeptide fragment having the amino acid sequence of SEQ ID NO: 4 (e.g., encoded by the nucleotide sequence of SEQ ID NO: 3), or a fragment thereof. In some embodiments, the PSA, for example, PSMA polypeptide fragment does not include the signal peptide sequence. In other embodiments, the modified antigen presenting cell is contacted with a PSPA, for example, PSMA polypeptide fragment comprising substitutions or deletions of amino acids in the polypeptide, and the fragment is sufficient to load antigen presenting cells.

A tumor antigen is any antigen such as, for example, a peptide or polypeptide, that triggers an immune response in a host against a tumor. The tumor antigen may be a tumor-associated antigen, that is associated with a neoplastic tumor cell.

A prostate cancer antigen, or PCA, is any antigen such as, for example, a peptide or polypeptide, that triggers an immune response in a host against a prostate cancer tumor. A prostate cancer antigen may, or may not, be specific to prostate cancer tumors. A prostate cancer antigen may also trigger immune responses against other types of tumors or neoplastic cells. A prostate cancer antigen includes, for example, prostate specific protein antigens, prostate specific antigens, and prostate specific membrane antigens.

The antigen presenting cell may be contacted with tumor antigen, such as PSA, for example, PSMA polypeptide, by various methods, including, for example, pulsing immature DCs with unfractionated tumor lysates, MHC-eluted peptides, tumor-derived heat shock proteins (HSPS), tumor associated antigens (TAAs (peptides or proteins)), or transfecting DCs with bulk tumor mRNA, or mRNA coding for TAAs (reviewed in Gilboa, E. & Vieweg, J., Immunol Rev 199, 251-63 (2004); Gilboa, E, Nat Rev Cancer 4, 401-11 (2004)).

For organisms that contain a DNA genome, a gene encoding a target antigen or immunological epitope thereof of interest is isolated from the genomic DNA. For organisms with RNA genomes, the desired gene may be isolated from cDNA copies of the genome. If restriction maps of the genome are available, the DNA fragment that contains the gene of interest is cleaved by restriction endonuclease digestion by routine methods. In instances where the desired gene has been previously cloned, the genes may be readily obtained from the available clones. Alternatively, if the DNA sequence of the gene is known, the gene can be synthesized by any of the conventional techniques for synthesis of deoxyribonucleic acids.

Genes encoding an antigen of interest can be amplified, for example, by cloning the gene into a bacterial host. For this purpose, various prokaryotic cloning vectors can be used. Examples are plasmids pBR322, pUC and pEMBL.

The genes encoding at least one target antigen or immunological epitope thereof can be prepared for insertion into the plasmid vectors designed for recombination with a virus by standard techniques. In general, the cloned genes can be excised from the prokaryotic cloning vector by restriction enzyme digestion. In most cases, the excised fragment will contain the entire coding region of the gene. The DNA fragment carrying the cloned gene can be modified as needed, for example, to make the ends of the fragment compatible with the insertion sites of the DNA vectors used for recombination with a virus, then purified prior to insertion into the vectors at restriction endonuclease cleavage sites (cloning sites).

Antigen loading of antigen presenting cells, such as, for example, dendritic cells, with antigens may be achieved, for example, by contacting antigen presenting cells, such as, for example, dendritic cells or progenitor cells with an antigen, for example, by incubating the cells with the antigen. Loading may also be achieved, for example, by incubating DNA (naked or within a plasmid vector) or RNA that code for the antigen; or with antigen-expressing recombinant bacterium or viruses (e.g., vaccinia, fowlpox, adenovirus or lentivirus vectors). Prior to loading, the antigen may be covalently conjugated to an immunological partner that provides T cell help (e.g., a carrier molecule). Alternatively, a dendritic cell may be pulsed with a non-conjugated immunological partner, separately or in the presence of the polypeptide. Antigens from cells or MHC molecules may be obtained by acid-elution or other methods (see Zitvogel L, et al., J Exp Med 1996. 183:87-97). The antigen presenting cells may be transduced or transfected with the chimeric protein-encoding nucleotide sequence according to the present methods either before, after, or at the same time as the cells are loaded with antigen. In particular embodiments, antigen loading is subsequent to transduction or transfection.

In further embodiments, the transduced antigen-presenting cell is transfected with tumor cell mRNA. The transduced transfected antigen-presenting cell is administered to an animal to effect cytotoxic T lymphocytes and natural killer cell anti-tumor antigen immune response and regulated using dimeric FK506 and dimeric FK506 analogs. The tumor cell mRNA may be, for example, mRNA from a prostate tumor cell.

In some embodiments, the transduced antigen-presenting cell may be loaded by pulsing with tumor cell lysates. The pulsed transduced antigen-presenting cells are administered to an animal to effect cytotoxic T lymphocytes and natural killer cell anti-tumor antigen immune response and regulated using dimeric FK506 and dimeric FK506 analogs. The tumor cell lysate may be, for example, a prostate tumor cell lysate.

### Immune Cells and Cytotoxic T Lymphocyte Response

T-lymphocytes may be activated by contact with the antigen-presenting cell that comprises the expression vector discussed herein, where the antigen-presenting cell has been challenged, transfected, pulsed, or electrofused with an antigen.

T cells express a unique antigen binding receptor on their membrane (T-cell receptor), which can only recognize antigen in association with major histocompatibility complex (MHC) molecules on the surface of other cells. There are several populations of T cells, such as T helper cells and T cytotoxic cells. T helper cells and T cytotoxic cells are primarily distinguished by their display of the membrane bound glycoproteins CD4 and CD8, respectively. T helper cells secret various lymphokines, that are crucial for the activation of B cells, T cytotoxic cells, macrophages and other cells of the immune system. In contrast, a naïve CD8 T cell that recognizes an antigen-MHC complex proliferates and differentiates into an effector cell called a cytotoxic CD8 T lymphocyte (CTL). CTLs eliminate cells of the body displaying antigen, such as virus-infected cells and tumor cells, by producing substances that result in cell lysis.

CTL activity can be assessed by methods discussed herein, for example. For example, CTLs may be assessed in freshly isolated peripheral blood mononuclear cells (PBMC), in a phytohaemaglutinin-stimulated IL-2 expanded cell line established from PBMC (Bernard et al., AIDS, 12(16):2125-2139, 1998) or by T cells isolated from a previously immunized subject and restimulated for 6 days with DC infected with an adenovirus vector containing antigen using standard 4 hour 51Cr release microtoxicity assays. One type of assay uses cloned T-cells. Cloned T-cells have been tested for their ability to mediate both perforin and Fas ligand-dependent killing in redirected cytotoxicity assays (Simpson et al., Gastroenterology, 115(4):849-855, 1998). The cloned cytotoxic T lymphocytes displayed both Fas- and perforin-dependent killing. Recently, an in vitro dehydrogenase release assay has been developed that takes advantage of a new fluorescent amplification system (Page, B., et al., Anticancer Res. 1998 July-August; 18(4A):2313-6). This approach is sensitive, rapid, and reproducible and may be used advantageously for mixed lymphocyte reaction (MLR). It may easily be further automated for large-scale cytotoxicity testing using cell membrane integrity, and is thus considered. In another fluorometric assay developed for detecting cell-mediated cytotoxicity, the fluorophore used is the non-toxic molecule AlamarBlue (Nociari et al., J. Immunol. Methods, 213(2): 157-167, 1998). The AlamarBlue is fluorescently quenched (i.e., low quantum yield) until mitochondrial reduction occurs, which then results in a dramatic increase in the AlamarBlue fluorescence intensity (i.e., increase in the quantum yield). This assay is reported to be extremely sensitive, specific and requires a significantly lower number of effector cells than the standard 51Cr release assay.

Other immune cells that can be induced by the present methods include natural killer cells (NK). NKs are lymphoid cells that lack antigen-specific receptors and are part of the innate immune system. Typically, infected cells are usually destroyed by T cells alerted by foreign particles bound to the cell surface MHC. However, virus-infected cells signal infection by expressing viral proteins that are recognized by antibodies. These cells can be killed by NKs. In tumor cells, if the tumor cells lose expression of MHC I molecules, then it may be susceptible to NKs.

### Formulations and Routes for Administration to Patients

Where clinical applications are contemplated, it will be necessary to prepare pharmaceutical compositions—expression constructs, expression vectors, fused proteins, transduced cells, activated DCs, transduced and loaded DCs—in a form appropriate for the intended application. Generally, this will entail preparing compositions that are essentially free of pyrogens, as well as other impurities that could be harmful to humans or animals.

One may generally desire to employ appropriate salts and buffers to render delivery vectors stable and allow for uptake by target cells. Buffers also may be employed when recombinant cells are introduced into a patient. Aqueous compositions comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

The active compositions may include classic pharmaceutical preparations. Administration of these compositions will be via any common route so long as the target tissue is available via that route. This includes, for example, oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, discussed herein.

The pharmaceutical forms suitable for injectable use include sterile aqueous solutions or dispersions and sterile powders for the extemporaneous preparation of sterile injectable solutions or dispersions. In all cases the form is sterile and is be fluid to the extent that easy syringability exists. It is stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In certain examples, isotonic agents, for example, sugars or sodium chloride may be included. Prolonged absorption of the injectable compositions can be brought about by the use in the compositions of agents delaying absorption, for example, aluminum monostearate and gelatin.

For oral administration, the compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including, for example: gels, pastes, powders and slurries. The active ingredient may be added in a therapeutically effective amount to a paste dentifrice that may include, for example, water, binders, abrasives, flavoring agents, foaming agents, and humectants.

The compositions may be formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

The administration schedule may be determined as appropriate for the patient and may, for example, comprise a dosing schedule where the cells are administered at week 0, followed by induction by administration of the chemical inducer of dimerization, followed by administration of additional cells and inducer at 2 week intervals thereafter for a total of, for example, 2, 4, 6, 8, 10, 12, 14, 16, 18, 20, 22, 24, 26, 28, or 30 weeks.

Other dosing schedules include, for example, a schedule where one dose of the cells and one dose of the inducer are administered. In another example, the schedule may comprise administering the cells and the inducer are administered at week 0, followed by the administration of additional cells and inducer at 4 week intervals, for a total of, for example, 4, 8, 12, 16, 20, 24, 28, or 32 weeks.

Administration of a dose of cells may occur in one session, or in more than one session, but the term dose may refer to the total amount of cells administered before administration of the ligand.

If needed, the method may further include additional leukaphereses to obtain more cells to be used in treatment.

### Methods for Treating a Disease

The present methods also encompass methods of treatment or prevention of a disease caused by pathogenic microorganisms and/or a hyperproliferative disease.

Diseases may be treated or prevented include diseases caused by viruses, bacteria, yeast, parasites, protozoa, cancer cells and the like. The pharmaceutical composition (transduced DCs, expression vector, expression construct, etc.) may be used as a generalized immune enhancer (DC activating composition or system) and as such has utility in treating diseases. Exemplary diseases that can be treated and/or prevented include, but are not limited, to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states which may be treated or prevented using the pharmaceutical composition (transduced DCs, expression vector, expression construct, etc.) include but are not limited to preneoplastic or hyperplastic states such as colon polyps, Crohn's disease, ulcerative colitis, breast lesions and the like.

Cancers, including solid tumors, which may be treated using the pharmaceutical composition include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Other hyperproliferative diseases, including solid tumors, that may be treated using DC activation system presented herein include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis. In the method of treatment, the administration of the pharmaceutical composition (expression construct, expression vector, fused protein, transduced cells, activated DCs, transduced and loaded DCs) may be for either “prophylactic” or “therapeutic” purpose. When provided prophylactically, the pharmaceutical composition is provided in advance of any symptom. The prophylactic administration of pharmaceutical composition serves to prevent or ameliorate any subsequent infection or disease. When provided therapeutically, the pharmaceutical composition is provided at or after the onset of a symptom of infection or disease. Thus the compositions presented herein may be provided either prior to the anticipated exposure to a disease-causing agent or disease state or after the initiation of the infection or disease.

Solid tumors from any tissue or organ may be treated using the present methods, including, for example, any tumor expressing PSA, for example, PSMA, in the vasculature, for example, solid tumors present in, for example, lungs, bone, liver, prostate, or brain, and also, for example, in breast, ovary, bowel, testes, colon, pancreas, kidney, bladder, neuroendocrine system, soft tissue, boney mass, and lymphatic system. Other solid tumors that may be treated include, for example, glioblastoma, and malignant myeloma.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition would be the amount that achieves this selected result of enhancing the immune response, and such an amount could be determined. For example, an effective amount of for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

A. Genetic Based Therapies

In certain embodiments, a cell is provided with an expression construct capable of providing a co-stimulatory polypeptide, such as CD40 to the cell, such as an antigen-presenting cell and activating CD40. The lengthy discussion of expression vectors and the genetic elements employed therein is incorporated into this section by reference. In certain examples, the expression vectors may be viral vectors, such as adenovirus, adeno-associated virus, herpes virus, vaccinia virus and retrovirus. In another example, the vector may be a lysosomal-encapsulated expression vector. Gene delivery may be performed in both in vivo and ex vivo situations. For viral vectors, one generally will prepare a viral vector stock. Examples of viral vector-mediated gene delivery ex vivo and in vivo are presented in the present application. For in vivo delivery, depending on the kind of virus and the titer attainable, one will deliver, for example, about 1, 2, 3, 4, 5, 6, 7, 8, or 9×10⁴, 1, 2, 3, 4, 5, 6, 7, 8, or 9×10⁵, 1, 2, 3, 4, 5, 6, 7, 8, or 9×10⁶, 1, 2, 3, 4, 5, 6, 7, 8, or 9×10⁷, 1, 2, 3, 4, 5, 6, 7, 8, or 9×10⁸, 1, 2, 3, 4, 5, 6, 7, 8, or 9×10⁹, 1, 2, 3, 4, 5, 6, 7, 8, or 9×10¹⁹, 1, 2, 3, 4, 5, 6, 7, 8, or 9×10¹¹ or 1, 2, 3, 4, 5, 6, 7, 8, or 9×10¹² infectious particles to the patient. Similar figures may be extrapolated for liposomal or other non-viral formulations by comparing relative uptake efficiencies. Formulation as a pharmaceutically acceptable composition is discussed below. The multimeric ligand, such as, for example, AP1903, may be delivered, for example at doses of about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight.

B. Cell Based Therapy

Another therapy that is contemplated is the administration of transduced antigen-presenting cells. The antigen-presenting cells may be transduced in vitro. Formulation as a pharmaceutically acceptable composition is discussed herein.

In cell based therapies, the transduced antigen-presenting cells may be, for example, transfected with target antigen nucleic acids, such as mRNA or DNA or proteins; pulsed with cell lysates, proteins or nucleic acids; or electrofused with cells. The cells, proteins, cell lysates, or nucleic acid may derive from cells, such as tumor cells or other pathogenic microorganism, for example, viruses, bacteria, protozoa, etc.

C. Combination Therapies

In order to increase the effectiveness of the expression vectors presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In further embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with the expression vector effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

In some embodiments, the chemotherapeutic agent may be Taxotere (docetaxel), or another taxane, such as, for example, cabazitaxel. The chemotherapeutic may be administered either before, during, or after treatment with the activated dendritic cell and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated dendritic cells. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of activated dendritic cells.

Administration of a chemotherapeutic agent may comprise the administration of more than one chemotherapeutic agent. For example, cisplatin may be administered in addition to Taxotere or other taxane, such as, for example, cabazitaxel.

### Optimized and Personalized Therapeutic Treatment

Treatment for solid tumor cancers, including, for example, prostate cancer, may be optimized by determining the concentration of IL-6, IL6-sR, or VCAM-1 during the course of treatment. IL-6 refers to interleukin 6. IL-6sR refers to the IL-6 soluble receptor, the levels of which often correlate closely with levels of IL-6. VCAM-1 refers to vascular cell adhesion molecule. Different patients having different stages or types of cancer, may react differently to various therapies. The response to treatment may be monitored by following the IL-6, IL-6sR, or VCAM-1 concentrations or levels in various body fluids or tissues. The determination of the concentration, level, or amount of a polypeptide, such as, IL-6, IL-6sR, or VCAM-1, may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating solid tumor cancers by allowing a clinician to track a biomarker, such as, for example, IL-6, IL-6sR, or VCAM-1, and determine whether a subsequent dose of a drug or vaccine for administration to a subject may be maintained, reduced or increased, and to determine the timing for the subsequent dose.

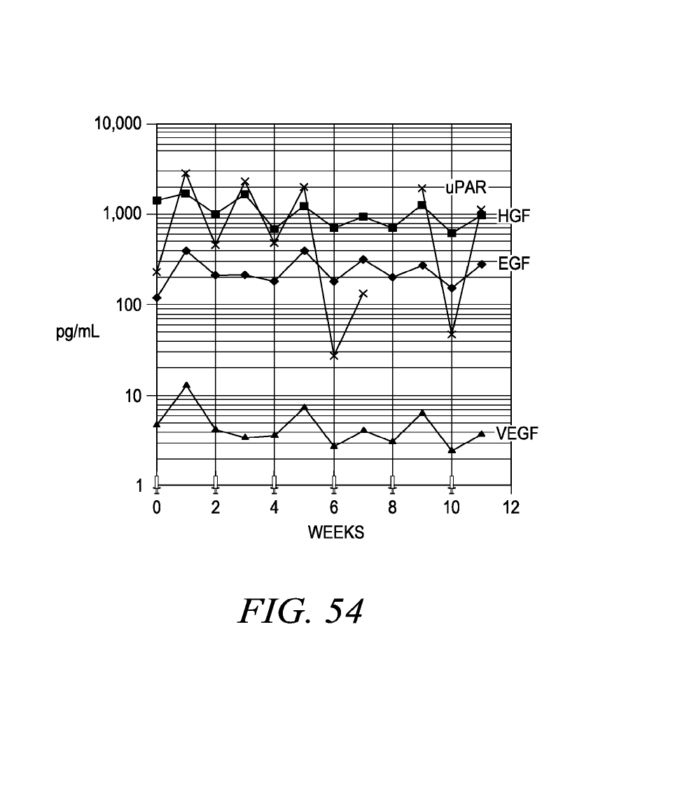

Treatment for solid tumor cancers, including, for example, prostate cancer, may also be optimized by determining the concentration of urokinase-type plasminogen activator receptor (uPAR), hepatocyte growth factor (HGF), epidermal growth factor (EGF), or vascular endothelial growth factor (VEGF) during the course of treatment. Different patients having different stages or types of cancer, may react differently to various therapies. FIG. 54 depicts the levels of uPAR, HGF, EGF, and VEGF over the course of treatment for subject 1003. Subject 1003 shows systemic perturbation of hypoxic factors in serum, which may indicate a positive response to treatment. Without limiting the interpretation of this observation, this may indicate the secretion of hypoxic factors by tumors in response to treatment. Thus, the response to treatment may be monitored, for example, by following the uPAR, HGF, EGF, or VEGF concentrations or levels in various body fluids or tissues. The determination of the concentration, level, or amount of a polypeptide, such as, uPAR, HGF, EGF, or VEGF may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating solid tumor cancers by allowing a clinician to track a biomarker, such as, for example, uPAR, HGF, EGF, or VEGF, and determine whether a subsequent dose of a drug or vaccine for administration to a subject may be maintained, reduced or increased, and to determine the timing for the subsequent dose.

For example, it has been determined that amount or concentration of certain biomarkers changes during the course of treatment of solid tumors. Predetermined target levels of such biomarkers, or biomarker thresholds may be identified in normal subject, are provided, which allow a clinician to determine whether a subsequent dose of a drug administered to a subject in need thereof, such as a subject with a solid tumor, such as, for example, a prostate tumor, may be increased, decreased or maintained. A clinician can make such a determination based on whether the presence, absence or amount of a biomarker is below, above or about the same as a biomarker threshold, respectively, in certain embodiments.

For example, determining that an over-represented biomarker level is significantly reduced and/or that an under-represented biomarker level is significantly increased after drug treatment or vaccination provides an indication to a clinician that an administered drug is exerting a therapeutic effect. By “level” is meant the concentration of the biomarker in a fluid or tissue, or the absolute amount in a tissue. Based on such a biomarker determination, a clinician could make a decision to maintain a subsequent dose of the drug or raise or lower the subsequent dose, including modifying the timing of administration. The term “drug” includes traditional pharmaceuticals, such as small molecules, as well as biologics, such as nucleic acids, antibodies, proteins, polypeptides, modified cells and the like. In another example, determining that an over-represented biomarker level is not significantly reduced and/or that an under-represented biomarker level is not significantly increased provides an indication to a clinician that an administered drug is not significantly exerting a therapeutic effect. Based on such a biomarker determination, a clinician could make a decision to increase a subsequent dose of the drug. Given that drugs can be toxic to a subject and exert side effects, methods provided herein optimize therapeutic approaches as they provide the clinician with the ability to “dial in” an efficacious dosage of a drug and minimize side effects. In specific examples, methods provided herein allow a clinician to “dial up” the dose of a drug to an therapeutically efficacious level, where the dialed up dosage is below a toxic threshold level. Accordingly, treatment methods discussed herein enhance efficacy and reduce the likelihood of toxic side effects.

Cytokines are a large and diverse family of polypeptide regulators produced widely throughout the body by cells of diverse origin. Cytokines are small secreted proteins, including peptides and glycoproteins, which mediate and regulate immunity, inflammation, and hematopoiesis. They are produced de novo in response to an immune stimulus. Cytokines generally (although not always) act over short distances and short time spans and at low concentration. They generally act by binding to specific membrane receptors, which then signal the cell via second messengers, often tyrosine kinases, to alter cell behavior (e.g., gene expression). Responses to cytokines include, for example, increasing or decreasing expression of membrane proteins (including cytokine receptors), proliferation, and secretion of effector molecules.

The term “cytokine” is a general description of a large family of proteins and glycoproteins. Other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on cells that secrete them (autocrine action), on nearby cells (paracrine action), or in some instances on distant cells (endocrine action).

Examples of cytokines include, without limitation, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 and the like), interferons (e.g., IFN-beta, IFN-gamma and the like), tumor necrosis factors (e.g., TNF-alpha, TNF-beta and the like), lymphokines, monokines and chemokines; growth factors (e.g., transforming growth factors (e.g., TGF-alpha, TGF-beta and the like)); colony-stimulating factors (e.g. GM-CSF, granulocyte colony-simulating factor (G-CSF) etc.); and the like.

A cytokine often acts via a cell-surface receptor counterpart. Subsequent cascades of intracellular signaling then alter cell functions. This signaling may include upregulation and/or downregulation of several genes and their transcription factors, resulting in the production of other cytokines, an increase in the number of surface receptors for other molecules, or the suppression of their own effect by feedback inhibition.

VCAM-1 (vascular cell adhesion molecule-1, also called CD106), contains six or seven immunoglobulin domains and is expressed on both large and small vessels only after the endothelial cells are stimulated by cytokines. Thus, VCAM-1 expression is a marker for cytokine expression.

Cytokines may be detected as full-length (e.g., whole) proteins, polypeptides, metabolites, messenger RNA (mRNA), complementary DNA (cDNA), and various intermediate products and fragments of the foregoing (e.g., cleavage products (e.g., peptides, mRNA fragments)). For example, IL-6 protein may be detected as the complete, full-length molecule or as any fragment large enough to provide varying levels of positive identification. Such a fragment may comprise amino acids numbering less than 10, from 10 to 20, from 20 to 50, from 50 to 100, from 100 to 150, from 150 to 200 and above. Likewise, VCAM-1 protein can be detected as the complete, full-length amino acid molecule or as any fragment large enough to provide varying levels of positive identification. Such a fragment may comprise amino acids numbering less than 10, from 10 to 20, from 20 to 50, from 50 to 100, from 100 to 150 and above.

In certain embodiments, cytokine mRNA may be detected by targeting a complete sequence or any sufficient fragment for specific detection. A mRNA fragment may include fewer than 10 nucleotides or any larger number. A fragment may comprise the 3′ end of the mRNA strand with any portion of the strand, the 5′ end with any portion of the strand, and any center portion of the strand.

The amino acid and nucleic acid sequences for IL-6, IL-6sR, and VCAM-1 are provided as SEQ ID NOs: 11-16.

Detection may be performed using any suitable method, including, without limitation, mass spectrometry (e.g., matrix-assisted laser desorption ionization mass spectrometry (MALDI-MS), electrospray mass spectrometry (ES-MS)), electrophoresis (e.g., capillary electrophoresis), high performance liquid chromatography (HPLC), nucleic acid affinity (e.g., hybridization), amplification and detection (e.g., real-time or reverse-transcriptase polymerase chain reaction (RT-PCR)), and antibody assays (e.g., antibody array, enzyme-linked immunosorbant assay (ELISA)). Examples of IL-6 and other cytokine assays include, for example, those provided by Millipore, Inc., (Milliplex Human Cytokine/Chemokine Panel). Examples of IL6-sR assays include, for example, those provided by Invitrogen, Inc. (Soluble IL-6R: (Invitrogen Luminex® Bead-based assay)). Examples of VCAM-1 assays include, for example, those provided by R & D Systems ((CD106) ELISA development Kit, DuoSet from R&D Systems (#DY809)).

### Sources of Biomarkers

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

A fluid or tissue sample often is obtained from a subject for determining presence, absence or amount of biomarker ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, tumor or area surrounding a tumor, and the like, in some embodiments. A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerebrospinal fluid, spinal fluid, lavage fluid (e.g., bronchoalveolar, gastric, peritoneal, ductal, ear, arthroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from region of muscle wasting and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection after a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point).

### Biomarker Detection

The presence, absence or amount of one or more biomarkers may be determined by any suitable method known in the art, and non-limiting determination methods are discussed herein.

Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

Biomarker detected in an assay can be full-length biomarker, a biomarker fragment, an altered or modified biomarker (e.g., biomarker derivative, biomarker metabolite), or sum of two or more of the foregoing, for example. Modified biomarkers often have substantial sequence identity to a biomarker discussed herein. For example, percent identity between a modified biomarker and a biomarker discussed herein may be in the range of 15-20%, 20-30%, 31-40%, 41-50%, 51-60%, 61-70%, 71-80%, 81-90% and 91-100%, (e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 percent identity). A modified biomarker often has a sequence (e.g., amino acid sequence or nucleotide sequence) that is 90% or more identical to a sequence of a biomarker discussed herein. Percent sequence identity can be determined using alignment methods known in the art.

Detection of biomarkers may be performed using any suitable method known in the art, including, without limitation, mass spectrometry, antibody assay (e.g., ELISA), nucleic acid affinity, microarray hybridization, Northern blot, reverse PCR and RT-PCR. For example, RNA purity and concentration may be determined spectrophotometrically (260/280>1.9) on a Nanodrop 1000. RNA quality may be assessed using methods known in the art (e.g., Agilent 2100 Bioanalyzer; RNA 6000 Nano LabChip® and the like).

### Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift sharply in response to a drug, (iii) low levels or high levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker. An amount of a biomarker can be a mean, median, nominal, range, interval, maximum, minimum, or relative amount, in some embodiments. An amount of a biomarker can be expressed with or without a measurement error window in certain embodiments. An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example. Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature. For example, an IL-6 or IL6-sR level of about 8-fold more than a normal level, or greater (e.g. about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold more than a normal level) may indicate that the dosage of the drug or the frequency of administration may be increased in a subsequent administration.

The term “dosage” is meant to include both the amount of the dose and the frequency of administration, such as, for example, the timing of the next dose. An IL-6 or IL-6sR level less than about 8-fold more than a normal level (e.g. about 7, 6, 5, 4, 3, 2, or 1-fold more than a normal level, or less than or equal to a normal level) may indicate that the dosage may be maintained or decreased in a subsequent administration. A VCAM-1 level of about 8 fold more than a normal level, or greater (e.g. e.g. about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold more than a normal level) may indicate that the dosage of the drug may be increased in a subsequent administration. A VCAM-1 level less than about 8-fold more than a normal level (e.g. about 7, 6, 5, 4, 3, 2, or 1-fold more than a normal level, or less than or equal to a normal level) may indicate that the dosage may be maintained or decreased in a subsequent administration. A normal level of IL-6, IL-6sR, or VCAM-1 may be assessed in a subject not diagnosed with a solid tumor or the type of solid tumor under treatment in a patient.

Other indications for adjusting or maintaining a drug dose include, for example, a perturbation in the concentration of an individual secreted factor, such as, for example, GM-CSF, MIP-1 alpha, MIP-1 beta, MCP-1, IFN-gamma, RANTES, EGF or HGF, or a perturbation in the mean concentration of a panel of secreted factors, such as two or more of the markers selected from the group consisting of GM-CSF, MIP-1 alpha, MIP-1 beta, MCP-1, IFN-gamma, RANTES, EGF and HGF. This perturbation may, for example, consist of an increase, or decrease, in the concentration of an individual secreted factor by at least 5%, 10%, 20%, 30%, 40%, 50%, 60%, 70%, 80%, 90%, or 100% or an increase or decrease in the mean relative change in serum concentration of a panel of secreted factors by at least 5%, 10%, 20%, 30%, 40%, 50%, 60%, 70%, 80%, 90%, or 100%. This increase may, or may not, be followed by a return to baseline serum concentrations before the next administration. The increase or decrease in the mean relative change in serum concentration may involve, for example, weighting the relative value of each of the factors in the panel. Also, the increase or decrease may involve, for example, weighting the relative value of each of the time points of collected data. The weighted value for each time point, or each factor may vary, depending on the state or the extent of the cancer, metastasis, or tumor burden. An indication for adjusting or maintaining the drug dose may include a perturbation in the concentration of an individual secreted factor or the mean concentration of a panel of secreted factors, after 1, 2, 3, 4, 5, 6, 7, 8, 9, or 10 or more administrations. For example, where it is observed that over the course of treatment, for example, 6 administrations of a drug or the vaccines or compositions discussed herein, that the concentration of an individual secreted factor or the mean concentration of a panel of secreted factors is perturbed after at least one administration, then this may be an indication to maintain, decrease, or increase the frequency of administration or the subsequent dosage, or it may be an indication to continue treatment by, for example, preparing additional drug, adenovirus vaccine, or adenovirus transfected or transduced cells.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker may be determined. Non-limiting examples of prescreens include identifying the presence or absence of a genetic marker (e.g., polymorphism, particular nucleotide sequence); identifying the presence, absence or amount of a particular metabolite. A prescreen result can be used by a clinician in combination with the presence, absence or amount of a biomarker to determine whether a subsequent drug dose may be adjusted or maintained.

### Antibodies and Small Molecules

In some embodiments, an antibody or small molecule is provided for use as a control or standard in an assay, or a therapeutic, for example. In some embodiments, an antibody or other small molecule configured to bind to a cytokine or cytokine receptor, including without limitation IL-6, IL-6sR, and alter the action of the cytokine, or it may be configured to bind to VCAM-1. In certain embodiments an antibody or other small molecule may bind to an mRNA structure encoding for a cytokine or receptor.

The term small molecule as used herein means an organic molecule of approximately 800 or fewer Daltons. In certain embodiments small molecules may diffuse across cell membranes to reach intercellular sites of action. In some embodiments a small molecule binds with high affinity to a biopolymer such as protein, nucleic acid, or polysaccharide and may sometimes alter the activity or function of the biopolymer. In various embodiments small molecules may be natural (such as secondary metabolites) or artificial (such as antiviral drugs); they may have a beneficial effect against a disease (such as drugs) or may be detrimental (such as teratogens and carcinogens). By way of non-limiting example, small molecules may include ribo- or deoxyribonucleotides, amino acids, monosaccharides and small oligomers such as dinucleotides, peptides such as the antioxidant glutathione, and disaccharides such as sucrose.

The term antibody as used herein is to be understood as meaning a gamma globulin protein found in blood or other bodily fluids of vertebrates, and used by the immune system to identify and neutralize foreign objects, such as bacteria and viruses. Antibodies typically include basic structural units of two large heavy chains and two small light chains.

Specific binding to an antibody requires an antibody that is selected for its affinity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with GM-CSF, TNF-alpha or NF-kappa-B modulating protein and not with other proteins. This selection may be achieved by subtracting out antibodies that cross-react with other molecules.

Methods as presented herein include without limitation the delivery of an effective amount of an activated cell, a nucleic acid. or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

### EXAMPLES

The examples set forth below illustrate certain embodiments and do not limit the technology.

### Example 1

### Materials and Methods

Discussed hereafter are materials and methods utilized in studies discussed in subsequent Examples.

Tumor Cell Lines and Peptides

NA-6-MeI, T2, SK-MeI-37 and LNCaP cell lines were purchased from the American Type Culture Collection (ATCC) (Manassas, Va.). HLA-A2-restricted peptides MAGE-3 p271-279 (FLWGPRALV) (SEQ ID NO: 19), influenza matrix (IM) p58-66 (GILGFVFTL) (SEQ ID NO: 20), and HIV-1 gag p77-85 (SLYNTVATL) (SEQ ID NO: 21) were used to analyze CD8+ T cell responses. In T helper cell polarization experiments, tetanus toxoid peptide TTp30 FNNFTVSFWLRVPKVSASHLE (SEQ ID NO: 22) was used. All peptides were synthesized by Genemed Synthesis Inc (San Francisco, Calif.), with an HPLC-determined purity of >95%.

Recombinant Adenovirus Encoding Human Inducible CD40

The human CD40 cytoplasmic domain was Pfu I polymerase (Stratagene, La Jolla, Calif.) amplified from human monocyte-derived DC cDNA using an Xho 1-flanked 5′ primer (5hCD40X), 5′-atatactcgagaaaaaggtggccaagaagccaacc-3′ (SEQ ID NO: 23), and a Sal I-flanked 3′ primer (3hCD40S), 5′-atatagtcgactcactgtctctcctgcactgagatg-3′(SEQ ID NO: 24). The PCR fragment was subcloned into Sal I-digested pSH1/M-FvFvls-E15 and sequenced to create pSH1/M-FvFvls-CD40-E. Inducible CD40 was subsequently subcloned into a non-replicating E1, E3-deleted Ad5/f35-based vector expressing the transgene under a cytomegalovirus early/immediate promoter. The iCD40-encoding sequence was confirmed by restriction digest and sequencing. Amplification, purification, and titration of all adenoviruses were carried out in the Viral Vector Core Facility of Baylor College of Medicine.

Western Blot

Total cellular extracts were prepared with RIPA buffer containing a protease inhibitor cocktail (Sigma-Aldrich, St. Louis, Mo.) and quantitated using a detergent-compatible protein concentration assay (Bio-Rad, Hercules, Calif.). 10-15 micrograms of total protein were routinely separated on 12% SDS-PAGE gels, and proteins were transferred to nitrocellulose membranes (Bio-Rad). Blots were hybridized with goat anti-CD40 (T-20, Santa Cruz Biotechnology, Santa Cruz, Calif.) and mouse anti-alpha-tubulin (Santa Cruz Biotechnology) Abs followed by donkey anti-goat and goat anti-mouse IgG-HRP (Santa Cruz Biotechnology), respectively. Blots were developed using the SuperSignal West Dura Stable substrate system (Pierce, Rockford, Ill.).

Generation and Stimulation of Human DCs

Peripheral blood mononuclear cells (PBMCs) from healthy donors were isolated by density centrifugation of heparinized blood on Lymphoprep (Nycomed, Oslo, Norway). PBMCs were washed with PBS, resuspended in CellGenix DC medium (Freiburg, Germany) and allowed to adhere in culture plates for 2 h at 37 degrees C. and 5% CO2. Nonadherent cells were removed by extensive washings, and adherent monocytes were cultured for 5 days in the presence of 500 U/ml hIL-4 and 800 U/ml hGM-CSF (R&D Systems, Minneapolis, Minn.). As assessed by morphology and FACS analysis, the resulting immature DCs (imDCs) were MHC-class I, Ilhi, and expressed CD40Io, CD80Io, CD83Io, CD86Io. The imDCs were CD14neg and contained <3% of contaminating CD3+ T, CD19+ B, and CD16+ NK cells.

Approximately 2×10⁶ cells/ml were cultured in a 24-well dish and transduced with adenoviruses at 10,000 viral particle (vp)/cell (˜160 MOI) for 90 min at 37 degrees C. and 5% CO₂. Immediately after transduction DCs were stimulated with MPL, FSL-1, Pam3CSK4 (InvivoGen, San Diego, Calif.), LPS (Sigma-Aldrich, St. Louis, Mo.), AP20187 (kind gift from ARIAD Pharmaceuticals, Cambridge, Mass.) or maturation cocktail (MC), containing 10 ng/ml TNF-alpha, 10 ng/ml IL-1 beta, 150 ng/ml IL-6 (R&D Systems, Minneapolis, Minn.) and 1 microgram/ml of PGE2 (Cayman Chemicals, Ann Arbor, Mich.). In T cell assays DCs were pulsed with 50 micrograms/ml of PSMA polypeptide or MAGE 3 peptide 24 hours before and after adenoviral transduction.

Surface Markers and Cytokine Production

Cell surface staining was done with fluorochrome-conjugated monoclonal antibodies (BD Biosciences, San Diego, Calif.). Cells were analyzed on a FACSCalibur cytometer (BD Biosciences, San Jose, Calif.). Cytokines were measured in culture supernatants using enzyme-linked immunosorbent assay kits for human IL-6 and IL-12p70 (BD Biosciences).

IFN-Gamma ELISPOT Assay

DCs from HLA-A2-positive healthy volunteers were pulsed with MAGE-3 A2.1 peptide (residues 271-279; FLWGPRALV) (SEQ ID NO: 19) on day 4 of culture, followed by transduction with Ad-iCD40 and stimulation with various stimuli on day 5. Autologous T cells were purified from PBMCs by negative selection (Miltenyi Biotec, Auburn, Calif.) and mixed with DCs at DC:T cell ratio 1:3. Cells were incubated in complete RPMI with 20 U/ml hIL-2 (R&D Systems) and 25 micrograms/ml of MAGE 3 A2.1 peptide. T cells were restimulated at day 7 and assayed at day 14 of culture.

ELISPOT Quantitation

Flat-bottom, 96-well nitrocellulose plates (MultiScreen-HA; Millipore, Bedford, Mass.) were coated with IFN-gamma mAb (2 μg/ml, 1-D1K; Mabtech, Stockholm, Sweden) and incubated overnight at 4° C. After washings with PBS containing 0.05% TWEEN 20, plates were blocked with complete RPMI for 2 h at 37° C. A total of 1×10⁵ presensitized CD8+ T effector cells were added to each well and incubated for 20 h with 25 micrograms/ml peptides. Plates were then washed thoroughly with PBS containing 0.05% TWEEN 20, and anti-IFN-mAb (0.2 microg/ml, 7-B6-1-biotin; Mabtech) was added to each well. After incubation for 2 h at 37° C., plates were washed and developed with streptavidin-alkaline phosphatase (1 microg/ml; Mabtech) for 1 h at room temperature. After washing, substrate (3-amino-9-ethyl-carbazole; Sigma-Aldrich) was added and incubated for 5 min. Plate membranes displayed dark-pink spots that were scanned and analyzed by ZellNet Consulting Inc. (Fort Lee, N.J.).

Chromium Release Assay

Antigen recognition was assessed using target cells labeled with Chromium-51 (Amersham) for 1 hour at 37° C. and washed three times. Labeled target cells (5000 cells in 50 microliters) were then added to effector cells (100 microliters) at the indicated effector:target cell ratios in V-bottom microwell plates at the indicated concentrations. Supernatants were harvested after 6-h incubation at 37° C., and chromium release was measured using MicroBeta Trilux counter (Perkin-Elmer Inc, Torrance Calif.). Assays involving LNCaP cells were run for 18 hours. The percentage of specific lysis was calculated as: 100*[(experimental−spontaneous release)/(maximum−spontaneous release)].

Tetramer Staining

HLA-A2 tetramers assembled with MAGE-3.A2 peptide (FLWGPRALV) (SEQ ID NO: 19) were obtained from Baylor College of Medicine Tetramer Core Facility (Houston, Tex.). Presensitized CD8+ T cells in 50 μl of PBS containing 0.5% FCS were stained with PE-labeled tetramer for 15 min on ice before addition of FITC-CD8 mAb (BD Biosciences). After washing, results were analyzed by flow cytometry.

Polarization of Naïve T Helper Cells

Naïve CD4+CD45RA+T-cells from HLA-DR11.5-positive donors (genotyped using FASTYPE HLA-DNA SSP typing kit; BioSynthesis, Lewisville, Tex.) were isolated by negative selection using naïve CD4+ T cell isolation kit (Miltenyi Biotec, Auburn, Calif.). T cells were stimulated with autologous DCs pulsed with tetanus toxoid (5 FU/ml) and stimulated with various stimuli at a stimulator to responder ratio of 1:10. After 7 days, T cells were restimulated with autologous DCs pulsed with the HLA-DR11.5-restricted helper peptide TTp30 and transduced with adenovector Ad-iCD40. Cells were stained with PE-anti-CD4 Ab (BD Biosciences), fixed and permeabilized using BD Cytofix/Cytoperm kit (BD Biosciences), then stained with hIFN-gamma mAb (eBioscience, San Diego, Calif.) and analyzed by flow cytometry. Supernatants were analyzed using human TH1/TH2 BD Cytometric Bead Array Flex Set on BD FACSArray Bioanalyzer (BD Biosciences).

PSMA Protein Purification

The baculovirus transfer vector, pAcGP67A (BD Biosciences) containing the cDNA of extracellular portion of PSMA (residues 44-750) was kindly provided by Dr Pamela J. Bjorkman (Howard Hughes Medical Institute, California Institute of Technology, Pasadena, Calif.). PSMA was fused with a hydrophobic secretion signal, Factor Xa cleavage site, and N-terminal 6×-His affinity tag (SEQ ID NO: 33). High titer baculovirus was produced by the Baculovirus/Monoclonal antibody core facility of Baylor College of Medicine. PSMA protein was produced in High 5 cells infected with recombinant virus, and protein was purified from cell supernatants using Ni-NTA affinity columns (Qiagen, Chatsworth, Calif.) as previously discussed (Cisco R M, Abdel-Wahab Z, Dannull J, et al. Induction of human dendritic cell maturation using transfection with RNA encoding a dominant positive toll-like receptor 4. J Immunol. 2004; 172:7162-7168). After purification the ˜100 kDa solitary band of PSMA polypeptide protein was detected by silver staining of acrylamide gels.

### Secreted Alkaline Phosphatase (SEAP) Assays

Reporters assays were conducted in human Jurkat-TAg (T cells) or 293 (kidney embryonic epithelial) cells or murine RAW264.7 (macrophage) cells. Jurkat-TAg cells (107) in log-phase growth were electroporated (950 mF, 250 V) with 2 mg expression plasmid and 2 mg of reporter plasmid NF-kB-SEAP or IFNb-TA-SEAP (see above). 293 or RAW264.7 cells (˜2×10⁵ cells per 35-mm dish) in log phase were transfected with 6 ml of FuGENE-6 in growth media. After 24 hr, transformed cells were stimulated with CID. After an additional 20 h, supernatants were assayed for SEAP activity as discussed previously (Spencer, D. M., et al., Science 262, 1019-1024 (1993)).

### Tissue Culture

Jurkat-TAg and RAW264.7 cells were grown in RPMI 1640 medium, 10% fetal bovine serum (FBS), 10 mM HEPES (pH 7.14), penicillin (100 U/ml) and streptomycin (100 mg/ml). 293 cells were grown in Dulbecco's modified Eagle's medium, 10% FBS, and pen-strep.

### Data Analysis

Results are expressed as the mean±standard error. Sample size was determined with a power of 0.8, with a one-sided alpha-level of 0.05. Differences between experimental groups were determined by the Student t test.

### Constructs

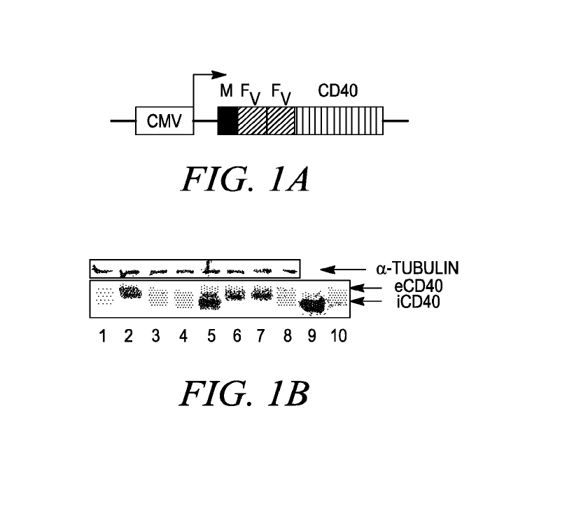

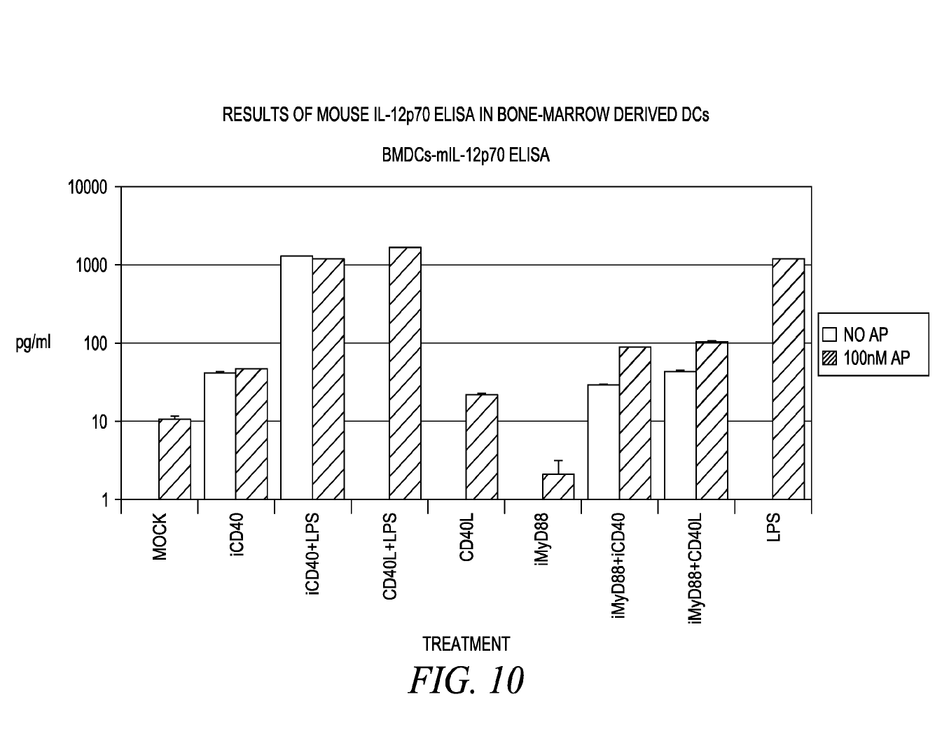

An inducible CD40 receptor based on chemical-induced dimerization (CID) and patterned after endogenous CD40 activation was produced to specifically target DCs (FIG. 1A). The recombinant CD40 receptor, termed iCD40, was engineered by rt-PCR amplifying the 228 bp CD40 cytoplasmic signaling domain from purified murine bone marrow-derived DCs (>95% CD11c+) and sub-cloning the resulting DNA fragment either downstream (i.e., M-FvFvlsCD40-E) or upstream (M-CD40-FvFvls-E) of tandem copies of the dimerizing drug binding domain, FKBP12(V₃₆) (FIG. 1B). Membrane localization was achieved with a myristoylation-targeting domain (M) and an HA epitope (E) tag was present for facile identification. To determine if the transcripts were capable of activating NFkappaB, the constructs were transiently transfected into Jurkat T cells and NFkappaB reporter assays were preformed in the presence of titrated dimerizer drug, AP20187 (FIG. 10). FIG. 10 showed that increasing levels of AP20187 resulted in significant upregulation of NF B transcriptional activity compared to the control vector, M-FvFvls-E, lacking CD40 sequence. Since the membrane-proximal version of iCD40, M-CD40-FvFvls-E, was less responsive to AP20187 in this assay system, the M-FvFvlsCD40-E construct was used in further studies, and heretofore referred to as “iCD40”. This decision was reinforced by the crystallographic structure of the CD40 cytoplasmic tail, which reveals a hairpin conformation that could be deleteriously altered by the fusion of a heterologous protein to its carboxyl-terminus (Ni 2000). The data also showed high drug dose suppression over 100 nM, likely due to the saturation of drug binding domains. This same phenomenon has been observed in other cell types expressing limiting levels of the iCD40 receptor. These results suggested that iCD40 was capable of inducing CID-dependent nuclear translocation of the NF B transcription factor.

Inducible iMyD88: Human TIR-containing inducible PRR adapter MyD88 (˜900-bp) was PCR-amplified from 293 cDNA using XhoI/SalI-linkered primers 5MyD88S (5′-acatcaactcgagatggctgcaggaggtcccgg-3′) (SEQ ID NO: 25) and 3MyD88S (5′-actcatagtcgaccagggacaaggccliggcaag-3′) (SEQ ID NO: 26) and subcloned into the XhoI and SalI sites of pSH1/M-Fv′-Fvls-E (Xie, X. et al., Cancer Res 61, 6795-804. (2001); Fan, L., et al., Human Gene Therapy 2273-2285 (1999)). to give pSH1/M-MyD88-Fv′-Fvls-E and pSH1/M-Fv′-Fvls-MyD88-E, respectively.

All inserts were confirmed by sequencing and for appropriate size by Western blot to the 3′ hemaglutinin (HA) epitope (E).

### Example 2

### Expression of iCD40 and Induction of DC Maturation

The human CD40 cytoplasmic signaling domain was cloned downstream of a myristoylation-targeting domain and two tandem domains (from human FKBP12(V₃₆), designated as “Fv′”), which bind dimerizing drug AP20187 (Clackson T, et al., Proc Natl Acad Sci USA. 1998; 95:10437-10442). Immature DCs expressed endogenous CD40, which was induced by LPS and CD40L. Transduction of Ad-iCD40 led to expression of the distinctly sized iCD40, which did not interfere with endogenous CD40 expression. Interestingly, the expression of iCD40 was also significantly enhanced by LPS stimulation, likely due to inducibility of ubiquitous transcription factors binding the “constitutive” CMV promoter.

One of the issues for the design of DC-based vaccines is to obtain fully matured and activated DCs, as maturation status is linked to the transition from a tolerogenic to an activating, immunogenic state (Steinman R M, et al., Annu Rev Immunol. 2003; 21:685-711; Hanks B A, et al., Nat. Med. 2005; 11:130-137; Banchereau J, et al., Nature. 1998; 392:245-252). It has been shown that expression of mouse variant Ad-iCD40 can induce murine bone marrow-derived DC maturation (Hanks B A, et al., Nat Med. 2005; 11:130-137). To determine whether humanized iCD40 affects the expression of maturation markers in DCs, DCs were transduced with Ad-iCD40 and the expression of maturation markers CD40, CD80, CD83, and CD86 were evaluated. TLR-4 signaling mediated by LPS or its derivative MPL is a potent inducer of DC maturation (Ismaili J, et al., J. Immunol. 2002; 168:926-932; Cisco R M, et al., J Immunol. 2004; 172:7162-7168; De Becker G, Moulin V, Pajak B, et al. The adjuvant monophosphoryl lipid A increases the function of antigen-presenting cells. Int Immunol. 2000; 12:807-815; Granucci F, et al., Microbes Infect. 1999; 1:1079-1084). It was also previously reported that endogenous CD40 signaling specifically up-regulates CD83 expression in human DCs (Megiovanni A M, et al., Eur Cytokine Netw. 2004; 15:126-134). Consistent with these previous reports, the expression levels of CD83 were upregulated upon Ad-iCD40 transduction, and CD83 expression was further upregulated following LPS or MPL addition.

### Example 3

### Inducible CD40 and MyD88 and Composite MyD88-CD40 Activate NF-kappaB in 293 Cells

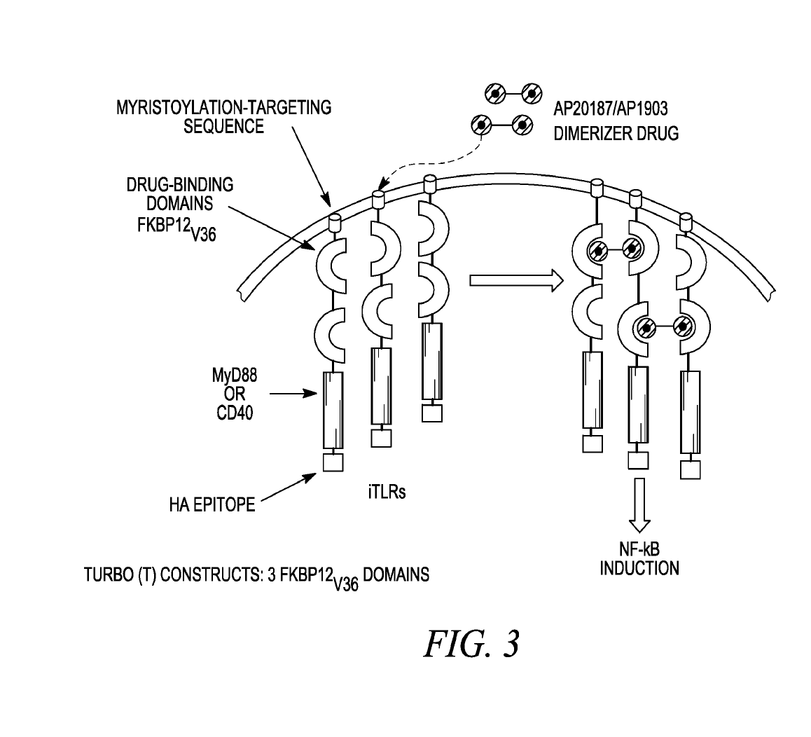

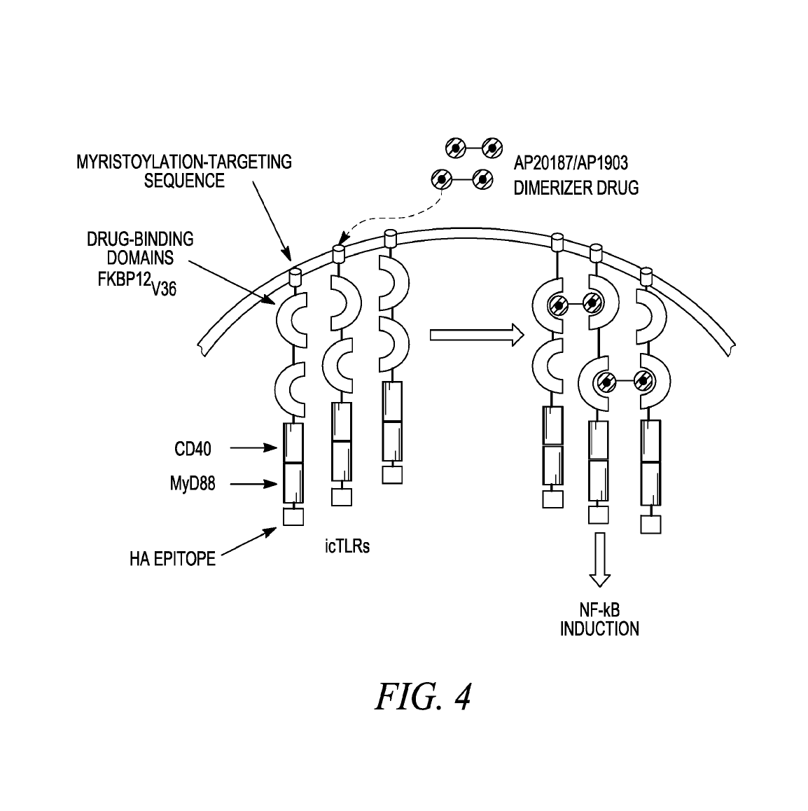

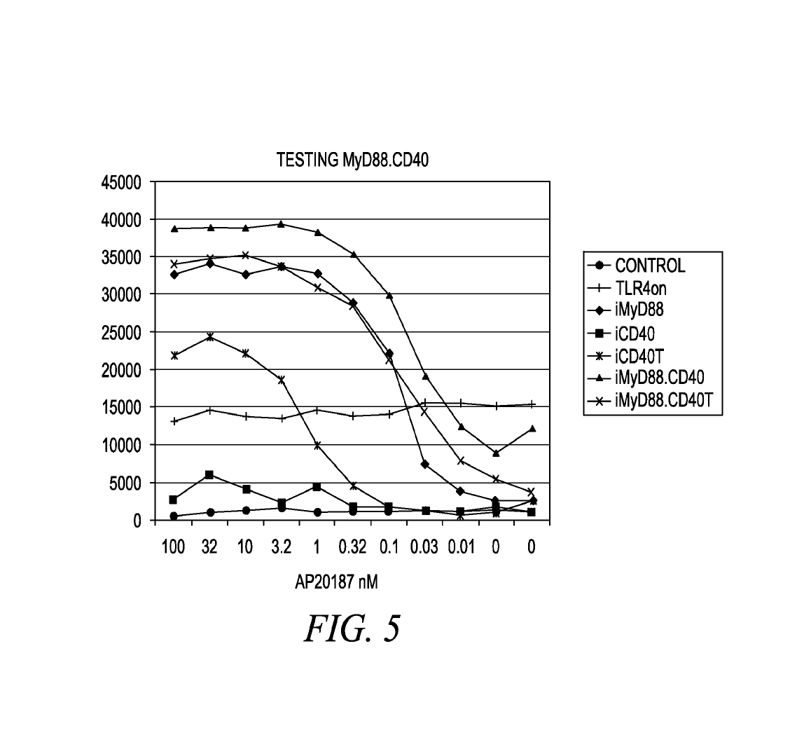

A set of constructs was designed to express inducible receptors, including a truncated version of MyD88, lacking the TIR domain. 293 cells were cotransfected with a NF-kappaB reporter and the SEAP reporter assay was performed essentially as discussed in Spencer, D. M., et al., Science 262, 1019-1024 (1993). The vector originally designed was pBJ5-M-MyD88L-Fv′Fvls-E. pShuttleX-M-MyD88L-Fv′Fvls was used to make the adenovirus. Both of these vectors were tested in SEAP assays. After 24 hours, AP20187 was added, and after 20 additional hours, the cell supernatant was tested for SEAP activity. Graphics relating to these chimeric constructs and activation are provided in FIGS. 3 and 4. The results are shown in FIG. 5.

### Constructs:

Control: Transfected with NF-kappaB Reporter Only.

TLR4 on: pShuttleX-CD4/TLR4-L3-E: CD4/TLR4L3-E is a constitutive version of TLR4 that contains the extracellular domain of mouse CD4 in tandem with the transmembrane and cytoplasmic domains of human TLR4 (as discussed in Medzhitov R, et al, Nature. 1997 Jul. 24; 388(6640):394-7.) followed by three 6-amino acid linkers and an HA epitope.

iMyD88: contains M-MyD88L-Fv′Fvls-E

iCD40: contains M-Fv′-Fvls-CD40-E

iCD40T: contains M-Fv′-Fv′-Fvls-CD40-E-iCD40T contains an extra Fv′ (FKBP with wobble at the valine)

iMyD88:CD40: contains M-MyD88L-CD40-Fv′Fvls-E

iMyD88:CD40T: contains M-MyD88LCD40-Fv′Fv′Fvls-E- contains an extra Fv′ compared to

iMyD88:CD40.

### Example 4

### Inducible CD40, CD40-MyD88, CD40-RIG-1, and CD40:NOD2

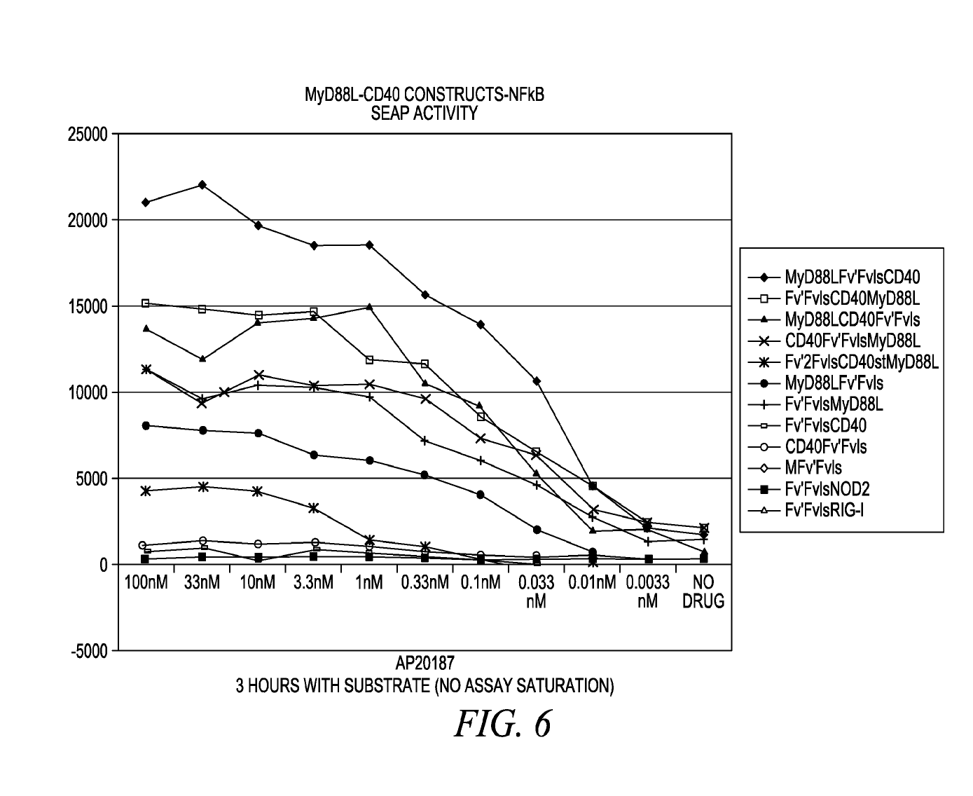

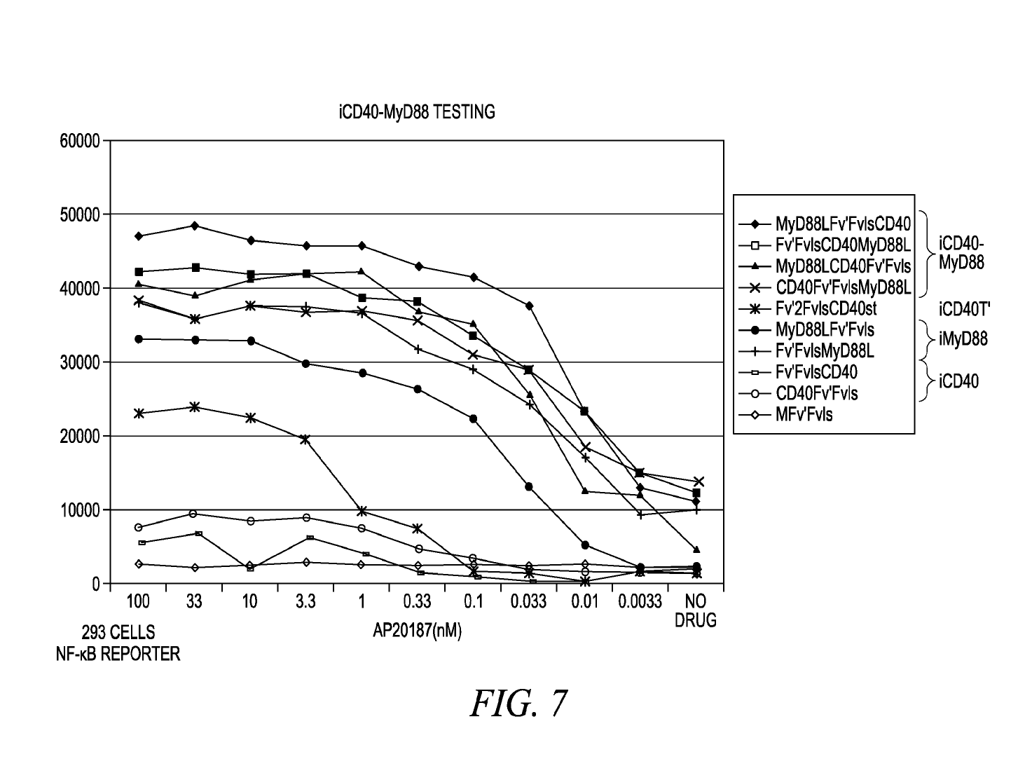

The following constructs were designed and assayed in the NF-kappaB reporter system. 293 cells were cotransfected with a NFkappaB reporter and one of the constructs. After 24 hours, AP20187 was added, and after an additional 3 hours (FIG. 6) or 22 hours (FIG. 7), the cell supernatant was tested for SEAP activity. About 20-24 hours after transfection, the cells were treated with dimer drug AP20187. About 20-24 hours following treatment with dimer drug, cells were treated with SEAP substrate 4-methylumbelliferyl phosphate (MUP). Following an overnight incubation (anywhere from 16-22 hrs), the SEAP counts were recorded on a FLUOStar OPTIMA machine.

MyD88LFv′FvlsCD40: was made in pBJ5 backbone with the myristoylation sequence upstream from MyD88L

Fv′FvlsCD40MyD88L: was made in pBJ5 backbone with the myristoylation sequence upstream from Fv′.

MyD88LCD40Fv′Fvls: was made in 2 vector backbone (pBJ5) with the myristoylation sequence upstream from the MyD88L.

CD40Fv′FvlsMyD88L: was made in pBJ5 backbone with the myristoylation sequence upstream from CD40.

Fv′2FvlsCD40stMyD88L: is a construct wherein a stop sequence after CD40 prevented MyD88L from being translated. Also named iCD40T′.

Fv′2Fvls includes 2 copies of Fv′, separated by a gtcgag sequence.

MyD88LFv′Fvls

Fv′FvlsMyD88L: was made in pBJ5 backbone with the myristoylation sequence upstream from the Fv′.

Fv′FvlsCD40: is available in pBJ5 and pShuttleX

CD40Fv′Fvls: is available in pBJ5 backbone with the myristoylation sequence upstream from the CD40.

MFv′Fvls:: is available in pBJ5 backbone with the myristoylation sequence indicated by the M. Fv″FvlsNOD2: pBJ5-Sn-Fv′Fvls-NOD2-E in pBJ5 backbone with no myristoylation sequence, contains 2 FKBPs followed by 2 CARD domains of NOD2 and the HA epitope.

Fv′FvlsRIG-1: pBJ5-Sn-Fv′Fvls-RIG-1-E in pBJ5 backbone with no myristoylation sequence, contains 2 FKBPs followed by 2 CARD domains of RIG-I and the HA epitope.

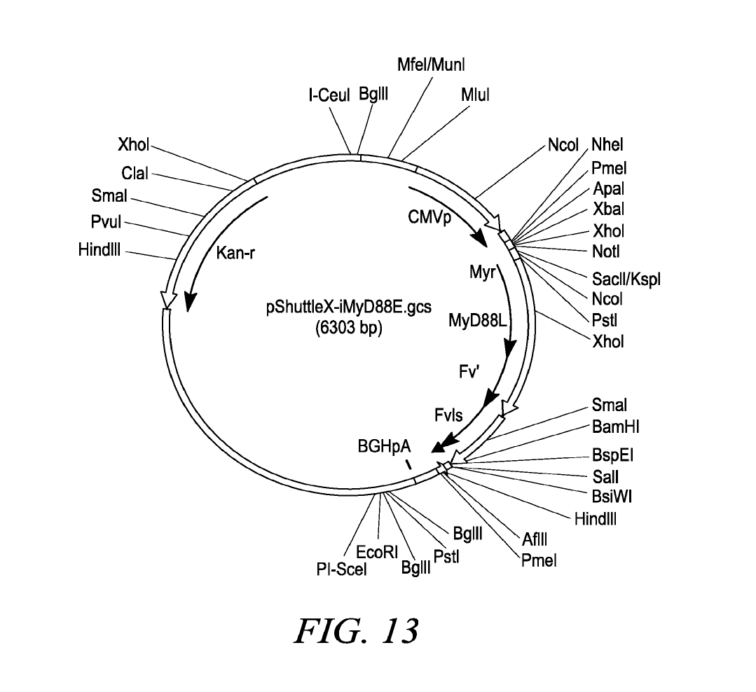

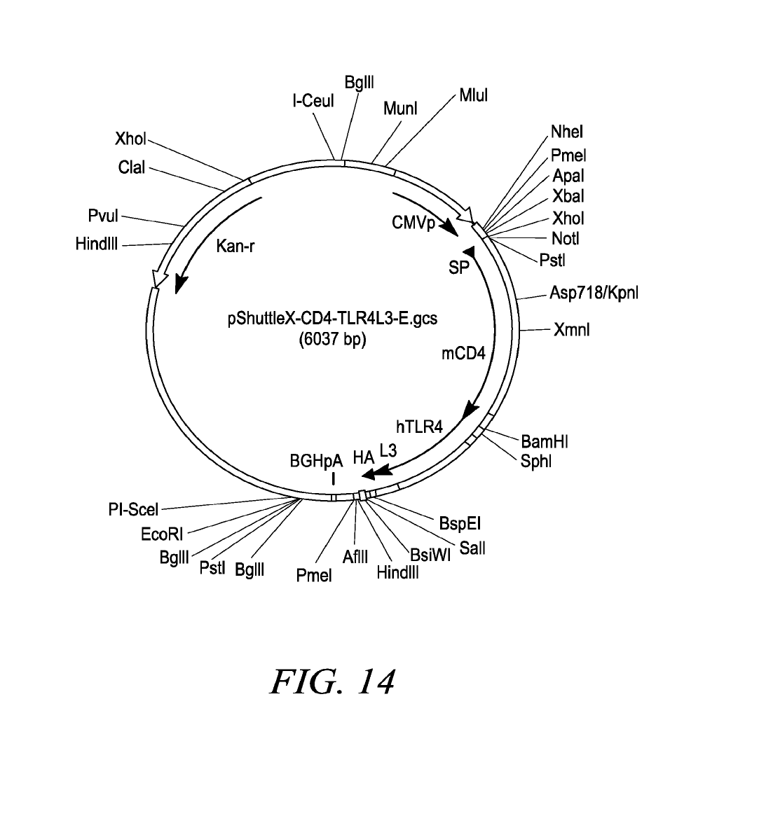

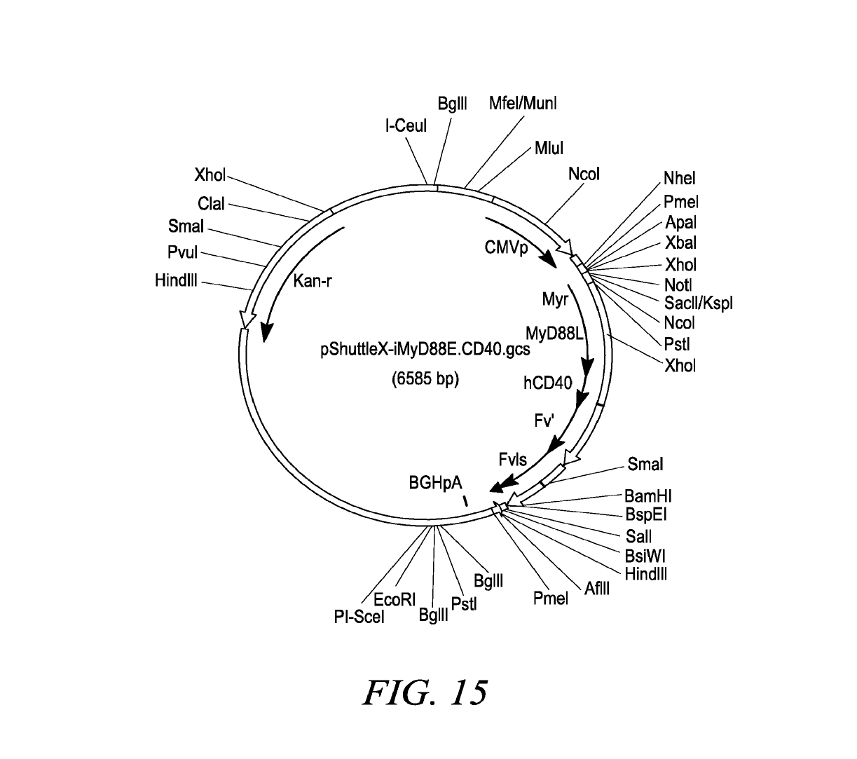

Examples of construct maps for pShuttleX versions used for Adenovirus production are presented in FIGS. 13, 14, and 15.

### Example 5

### MyD88L Adenoviral Transfection of 293T Cells Results in Protein Expression

The following pShuttleX constructs were constructed for adenovirus production:

pShuttleX-MyD88L-Fv′Fvls-E

pShuttleX-MyD88LCD40-Fv′Fvls-E

pShuttleX-CD4/TLR4-L3-E

L3 indicates three 6 amino acid linkers, having the DNA sequence:

(SEQ ID NO: 27)
GGAGGCGGAGGCAGCGGAGGTGGCGGTTCCGGAGGCGGAGGTTCT
Protein sequence:
(SEQ ID NO: 28)
GlyGlyGlyGlySerGlyGlyGlyGlySerGlyGlyGlyGlySer

E is an HA epitope.

Recombinant adenovirus was obtained using methods essentially as discussed in He, T. C., et al. (1998) Proc. Natl. Acad. Sci. USA 95(5):2509-14.

For each of the adenovirus assays, crude lysates from several virus plaques were assayed for protein expression by Western blotting. Viral particles were released from cell pellets supplied by the Vector Core at Baylor College of Medicine (world wide web address of http://vector.bcm.tmc.edu/) by freeze thawing pellets three times. 293T cells were plated at 1×10⁶ cells per well of a 6 well plate. 24 hours following culture, cells were washed twice with serum-free DMEM media with antibiotic, followed by the addition of 25 microliters or 100 microliters virus lysate to the cell monolayer in 500 microliters serum-free media. 2 hours later, 2.5 ml of serum-supplemented DMEM was added to each well of the 6-well plate.

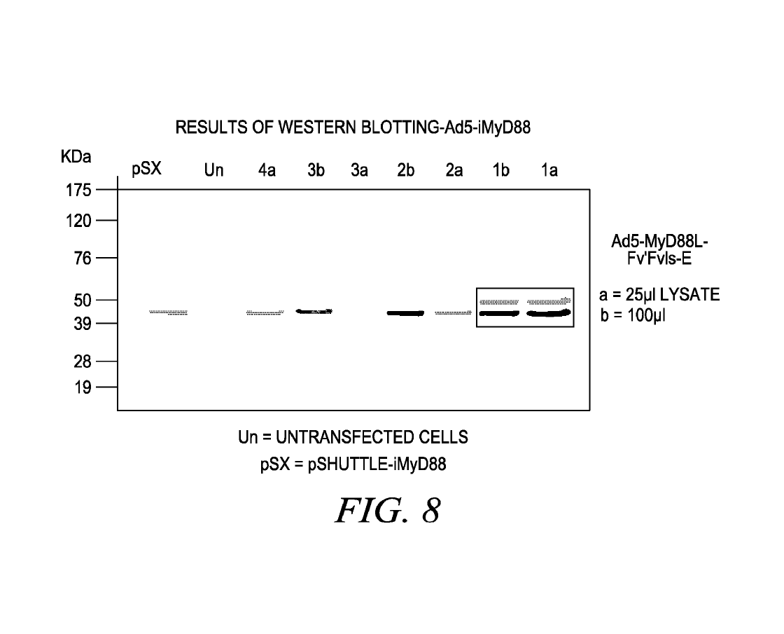

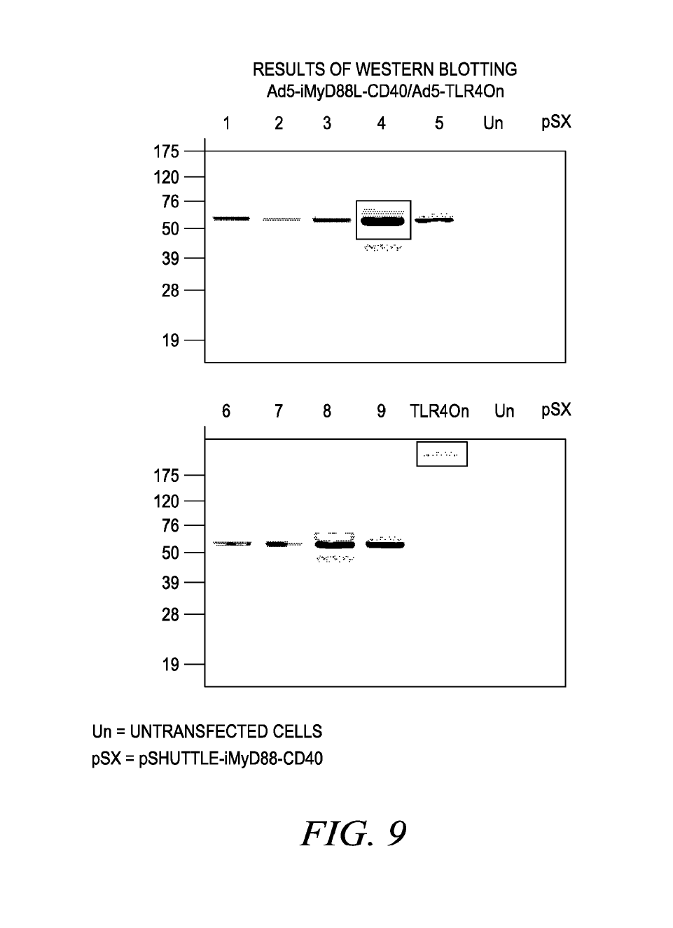

24-48 hours later, cells were harvested, washed twice with 1×PBS and resuspended in RIPA lysis buffer (containing 100 micromolar PMSF) (available from, for example, Millipore, or Thermo Scientific). Cells were incubated on ice for 30 minutes with mixing every 10 minutes, followed by a spin at 10,000 g for 15 minutes at 4° C. The supernatants were mixed with SDS Laemmli buffer plus beta-mercaptoethanol at a ratio of 1:2, incubated at 100 C for 10 minutes, loaded on a SDS gel, and probed on a nitrocellulose membrane using an antibody to the HA epitope. Results are shown in FIGS. 8 and 9. Remaining cell lysates were stored at −80 C for future use. The cells were transduced separately with each of the viruses, viz., Ad5-iMyD88 and Ad5-TLRon separately.

### Example 6

### IL-12p70 Expression in CD40 and MyD88L-Adenoviral Transduced Cells

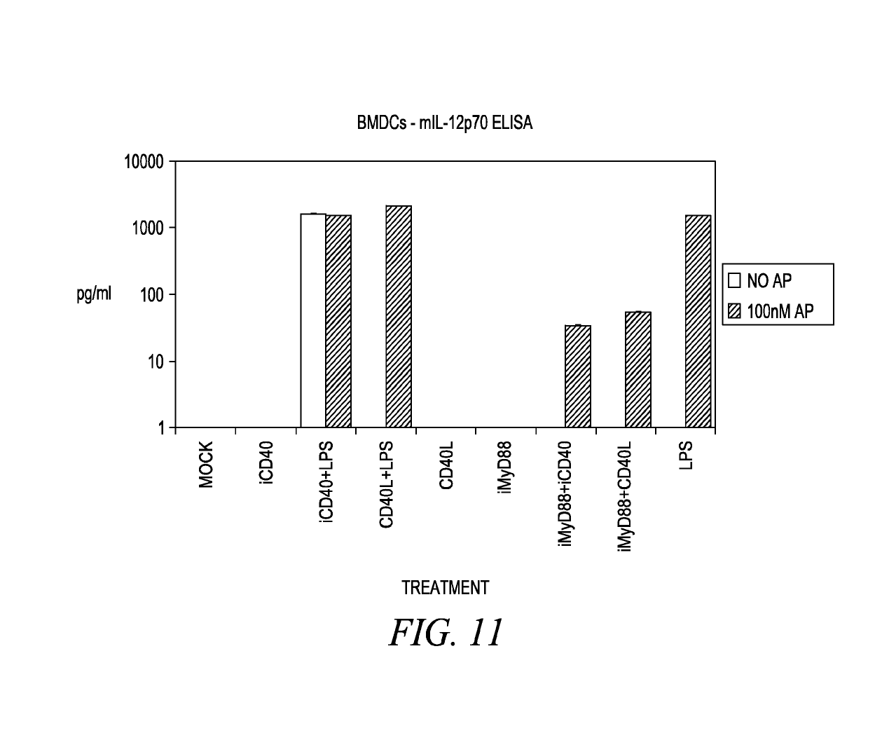

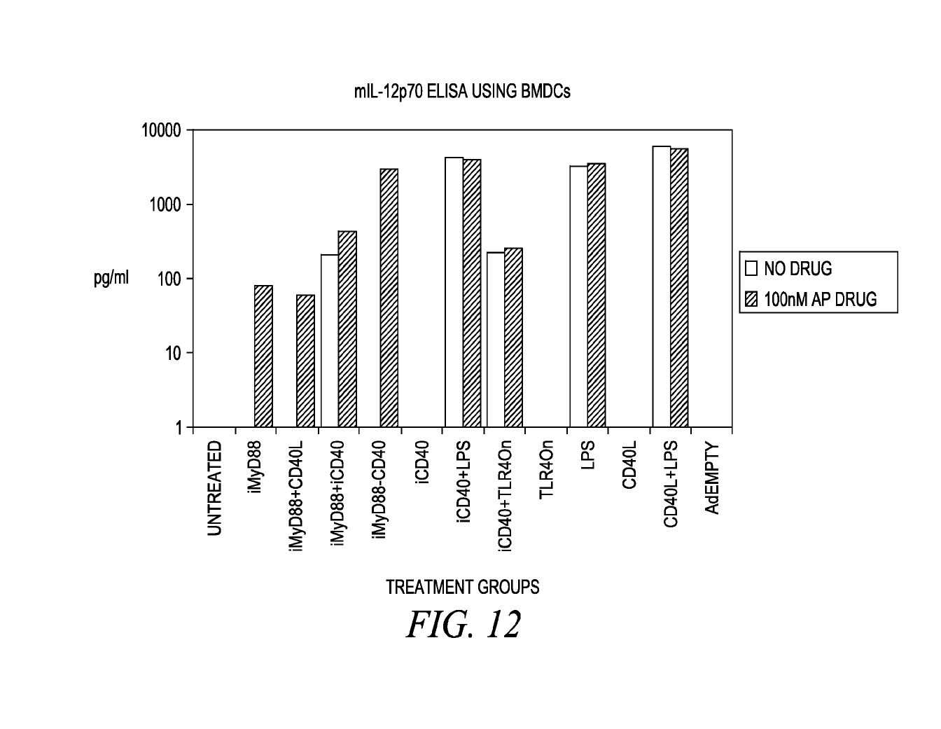

Bone marrow-derived dendritic cells (BMDCs) were plated at 0.25×10⁶ cells per well of a 48-well plate after washing twice with serum-free RPMI media with antibiotic. Cells were transduced with 6 microliters crude virus lysate in 125 microliters serum-free media. 2 hours later, 375 microliters of serum-supplemented RPMI was added to each well of the 48-well plate. 48 hours later, supernatants were harvested and analyzed using a mouse IL-12p70 ELISA kit (BD OptEIA (BD BioSciences, New Jersey). Duplicate assays were conducted for each sample, either with or without the addition of 100 nM AP21087. CD40-L is CD40 ligand, a TNF family member that binds to the CD40 receptor. LPS is lipopolysaccharide. The results are shown in FIG. 10. Results of a repeat of the assay are shown in FIG. 11, crude adenoviral lysate was added at 6.2 microliters per 0.25 million cells. FIG. 12 shows the results of an additional assay, where more viral lysate, 12.5 microliters per 0.25 million cells was used to infect the BMDCs.

### Example 7

### IL-12p70 Expression in MyD88L-Adenoviral Transduced Human Monocyte-Derived Dendritic Cells

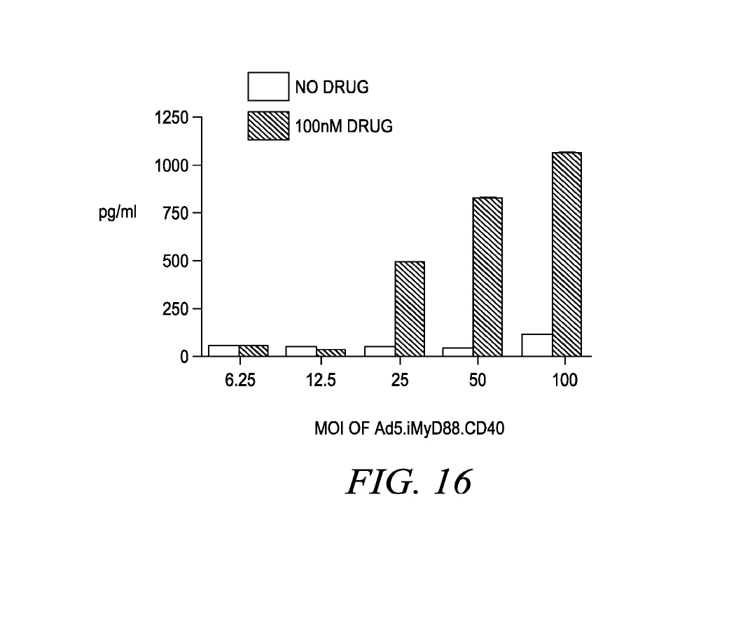

Immature human monocyte-derived dendritic cells (moDCs) were plated at 0.25×10⁶ cells per well of a 48-well plate after washing twice with serum-free RPMI media with antibiotic. Cells were transduced with different multiplicity of infections (MOI) of adenovirus AD5-iMyD88.CD40 and stimulated with 100 nM dimer drug AP20187. The virus used was an optimized version of the viral lysate used in the previous examples. 48 hours later, supernatants were harvested and assayed in an IL12p70 ELISA assay. FIG. 16 depicts the results of this titration.

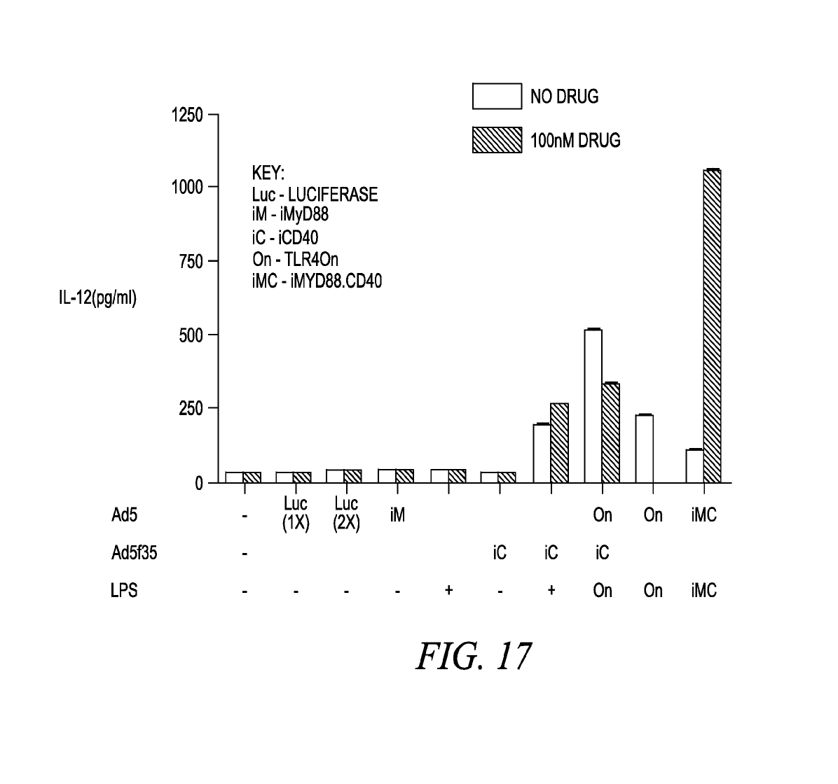

Immature human moDCs were plated at 0.25×10⁶ cells per well of a 48-well plate after washing twice with serum-free RPMI media with antibiotic. Cells were then transduced with either Ad5f35-iCD40 (10,000 VP/cell); Ad5-iMyD88.CD40 (100 MOI); Ad5.1MyD88 (100 MOI) or Ad5-TLR4 on (100 MOI) and stimulated with 1 microgram/milliliter LPS where indicated and 100 nM dimer drug AP20187 where indicated in FIG. 17. 48 hours later, supernatants were harvested and assayed in an IL12p70 ELISA assay.

Ad5f35-iCD40 was produced using pShuttleX-ihCD40 (also known as M-Fv′-Fvls-hCD40; pShuttleX-M-Fv′-Fvls-hCD40). MyD88, as indicated in FIGS. 16 and 17, is the same truncated version of MyD88 as the version indicated as MyD88L herein. The adenovirus indicated as Ad5.1MyD88 was produced using pShuttleX-MyD88L-Fv′Fvls-E. The adenovirus indicated as Ad5-iMyD88.Cd40 was produced using pShuttleX-MyD88LCD40-Fv′Fvls-E. The adenovirus indicated as Ad5-TLR40n was produced using pShuttleX-CD4/TLR4-L3-E.

### Example 8

### Non-Viral Transformation of Dendritic Cells

A plasmid vector is constructed comprising the iMyD88-CD40 sequence operably linked to the Fv′Fvls sequence, such as, for example, the pShuttleX-MyD88LCD40-Fv′Fvls-E Insert. The plasmid construct also includes the following regulatory elements operably linked to the MyD88ICD40-Fv′Fvls-E sequence: promoter, initiation codon, stop codon, polyadenylation signal. The vector may also comprise an enhancer sequence. The MyD88L, CD40, and FvFvls sequences may also be modified using synthetic techniques known in the art to include optimized codons.

Immature human monocyte-derived dendritic cells (MoDCs) are plated at 0.25×10⁶ cells per well of a 48-well plate after washing twice with serum-free RPMI media with antibiotic. Cells are transduced with the plasmid vector using any appropriate method, such as, for example, nucleofection using AMAXA kits, electroporation, calcium phosphate, DEAE-dextran, sonication loading, liposome-mediated transfection, receptor mediated transfection, or microprojectile bombardment.

DNA vaccines are discussed in, for example, U.S. Patent Publication 20080274140, published Nov. 6, 2008. The iMyD88-CD40 sequence operably linked to the Fv′Fvls sequence is inserted into a DNA vaccine vector, which also comprises, for example, regulatory elements necessary for expression of the iMyD88-Cd40 Fv′Fvls chimeric protein in the host tissue. These regulatory elements include, but are not limited to, promoter, initiation codon, stop codon, polyadenylation signal, and enhancer, and the codons coding for the chimeric protein may be optimized.

### Example 9

### Evaluation of CD40 and MyD88CD40 Transformed Dendritic Cells In Vivo Using a Mouse Tumor Model

Bone marrow dendritic cells were transduced using adenoviral vectors as presented in the examples herein. These transduced BMDCs were tested for their ability to inhibit tumor growth in a EG.7-OVA model. EG.7-OVA cells (5×10⁵ cells/100 ml) were inoculated into the right flank of C57BL/6 female mice. BMDCs of all groups were pulsed with 50 microgram/ml of ovalbumin protein and activated as described above. Approximately 7 days after tumor cell inoculation, BMDCs were thawed and injected subcutaneously into the hind foot-pads of mice.

Tumor growth was monitored twice weekly in mice of all groups. Peripheral blood from random mice of all groups was analyzed by tetramer staining and by in vivo CTL assays. Table 1 presents the experimental design, which includes non-transduced dendritic cells (groups 1 and 2), dendritic cells transduced with a control adenovirus vector (group 3), dendritic cells transduced with a CD40 cytoplasmic region encoding vector (group 4), dendritic cells transduced with a truncated MyD88 vector (groups 5 and 6), and dendritic cells transduced with the chimeric CD40-truncated MyD88 vector (groups 7 and 8). The cells were stimulated with AP-1903, LPS, or CD40 ligand as indicated.

TABLE 1

| | | | | | | Other | Route of | Route of | |
|---|---|---|---|---|---|---|---|---|---|
| | | Dose | ADV | | [AP1903] | reagents | Administration | Administration | |
| Group | Treatment | Level | vp/cell | [LPS] | (in vitro) | (in vitro) | (Vaccine) | (AP1903) | N |
| 1 | PBS | NA | | | N/A | | SC | N/A | 6 |
| 2 | DCs + CD40L + LPS | 1.5e6 | | 200 ng/ml | N/A | CD40L | SC | N/A | 6 |
| | | cells | | | | 2 μg/ml | | | |
| 3 | DCs + Ad-Luc + | 1.5e6 | 20K wGJ | 200 ng/ml | 100 nM | | SC | IP | 6 |
| | LPS + AP1903 | cells | | | | | | | |
| | | 5 mg/kg | | | | | | | |
| | | (AP1903) | | | | | | | |
| 4 | DCs + Ad-iCD40 + | 1.5e6 | 20K wGJ | 200 ng/ml | 100 nM | | SC | IP | 6 |
| | LPS + AP1903 | cells | | | | | | | |
| | | 5 mg/kg | | | | | | | |
| | | (AP1903) | | | | | | | |
| 5 | DCs + Ad-iMyD88 + | 1.5e6 | 20K wGJ | | 100 nM | | SC | IP | 6 |
| | AP1903 | cells | | | | | | | |
| | | 5 mg/kg | | | | | | | |
| | | (AP1903) | | | | | | | |
| 6 | DCs + Ad-iMyD88 | 1.5e6 | 20K wGJ | | N/A | | SC | N/A | 6 |
| | | cells | | | | | | | |
| 7 | DCs + Ad- | 1.5e6 | 20K wGJ | | 100 nM | | SC | IP | 6 |
| | iMyD88.CD40 + | cells | | | | | | | |
| | AP1903 | 5 mg/kg | | | | | | | |
| | | (AP1903) | | | | | | | |
| 8 | DCs + Ad- | 1.5e6 | 20K wGJ | | N/A | | SC | N/A | 6 |
| | iMyD88.CD40 | cells | | | | | | | |

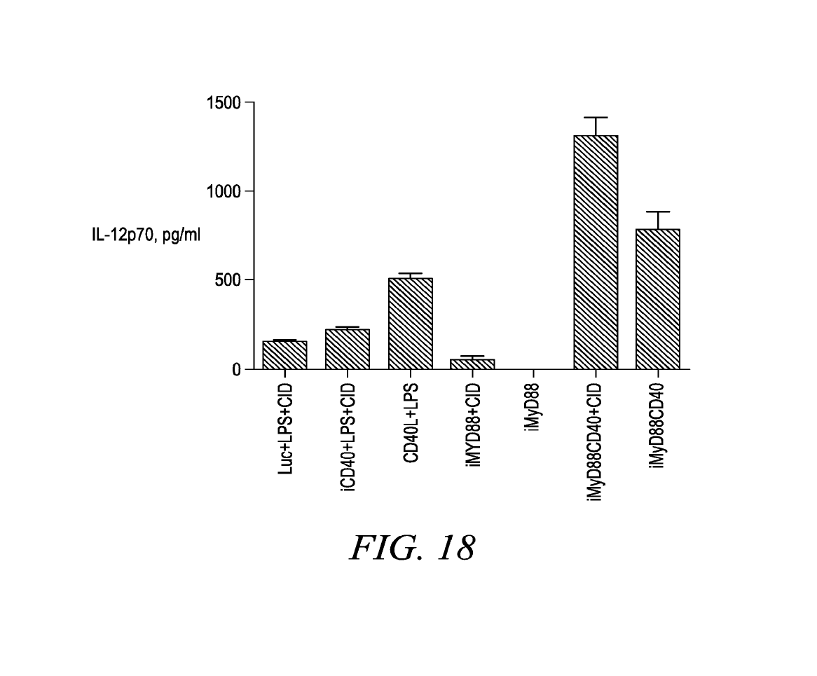

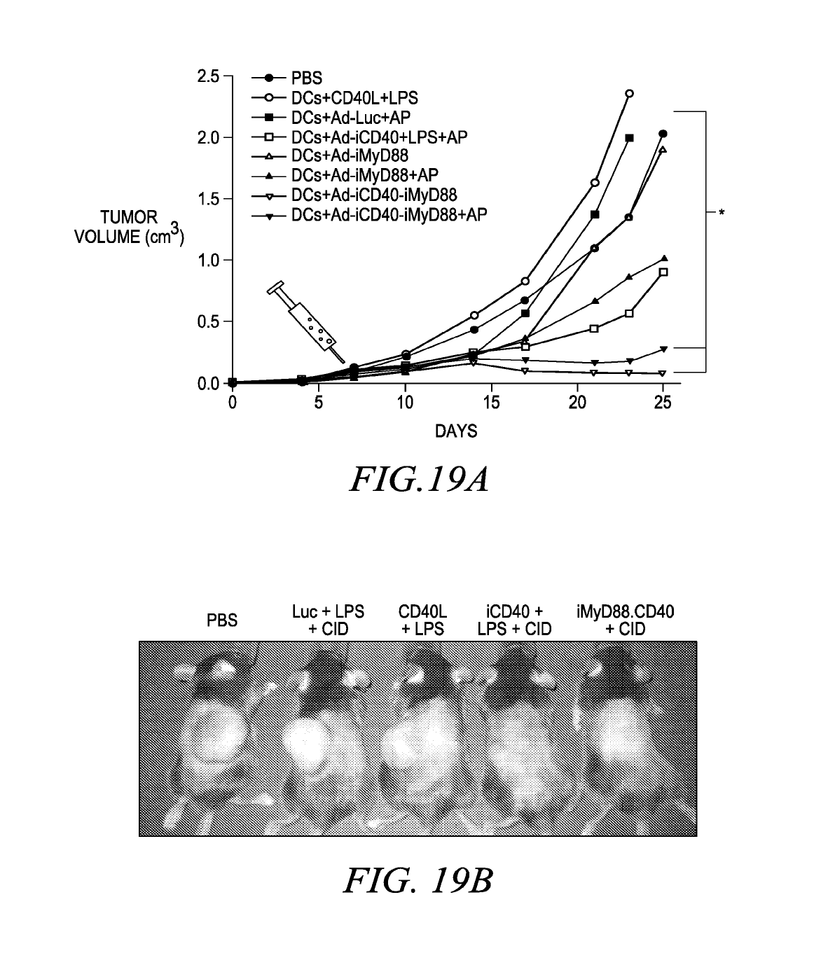

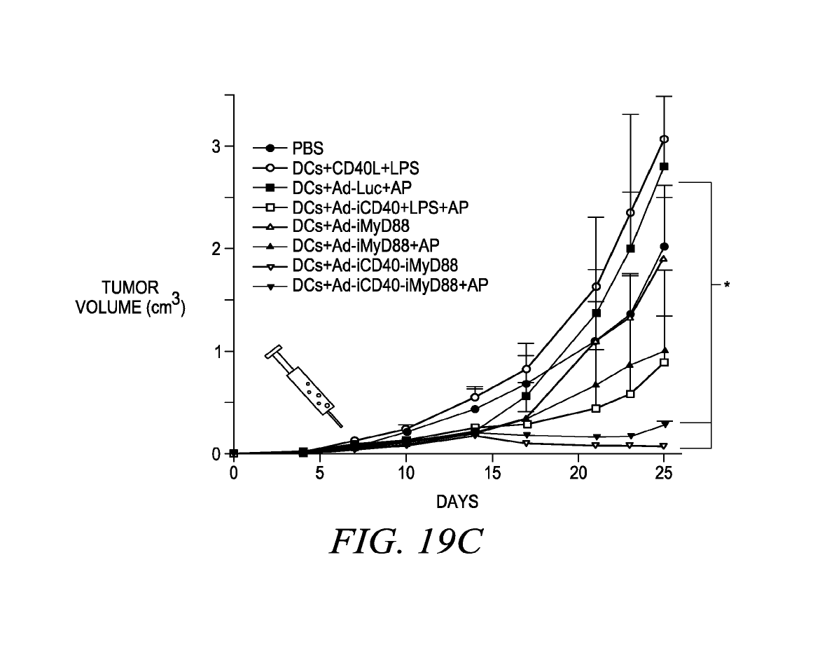

Prior to vaccination of the tumor-inoculated mice, the IL-12p70 levels of the transduced dendritic cells were measured in vitro. The IL-12p70 levels are presented in FIG. 18. FIG. 19 shows a chart of tumor growth inhibition observed in the transduced mice. Inoculation of the MyD88 transduced and AP1903 treated dendritic cells resulted in a cure rate of ⅙, while inoculation of the MyD88-CD40 transduced dendritic cells without AP1903 resulted in a cure rate of 4/6, indicating a potential dimerizer-independent effect. The asterix indicates a comparison of Luc+LPS+AP and iCD40MyD88+LPS+/−AP1903. FIG. 19 also provides photographs of representative vaccinated mice.

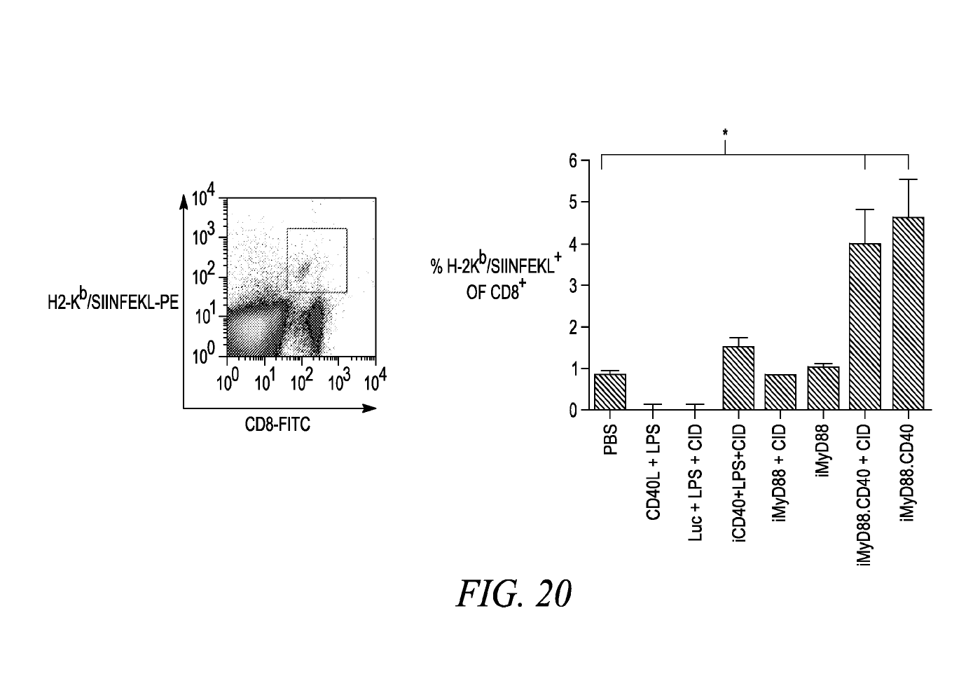

FIG. 20 presents an analysis of the enhanced frequency of Ag-Specific CD8+ T cell induction in mice treated with iMyD88-CD40 transduced dendritic cells. Peripheral bone marrow cells from treated mice were harvested ten days after vaccination on day 7. The PBMCs were stained with anti-mCD8-FITC and H2-Kb-SIINFEKL-tetramer-PE (“SIINFEKL” disclosed as SEQ ID NO: 29) and analyzed by flow cytometry.

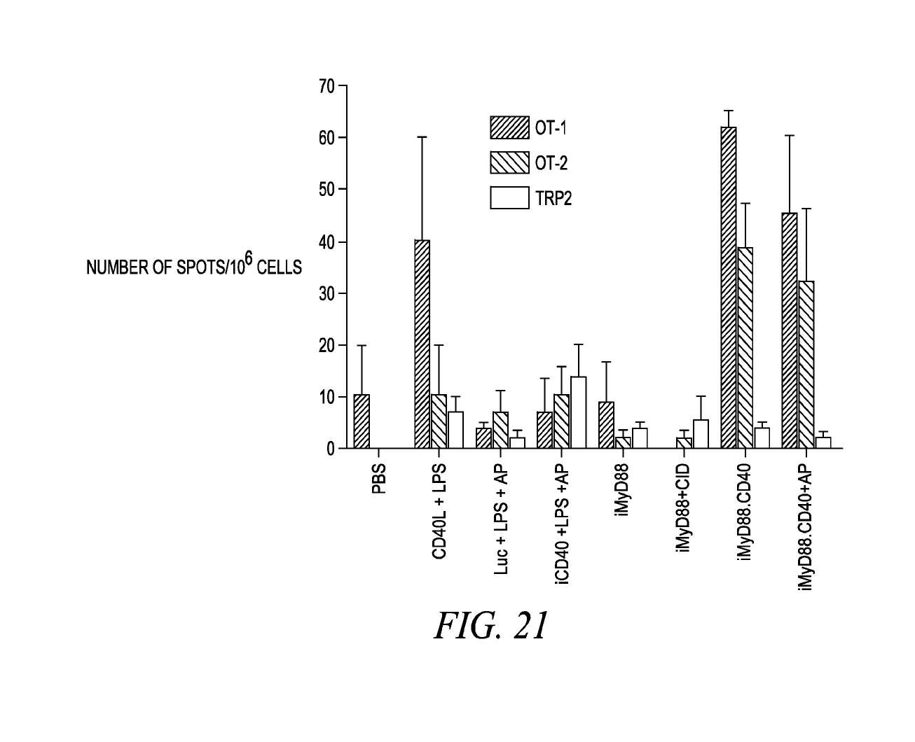

FIG. 21 presents the enhanced frequency of Ag-specific CD8+ T cell and CD4+ TH1 cells induced in mice after treatment iMyD88-CD40-transduced dendritic cells. Three mice of all experimental groups were sacrificed 18 days after the vaccination. Splenocytes of three mice per group were “pooled” together and analyzed by IFN-gamma ELISPOT assay. Millipore MultiScreen-HA plates were coated with 10 micrograms/ml anti-mouse IFN-gamma AN18 antibody (Mabtech AB, Inc., Nacka, Sweden). Splenocytes were added and cultured for 20 hours at 37 degrees C. in 5% CO2 in complete ELISpot medium (RPMI, 10% FBS, penicillin, streptomycin). Splenocytes were incubated with 2 micrograms/ml OT-1 (SIINFEKL) (SEQ ID NO: 29), OT-2 (ISQAVHAAHAEINEAGR) (SEQ ID NO: 30) or TRP-2 peptide (control non-targeted peptide). After washes, a second biotinylated monoclonal antibody to mouse IFN-gamma (R4-6A2, Mabtech AB) was applied to the wells at a concentration of 1 microgram/ml, followed by incubation with streptavidin-alkaline phosphatase complexes (Vector Laboratories, Ltd., Burlingame, Calif.). Plates were then developed with the alkaline phosphatase substrate, 3-amino-9 ethylcarbazole (Sigma-Aldrich, Inc., St. Louis, Mo.). The numbers of spots in the wells were scored by ZellNet Consulting, Inc. with an automated ELISPOT reader system (Carl Zeiss, Inc, Thornwood N.Y.).

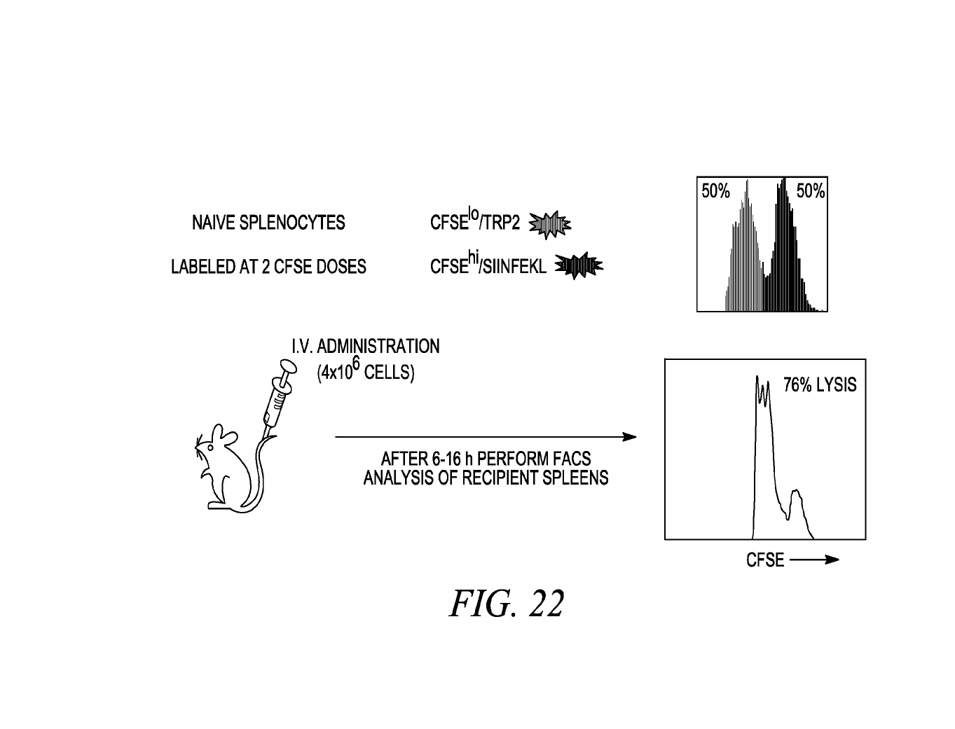

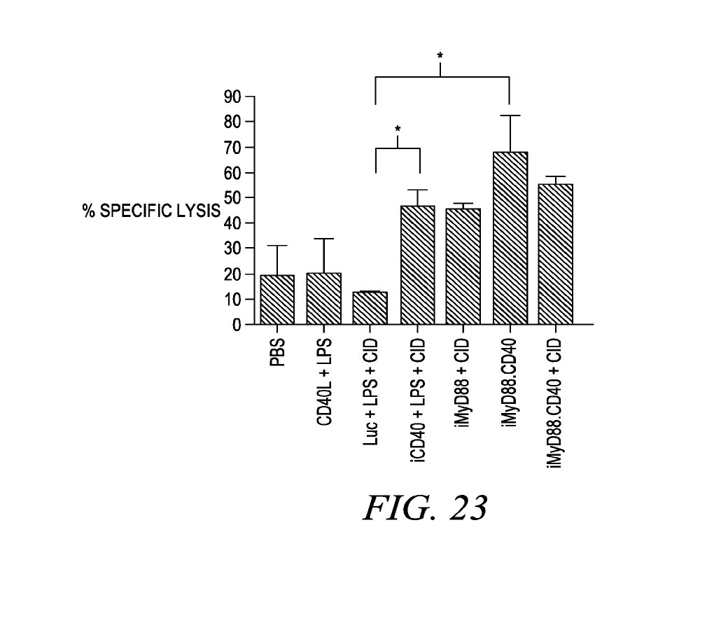

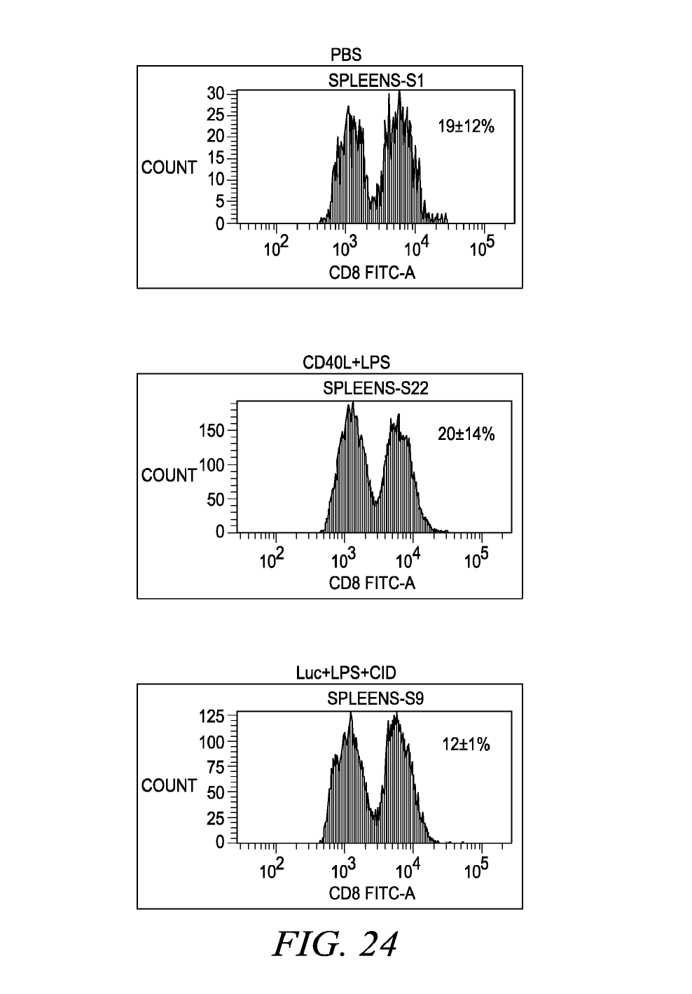

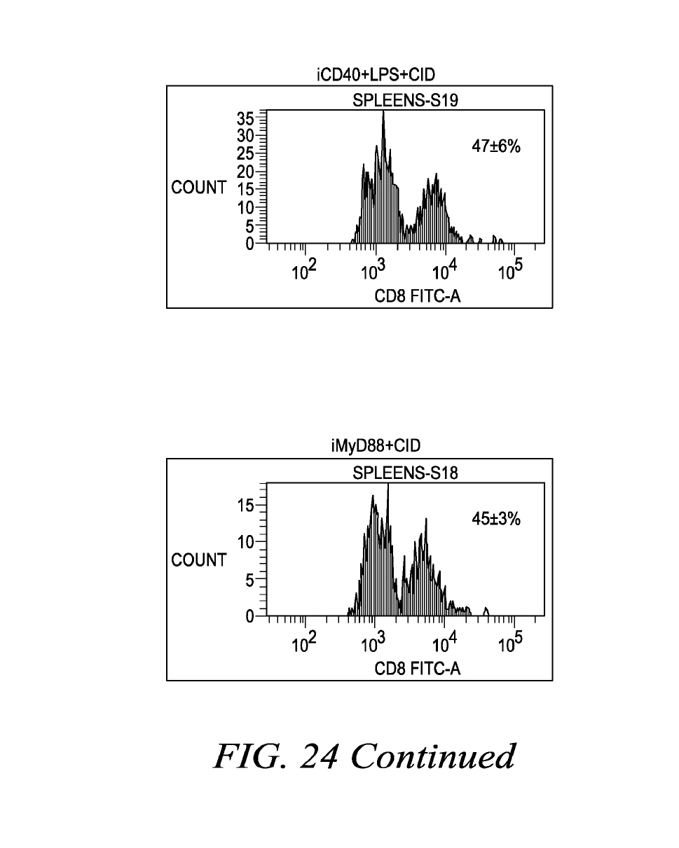

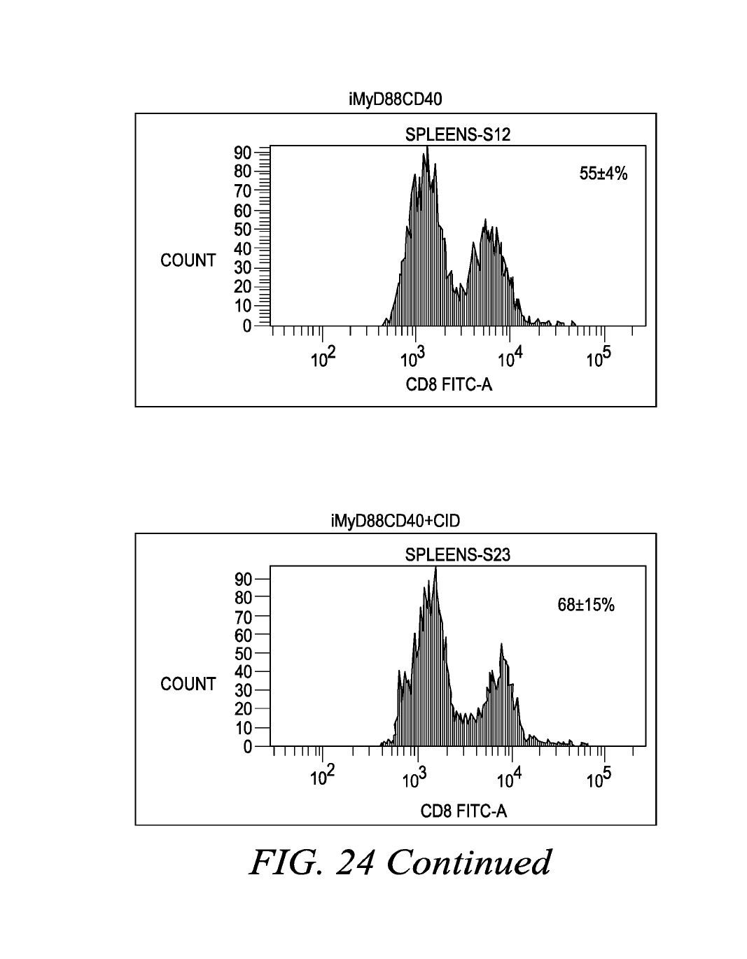

FIG. 22 presents a schematic and the results of an in vivo cytotoxic lymphocyte assay. Eighteen days after DC vaccinations an in vivo CTL assay was performed. Syngeneic naive splenocytes were used as in vivo target cells. They were labeled by incubation for 10 minutes at 37 degrees C. with either 6 micromolar CFSE (CFSEhi cells) or 0.6 micromolar CFSE in CTL medium (CFSElo cells). CFSEhi cells were pulsed with OT-1 SIINFEKL peptide (SEQ ID NO: 29), and CFSElo cells were incubated with control TRP2 peptide. A mixture of 4×10⁶ CFSEhi plus 4×10⁶ CFSElo cells was injected intravenously through the tail vein. After 16 hours of in vivo incubation, splenocytes were collected and single-cell suspensions are analyzed for detection and quantification of CFSE-labeled cells. FIG. 23 is a chart presenting the enhanced CTL activity induced by iMyD88-CD40-transduced dendritic cells in the inoculated mice. FIG. 24 shows the raw CTL histograms for select samples, indicating the enhanced in vivo CTL activity induced by the iMyD88-CD40 transduced dendritic cells.

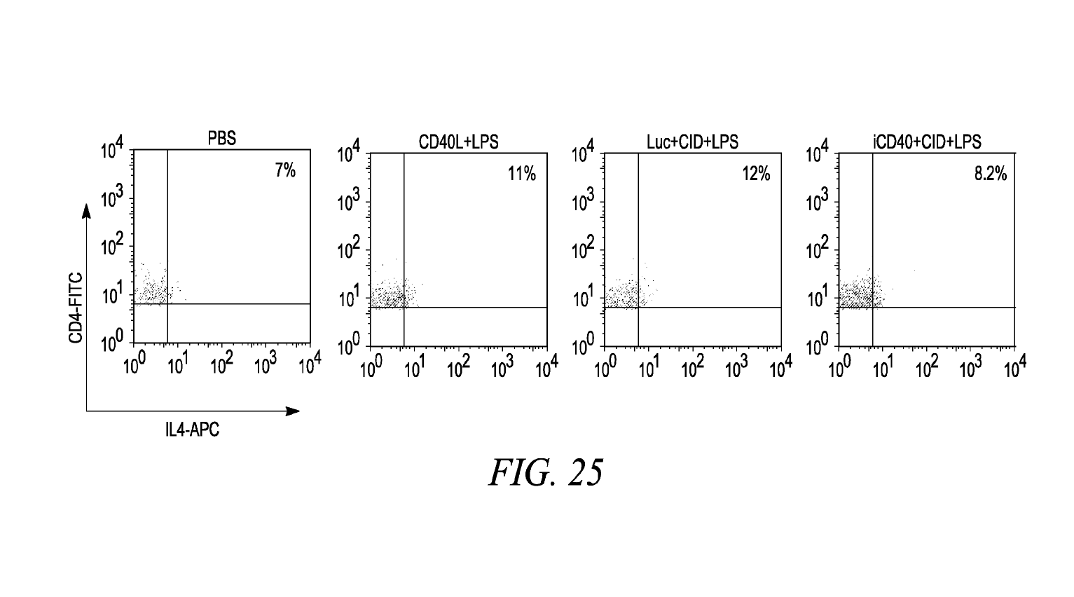

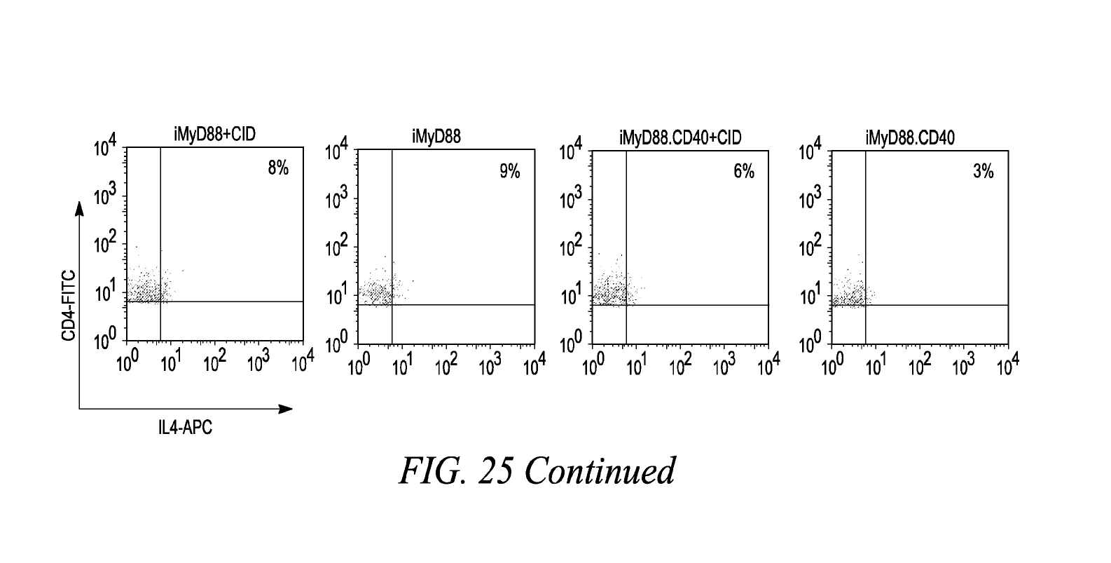

FIG. 25 presents the results of intracellular staining for IL-4 producing TH2 cells in the mice vaccinated with the transduced cells. Splenocytes of mice (pooled cells from three mice) were reconstituted with 2 micrograms/ml of OT-2 peptide. Cells were incubated for 6 hours with 10 micrograms/ml of brefeldin A to suppress secretion. Then cells were fixed and permealized and analyzed by intracellular staining with anti-mIL-4-APC and anti-mCD4-FITC.

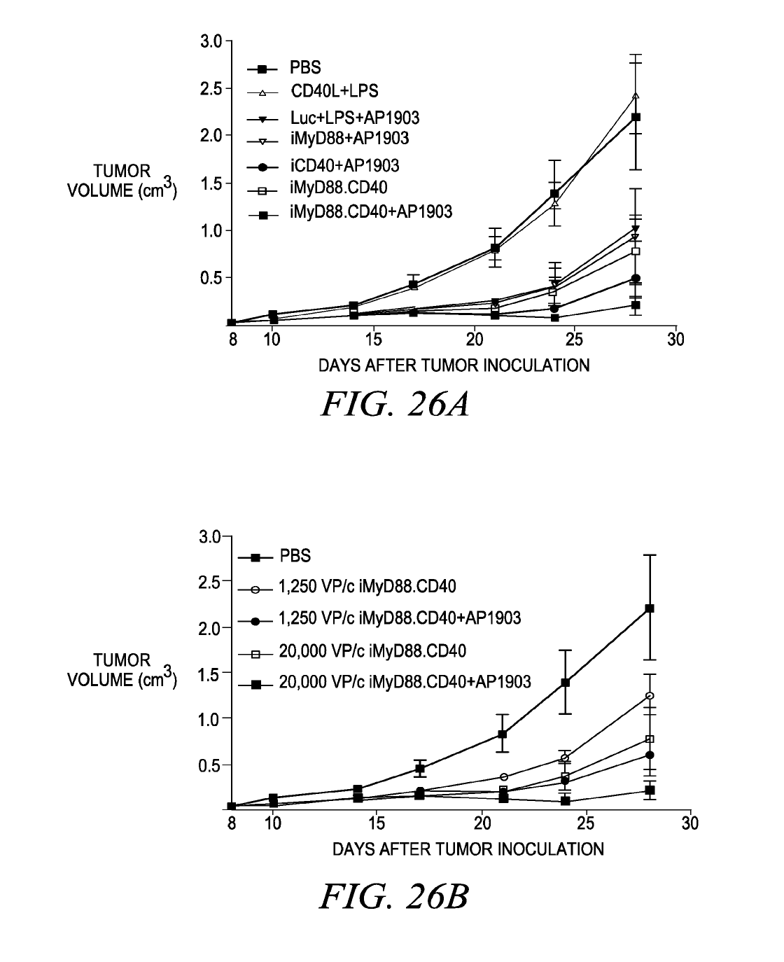

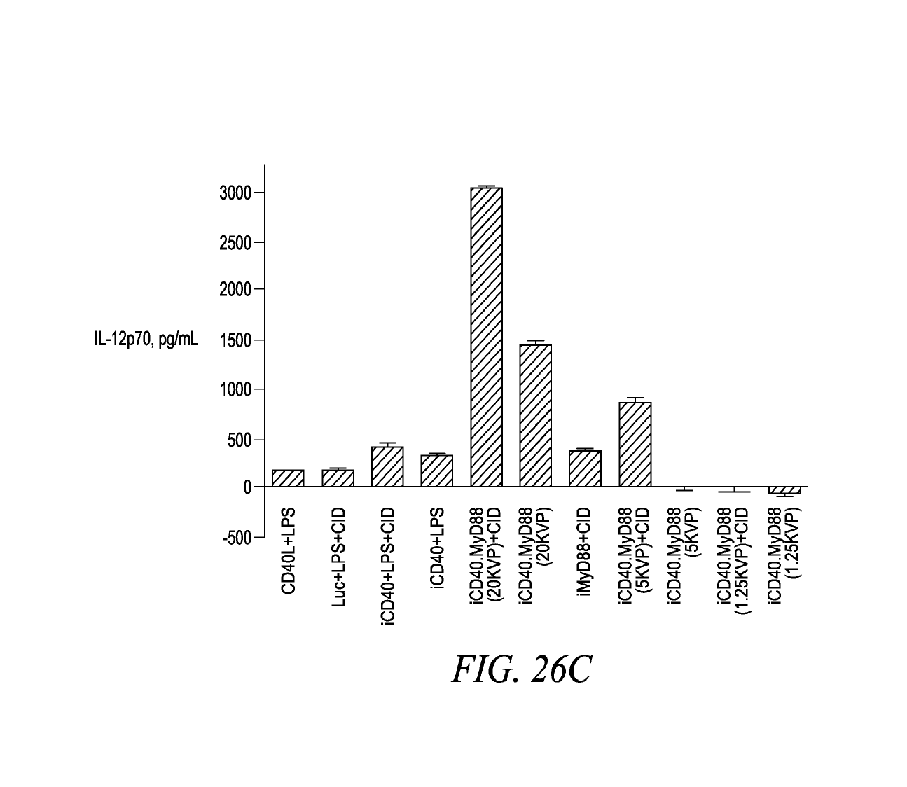

The adenoviral vector comprising the iCD40-MyD88 sequence was again evaluated for its ability to inhibit tumor growth in a mouse model. In the first experiment, drug-dependent tumor growth inhibition was measured after inoculation with dendritic cells modified with the inducible CD40-truncated MyD88 vector (Ad-iCD40.MyD88). Bone marrow-derived dendritic cells from C57BL/6 mice were pulsed with 10 micrograms/ml of ovalbumin and transduced with 20,000 viral particles/cell (VP/c) of the adenovirus constructs Ad5-iCD40.MyD88, Ad5-iMyD88 or Ad5-Luc (control). Cells were activated with either 2 micrograms/ml CD40L, 200 ng/ml LPS, or 50 nM AP1903 dimerizer drug. 5×10⁵E.G7-OVA thymoma cells were inoculated into the backs of C57BL/6 mice (N=6/group). When tumors reached ˜5 mm in diameter (day 8 after inoculation), mice were treated with subcutaneous injections of 2×10⁶ BMDCs. The next day, after cellular vaccinations, mice were treated with intraperitoneal injections of 5 mg/kg AP1903. Tumor growth was monitored twice weekly. The results are shown in FIG. 26A. In another set of experiments, E.G7-OVA tumors were established as described above. Mice (N=6/group) were treated with 2×10⁶ BMDCs (ovalbumin pulsed) and transduced with either 20,000 or 1,250 VP/c of Ad5-iCD40.MyD88. BMDCs of AP1903 groups were treated in vitro with 50 nM AP1903. The next day, after cellular vaccinations, mice of AP1903 groups were treated by intraperitoneal injection with 5 mg/kg AP1903. The results are shown in FIG. 26B. FIG. 26C depicts relative IL-12p70 levels produced following overnight culture of the various vaccine cells prior to cryopreservation. IL-12p70 was assayed by ELISA assay.

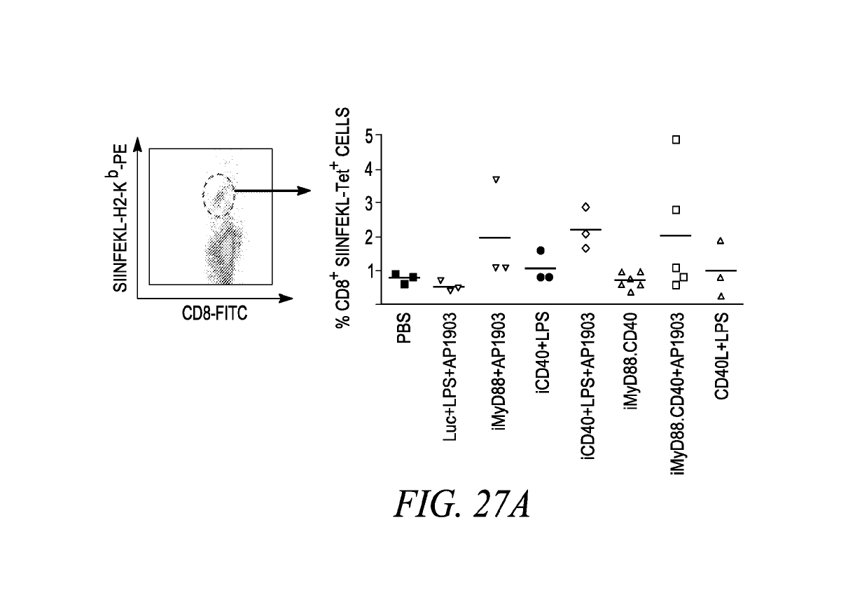

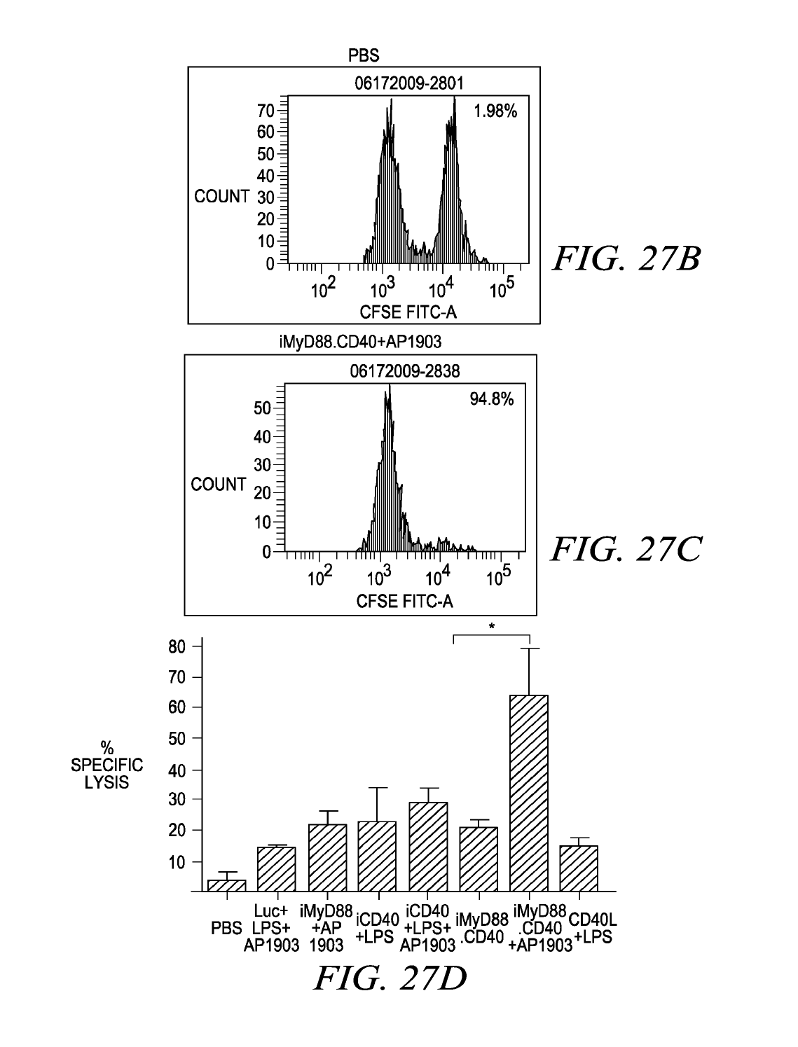

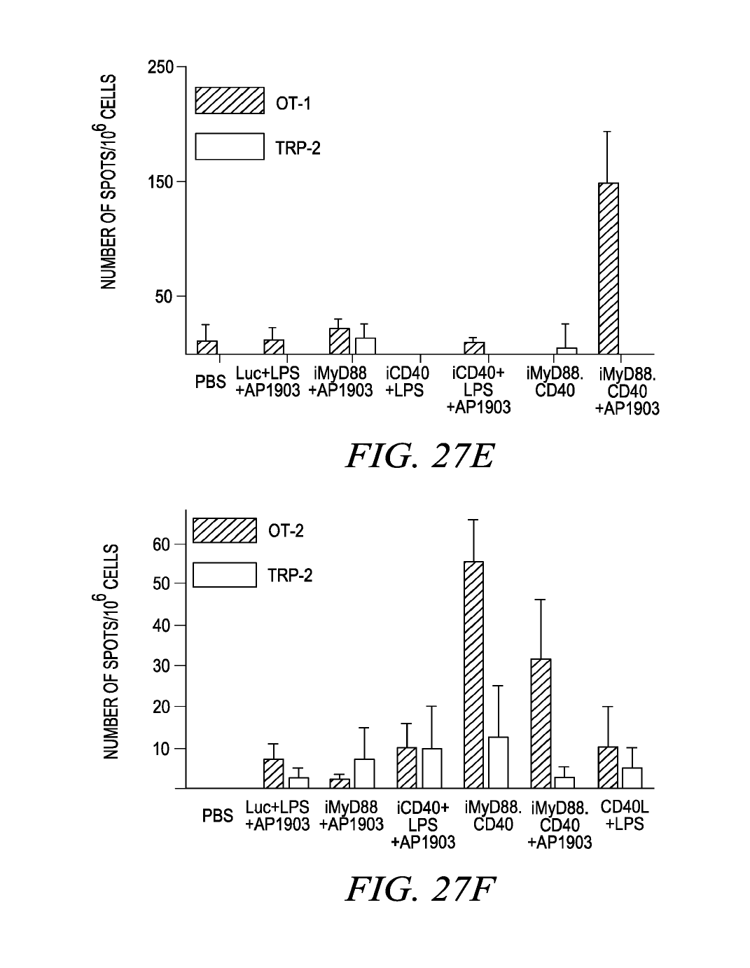

Blood from mice immunized with the modified bone marrow dendritic cells was analyzed for the frequency and function of tumor specific T cells using tetramer staining. FIG. 27A shows the results of an experiment in which mice (N=3-5) were immunized subcutaneously with BMDCs pulsed with ovalbumin and activated as described in FIG. 26A-FIG. 26C. One week after the vaccination, peripheral blood mononuclear cells (PBMCs) were stained with anti-mCD8-FITC and SIINFEKL-H2-Kb-PE (“SIINFEKL” disclosed as SEQ ID NO: 29) and analyzed by flow cytometry. FIG. 27B shows the results of an in vivo CTL assay that was performed in mice vaccinated with BMDCs as described above. Two weeks after the BMDC immunization, splenocytes from syngeneic C57BL/6 mice were pulsed with either TRP-2 control peptide, SVYDFFVWL (SEQ ID NO: 31), or target peptide, SINFEKL (SEQ ID NO: 32) target, and were used as in vivo targets. Half of the splenocytes were labeled with 6 micromolar CFSE (CFSEhi cells) or 0.6 micromolar CFSE (CFSElo cells). CFSEhi cells were pulsed with OT-1 (SIINFEKL) (SEQ ID NO: 29) peptide and CFSElo cells were incubated with control TRP-2 (SVYDFFVWL) (SEQ ID NO: 31) peptide. A mixture of 4×10⁶ CFSEhi plus 4×10⁶ CFSElo cells was injected intravenously through the tail vein. The next day, splenocytes were collected and single-cell suspensions were analyzed for detection and quantification of CFSE-labeled cells. FIGS. 27C and 27D show the results of an IFN-gamma assay. Peripheral blood mononuclear cells (PBMCs) from E.G7-OVA-bearing mice treated as described in FIG. 26A-FIG. 26C, were analyzed in IFN-gamma ELISpot assays with 1 microgram/ml of SIINFEKL (SEQ ID NO: 29) peptide (OT-1), ISQAVHAAHAEINEAGR (SEQ ID NO: 32) (OT-2) and TRP-2 (irrelevant H2-Kb-restricted) peptides. The number of IFN-gamma-producing lymphocytes was evaluated in triplicate wells. Cells from three mice per group were pooled and analyzed by IFN-gamma ELISpot in triplicate wells. The assays were performed twice.

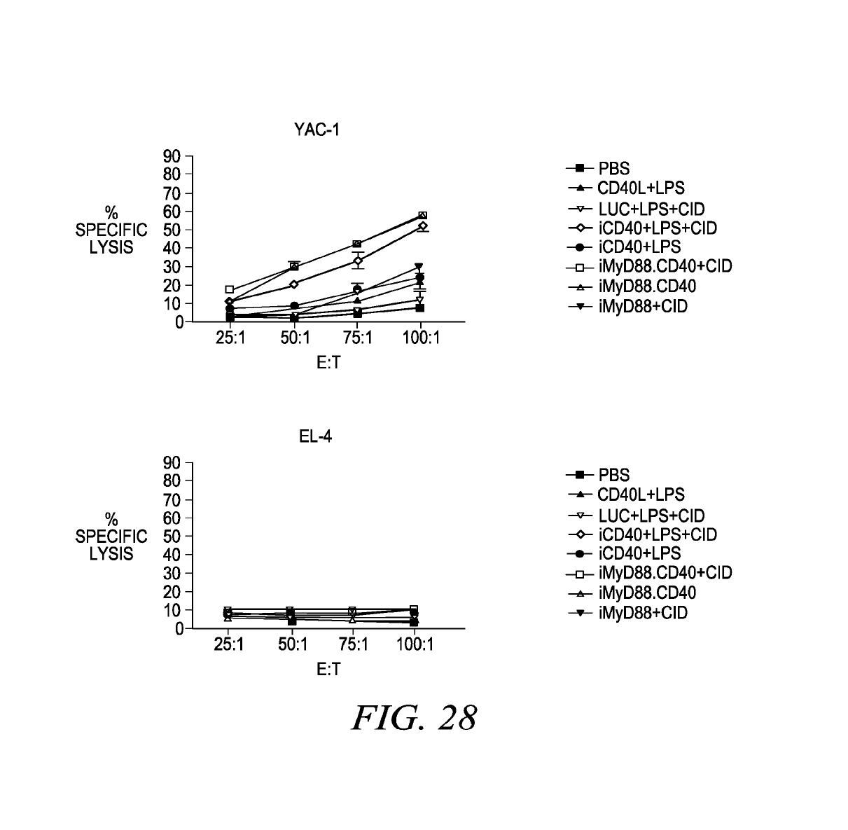

FIG. 28 presents the results of a natural killer cell assay performed using the splenocytes from mice treated as indicated in this example. Splenocytes obtained from mice (3 per group) were used as effectors (E). Yac-1 cells were labeled with 51Cr and used as targets (T). The EL-4 cell line was used as an irrelevant control.

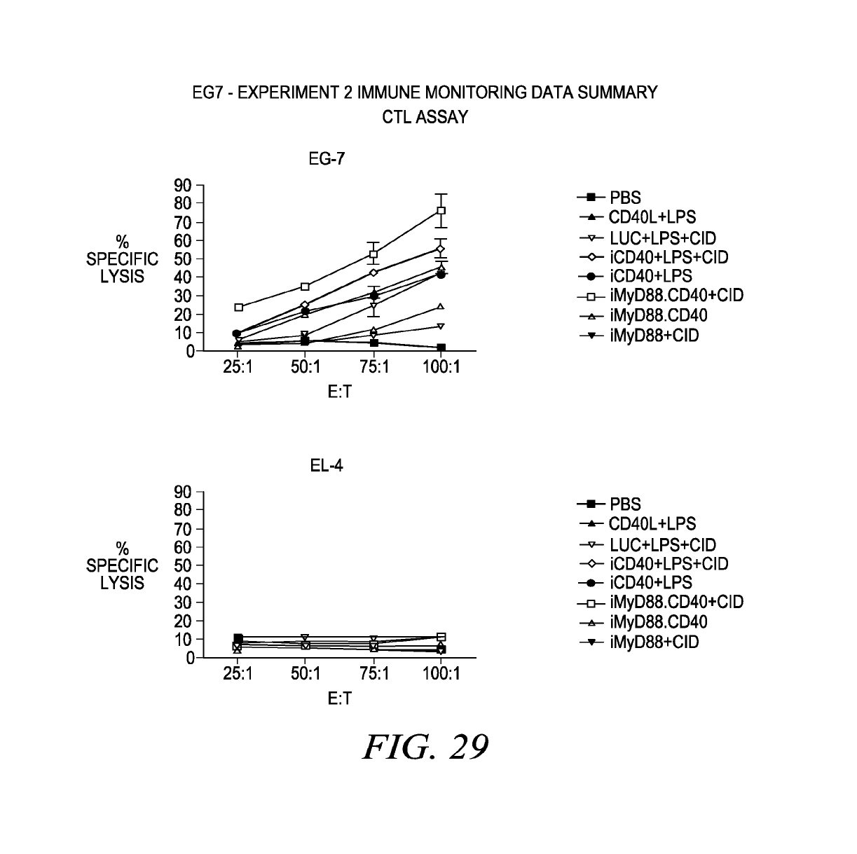

FIG. 29 presents the results of an assay for detection of antigen-specific cytotoxic lymphocytes. Splenocytes obtained from mice (3 per group) were used as effectors. EG.7-Ova cells were labeled with 51Cr and used as targets (T). The EL-4 cell line was used as an irrelevant control.

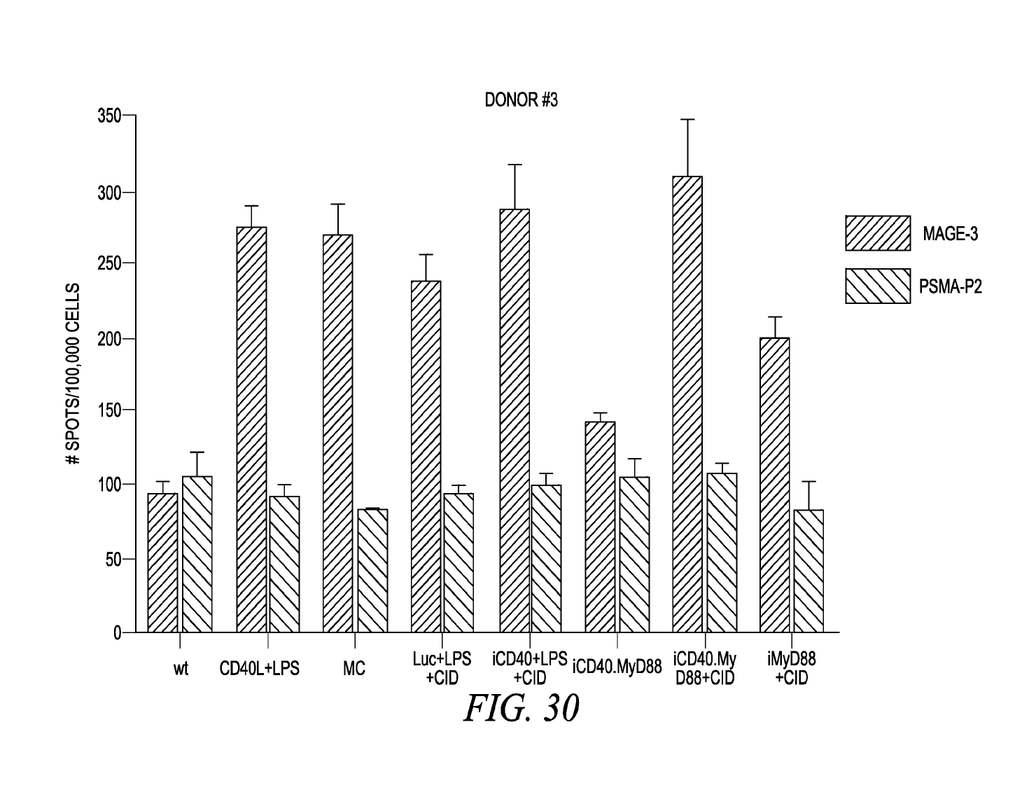

FIG. 30 presents the results of the activation of human cells transduced with the inducible CD40-truncated MyD88 (iCD40.MyDD) adenovirus vector. Dendritic cells (day 5 of culture) from three different HLA-A2+ donors were purified by the plastic-adhesion method and transduced with 10,000 VP/cell of Ad5-iCD40.MyD88, Ad5-iMyD88 or Ad5-Luc. Cells were activated with 100 nM AP1903 or 0.5 micrograms/ml of CD40L and 250 ng/ml of LPS or standard maturation cocktail (MC), containing TNF-alpha, IL-1beta, IL-6, and prostaglandin E2 (PGE2). Autologous CD8+ T cells were purified by negative selection using microbeads and co-cultured with DCs pulsed with 10 micrograms/ml of HLA-A2-restricted FLWGPRALV MAGE-3 (SEQ ID NO: 19) peptide at 1:5 (DC:T) ratio for 7 days. Five days after the second of round of stimulation with DCs (on day 7) T cells were assayed in standard IFN-gamma ELISpot assay. Cells were pulsed with 1 micrograms/ml of MAGE-3 or irrelevant HLA-A2-restricted PSMA polypeptide (PSMA-P2). Experiments were performed in triplicate.

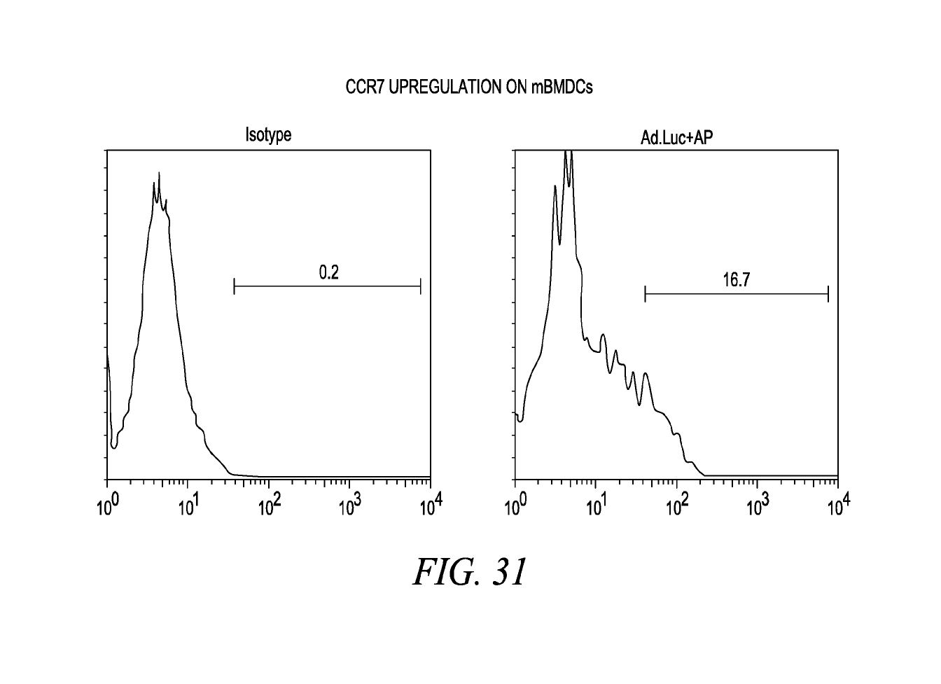

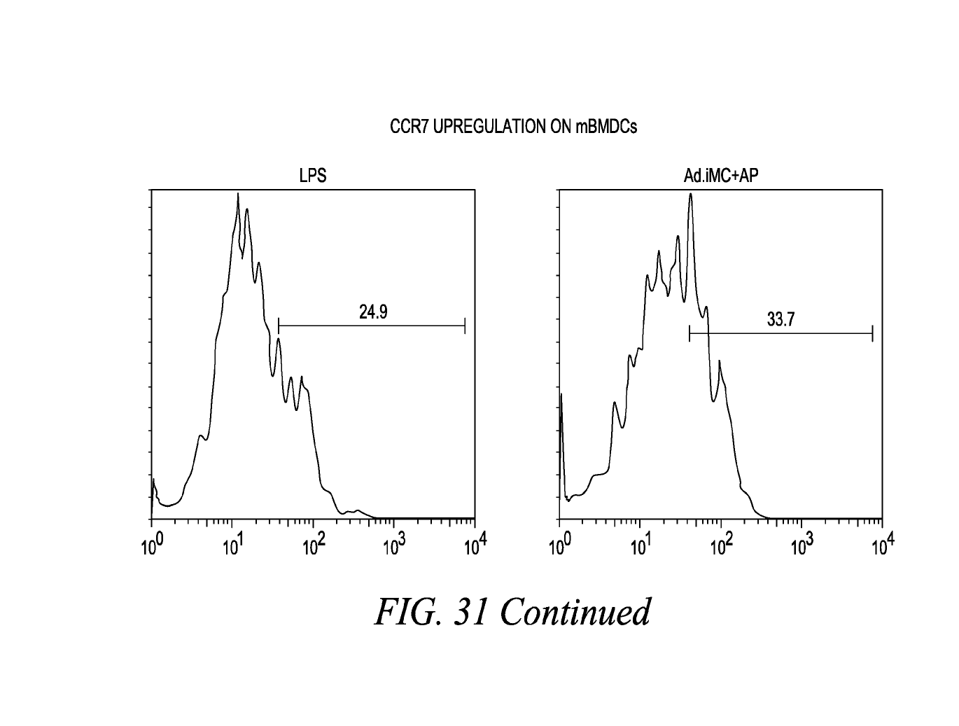

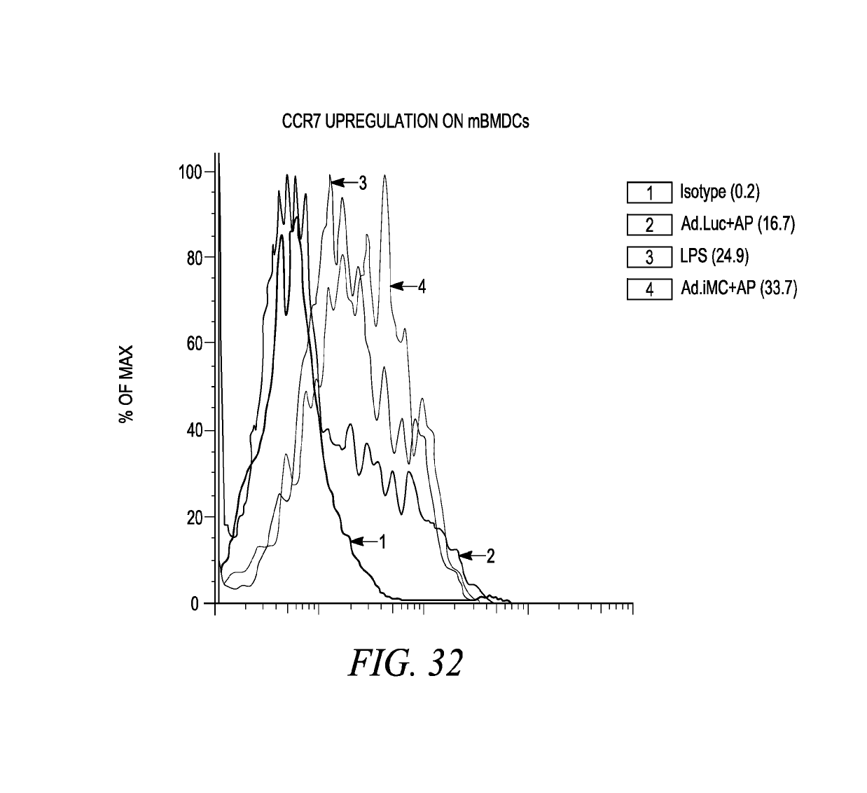

FIGS. 31 and 32 present the results of a cell migration assay. mBMDCs were transduced with 10,000 VP/cell of Ad5.Luciferase or Ad5.1MyD88.CD40 in the presence of Gene Jammer (Stratagene, San Diego, Calif.) and stimulated with 100 nM AP1903 (AP) or LPS (1 microgram/ml) for 48 hours. CCR7 expression was analyzed on the surface of CD11c+dendritic cells by intracellular staining using a PerCP.Cy5.5 conjugated antibody. FIG. 31 shows the results of the experiment, with each assay presented separately; FIG. 32 provides the results in the same graph.

### Example 10

### Examples of Particular Nucleic Acid and Amino Acid Sequences

```
SEQ ID NO: 1 (nucleic acid sequence encoding human CD40; Genbank accession no.
NM_001250; cytoplasmic region indicated in bold).
      1 gccaaggctg gggcagggga gtcagcagag gcctcgctcg ggcgcccagt ggtcctgccg
    61 cctggtctca cctcgctatg gttcgtctgc ctctgcagtg cgtcctctgg ggctgcttgc
  121 tgaccgctgt ccatccagaa ccacccactg catgcagaga aaaacagtac ctaataaaca
  181 gtcagtgctg ttctttgtgc cagccaggac agaaactggt gagtgactgc acagagttca
  241 ctgaaacgga atgccttcct tgcggtgaaa gcgaattcct agacacctgg aacagagaga
  301 cacactgcca ccagcacaaa tactgcgacc ccaacctagg gcttcgggtc cagcagaagg
  361 gcacctcaga aacagacacc atctgcacct gtgaagaagg ctggcactgt acgagtgagg
  421 cctgtgagag ctgtgtcctg caccgctcat gctcgcccgg ctttggggtc aagcagattg
  481 ctacaggggt ttctgatacc atctgcgagc cctgcccagt cggcttcttc tccaatgtgt
  541 catctgcttt cgaaaaatgt cacccttgga caagctgtga gaccaaagac ctggttgtgc
  601 aacaggcagg cacaaacaag actgatgttg tctgtggtcc ccaggatcgg ctgagagccc
  661 tggtggtgat ccccatcatc ttcgggatcc tgtttgccat cctcttggtg ctggtcttta
  721 tcaaaaaggt ggccaagaag ccaaccaata aggcccccca ccccaagcag gaaccccagg
  781 agatcaattt tcccgacgat cttcctggct ccaacactgc tgctccagtg caggagactt
  841 tacatggatg ccaaccggtc acccaggagg atggcaaaga gagtcgcatc tcagtgcagg
  901 agagacagtg aggctgcacc cacccaggag tgtggccacg tgggcaaaca ggcagttggc
  961 cagagagcct ggtgctgctg ctgctgtggc gtgagggtga ggggctggca ctgactgggc
1021 atagctcccc gcttctgcct gcacccctgc agtttgagac aggagacctg gcactggatg
1081 cagaaacagt tcaccttgaa gaacctctca cttcaccctg gagcccatcc agtctcccaa
1141 cttgtattaa agacagaggc agaagtttgg tggtggtggt gttggggtat ggtttagtaa
1201 tatccaccag accttccgat ccagcagttt ggtgcccaga gaggcatcat ggtggcttcc
1261 ctgcgcccag gaagccatat acacagatgc ccattgcagc attgtttgtg atagtgaaca
1321 actggaagct gcttaactgt ccatcagcag gagactggct aaataaaatt agaatatatt
1381 tatacaacag aatctcaaaa acactgttga gtaaggaaaa aaaggcatgc tgctgaatga
1441 tgggtatgga actttttaaa aaagtacatg cttttatgta tgtatattgc ctatggatat
1501 atgtataaat acaatatgca tcatatattg atataacaag ggttctggaa gggtacacag
1561 aaaacccaca gctcgaagag tggtgacgtc tggggtgggg aagaagggtc tggggg
SEQ ID NO: 2 (amino acid sequence encoding human CD40; cytoplasmic region indicated in bold).
MVRLPLQCVLWGCLLTAVHPEPPTACREKQYLINSQCCSLCQPGQKLVSDCTEFTETECLPCGES
EFLDTWNRETHCHQHKYCDPNLGLRVQQKGTSETDTICTCEEGWHCTSEACESCVLHRSCSPGF
GVKQIATGVSDTICEPCPVGFFSNVSSAFEKCHPWTSCETKDLVVQQAGTNKTDVVCGPQDRLR
ALVVIPIIFGILFAILLVLVFIKKVAKKPTNKAPHPKQEPQEINFPDDLPGSNTAAPVQETLHGCQPV
TQEDGKESRISVQERQ
SEQ ID NO: 3 (nucleotide sequence encoding PSMA)
Key: Signal Peptide (upper case, bold, underline) (gp67), BamHI site/spacer (upper case, underline),
6xHis (upper case) (SEQ ID NO: 33), Factor Xa cleavage site (upper and lower case, bold),
PSMA (lower case)(44-750)
ATGCTACTAGTAAATCAGTCACACCAAGGCTTCAATAAGGAACACACAAGCAAGATGGTAAGCGCTATTGTTTTATA
TGTGCTTTTGGCGGCGGCGGCGCATTCTGCCTTTGCGGCGGATCCGCATCATCATCATCATCACAGCtccggaATCGAG
GGACGTGGTaaatcctccaatgaagctactaacattactccaaagcataatatgaaagcatttttggatgaattgaaagctgagaacatcaagaagtt
cttatataattttacacagataccacatttagcaggaacagaacaaaactttcagcttgcaaagcaaattcaatcccagtggaaagaatttggcctggattc
tgttgagctagcacattatgatgtcctgttgtcctacccaaataagactcatcccaactacatctcaataattaatgaagatggaaatgagattttcaacaca
tcattatttgaaccacctcctccaggatatgaaaatgtttcggatattgtaccacctttcagtgctttctctcctcaaggaatgccagagggcgatctagtgt
atgttaactatgcacgaactgaagacttctttaaattggaacgggacatgaaaatcaattgctctgggaaaattgtaattgccagatatgggaaagttttcag
aggaaataaggttaaaaatgcccagctggcaggggccaaaggagtcattctctactccgaccctgctgactactttgctcctggggtgaagtcctatccagat
ggttggaatcttcctggaggtggtgtccagcgtggaaatatcctaaatctgaatggtgcaggagaccctctcacaccaggttacccagcaaatgaatatgct
tataggcgtggaattgcagaggctgttggtcttccaagtattcctgttcatccaattggatactatgatgcacagaagctcctagaaaaaatgggtggctca
gcaccaccagatagcagctggagaggaagtctcaaagtgccctacaatgttggacctggctttactggaaacttttctacacaaaaagtcaagatgcacat
ccactctaccaatgaagtgacaagaatttacaatgtgataggtactctcagaggagcagtggaaccagacagatatgtcattctgggaggtcaccgggact
catgggtgtttggtggtattgaccctcagagtggagcagctgttgttcatgaaattgtgaggagctttggaacactgaaaaaggaagggtggagacctaga
agaacaattttgtttgcaagctgggatgcagaagaatttggtcttcttggttctactgagtgggcagaggagaattcaagactccttcaagagcgtggcgtg
gcttatattaatgctgactcatctatagaaggaaactacactctgagagttgattgtacaccgctgatgtacagcttggtacacaacctaacaaaagagctg
aaaagccctgatgaaggctttgaaggcaaatctctttatgaaagttggactaaaaaaagtccttccccagagttcagtggcatgcccaggataagcaaatt
gggatctggaaatgattttgaggtgttcttccaacgacttggaattgcttcaggcagagcacggtatactaaaaattgggaaacaaacaaattcagcggct
atccactgtatcacagtgtctatgaaacatatgagttggtggaaaagttttatgatccaatgtttaaatatcacctcactgtggcccaggttcgaggagggat
ggtgtttgagctagccaattccatagtgctcccttttgattgtcgagattatgctgtagttttaagaaagtatgctgacaaaatctacagtatttctatgaaa
catccacaggaaatgaagacatacagtgtatcatttgattcacttttttctgcagtaaagaattttacagaaattgcttccaagttcagtgagagactccagg
actttgacaaaagcaacccaatagtattaagaatgatgaatgatcaactcatgtttctggaaagagcatttattgatccattagggttaccagacaggccttt
ttataggcatgtcatctatgctccaagcagccacaacaagtatgcaggggagtcattcccaggaatttatgatgctctgtttgatattgaaagcaaagtgga
cccttccaaggcctggggagaagtgaagagacagatttatgttgcagccttcacagtgcaggcagctgctgagactttgagtgaagtagcctaa
SEQ ID NO: 4 (PSMA amino acid sequence)
MWNLLHETDSAVATARRPRWLCAGALVLAGGFFLLGFLFGWFIKSSNEATNITPKHNMKAFLDEL
KAENIKKFLYNFTQIPHLAGTEQNFQLAKQIQSQWKEFGLDSVELAHYDVLLSYPNKTHPNYISIINE
DGNEIFNTSLFEPPPPGYENVSDIVPPFSAFSPQGMPEGDLVYVNYARTEDFFKLERDMKINCSGK
IVIARYGKVFRGNKVKNAQLAGAKGVILYSDPADYFAPGVKSYPDGWNLPGGGVQRGNILNLNGA
GDPLTPGYPANEYAYRRGIAEAVGLPSIPVHPIGYYDAQKLLEKMGGSAPPDSSWRGSLKVPYNV
GPGFTGNFSTQKVKMHIHSTNEVTRIYNVIGTLRGAVEPDRYVILGGHRDSWVFGGIDPQSGAAV
VHEIVRSFGTLKKEGWRPRRTILFASWDAEEFGLLGSTEWAEENSRLLQERGVAYINADSSIEGNY
TLRVDCTPLMYSLVHNLTKELKSPDEGFEGKSLYESWTKKSPSPEFSGMPRISKLGSGNDFEVFF
QRLGIASGRARYTKNWETNKFSGYPLYHSVYETYELVEKFYDPMFKYHLTVAQVRGGMVFELAN
SIVLPFDCRDYAVVLRKYADKIYSISMKHPQEMKTYSVSFDSLFSAVKNFTEIASKFSERLQDFDKS
KHVIYAPSSHNKYAGESFPGIYDALFDIESKVDPSKAWGEVKRQIYVAAFTVQAAAETLSEVA
SEQ ID NO: 5 (nucleotide sequence of MyD88L with SalI linkers)
gtcgacatggctgcaggaggtcccggcgcggggtctgcggccccggtctcctccacatcctcccttcccctggctgctctcaacatgcgag
tgcggcgccgcctgtctctgttcttgaacgtgcggacacaggtggcggccgactggaccgcgctggcggaggagatggactttgagtactt
ggagatccggcaactggagacacaagcggaccccactggcaggctgctggacgcctggcagggacgccctggcgcctctgtaggcc
gactgctcgagctgcttaccaagctgggccgcgacgacgtgctgctggagctgggacccagcattgaggaggattgccaaaagtatatct
tgaagcagcagcaggaggaggctgagaagcctttacaggtggccgctgtagacagcagtgtcccacggacagcagagctggcgggc
atcaccacacttgatgaccccctggggcatatgcctgagcgtttcgatgccttcatctgctattgccccagcgacatcgtcgac
SEQ ID NO: 6 (amino acid sequence of MYD88L)
MAAGGPGAGSAAPVSSTSSLPLAALNMRVRRRLSLFLNVRTQVAADWTALAEEMDFEYLEIRQLE
TQADPTGRLLDAWQGRPGASVGRLLELLTKLGRDDVLLELGPSIEEDCQKYILK
QQQEEAEKPLQVAAVDSSVPRTAELAGITTLDDPLGHMPERFDAFICYCPSDI
SEQ ID NO: 7 (nucleotide sequence of Fv'Fvls with XhoI/SalI linkers, (wobbled codons lowercase in Fv'))
ctcgagGGcGTcCAaGTcGAaACcATtagtCCcGGcGAtGGcaGaACaTTtCCtAAaaGgGGaCAaACaTGt
GTcGTcCAtTAtACaGGcATGtTgGAgGAcGGcAAaAAgGTgGAcagtagtaGaGAtcGcAAtAAaCCtTTc
AAaTTcATGtTgGGaAAaCAaGAaGTcATtaGgGGaTGGGAgGAgGGcGTgGCtCAaATGtccGTcGGc
CAacGcGCtAAgCTcACcATcagcCCcGAcTAcGCaTAcGGcGCtACcGGaCAtCCcGGaATtATtCCcC
CtCAcGCtACctTgGTgTTtGAcGTcGAaCTgtTgAAgCTcGAagtcgagggagtgcaggtggaaaccatctccccag
gagacgggcgcaccttccccaagcgcggccagacctgcgtggtgcactacaccgggatgcttgaagatggaaagaaagttgattcctc
ccgggacagaaacaagcctttaagtttatgctaggcaagcaggaggtgatccgaggctgggaagaaggggttgcccagatgagtgtg
ggtcagagagccaaactgactatatctccagattatgcctatggtgccactgggcacccaggcatcatcccaccacatgccactctcgtctt
cgatgtggagcttctaaaactggaatctggcggtggatccggagtcgag
SEQ ID NO: 8 (FV'FVLS amino acid sequence)
GlyValGlnValGluThrIleSerProGlyAspGlyArgThrPheProLysArgGlyGlnThrCysValValHisTyrThrGlyMe
tLeuGluAspGlyLysLysValAspSerSerArgAspArgAsnLysProPheLysPheMetLeuGlyLysGlnGluValIleA
rgGlyTrpGluGluGlyValAlaGlnMetSerValGlyGlnArgAlaLysLeuThrIleSerProAspTyrAlaTyrGlyAlaThrG
lyHisProGlyIleIleProProHisAlaThrLeuValPheAspValGluLeuLeuLysLeuGlu(ValGlu)
GlyValGlnValGluThrIleSerProGlyAspGlyArgThrPheProLysArgGlyGlnThrCysValValHisTyrThrGlyMe
tLeuGluAspGlyLysLysValAspSerSerArgAspArgAsnLysProPheLysPheMetLeuGlyLysGlnGluValIleA
rgGlyTrpGluGluGlyValAlaGlnMetSerValGlyGlnArgAlaLysLeuThrIleSerProAspTyrAlaTyrGlyAlaThrG
lyHisProGlyIleIleProProHisAlaThrLeuValPheAspValGluLeuLeuLysLeuGluSerGlyGlyGlySerGly
SEQ ID NO: 9 (MyD88 nucleotide sequence)
atggctgcaggaggtcccggcgcggggtctgcggccccggtctcctccacatcctcccttcccctggctgctctcaacatgcgagtgcggc
gccgcctgtctctgttcttgaacgtgcggacacaggtggcggccgactggaccgcgctggcggaggagatggactttgagtacttggagat
ccggcaactggagacacaagcggaccccactggcaggctgctggacgcctggcagggacgccctggcgcctctgtaggccgactgct
cgagctgcttaccaagctgggccgcgacgacgtgctgctggagctgggacccagcattgaggaggattgccaaaagtatatcttgaagc
agcagcaggaggaggctgagaagcctttacaggtggccgctgtagacagcagtgtcccacggacagcagagctggcgggcatcacc
acacttgatgaccccctggggcatatgcctgagcgtttcgatgccttcatctgctattgccccagcgacatccagtttgtgcaggagatgatcc
ggcaactggaacagacaaactatcgactgaagttgtgtgtgtctgaccgcgatgtcctgcctggcacctgtgtctggtctattgctagtgagct
catcgaaaagaggtgccgccggatggtggtggttgtctctgatgattacctgcagagcaaggaatgtgacttccagaccaaatttgcactc
agcctctctccaggtgcccatcagaagcgactgatccccatcaagtacaaggcaatgaagaaagagttccccagcatcctgaggttcatc
actgtctgcgactacaccaacccctgcaccaaatcttggttctggactcgccttgccaaggccttgtccctgccc
SEQ ID NO: 10 (MyD88 amino acid sequence)
M A A G G P G A G S A A P V S S T S S L P L A A L N M R V R R R L S L F L N V R T Q V A A D
W T A L A E E M D F E Y L E I R Q L E T Q A D P T G R L L D A W Q G R P G A S V G R L L E L
L T K L G R D D V L L E L G P S I E E D C Q K Y I L K Q Q Q E E A E K P L Q V A A V D S S V P
R T A E L A G I T T L D D P L G H M P E R F D A F I C Y C P S D I Q F V Q E M I R Q L E Q T N
Y R L K L C V S D R D V L P G T C V W S I A S E L I E K R C R R M V V V V S D D Y L Q S K E
C D F Q T K F A L S L S P G A H Q K R L I P I K Y K A M K K E F P S I L R F I T V C D Y T N C P
T K S W F W T R L A K A L S L P
SEQ ID NO: 11 (VCAM-1 nucleotide sequence: NM_001078)
atgcctgggaagatggtcgtgatccttggagcctcaaatatactttggataatgtttgcagcttctcaagcttttaaaatcgagaccaccccag
aatctagatatcttgctcagattggtgactccgtctcattgacttgcagcaccacaggctgtgagtccccatttttctcttggagaacccagata
gatagtccactgaatgggaaggtgacgaatgaggggaccacatctacgctgacaatgaatcctgttagttttgggaacgaacactcttacc
tgtgcacagcaacttgtgaatctaggaaattggaaaaaggaatccaggtggagatctactcttttcctaaggatccagagattcatttgagtg
gccctctggaggctgggaagccgatcacagtcaagtgttcagttgctgatgtatacccatttgacaggctggagatagacttactgaaagg
agatcatctcatgaagagtcaggaatttctggaggatgcagacaggaagtccctggaaaccaagagtttggaagtaacctttactcctgtc
attgaggatattggaaaagttcttgtttgccgagctaaattacacattgatgaaatggattctgtgcccacagtaaggcaggctgtaaaagaa
ttgcaagtctacatatcacccaagaatacagttatttctgtgaatccatccacaaagctgcaagaaggtggctctgtgaccatgacctgttcc
agcgaggtctaccagctccagagattttctggagtaagaaattagataatgggaatctacagcacctttctggaaatgcaactctcacctt
aattgctatgaggatggaagattctggaatttatgtgtgtgaaaggagttaatttgattgggaaaaacagaaaagaggtggaattaattgttca
agagaaaccatttactgttgagatctcccctggaccccggattgctgctcagattggagactcagtcatgttgacatgtagtgtcatgggctgt
gaatccccatctttctcctggagaacccagatagacagccctctgagcgggaaggtgaggagtgaggggaccaattccacgctgaccct
gagccctgtgagttttgagaacgaacactcttatctgtgcacagtgacttgtggacataagaaactggaaaagggaatccaggtggagctc
tactcattccctagagatccagaaatcgagatgagtggtggcctcgtgaatgggagctctgtcactgtaagctgcaaggttcctagcgtgta
cccccttgaccggctggagattgaattacttaagggggagactattctggagaatatagagtttttggaggatacggatatgaaatctctaga
gaacaaaagtttggaaatgaccttcatccctaccattgaagatactggaaaagctcttgtttgtcaggctaagttacatattgatgacatgga
attcgaacccaaacaaaggcagagtacgcaaacactttatgtcaatgttgcccccagagatacaaccgtcttggtcagcccttcctccatc
ctggaggaaggcagttctgtgaatatgacatgcttgagccagggctttcctgctccgaaaatcctgtggagcaggcagctccctaacgggg
agctacagcctctttctgagaatgcaactctcaccttaatttctacaaaaatggaagattctggggtttatttatgtgaaggaattaaccaggct
ggaagaagcagaaaggaagtggaattaattatccaagttactccaaaagacataaaacttacagcttttccttctgagagtgtcaaagaa
ggagacactgtcatcatctcttgtacatgtggaaatgttccagaaacatggataatcctgaagaaaaaagcggagacaggagacacagt
actaaaatctatagatggcgcctataccatccgaaaggcccagttgaaggatgcgggagtatatgaatgtgaatctaaaaacaaagttgg
ctcacaattaagaagtttaacacttgatgttcaaggaagagaaaacaacaaagactatttttctcctgagcttctcgtgctctattttgcatcctc
cttaataatacctgccattggaatgataatttactttgcaagaaaagccaacatgaaggggtcatatagtcttgtagaagcacagaaatcaa
aagtg
SEQ ID NO: 12 (VCAM-1 amino acid sequence)
MPGKMVVILGASNILWIMFAASQAFKIETTPESRYLAQIGDSVS
LTCSTTGCESPFFSWRTQIDSPLNGKVTNEGTTSTLTMNPVSFGNEHSYLCTATCESR
KLEKGIQVEIYSFPKDPEIHLSGPLEAGKPITVKCSVADVYPFDRLEIDLLKGDHLMK
SQEFLEDADRKSLETKSLEVTFTPVIEDIGKVLVCRAKLHIDEMDSVPTVRQAVKELQ
VYISPKNTVISVNPSTKLQEGGSVTMTCSSEGLPAPEIFWSKKLDNGNLQHLSGNATL
TLIAMRMEDSGIYVCEGVNLIGKNRKEVELIVQEKPFTVEISPGPRIAAQIGDSVMLT
CSVMGCESPSFSWRTQIDSPLSGKVRSEGTNSTLTLSPVSFENEHSYLCTVTCGHKKL
EKGIQVELYSFPRDPEIEMSGGLVNGSSVTVSCKVPSVYPLDRLEIELLKGETILENI
EFLEDTDMKSLENKSLEMTFIPTIEDTGKALVCQAKLHIDDMEFEPKQRQSTQTLYVN
VAPRDTTVLVSPSSILEEGSSVNMTCLSQGFPAPKILWSEQLPNGELQPLSENATLTL
ISTKMEDSGVYLCEGINQAGRSRKEVELIIQVTPKDIKLTAFPSESVKEGDTVIISCT
CGNVPETWIILKKKAETGDTVLKSIDGAYTIRKAQLKDAGVYECESKNKVGSQLRSLT
LDVQGRENNKDYFSPELLVLYFASSLIIPAIGMIIYFARKANMKGSYSLVEAQKSKV
SEQ ID NO: 13 (IL-6 nucleotide sequence NM_000600)
atgaactccttctccacaagcgccttcggtccagttgccttctccctggggctgctcctggtgttgcctgctgcccttccctgccccagtaccccc
aggagaagattccaaagatgtagccgccccacacagacagccactcacctcttcagaacgaattgacaaacaaattcggtacatcctcg
acggcatctcagccctgagaaaggagacatgtaacaagagtaacatgtgtgaaagcagcaaagaggcactggcagaaaacaacctg
aaccttccaaagatggctgaaaaagatggatgcttccaatctggattcaatgaggagacttgcctggtgaaaatcatcactggtcttttggag
tttgaggtatacctagagtacctccagaacagatttgagagtagtgaggaacaagccagagctgtgcagatgagtacaaaagtcctgatc
cagttcctgcagaaaaaggcaaagaatctagatgcaataaccacccctgacccaaccacaaatgccagcctgctgacgaagctgcag
gcacagaaccagtggctgcaggacatgacaactcatctcattctgcgcagctttaaggagttcctgcagtccagcctgagggctcttcggc
aaatg
SEQ ID NO: 14 (IL-6 amino acid sequence)
MNSFSTSAFGPVAFSLGLLLVLPAAFPAPVPPGEDSKDVAAPHR
QPLTSSERIDKQIRYILDGISALRKETCNKSNMCESSKEALAENNLNLPKMAEKDGCF
QSGFNEETCLVKIITGLLEFEVYLEYLQNRFESSEEQARAVQMSTKVLIQFLQKKAKN
LDAITTPDPTTNASLLTKLQAQNQWLQDMTTHLILRSFKEFLQSSLRALRQM
SEQ ID NO: 15 (IL-6R nucleotide sequence: IL-6R: NM_000565) IL-6sR is derived from IL-6R sequence.
atgctggccgtcggctgcgcgctgctggctgccctgctggccgcgccgggagcggcgctggccccaaggcgctgccctgcgcaggagg
tggcgagaggcgtgctgaccagtctgccaggagacagcgtgactctgacctgcccgggggtagagccggaagacaatgccactgttca
ctgggtgctcaggaagccggctgcaggctcccaccccagcagatgggctggcatgggaaggaggctgctgctgaggtcggtgcagctc
cacgactctggaaactattcatgctaccgggccggccgcccagctgggactgtgcacttgctggtggatgttccccccgaggagccccag
ctctcctgcttccggaagagccccctcagcaatgttgtttgtgagtggggtcctcggagcaccccatccctgacgacaaaggctgtgctcttg
gtgaggaagtttcagaacagtccggccgaagacttccaggagccgtgccagtattcccaggagtcccagaagttctcctgccagttagca
gtcccggagggagacagctctttctacatagtgtccatgtgcgtcgccagtagtgtcgggagcaagttcagcaaaactcaaacctttcagg
gttgtggaatcttgcagcctgatccgcctgccaacatcacagtcactgccgtggccagaaacccccgctggctcagtgtcacctggcaag
acccccactcctggaactcatctttctacagactacggtttgagctcagatatcgggctgaacggtcaaagacattcacaacatggatggtc
aaggacctccagcatcactgtgtcatccacgacgcctggagcggcctgaggcacgtggtgcagcttcgtgcccaggaggagttcgggca
aggcgagtggagcgagtggagcccggaggccatgggcacgccttggacagaatccaggagtcctccagctgagaacgaggtgtcca
cccccatgcaggcacttactactaataaagacgatgataatattctcttcagagattctgcaaatgcgacaagcctcccagtgcaagattctt
cttcagtaccactgcccacattcctggttgctggagggagcctggccttcggaacgctcctctgcattgccattgttctgaggttcaagaagac
gtggaagctgcgggctctgaaggaaggcaagacaagcatgcatccgccgtactctttggggcagctggtcccggagaggcctcgaccc
accccagtgcttgttcctctcatctccccaccggtgtcccccagcagcctggggtctgacaatacctcgagccacaaccgaccagatgcca
gggacccacggagcccttatgacatcagcaatacagactacttcttccccaga
SEQ ID NO: 16 (IL-6sR amino acid sequence) IL-6sR is derived from IL-6R sequence.
MLAVGCALLAALLAAPGAALAPRRCPAQEVARGVLTSLPGDSVT
LTCPGVEPEDNATVHWVLRKPAAGSHPSRWAGMGRRLLLRSVQLHDSGNYSCYRAGRP
AGTVHLLVDVPPEEPQLSCFRKSPLSNVVCEWGPRSTPSLTTKAVLLVRKFQNSPAED
FQEPCQYSQESQKFSCQLAVPEGDSSFYIVSMCVASSVGSKFSKTQTFQGCGILQPDP
PANITVTAVARNPRWLSVTWQDPHSWNSSFYRLRFELRYRAERSKTFTTWMVKDLQHH
CVIHDAWSGLRHVVQLRAQEEFGQGEWSEWSPEAMGTPWTESRSPPAENEVSTPMQAL
TTNKDDDNILFRDSANATSLPVQDSSSVPLPTFLVAGGSLAFGTLLCIAIVLRFKKTW
KLRALKEGKTSMHPPYSLGQLVPERPRPTPVLVPLISPPVSPSSLGSDNTSSHNRPDA
RDPRSPYDISNTDYFFPR
```

### Example 11

### Clinical Treatment of Patients with Dendritic Cells Transfected with iCD40

Summary of Methods

Men with progressive metastatic castration resistant prostate cancer were enrolled in a 3+3 dose escalation Phase I/11a trial evaluating BPX-101. BPX-101 is produced from a single leukapheresis product by elutriation, differentiation of monocytes into DCs, transduction with Ad5f35-inducible human (ih)-CD40, brief treatment with lipopolysaccharide, and antigen loading with a form of PSMA polypeptide (Prostate Specific Membrane Antigen). BPX-101 was administered intradermally every 2 wks for 6 doses. 24 hrs after each dose, one dose of activating agent AP1903 (0.4 mg/kg) was infused. Exploratory clinical and immunological assessments were performed during the acute phase including serum PSA every 4 weeks, CT/MRI and radionuclide bone scan every 12 weeks, injection site DTH skin biopsy and assay for antigen specific immune response at Week 5, and measurement of serum cytokines for systemic immune response and IL-6 weekly. Of the 12 subjects enrolled in the study, the average Halabi-predicted survival was 13.8 months.

Vaccine

Ad5f35-ihCD40

Inducible human CD40 receptor was cloned into a replication-deficient Ad5-based vector derived from adenovirus serotype 35 (Ad35). The Ad5f35 adenovirus has been cloned into the versatile AdEasy system (Gittes, R. F., New England Journal of Medicine 324, 236-45 (1991)) and contains an engineered gene consisting of the Ad5 fiber tail domain and the Ad35 fiber shaft and knob domains. The Ad5f35 virus has an efficient tropism for cells of hematopoietic origin, as it utilizes ubiquitously expressed CD46 as a receptor for entry into host cells (Crawford, E. D. et al., [erratum appears in N Engl J Med 1989 Nov. 16; 321(20):1420]. New England Journal of Medicine 321, 419-24 (1989)).

The Ad5f35-ihCD40 encodes a single transgene comprising multiple components:
One copy of the myristoylation—targeting domain from human c-Src (Myr)
One copy of human FKBP12(V36) containing “wobbled” codons (Fv′)
One copy of FKBP12 (V36) (Fv)
Short G-S linker (Is)
Cytoplasmic domain of human CD40 (CD40c)

The expression of the transgene is controlled by a cytomegalovirus (CMV)-derived promoter.

The N-terminal myristoylated membrane localization domain of c-Src (14 a.a.) is used to localize the iCD40 receptor to intracellular membranes. The myristoylation-targeting sequence from c-Src was originally designed as a PCR oligonucleotide containing convenient restriction sites for subcloning and joining onto the FKBP domains.

FKBP12(V36): The human 12 kDa FK506-binding protein with an F36 to V substitution, the complete mature coding sequence (amino acids 1-107), provides a binding site for synthetic dimerizer drug AP1903 (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein are included in the construct so that higher-order oligomers are induced upon cross-linking by AP1903; the activation of CD40 normally requires formation of receptor trimers.

F36V′-FKBP: F36V′-FKBP is a codon-wobbled version of F36V-FKBP. It encodes the identical polypeptide sequence as F36V-FKPB but has only 62% homology at the nucleotide level. F36V′-FKBP was designed to reduce recombination in retroviral vectors (Schellhammer, P. F. et al., J. Urol. 157, 1731-5 (1997)). F36V′-FKBP was constructed by a PCR assembly procedure. The transgene contains one copy of F36V′-FKBP linked directly to one copy of F36V-FKBP.

CD40: The CD40 receptor cDNA sequence encodes the entire 62 amino acid cytoplasmic domain of the human CD40 gene (188 a.a.). This region includes multiple binding sites for TNF receptor associated factors 2, 3 and 6 (TRAFs 2, 3 and 6), which are adapter proteins that bridge receptors of the TNF family to downstream signaling molecules, such as NF-κB (Small, E. J. & Vogelzang, N. J., Journal of Clinical Oncology 15, 382-8 (1997); Scher, H. I., et al., Journal of the National Cancer Institute 88, 1623-34 (1996)).

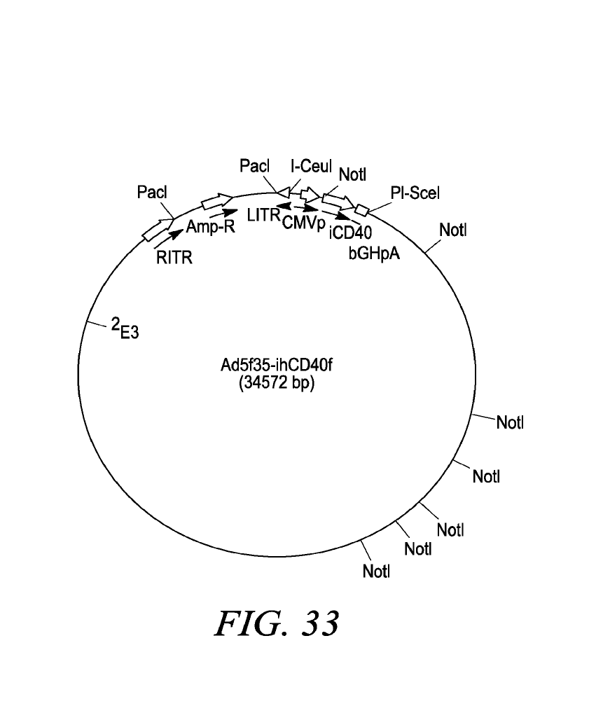

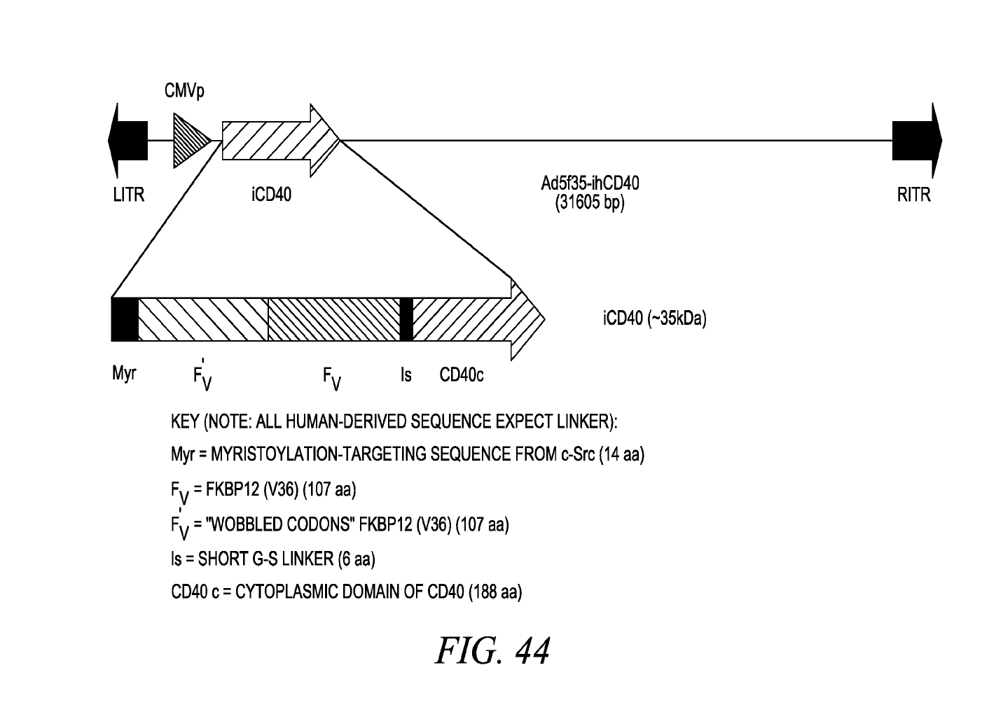

Inducible CD40 was subsequently subcloned into a non-replicating E1, E3-deleted Ad5f35-based vector in the vector core facility at the Center for Cell and Gene Therapy and subsequently replaque-purified and amplified in the associated GMP Vector Production Facility. FIG. 44 presents a map of a CD40 expression vector, and FIG. 33 presents a map of the plasmid Ad5f35ihCD40.

PSMA

The extracellular domain of PSMA protein is used to pulse MoDCs. Initially, most of the extracellular portion of PSMA was PCR-amplified from PSMA clone ID 520715 (Invitrogen) to get 2100 bp. This fragment was subcloned into a transfer vector, containing a baculovirus-derived promoter and amino-terminal hydrophobic secretion signal peptide from abundant envelope surface glycoprotein, gp67. To add exon 18 found in the prostate form of PSMA, containing potential additional immunogenic epitopes, cDNA from human LNCaP cells was PCR-amplified to get 408-bp fragment, containing the 3′ end of PSMA (residues 620-750). This fragment was subcloned to get full-length, pAcGP67.XPSMAx18, which was sequenced throughout the open-reading frame. To make recombinant phage, the plasmid pAcGP67.XPSMAx18 was cotransfected with BD BaculoGold™ DNA (BD Pharmingen) into Sf9 insect cells (Invitrogen, 11496-015). The viral stock was harvested and subjected to two rounds of plaque purification. One plaque was chosen and expanded rendering the P1 viral stock, which was amplified to generate the P2 viral stock used for generating a high titer stock. Cells were grown at all times in serum-free insect medium (Sf 900 IISFM, Gibco). The PSMA expressing Baculovirus stock was used to infect serum-free cultures of expressSf+(Protein Sciences Corp.) cells in Wave Bioreactors. Once expressed, and subject to post-translational modification, the amino acid sequence no longer includes the signal peptide sequence. The supernatant was harvested and clarified, then concentrated by tangential ultrafiltration (UF) and diafiltered into the loading buffer for the column to be used in the following step, filtered through a 0.2 μm membrane and purified by Nickel affinity chromatography. The eluted PSMA was collected and buffer exchanged into PBS. This material was nano filtered, sterile filtered and aliquoted into vials at a concentration of approximately 0.4 mg/mL and stored at −80° C.

LPS

LPS is a TLR-4 ligand and a critical component for the full functional activation of BPGMAX-CD1. LPS from Salmonella typhosa (Sigma-Aldrich) is purified by gel-filtration chromatography, γ-irradiated, and cell culture tested. A single lot is used to co-activate the MoDCs of BPX-101.

Autologous Cell Processing

Donor mononuclear cells are obtained by apheresis and dendritic cell precursors are selected by elutriation. MoDCs are generated by stimulation of precursor cells in culture with 800 U/mL human GM-CSF and 500 U/mL human IL-4 for in serum-free CellGenix DC medium. Immature DCs are harvested and pulsed with PSMA protein (˜10 μg/mL) and then transduced with Ad5f35-ihCD40 and activated with LPS and AP1903 dimerizer. drug. Thereafter, mature MoDCs are extensively washed, harvested and cryopreserved as the final product, BPX-101.

Following full BPX-101 activation (24 hours after LPS addition), noninternalized LPS is removed by extensive washing. The release testing of each batch of BPX-101 drug substance includes endotoxin quantitation as an evaluation of purity.

Stability and Storage

The drug product vaccine, BPX-101, is directly and immediately prepared by adjusting the drug substance cell suspension to a formulation amenable to freezing and maintenance of cell integrity until clinical use. This is accomplished by carefully adding adequate amounts of preservative (HSA), Cryoserve-Dimethyl Sulfoxide (DMSO) and PlasmaLyte and submitting the final cell suspension to a controlled freezing procedure. The first step of the formulation of the fully activated cell preparation (drug substance) is adjusting the concentration to achieve the target dose (4, 12.5 or 40×106 viable cells/mL) based on the total cell counts and viability data (Drug substance release tests) by adding PlasmaLyte-A containing 3% HSA. The cell preparation is then cooled down to 1-6° C. in a monitored refrigerator for at least 15 minutes. Chilled cryoprotectant solution (DMSO/25% HSA/PlasmaLyte-A, 15:35:50 v/v/v) is added to the cell product at a controlled rate in a 1:1 volume ratio (final 7.5% by volume DMSO). The chilled cell preparation is appropriately aliquoted into individual doses in prelabeled cryobags (Cryocyte™, Baxter, now Fenwall Blood Technologies or VueLife™, American Fluoroseal Corporation). This final product is cryopreserved using a standard controlled rate freezing process and is then transferred to a continuously monitored liquid nitrogen storage chamber for storage in vapor phase until sent to the clinic for use.

BPX-101 Preparation and Administration

Leukapheresis and Collection of APC Precursors:

Patients undergo a standard, up to 12 L (˜1.5-2.5× blood volume) leukapheresis procedure over approximately 4 hours to harvest peripheral blood mononuclear cells (lymphocytes and monocytes), yielding a range of 1−30×109 peripheral blood mononuclear cells (PBMCs), 4 weeks before the first 6 vaccinations.

Prior to the leukapheresis procedure, ˜5 mL of blood is drawn for use for establishment of lymphoblastoid cell lines (LCLs).

The patient may be instructed to eat calcium-rich foods the morning of the leukapheresis appointment. Following leukapheresis, the product is transported to the cell processing center. BPX-101 is prepared from the leukapheresis product and subsequently released for administration approximately 4 weeks following the leukapheresis procedure.

Immediately after collection, the leukapheresis product is transported to the cell processing center, for processing into BPX-101. BPX-101 is comprised of antigen-presenting cells (APCs), transduced with Ad5f35-ihCD40 and antigen-loaded with 10 micrograms/ml PA001 (PSMA) containing the extracellular domain of human prostate-specific membrane antigen (PSMA), and then activated with 100 nM AP1903 dimerizer drug and 250 ng/ml lipopolysaccharide (LPS). After vaccine preparation, PA001-loaded genetically-modified monocyte-derived DCs (MoDCs, the biologically active component of BPX-101) are diluted with PlasmaLyte-A/HSA/DMSO to achieve individual target doses of 4, 12.5 or 40×10⁶ viable MoDCs, divided into 5 or 8 aliquots of 2004 each (concentrations of 0.8, 2.5 and 3.1×10⁶ cells per 2004 aliquot, respectively). BPX-101 is subsequently released for administration approximately 4 weeks following the leukapheresis procedure. Quality control testing of the cell product is performed prior to its release (i.e., viability, sterility, endotoxins, contaminants).

BPX-101 is comprised of matured, antigen-expressing DCs derived from monocytes collected during an out-patient leukapheresis procedure. By the end of a six day process conducted in a central GMP processing facility, these cells have been transduced with an adenovector encoding iCD40, incubated with recombinant PSMA, and pre-activated with AP1903 and LPS. The resulting vaccine cells are washed and cryopreserved in individual doses (sufficient for about one year of treatment). Each dosing event consists of BPX-101 vaccine administration via multiple intradermal injections, followed 24 hours later by AP1903 administration via intravenous infusion

Storage and Product Stability:

Prior to administration BP-GMX-CD1 vaccine is stored frozen at −70° C.

BPX-101 Administration

Patients are premedicated with acetaminophen (1,000 mg) PO and diphenhydramine (Benadryl or generic, 25-50 mg PO) or according to institutional standards, 30 minutes prior to vaccine administration. BPX-101 is thawed immediately prior to use in a 35-39° C. water bath, then stored at 2-8° C., and administered as soon as possible after thawing.

Treatment begins at 4×10⁶ cells (Cohort 1), then 12.5×10⁶ cells (Cohort 2), and then 25×10⁶ cells (Cohort 3) every other week. BPX-101 is administered as a 1 mL total dose for Cohort 1 and 2 and as a 1.6 mL total dose for Cohort 3, in 200 μL increments in the dorsal forearm, upper arm and upper leg, alternating between upper arm and dorsal forearm, and between sides with each vaccine booster for Cohort 1 and 2; and in the dorsal forearm, upper arm and upper leg alternating between sides with each vaccine booster for Cohort 3. Each injection is administered at least 2 cm apart. At least two injections are given in each location; i.e., 4 injections in one location and 1 injection in another location is not acceptable. The vaccine is administered at 3 angles at each injection site to ensure maximum volume acceptance.

Each injection site may be circled and numbered with an indelible marker. Injections are given at a minimum of 2 cm apart. Injections are given in the same location at one visit, alternating to another location at the next visit.

Patients are observed for 30 minutes following the injections for untoward adverse effects.

AP1903 for Injection

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (˜10 mg AP1903 for Injection total per vial).

AP1903 is removed from the refrigerator the night before the patient is dosed and stored at a temperature of approximately 21° C. overnight, so that the solution is clear prior to dilution. The solution is prepared within 30 minutes of the start of the infusion in glass or polyethylene bottles or non-DEHP bags and stored at approximately 21° C. prior to dosing.

All study medication is maintained at a temperature between 2 degrees C. and 8 degrees C., protected from excessive light and heat, and stored in a locked area with restricted access.

### Administration

At 24 hours (±4 hours) after each vaccination cycle, patients are administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not be recalculated unless body weight fluctuates by ≧10%. The calculated dose is diluted in 100 mL in 0.9% normal saline before infusion.

Patients are observed for 15 minutes following the end of the infusion for untoward adverse effects.

All patients in the study receive a total of 11 vaccinations, if no progression is noted by Week 13 or after. Patients receive their last dose at week 51. Week 1 is defined as the week of the first vaccination with BPX-101.

BPX-101 is administered in a total of 5×2004 ID injections for a total vaccination dose level of 4 or 12.5×10⁶ cells, or in a total of 8×2004 ID injections for a maximum total vaccination dose level of 25×10⁶ cells. The maximum dose was chosen as the highest level of DCs that could be obtained from a standard ˜12 L leukapheresis, which can generate up to 5.4×10⁸ DCs following elutriation of apheresis product and GM-CSF/IL-4-mediated differentiation of monocyte precursors. The maximum dose chosen for the study (˜0.53×10⁶ cells/kg) is approximately 240-fold below the highest dose of modified DCs, used in the murine pharmacology models (80×10⁶ cells/kg).

In a previous Phase I study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cmaxvalues ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. AP1903 for Injection was shown to be safe and well tolerated at all dose levels and demonstrated a favorable pharmacokinetic profile. Iuliucci J D, et al., J Clin Pharmacol. 41: 870-9, 2001.

The fixed dose of AP1903 for Injection used in this study is 0.4 mg/kg intravenously infused over 2 hours. The amount of AP1903 needed in vitro for effective signaling of cells is 10-100 nM (1600 Da MW). This equates to 16-160 μg/L or ˜0.016-1.6 mg/kg (1.6-160 μg/kg). Doses up to 1 mg/kg were well-tolerated in the Phase I study of AP1903 described above. Therefore, 0.4 mg/kg may be a safe and effective dose of AP1903 for this Phase I study in combination with BPX-101.

### Clinical Study Design

Three cohorts are included in the clinical study.

Dose Levels:

Cohort 1: BPX-101, 4×10⁶ cells in 1.0 mL
Cohort 2: BPX-101, 12.5×10⁶ cells in 1.0 mL
Cohort 3: BPX-101, 25×10⁶ cells in 1.6 mL BPX-101 therapeutic vaccine is administered at doses of 4 or 12.5×10⁶ cells in 5 ID injections, or 25×10⁶ cells in 8 ID injections.

### Example 12

### Clinical Evaluation

Assays

Methods: Blood was collected immediately prior to and one week after each vaccination. Centrifuged (1500 g) serum samples were aliquoted and stored in liquid nitrogen for later batch testing. Undiluted samples were analyzed in duplicate using the Milliplex Human

Cytokine/Chemokine Panel kit (Millipore, Inc), which includes analytes for GM-CSF, IFN-γ, IL-10, IL-12 (p70), IL-1α, IL-1β, IL-2, IL-4, IL-5, IL-6, IP-10 (CXCL10), MCP-1, MIP-1α, MIP-1β, RANTES, and TNF-α. Data was analyzed using Bio-Plex software (Bio-Rad Laboratories, Inc). All markers falling at least partially inside the standard range (3.2-10,000 pg/mL) are included in each chart.

Interferon Gamma (IFN-Gamma)

Serial levels of IFN-gamma-producing T cells is determined by ELISpot assay. Descriptive analysis is used to summarize IFN-gamma-producing T cell data. These analyses are based on the following measures: change from baseline at each assessment time, average area under the curve minus baseline (AAUCMB) at each assessment time, AAUCMB for the first 6 vaccinations, AAUCMB for all assessments, the maximum value following the first 6 vaccinations and among all assessments, and the time to maximum value.

Statistical modeling is performed to assess the dependence between IFN-gamma-producing T cells and objective response rate. A Cox proportional hazard regression model is used to assess this dependence. An “event” is the initial achievement of a confirmed CR or PR, and time to this event is measured from the first dose of study drug. IFN-gamma-producing cell data used in this analysis is limited to those values collected after initiation of study treatment and no later than the last valid assessment of objective response rate; in the event of a response, only cell data up to and inclusive of the date of the event is used. The model is parameterized to include terms for dose, baseline IFN-gamma cell level, and a time-dependent covariate for IFN-gamma-producing cell level.

Of further interest is the identification of a single IFN-gamma-producing cell value that is predictive of response. A cut-point analysis, based on the log rank statistic, is applied to aid in the selection of this single value among all patients. (Cristofanilli M, et al., N Engl J. Med. 351: 781-91, 2004). The best objective response is the outcome variable and the maximum change from baseline in cell count up to and including the date of best response is the “risk” factor of interest. Due to the small sample size, a p-value of 0.10 is used in selecting the cut-point.

CTL Response

A CTL response may be determined by conventional methods. In this example, autologous LCLs pulsed with PSMA polypeptide is used as APCs in cytotoxicity assays, as well as in the assays requiring T cell re-stimulation in vitro. LCLs is established for each patient by exogenous virus transformation of peripheral B cells by using Epstein Barr Virus-containing supernatants produced by the B95-8 cell line. LCLs are maintained in RPMI 1640, 10% FBS. LCL generation requires 5 ml of blood obtained at the time of enrollment into the clinical trial.

CTL response, as calculated by percent specific lysis, is determined at each study time point and compared to baseline levels. Analysis of these data is based on descriptive statistics and is summarized at each assessment time. Depending on the extent of non-missing, exploratory analyses to assess the dependency of objective response rate on CTL response is made in a manner similar to that proposed for the IFN-gamma-producing cell data.

Optional assay: Only HLA-A2+ patients are included in this optional assay. LNCaP cells (HLA-A2+/PSMA+) is used as a target cell and SK-MeI-37 cells (A2+/PSMA-) will act as a negative control. PSMA antigen recognition is assessed using target cells labeled with ⁵¹Cr (Amersham) for 1 hour at 37° C. and washed three times. Labeled target cells (5000 cells in 50 μL) is added to effector CD8+ cells (1004) at the 5:1, 10:1, 25:1, and 50:1 effector:target cell ratios. Chromium release is measured in supernatants harvested after 4 hours incubation at 37° C. The percentage of specific lysis is calculated as: 100×[(experimental−spontaneous release)/(maximum−spontaneous release)].

Following BPX-101 +AP1903 administration, 6 of 6 patients in Cohort 1 developed erythema and induration at one or more vaccination sites, indicative of delayed-type hypersensitivity (DTH) reactions. T cells were expanded from a single injection site biopsy (6 mm), collected 1 week after the third vaccination. After 4 weeks of culture in IL-2-containing media, flow cytometry revealed ˜30 to 60% CD4+ T cells and 2-10% CD8+ T cells. Antigen-specific responses were analyzed at various ratios of T cells and autologous, EBV-transformed lymphoblastoid cell lines (LCLs) as antigen presenting cells in the presence of (a) PSMA or (b) ovalbumin (control) protein (10 mg/ml) or (c) Ad5f35-empty adenovirus (500 viral particles (VP)/LCL). Supernatants were analyzed in duplicates using the Milliplex Human Cytokine/Chemokine Panel (Millipore, Inc), which includes analytes for GM-CSF, IFN-γ, IL-10, IL-12 (p70), IL-1α, IL-1β, IL-2, IL-4, IL-5, IL-6, IP-10 (CXCL10), MCP-1, MIP-1α, MIP-1β, RANTES, and TNF-α. Chart shows fold increase in cytokine level in group containing T Cells, LCLs and antigen, compared to T cells and LCLs with no antigen. P values are calculated for each antigen by one-way ANOVA with Bonferroni's multiple comparison post-test between T+LCL+antigen vs T+LCL.

Cytokines

BPX-101 from each donor is co-cultured with autologous T cells (at DC:T cell ratio 1:10) for 7 days and (re-stimulated at day 8 with BPX-101). Supernatants are harvested and analyzed by BD Cytometric Bead Array Flex Set for expression of Th1 (IFN-gamma, TNF-alpha) and Th2 (IL-4, IL-5, and IL-10) cytokines.

Serum from patients collected at different time points is analyzed using a Human Cytokine LINCOplex Kit (Millipore Inc) to determine the levels of Th1/Th2 cytokines, such as (IL-2, IFN-gamma, TNF-alpha, IL-4, IL-5, IL-6, and IL-10) on Luminex 100 IS (Bio-Rad Laboratories). Biopsies from 4 of 6 subjects were evaluable for antigen specificity, and all were positive. Subject #1004 (above) and #1001 elicited increases in cytokines suggestive of a TH1 response, whereas subjects 1005 and 1006 were suggestive of a TH2 response. However, this data is generated after three doses, which may be insufficient to elicit a TH1 response in all subjects.

Activation Markers

Peripheral blood leukocytes are incubated for 24 hours with BPX-101 and stained with a panel of antibodies specific for T cell type (CD4 [helper] or CD8 [cytotoxic]) and activation state (CD25 [early activation and TREG subset], CD45R0 [activation and memory subset], and CD69 [early activation]) prior to flow cytometry analysis.

Analysis of these data is based on descriptive statistics and is summarized at each assessment time. Graphical methods are used to further explore changes over time. Measures to be evaluated include actual and change from baseline in the following T cell types: CD4 (helper), CD8 (cytotoxic) and activation state (CD25 [early activation and TREG subset], CD45R0 [activation and memory subset], and CD69 [early activation]).

Other Immunological Markers

Natural Killer (NK) cell activity in the peripheral blood of patients is determined by a simple NK cell assay. Patient leukocytes are cultured at different dilutions for 2-4 hours with universal NK target, K562 cells. The extent of K562 killing is then determined by the loss of propidium iodide exclusion using a flow cytometer.

The extent of injection-site erythema (if any) will also be determined as a direct measurement of the diameter of inflamed tissue. A punch biopsy is scheduled to occur 2-3 days after the 4th vaccination, to be taken from whichever site shows the most inflammation. If no or little (<1 cm) inflammation is observed a biopsy is taken from any one of the injection sites. Infiltration of lymphocytes is determined by histology and immunohistochemistry. The obtained biopsy is split into two approximately equal sections. One part is cryopreserved for immunohistochemistry using anti-CD8, anti-granzyme B, and other possible markers. The second part is cut into small pieces and placed in culture with RPMI 1640, 10% FBS. Leukocytes emigrating from these tissue pieces is cultured with IL-2. After 2 weeks of culturing, T cells are tested for production of Th1/Th2 cytokines upon stimulation with autologous APCs.

Regular weekly blood draws from each patient were evaluated in a broad panel of serum cytokines/chemokines. 4 of 6 subjects (including #1003 (Panel A), #1004 (Panel C), #1005 and #1006) demonstrated systemic up-regulation of IFN-γ, GM-CSF, RANTES, MIP-1α, MIP-1β and MCP-1 one week after each vaccination. TNF-α and IP-10 are detect-able in all subjects but show minimal dose-related change in any subject.

2 of 6 subjects (#1001 (Panel B) and #1002) demonstrated no consistent pattern of detectable serum cytokine changes. However, these subjects had the lowest overall tumor burden, and at least one (#1001) demonstrated an antigen-specific response (#1002 was not assessable). This may suggest that tumor-specific responses in patients with low volume disease may not effect serum cytokine levels.

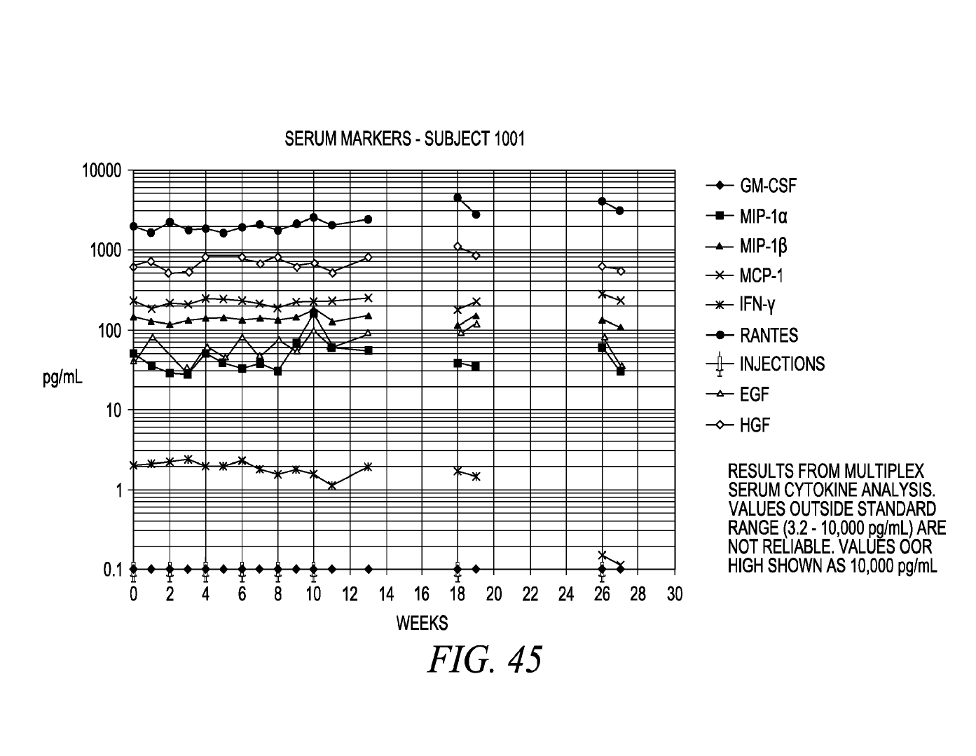

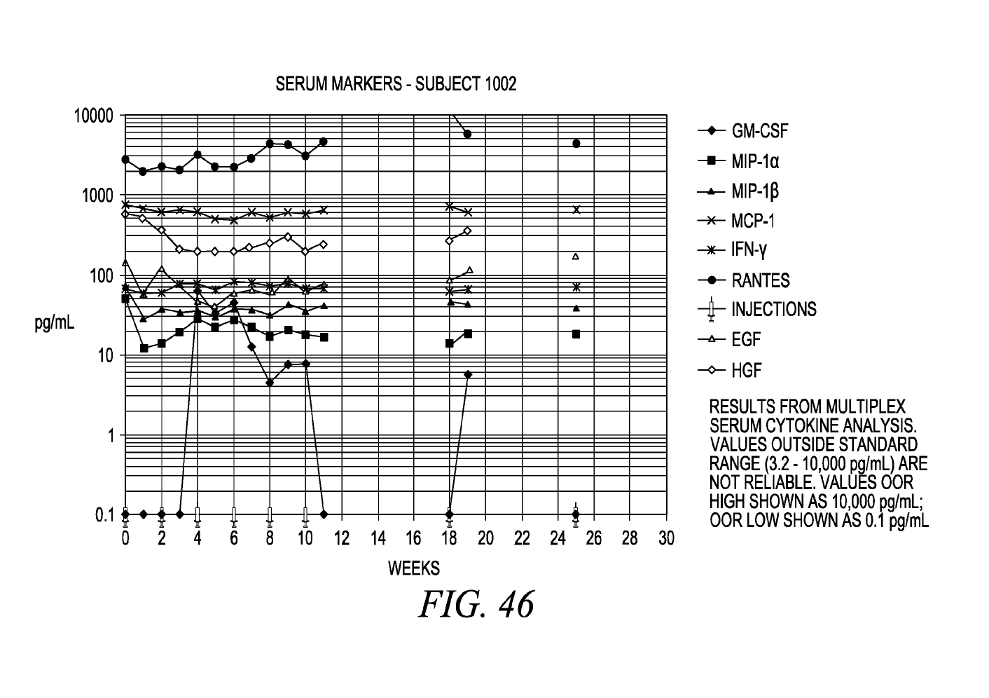

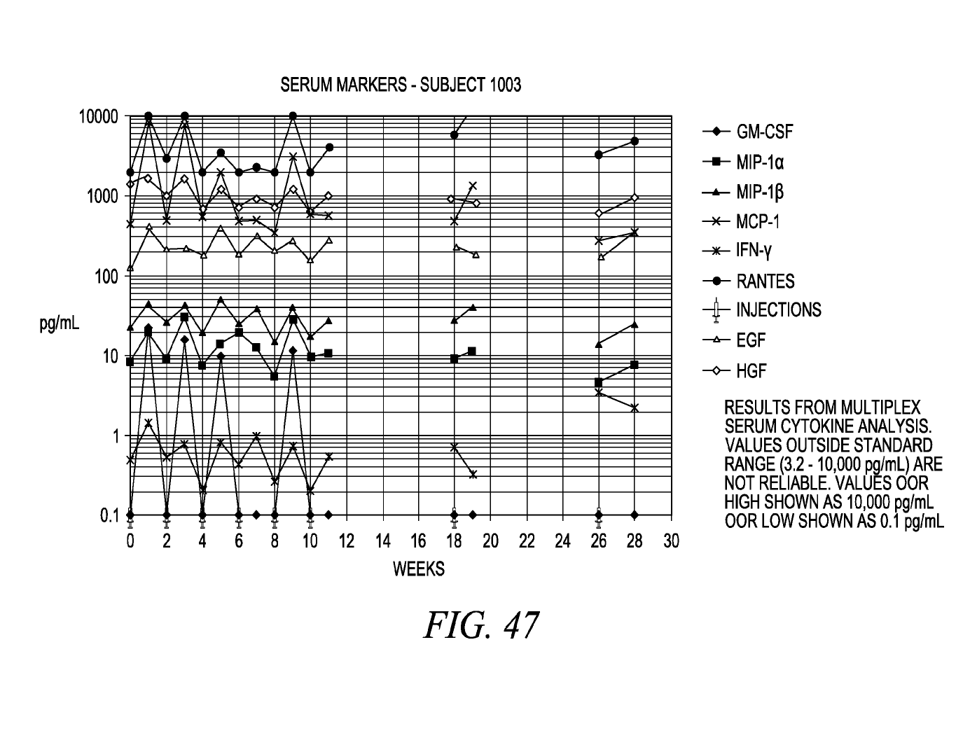

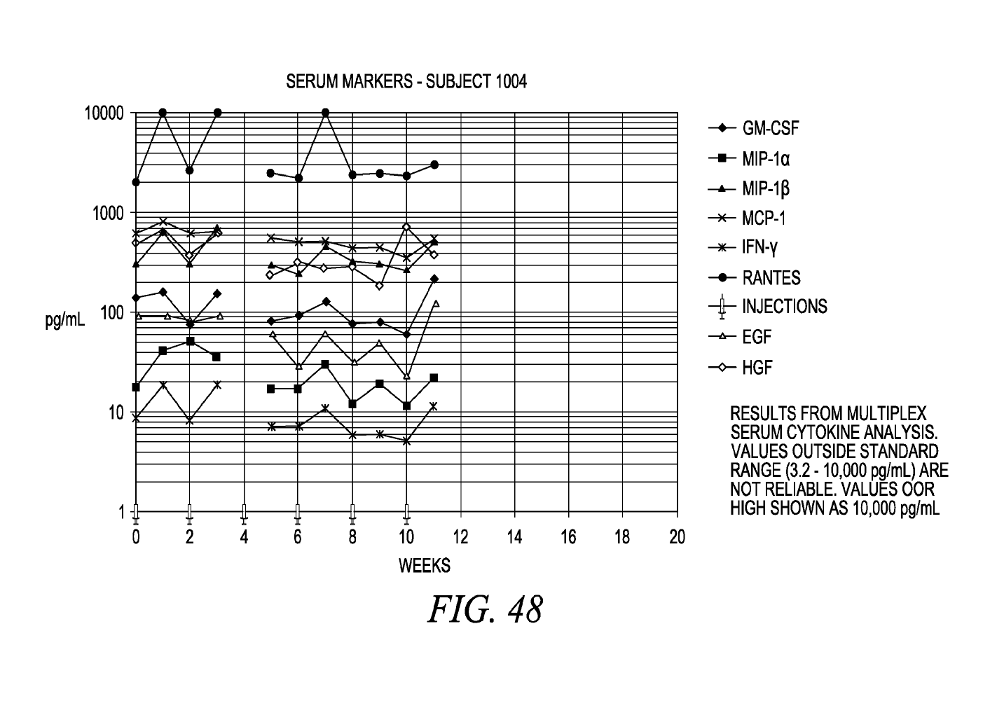

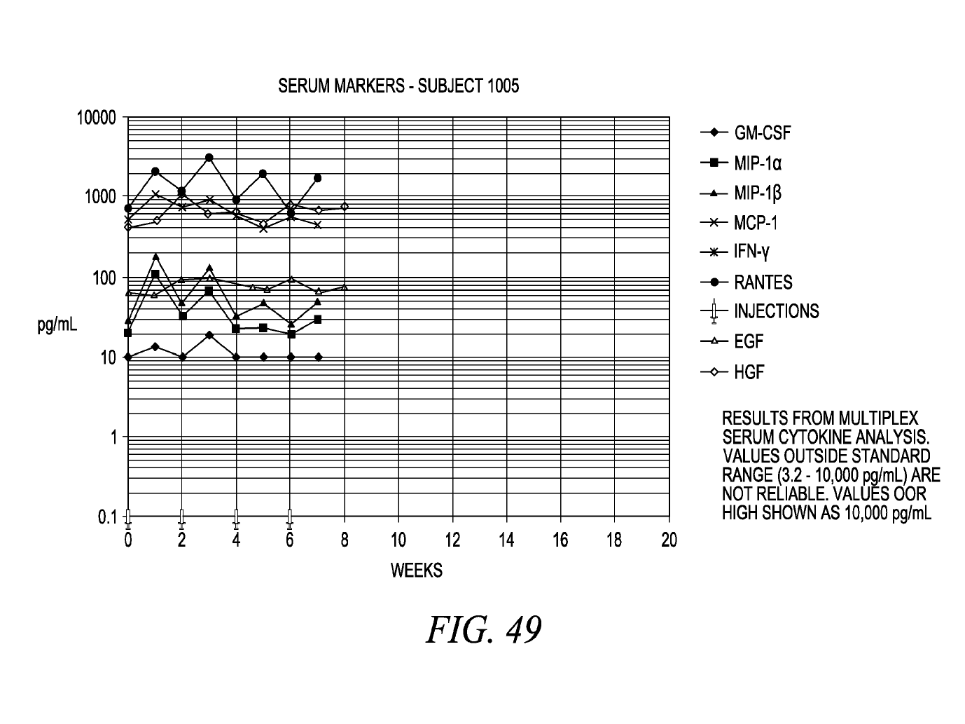

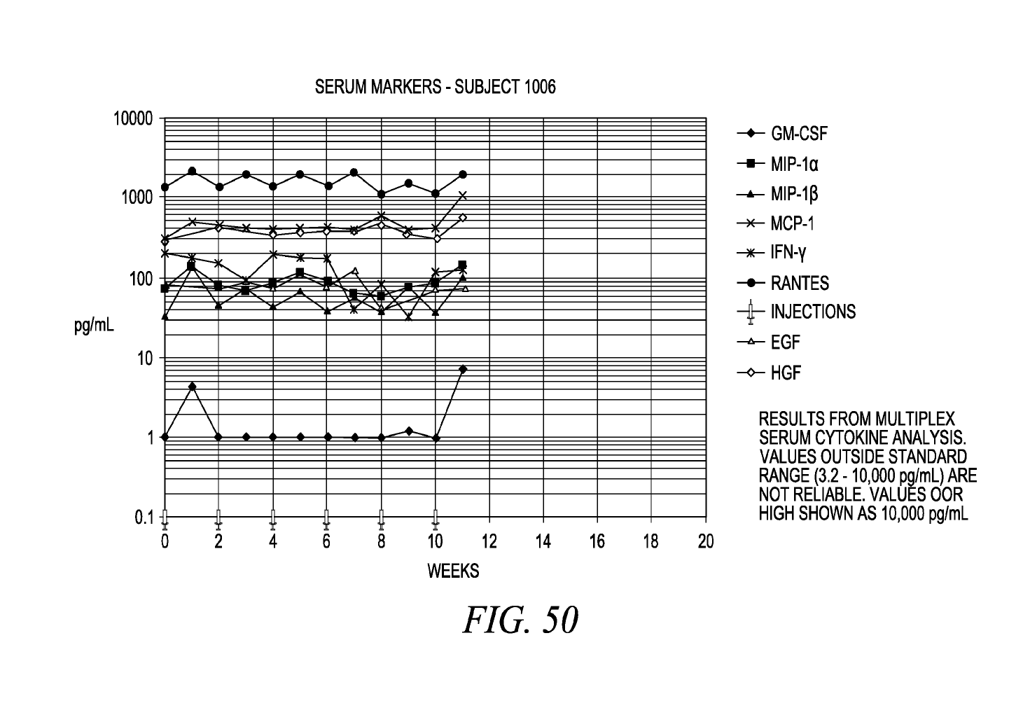

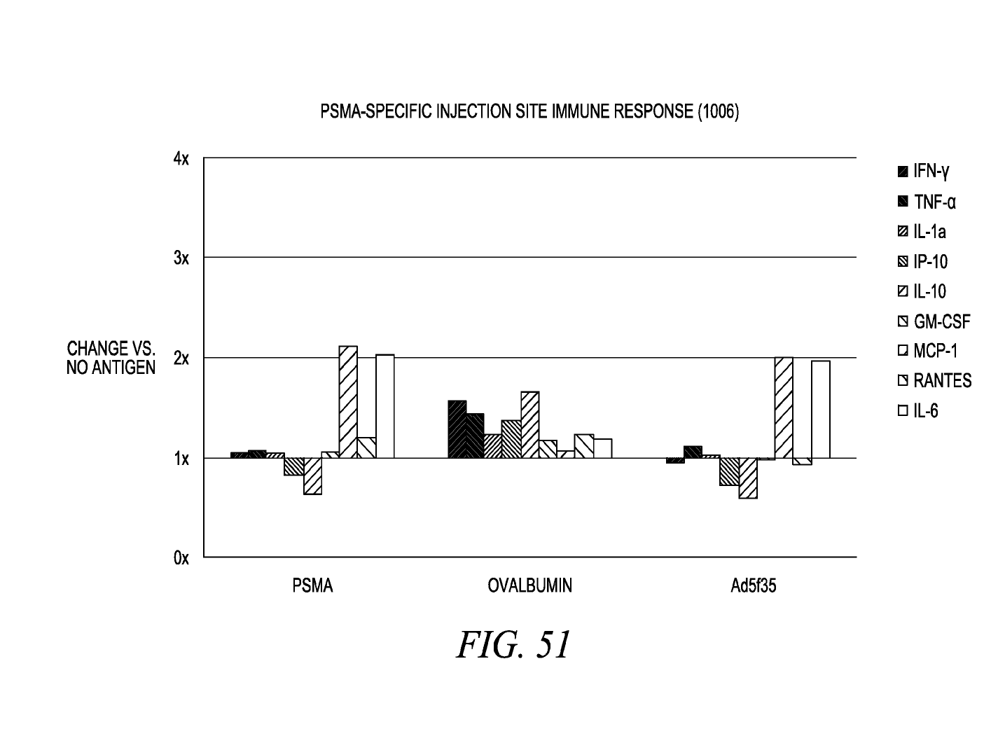

Dose-related cytokine changes were quantified by calculating the unweighted mean change in cytokine level after each dose, for all cytokines and all six doses. This analysis confirms that with a mean post-dose change of −2% and −6%, respectively, neither #1001 nor #1002 exhibited a consistent pattern of serum changes. Also, 3 of 3 subjects in the mid dose cohort exhibited significant increases in serum cytokines 1 week after each dose (mean change range +42% to +72%). In addition, subject 1003 exhibited dramatic serum cytokine perturbation (mean change +283%). MCP-1 levels spiked 17.5-, 17.2-, 4.2- and 6.8-fold over baseline levels one week after vaccination #s 1, 2, 3, and 5, respectively, and returned to within 8-30% of baseline levels the following week in each case. IFN-γ and GM-CSF levels followed a similar pattern; GM-CSF spiked from undetectable baseline levels to 22.0, 16.2, 9.9, and 11.7 pg/ml one week after vaccination #s 1, 2, 3, and 5, respectively, and returned to undetectable levels the following week. The dose-related changes in a panel of secreted factors are shown in FIGS. 45-50. This panel includes GM-CSF, MIP-1alpha, MIP-1beta, MCP-1, IFN-gamma, RANTES, EGF and HGF.

Pharmacokinetic Endpoints

Mean plasma concentrations of AP1903 are determined at each time point. Because plasma concentrations of AP1903 are determined at a limited number of time points during the study, a determination of pharmacokinetic parameters will not be possible.

Biomarker Endpoints

PSA-Based Outcomes

PSA response (proportion of patients achieving a ≧30% and a ≧50% reduction) is summarized at 3 Months and using each patient's maximum change from baseline. Waterfall plots may be used to display changes in PSA. PSA dynamics (change in velocity and doubling time) are summarized using descriptive statistics. Additionally, post-treatment PSA doubling time is compared to pre-treatment PSA doubling time; the proportion of patients experiencing a ≧25% increase in PSA doubling time (change in PSA slope/PSA velocity) is tabulated. Other forms of PSA, if measured, will also be summarized.

PSA Disease Progression

For patients who experience a decline in PSA post-therapy, the first PSA increase that is a ≧25% increase and ≧2 ng/mL absolute increase in PSA level from the nadir value is documented on at least one additional determination at least 3 weeks apart. Once confirmed, the date of the first PSA fitting this progression criteria becomes the date of PSA progression.

If there is no decline from baseline, a ≧25% increase and ≧2 ng/mL absolute increase in PSA level from the pre-treatment value, documented at least 12 weeks from the initiation of therapy.

PSA Doubling Time: PSA doubling time is calculated using the following equation: PSA doubling time=[log(2)×t]÷[log(final PSA)−log(initial PSA)], in which ‘log’ is the natural logarithm function and T is the time from the initial to the final PSA level. The last PSA level measured before initiation of study treatment is defined as the initial PSA. The final PSA value is the last level measured following the initiation of study treatment and before the time point of interest. PSA doubling time is assessed prior to therapy as well as at all times after the initiation of therapy. An additional analysis is performed using the time at which patients are considered to have PSA progression.

PSA Velocity and Slope: The pre-treatment annual PSA velocity (the rate of change in PSA per year) and slope are calculated by simple linear regression from 3 or more PSA measurements before therapy on trial. PSA measurements with complete dates aroused to determine the pre-treatment PSA velocity and slope. Post-treatment PSA velocity during the first 3 months of the study is computed using linear regressions (for patients with two or more PSA measurements in addition to the baseline measurement) and by the ratio of change in the logarithm of PSA (for patients with only one PSA value in addition to the baseline measurement). The slope of the resulting line of best fit is used to determine the PSA velocity and is used to evaluate PSA velocity and slope is assessed prior to therapy as well as at 3 months after the initiation of therapy.

Circulating Tumor Cells (CTCs)

Intact (and apoptotic) CTCs are concentrated from fresh peripheral blood of PCa patients and analyzed for the presence of epithelial cells using the CellSearch technique for immunomagnetic capture of EpCAM+ cells followed by immunostaining for nucleated CD45 negative and cytokeratin (8,18,19) positive cells. (Shaffer D R, et al., Clin Cancer Res. 13: 2023-9, 2007). Typically, fewer than 5 CTCs/10 mL blood sample are found in healthy volunteers and >5 are found in PCa patients. The CellSearch method has been used successfully in diagnosing breast cancer occurrence and progression. (Scher H I, et al., J Clin Oncol. 2008 Mar. 1; 26(7):1148-59). It is FDA approved for breast cancer and more recently for prostate cancer, and is available commercially through Quest Diagnostics. The assay for PCa is basically identical to breast cancer as they are both EpCam+ cells.

Actual and mean change from baseline in CTC is determined for each assessment time point and summarized descriptively. Additionally, where the data permit, the proportion of patients with a ≧50% and ≧90% reduction is determined. Patients are tested before treatment to establish a baseline, before the fourth vaccine, after the first 6 vaccines, after 4-6 months (i.e. 1-2 boosts) and after 10 months.

Efficacy Analyses

Primary efficacy analyses are performed using the FAS; any analyses performed using the PPS are considered supplemental.

Maximum likelihood methods are used to calculate point and interval estimates of treatment effect. Per RECIST criteria, baseline evaluations shall be performed no more than 4 weeks before beginning of the treatment; however, the efficacy endpoints of this Phase I trial are only exploratory. Therefore, results from the screening scans are used as baseline. The best objective response rate and the Week 13 response rate are calculated as the total number of patients having a confirmed CR, or PR divided by the FAS (or PPS as a supplemental analysis). Separate analyses are also conducted for those subjects achieving a confirmed CR. Patients not evaluable following the start of treatment are classified as treatment failures in the FAS dataset.

TTR and duration of response are calculated only for those patients who have a CR or PR. TTR reflects the difference (in days) between the first date of study drug administration and the first date at which objective response criteria are met. Duration of response reflects the difference (in days) between the first date at which response criteria are met and the first date of meeting objective criteria for disease progression or death, whichever event is earlier. Patients not meeting progression criteria may have their event times censored at the last date at which a valid assessment confirmed lack of disease progression.

Patients lacking a tumor assessment post-treatment may have their PFS times censored on the first day that study drug was administered. Sensitivity analyses is conducted to assess the robustness of estimates relative to missed or off-schedule assessments. PFS is estimated for both the FAS and PPS patient populations.

OS is calculated as the difference between the first date that study drug was received and the date of death. Patients who have not died as of the last follow-up may have their times censored on the last known date of contact. OS is summarized for the FAS population; patients lacking survival data beyond the start of treatment will have their observations censored on Day 1.

Choi's GIST criteria (Appendix D) is used as a second criteria for response. The proportion of patients experiencing an objective response (CR or PR) is summarized.

For calculations of duration of response, progression-free survival, and overall survival, one day is added to each calculation. Kaplan-Meier statistics is used to analyze these data and, depending on maturation of the event process, point estimates of the median event rate and 95% confidence interval of the median is provided.

### LIST OF ABBREVIATIONS

The following abbreviations may be used herein, or in the Figures:

| Abbreviation | Definition |
|---|---|
| AAUCMB | Area under the curve minus baseline |
| ADT | Androgen deprivation therapy |
| AE | Adverse event |
| ALT | Alanine transaminase |
| ANC | Absolute neutrophil count |
| APC | Antigen presenting cell |
| AST | Aspartate transaminase |
| BP | Binding protein |
| BPI | Brief Pain Inventory |
| BUN | Blood urea nitrogen |
| CAGT | Center for Cell and Gene Therapy |
| CD | Cluster of differentiation |
| CFR | Code of Federal Regulations |
| CI | Confidence interval |
| CR | Complete response |
| CRF | Case report form |
| CRPC | Castrate resistant prostate cancer |
| CT | Computed tomography |
| CTC | Circulating tumor cell |
| CTCAE | Common terminology criteria for adverse events |
| CTL | Cytotoxic T lymphocyte |
| DCs | Dendritic cells |
| DLT | Dose-limiting toxicity |
| DSMB | Data Safety Monitoring Board |
| EOW | Every other week |
| FAS | Full analysis set |
| FDA | Food and Drug Administration |
| GCP | Good Clinical Practice |
| GM-CSF | Granulocyte-macrophage colony stimulating factor |
| HBsAg | Hepatitis B surface antigen |
| HCV | Hepatitis C virus |
| HIV | Human immunodeficiency virus |
| HTLV | Human T-cell lymphotropic virus |
| ID | Intradermal |
| IEC | Independent ethics community |
| IL | Interleukin |
| IND | Investigational New Drug |
| IRB | Institutional review board |
| IV | Intravenous |
| KPS | Karnofsky Performance Status |
| LDH | Lactate dehydrogenase |
| LN | Lymph Node |
| LPS | Lipopolysaccharide |
| MedDRA | Medical Dictionary for Regulatory Activities |
| MRI | Magnetic resonance imaging |
| mRNA | Messenger ribonucleic acid |
| MTD | Maximum tolerated dose |
| NK | Natural killer |
| NOEL | No observable effect level |
| OS | Overall survival |
| PA001 | Prostate antigen |
| PAP | Prostatic Acid Phosphate |
| PBMC | Peripheral blood mononuclear cell |
| PD | Progressive disease |
| PFS | Progression-free survival |
| PO | Per os |
| PSMA | Prostate-specific membrane antigen |
| PPS | Per protocol set |
| PR | Partial response |
| PSA | Prostate specific antigen |
| RBC | Red blood cell |
| RECIST | Response Evaluation Criteria in Solid Tumors |
| SAE | Serious adverse event |
| SAS | Statistical Analysis System |
| SD | Stable disease/Standard deviation |
| SOC | System organ class |
| TEAE | Treatment-emergent adverse event |
| TTR | Time to response |
| ULN | Upper limit of normal |
| WBC | White blood cell |

### Example 13

### Interim Clinical Data Summary

Summary of Results

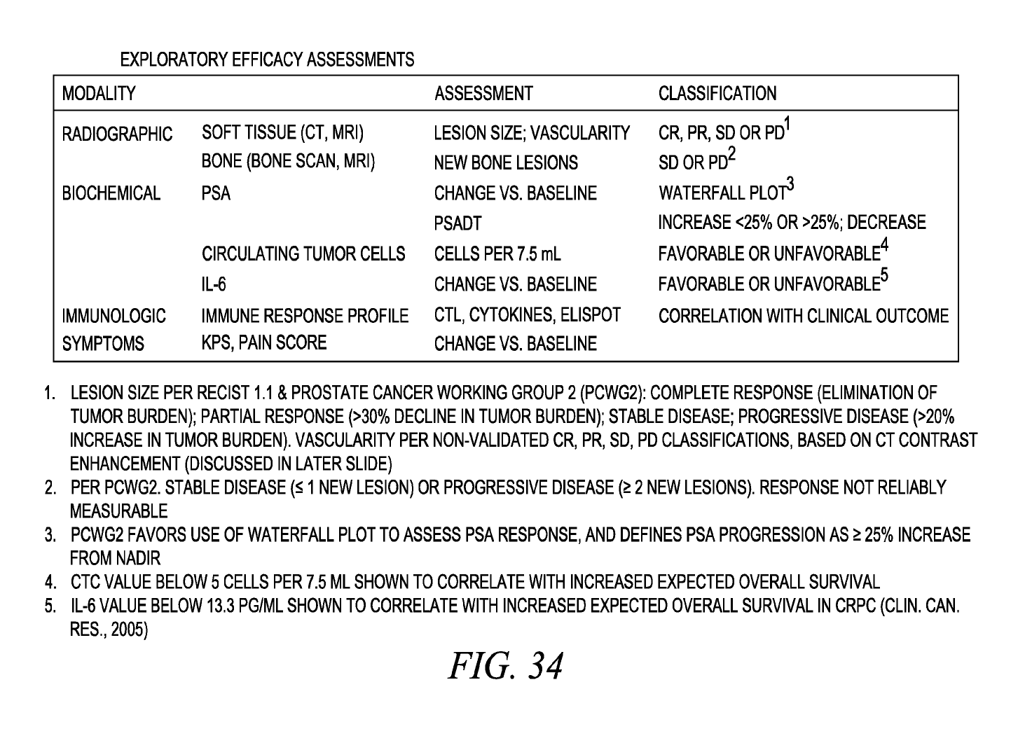

Results: Results: Of 6 subjects enrolled to date, 3 of 3 in the low dose cohort and 2 of 3 in the mid dose cohort completed at least 12 weeks of therapy (median 26, range 12-36), and 4 remain on study with stable disease with no dose limiting toxicity observed. One patient in the mid dose cohort developed impending spinal cord compression due to disease progression and was taken off study at Week 7, after 4 doses were administered, and a second patient was deemed to have disease progression at the end of the acute phase of treatment and was taken off study. The patients were assessed for radiologic, biochemical, immunologic, and symptomatic changes, as summarized in FIG. 34, according to the methods of the clinical protocol.

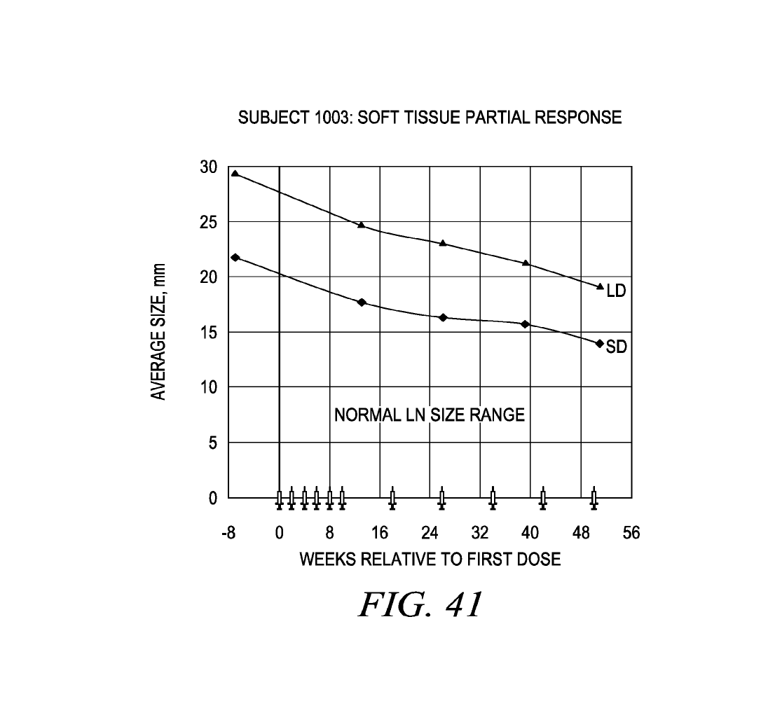

Clinical biomarker responses were evident in both low and mid dose cohorts. 4 of 6 subjects achieved a maximal serum PSA decline ≧10%, including 1 subject (#1003) who achieved ˜50% serum PSA decline by 8 weeks. And 5 of 6 patients experienced a significant prolongation of PSADT. Clinical responses per RECIST 1.1 were observed in 2 of 3 subjects with measurable metastatic disease at baseline, with one subject (#1003) experiencing a 20% decline in measurable disease at 3 months, improving further to a 25% decline at 6 months, tracking towards a Partial Response. FIG. 41 presents a graph of a soft-tissue partial response in subject 1003. Subject 1003 had 8 measurable lymph node lesions at baseline, and demonstrated a steady decrease in all 8 lymph nodes over >1 year. A partial response (PR) per RECIST criteria was found at the 1 year time point. The greatest rate of decrease was seen during induction treatment phase. It is likely that the subject had tumor growth between baseline and the first dose (7 weeks). The third subject with measurable disease progressed, but his PSA stabilized after dose #5.

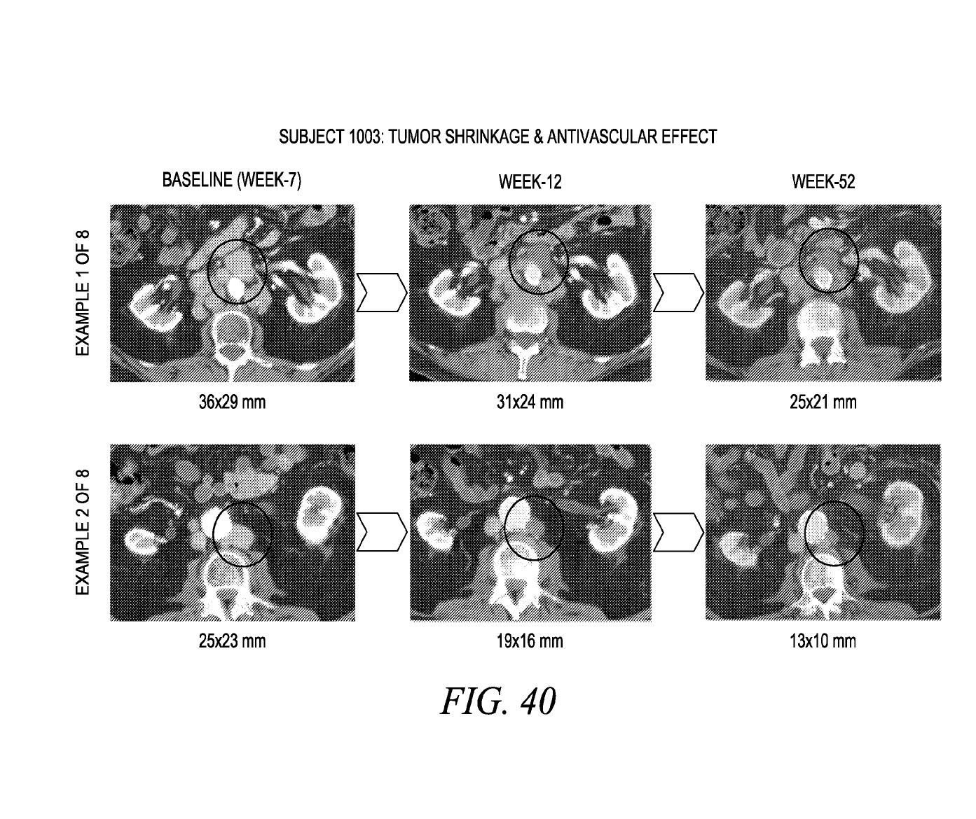

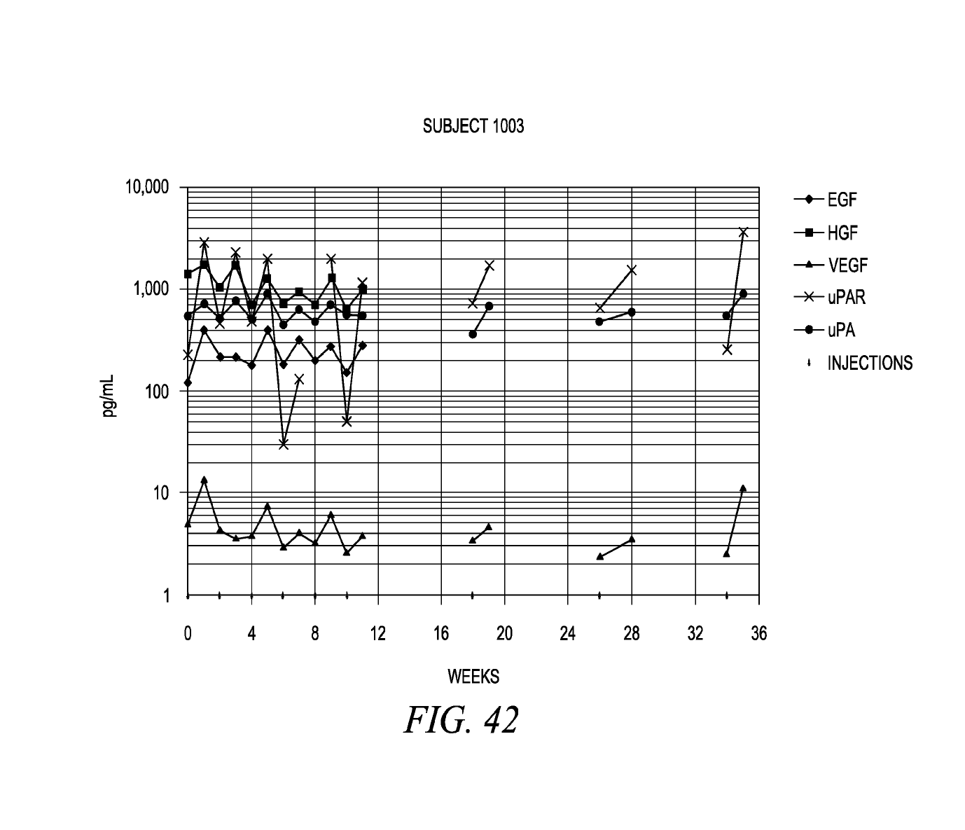

A reduction in tumor vasculature was observed in 3 of 3 subjects with measurable metastatic disease, including the subject whose disease progressed. FIG. 42 presents a graph of various serum markers, demonstrating an anti-vasculature effect. CT contrast enhancement showed a decrease in vascularity in all subjects with MMD. A serum analysis in these subjects revealed a dose-related upregulation of hypoxic factors. PSMA is expressed in solid tumor vasculature and is proposed as anti-vasculature target. Examples of lymph node responses are depicted in FIG. 40, including two nodes that decreased in size and vascularity, measuring 36×29 mm (abnormal >15 mm short axis by RECIST 1.1) and 122 Hounsfield Units (HU) at baseline and 29×24 mm and 40 HU at Week 26 (Example 1), and measuring 25×23 mm and 120 HU at baseline and 17×14 mm and 41 HU at Week 26 (Example 2); and one node that exhibited a complete response, measuring 24×17 mm at baseline and 12×6 mm (normal <10 mm short axis by RECIST 1.1) at Week 26 (Example 3).

4 of 6 subjects demonstrated systemic up-regulation of IFN-γ, GM-CSF, RANTES, MIP-1a, MIP-1β and MCP-1 one week after each vaccination. TNF-α and IP-10 were detectable in all subjects but showed minimal dose-related change in any subject. 2 of 6 subjects demonstrated no consistent pattern of detectable serum cytokine changes, but these subjects had the lowest overall tumor burden. 4 of 6 evaluable subjects showed antigen specific immune responses after three doses, with 2 suggestive of a TH1 response and 2 suggestive of a TH2 response.

Conclusions:

Treatment with BPX-101 and AP1903 elicits both clinical and antigen specific, systemic immune responses. Clinical responses appear to correlate with significant dose-related perturbations in serum cytokines, and a decline in PSA. In 2 of 3 subjects completing 12 wks of therapy at the lowest dose, dramatic spikes in serum inflammatory cytokine levels correlated with PSA declines in both and measurable disease decline in one. Tumor vascularization also decreased in 3 of 3 patients with measurable metastatic disease.

Analysis

Six patients were assessed for progression of disease, after receiving treatment according to the methods of the clinical protocol. The patients were assessed for radiologic, biochemical, immunologic, and symptomatic changes, as summarized in FIG. 34, according to the methods of the clinical protocol.

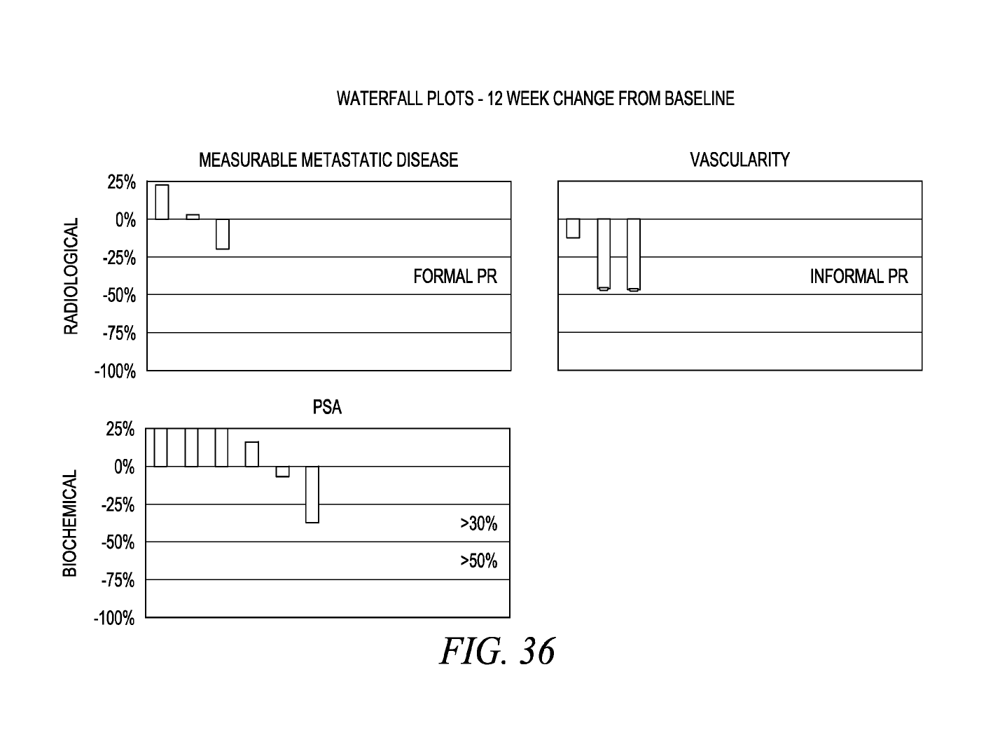

FIG. 34 is a chart presenting exploratory efficacy assessments. FIG. 36 presents a summary of the analysis of a 12 week change in measurable metastatic disease, vascularity, and PSA.

Radiologic

FIG. 40 presents the results of a CT scan of patient 1003 (scan example 1).

Objective clinical responses (soft tissue, per RECIST 1.1) were observed in 2 of 3 subjects with measurable metastatic disease at baseline:
1 subject remained with Stable Disease >6 months.
A second subject (#1003) experienced a 20% decline in measurable disease at 3 months, improving further to a 25% decline at 6 months, tracking towards a Partial Response.

Subject 1003 underwent baseline scans 7 weeks prior to initiation of the acute phase of vaccination at Week 0. Repeat scans at the end of acute phase of treatment, obtained at Week 12, 19 weeks after initiation of therapy showed a 20% decrease in measurable target (2 lymph nodes) and non-target (5 lymph nodes) disease. By week 26 scans, 8 months after baseline scans, all 7 measurable lesions exhibited further reductions in size reaching a 25% reduction in overall measurable disease. Three examples of lymph node responses are depicted above, including one node that exhibited a complete response, measuring 24×17 mm (abnormal >15 mm short axis by RECIST 1.1) at baseline and 12×6 mm (normal <10 mm short axis by RECIST 1.1) at Week 26 (Example 3).

Biochemical

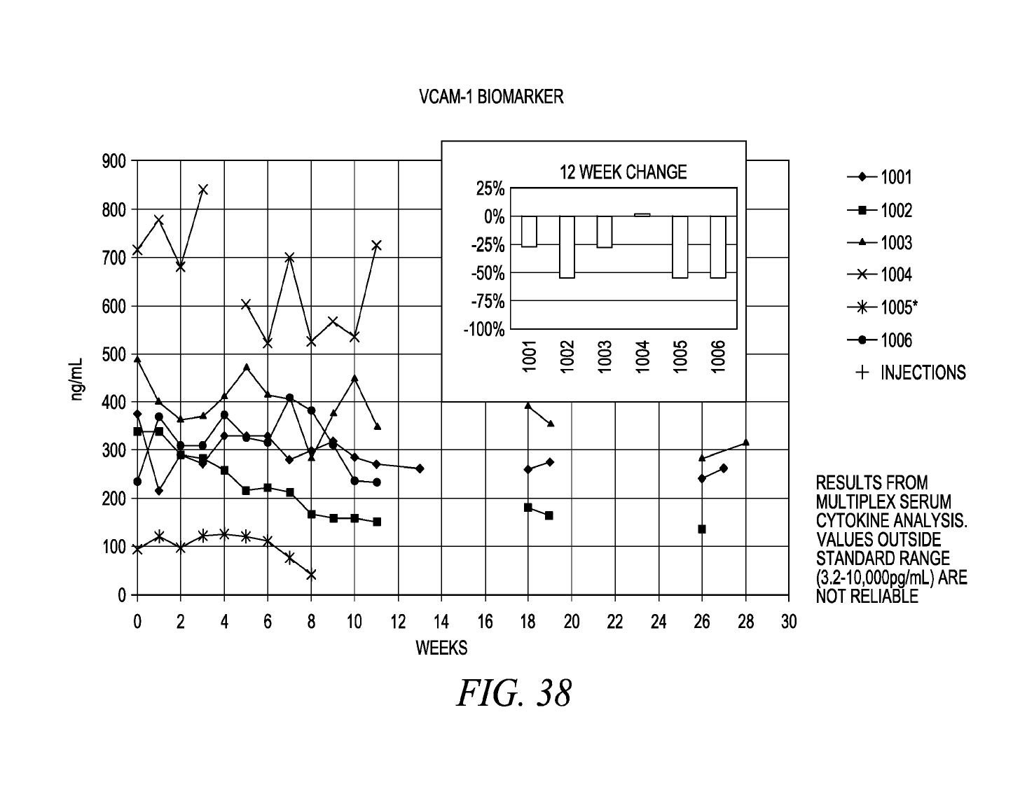

FIG. 38 shows the results of a VCAM-1 serum analysis. A decrease in VCAM-1 concentration was observed after treatment.

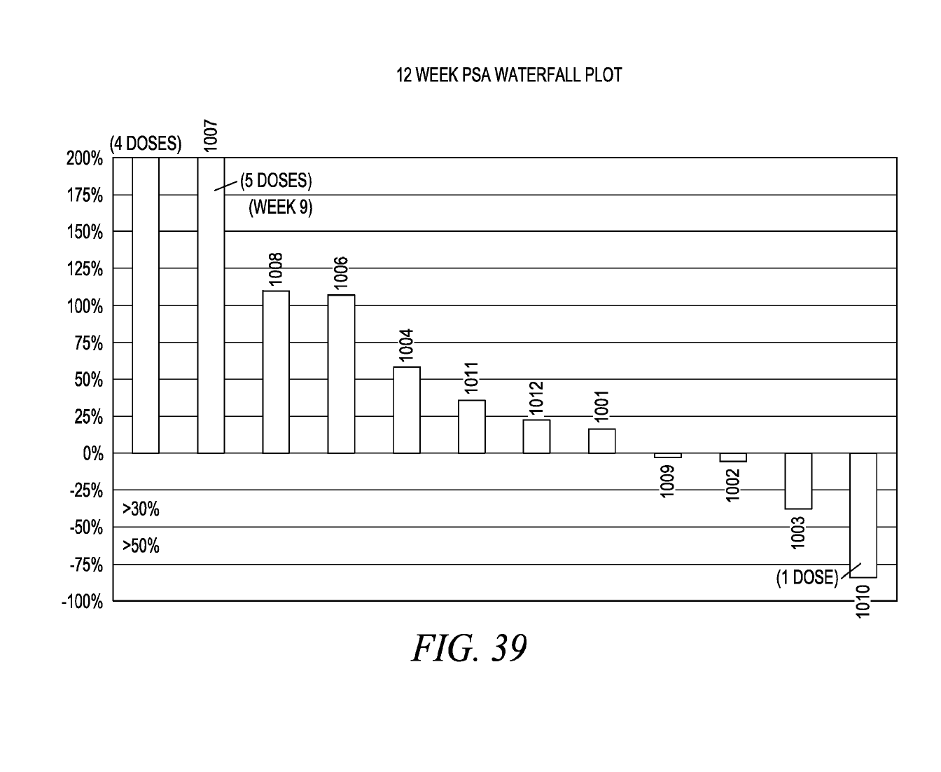

The presence of prostate specific antigen (PSA) was also assessed. FIG. 39 presents a waterfall plot of PSA levels at 12 weeks.

Immunologic

The patients were assessed for various immunologic markers. The significance and the desired outcome is summarized below for each marker.

GM-CSF; Stimulates stem cell differentiation into granulocytes and monocytes, which can further differentiate into macrophages and DCs. Desired outcome: increase.

IFN-gamma: Produced predominantly by activated NK, NKT, T Helper 1 and CTLs. Immunostimulatory, anti-viral, and anti-tumor properties. Desired outcome: increase.

MCP-1: Helps recruit monocytes, memory T cells and DCs to sites of injury or inflammation. Desired outcome: Increase

MIP-1α,β: Produced by activated macrophages to activate chemotaxis in granulocytes and other leukocytes and to induce other pro-inflammatory cytokines (e.g. IL-1, IL-6, TNF-α). Desired outcome: Increase

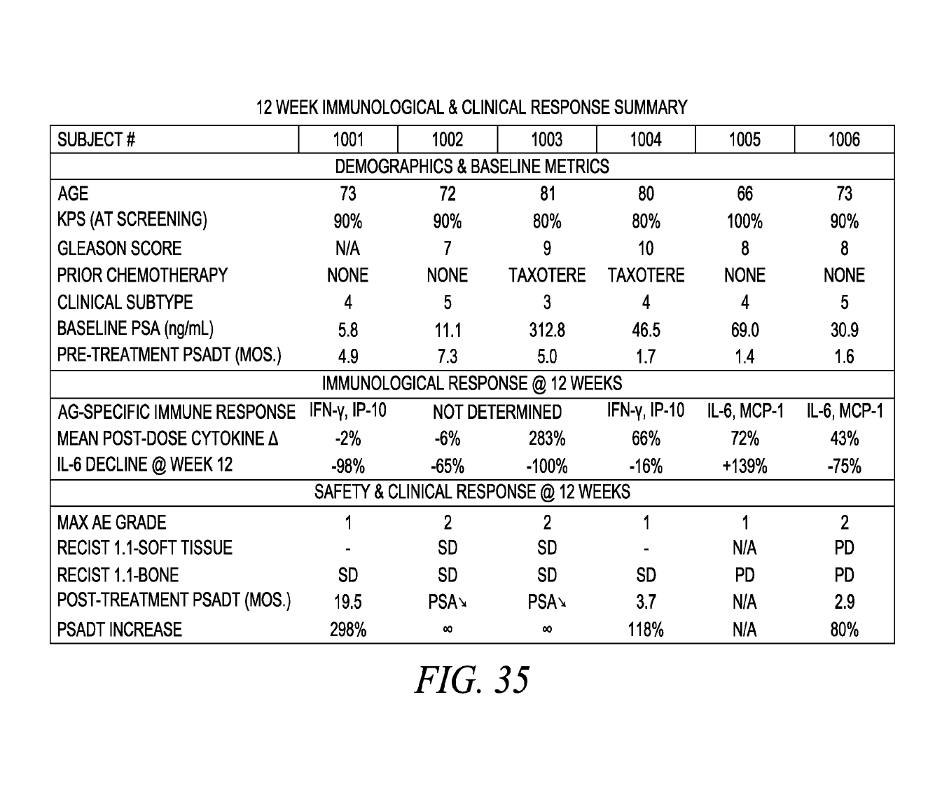

FIG. 35 presents a 12 week immunological and clinical response summary.

FIGS. 45-50 are graphs of serum marker analyses in patients 1001-1006, respectively.

Clinical Biomarkers

Clinical biomarker responses were evident in both low- and mid-dose cohorts. 4 of 6 subjects achieved a maximal serum PSA decline ≧10%, including 1 subject (#1003) who achieved ˜50% serum PSA decline by 8 weeks.

PSA declines were observed in 3 of 3 subjects in the low dose cohort, all of whom had relatively longer PSA doubling times at baseline (4.9-7.3 months), in contrast to the mid dose subjects, all of whom have baseline PSADTs <2 months (1.4-1.7 months).

Symptomatic

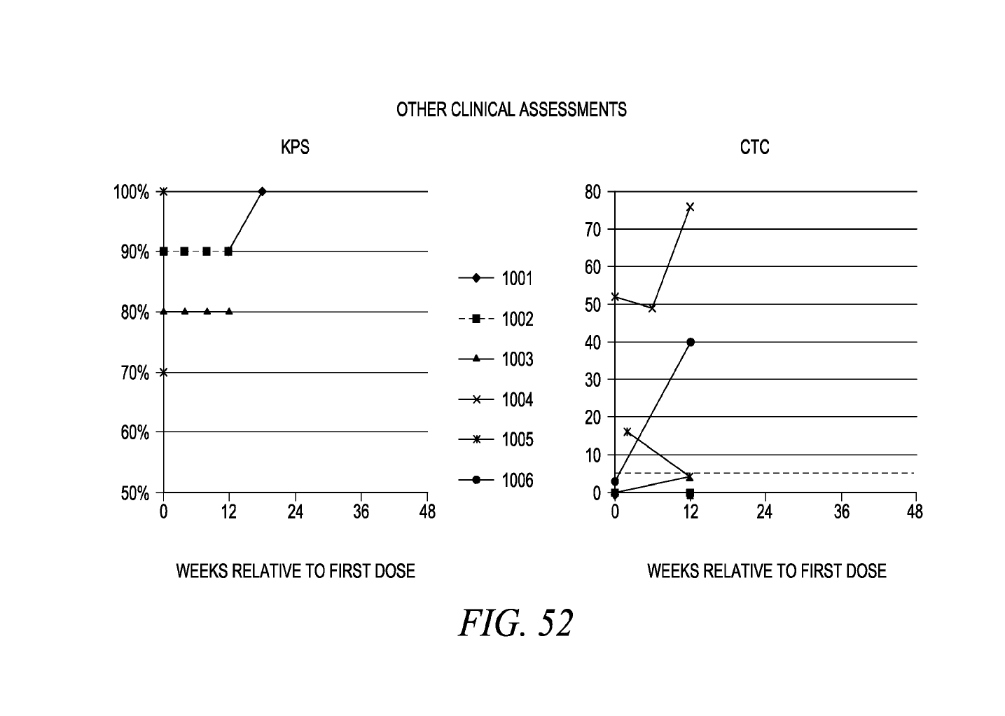

FIG. 52 presents graphs of KPS and CTC assessments.

Treatment with BPX-101 and AP1903 elicits immunological and clinical responses:
Antigen (PSMA)-specific T-cell response, as observed in DTH biopsies of 4/4 patients. Elaborated cytokines reflected either a TH1 or TH2 bias after three doses.
Regular, periodic up-regulation of several soluble factors in 4/6 patients, including changes IFN-γ, GM-CSF, RANTES, MIP-1α, MIP-1β and MCP-1

Objective clinical response appears to correlate with significant dose-related perturbation in serum cytokines, and decline in PSA.

Interim Conclusions

Subject #1003, enrolled in the low dose cohort with features of high-risk, progressive mCRPC, including a high PSA (>300), Gleason Score 9, a serum IL-6 level of >13.3 μg/mL, and failure of prior docetaxel chemotherapy, exhibited a rapid clinical response, including a ˜50% drop in PSA beginning after just 2 vaccinations, and a measurable disease decline of 20% at the end of 12 weeks of therapy and 25% at 6 months, tracking towards a Partial Response (RECIST 1.1). This response correlated with surges in serum cytokines consistent with a systemic immune response resulting from each vaccination cycle. Antigen specificity was not determined in this subject.

Subject #1005, enrolled in the mid dose cohort with extensive bone metastases, Gleason Score 8, and rapidly rising PSA (1.4 months PSADT), exhibited cytokine perturbation after only the first two doses, with a TH2 bias. There was no change in his PSA trajectory. He progressed after 7 weeks.

This and other patient data suggests that the present methods may induce short-term disease responses, leading to a more significant survival benefit without treatment related toxicity.

### Example 14

### Combination Therapy

Metastatic castrate resistant prostate cancer patients have been treated with combinations of chemotherapeutics. When treated with the combination of docetaxel and estramustine phosphate, plus other agents, 29% of the treated metastatic castrate resistant prostate cancer patients had a greater than 90% drop in PSA. Nakagami, Y., et al., Safety and efficacy of docetaxel, estramustine phosphate and hydrocortisone in hormone-refractory prostate cancer patients. Int. J. Urology (early view, Apr. 26, 2010, digital object identifier 10.1111/j.1442-2042.2010.02544.x). In a randomized trial comparing docetaxel vs docetaxel plus estramustine, 41% achieved a PSA <4 ng/mL but there was no improvement in survival over docetaxel alone. Machiels, J.-P. et al., 2008, J. Clin. Oncol. 32: 5261-68. Chemotherapeutics such as, for example, taxanes and non-steroidal hormonal agents may be used in combination with vaccine therapy, either prior to, or following, vaccine therapy.

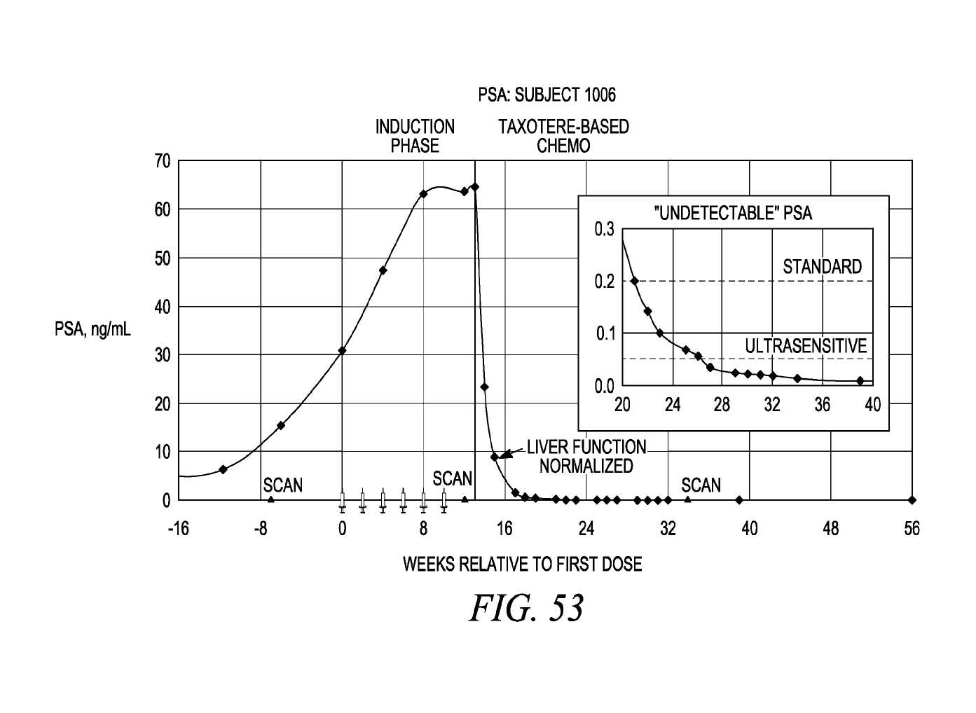

Subject #1006 was administered a combination of chemotherapeutics and the vaccine therapy discussed in this example. Subject #1006 discontinued vaccine therapy after exhibiting symptoms of disease progression. The patient was then treated with chemotherapeutic agents including docetaxel, as well as carboplatin, Estramustine phosphate, thalidomide, decadron, Proscar, Avodart and Leukine. Following therapy, concentration of PSA dropped significantly, to less than 0.2 ng/ml, a drop in serum level of greater than 99%. Serum concentrations of PSA over the course of treatment are indicated in FIG. 53.

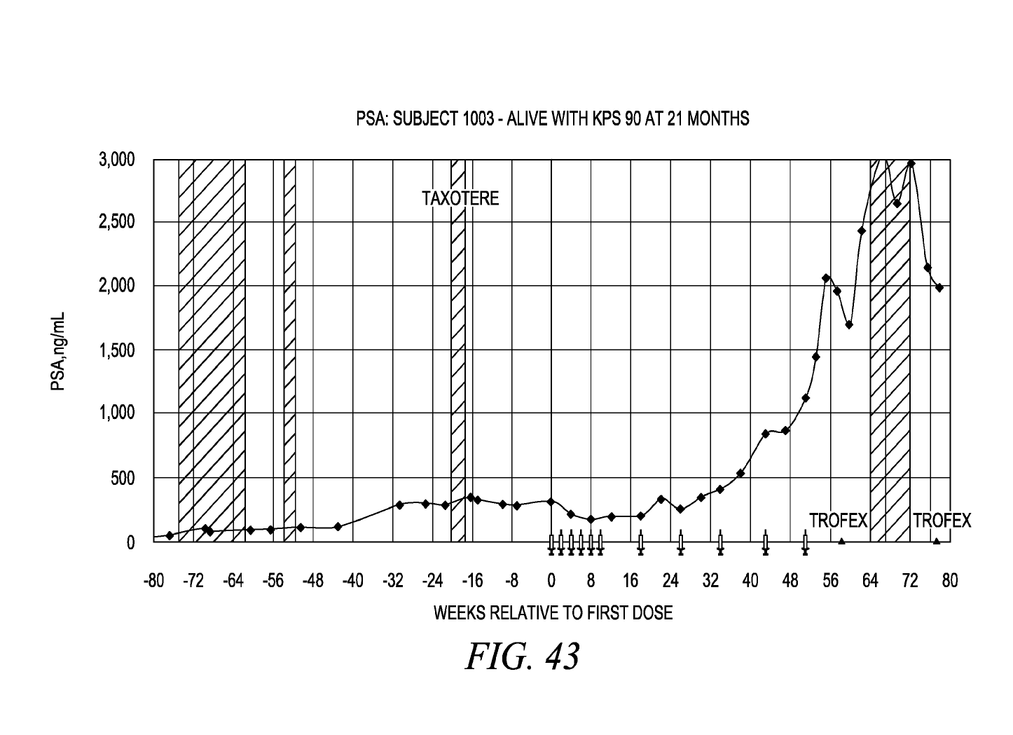

Subject 1003 was treated with Taxotere, followed by vaccine therapy, as shown in FIG. 43. This subject, with a KPS of 90, was alive 21 months following vaccine therapy.

Subject 1007 was treated with Abiraterone, a non-steroidal hormonal agent, before vaccine therapy, and responded to chemotherapy following vaccine treatment.

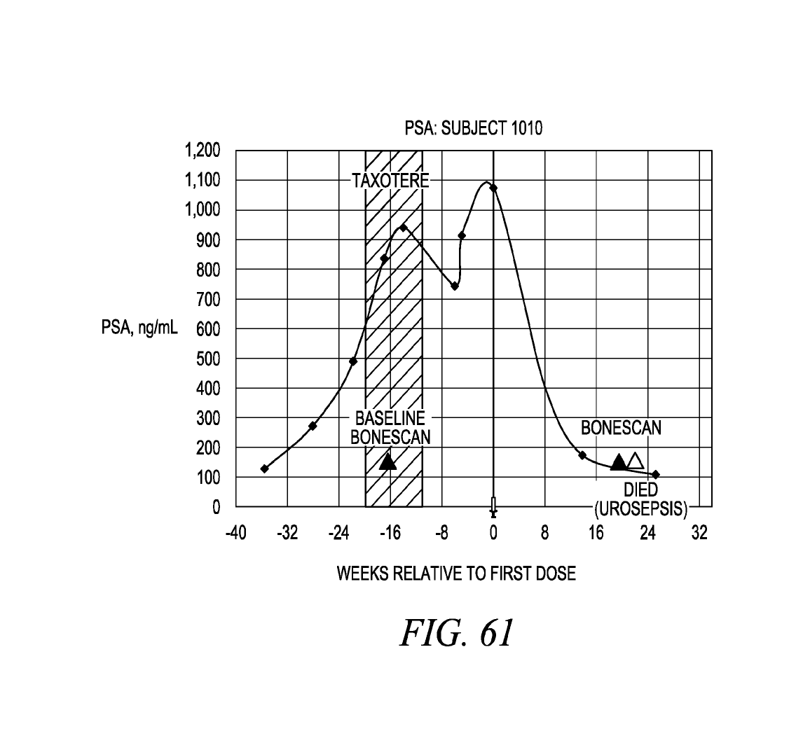

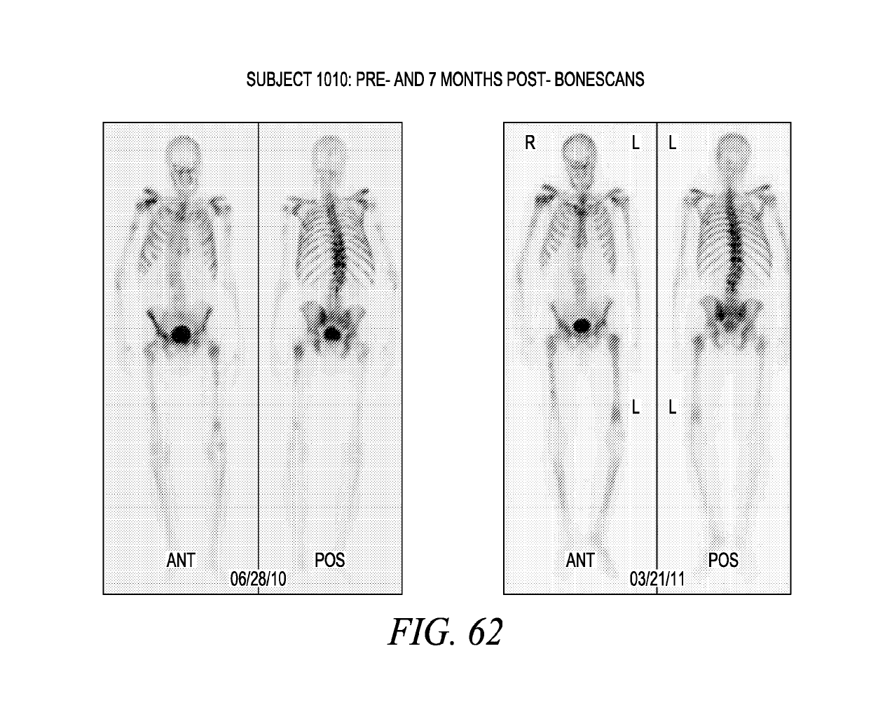

Subject 1010 (FIGS. 61 and 62) was enrolled with a history of Gleason 9 mCRPC, with widespread bone and LN metastases, after failing prior docetaxel chemotherapy, with a rapidly rising PSA >1000 ng/ml (PSADT 1.8 months), CTC 49 and KPS of 80. He withdrew after one dose due to a rapidly declining KPS to 60, and was admitted to home hospice care with no further active therapy except for LHRH agonists. He was projected to survive <1 month. However, 4 months later the patient's status was improving, with increased self-ambulation, appetite, weight gain, and a KPS back over 80-90, and his PSA had dropped to 169 ng/mL (84% decline). H is condition continued to improve and 3 months later, his PSA had fallen further, to 104.3 ng/mL (90% decline). Bonescan at 32 weeks showed significant improvement of diffuse metastases in the ribs, left scapula and left humerus without any new lesions. Shortly thereafter, he developed sudden fever and was diagnosed with urosepsis by his family PCP in Oxford, England. He was treated with oral antibiotics but deteriorated rapidly and expired at 33 weeks at home.

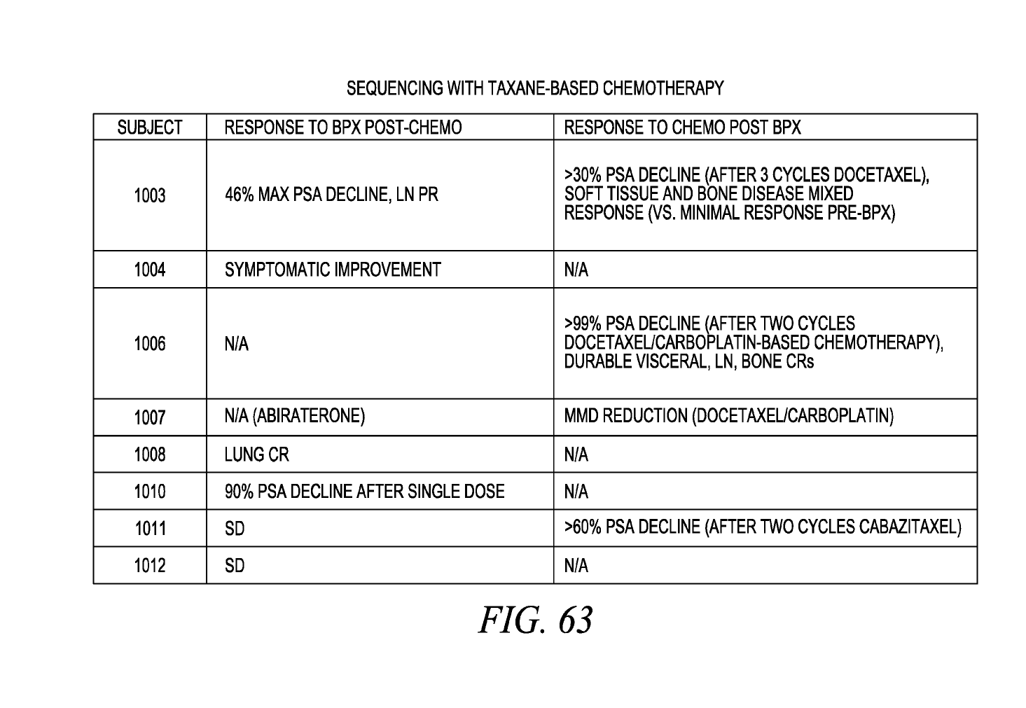

FIG. 63 presents an analysis of combination therapy comprising taxane chemotherapy and vaccine therapy. Synergy between the two therapies is shown using several examples of dosage and sequencing of the therapy.

This demonstrates potential single or more limited dosing activity, and synergy with docetaxel.

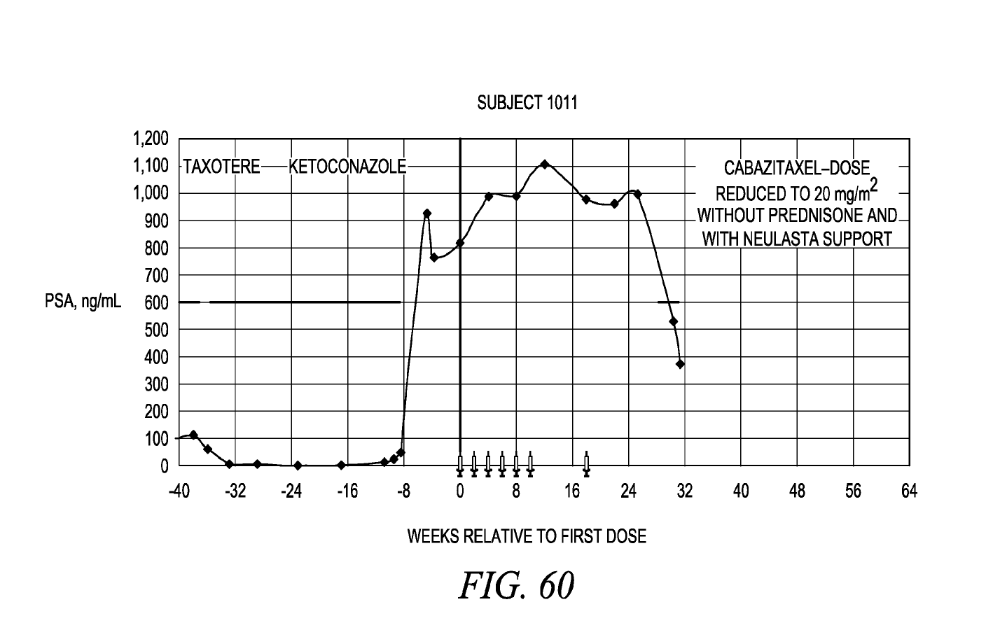

Subject 1011 was administered a combination of chemotherapeutics and the vaccine therapy discussed in this example. As shown in FIG. 60, Subject 1011 was treated with taxotere and ketoconazole before vaccine therapy, and was treated with cabazitaxel after vaccine therapy.

### Example 15

### Second Interim Clinical Data Summary

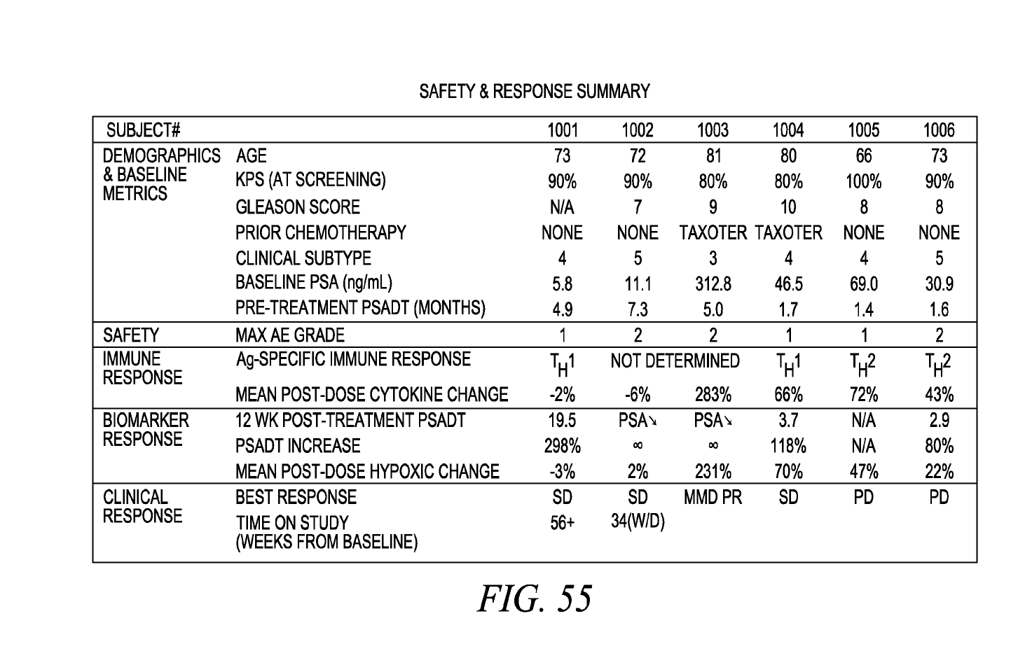

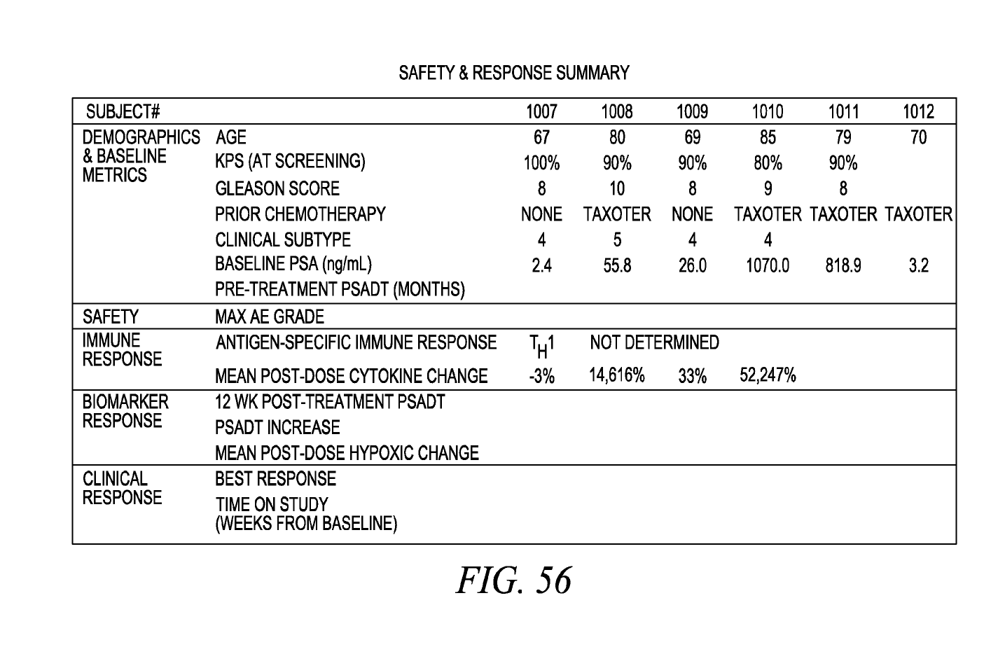

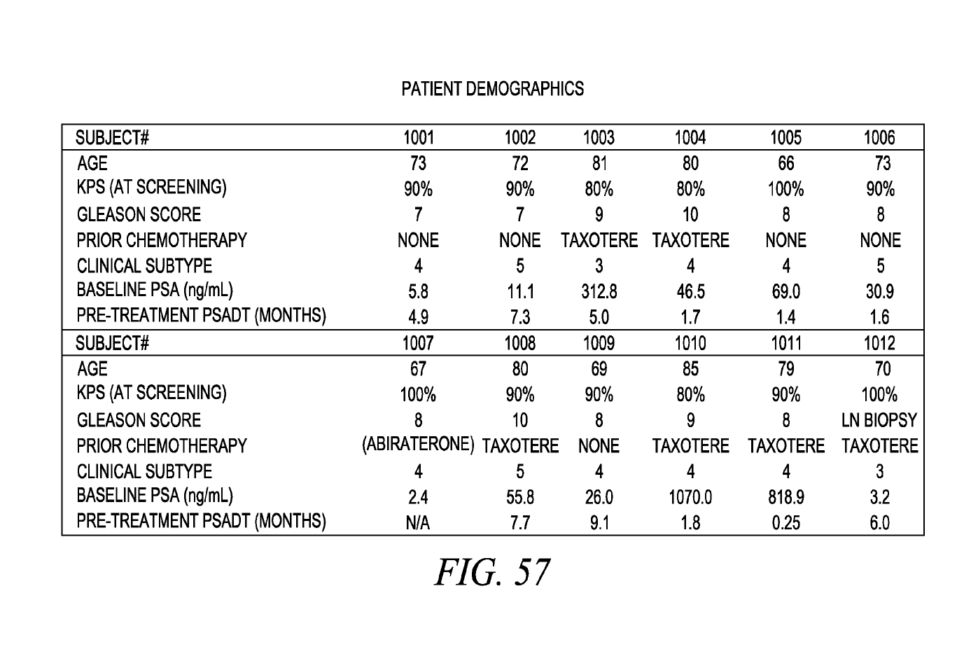

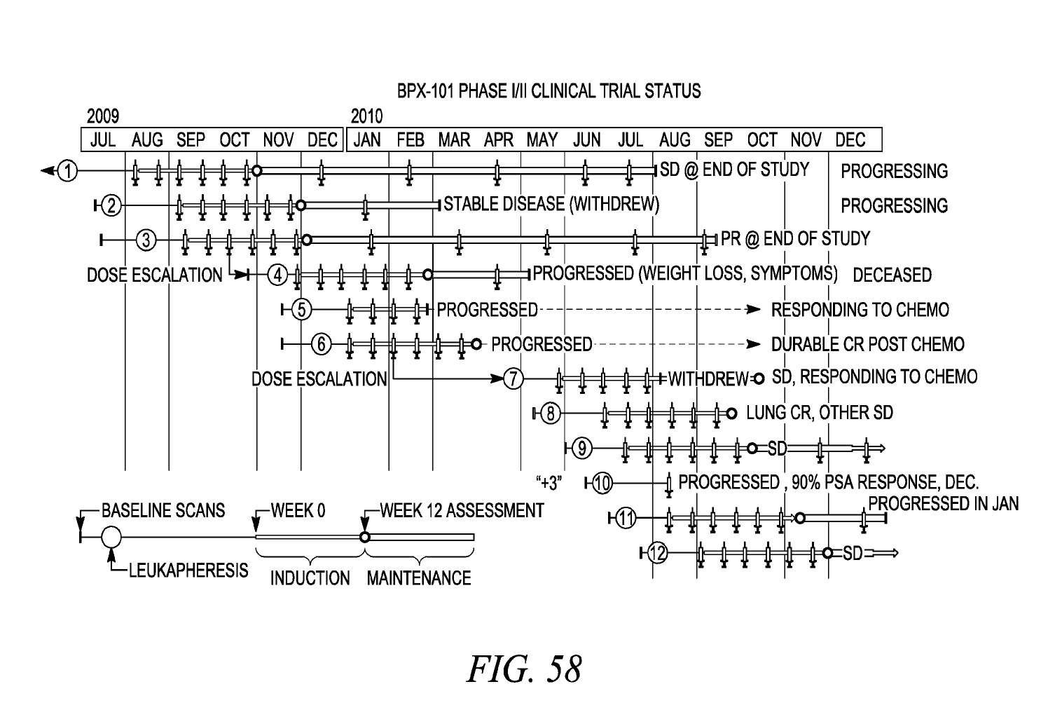

Further clinical trials were performed involving a high dose cohort (subjects 1007-1012) (25×10⁶ cells in 1.6 mL). Additional tests were also obtained from the low (subjects 1001-1003) (4×10⁶ cells in 1.0 mL) and mid dose (subjects 1004-1006) (12.5×10⁶ cells in 1.0 mL) cohorts. FIG. 55 presents a Safety and Response summary from the low and mid dose cohorts, and FIG. 56 presents a Safety and Response summary from the high dose cohort. The patient demographics for all subjects are presented in FIG. 57. The clinical trial status of the patients as of December, 2010 is presented in FIG. 58.

Summary of Results

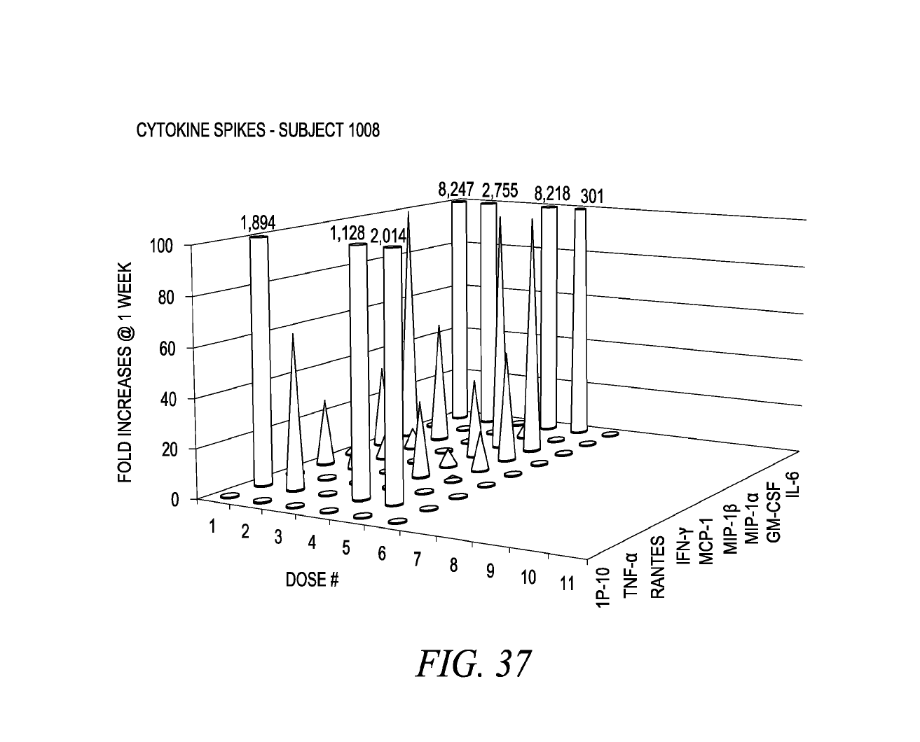

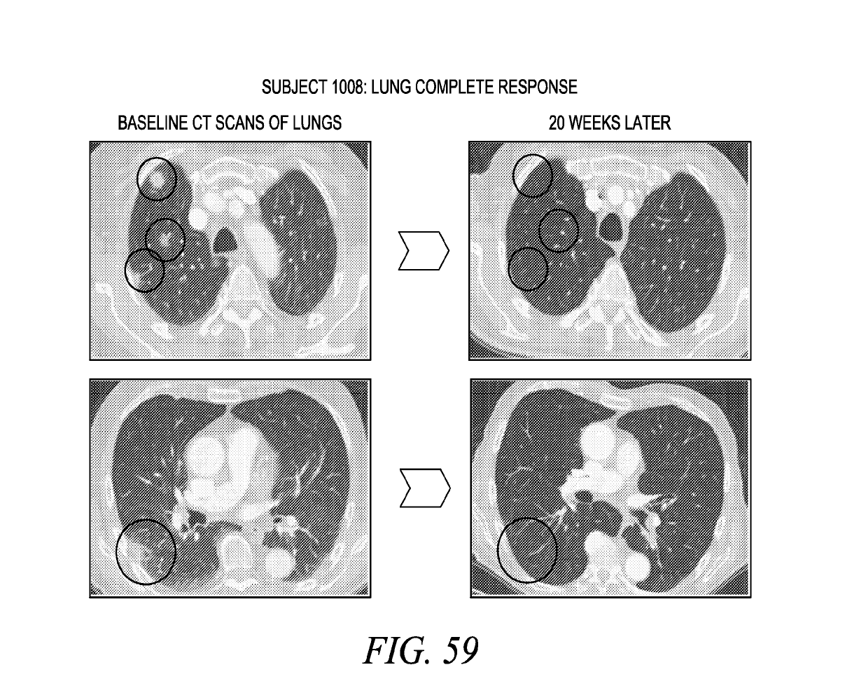

Results: Results: All 3 of the subjects enrolled in the low dose cohort had either stable disease or a partial response at the 12 months after the start of the study. For all three, progression of the disease was delayed 12 months. Subject 1006 of the mid dose cohort, had a complete response after participating in the clinical trial followed by chemotherapy. Subject 1006 was treated with docetaxel, indicating a possible synergistic response from combination therapy. Subject 1008 of the high dose cohort had a complete response for lung tumors, and maintained stable disease measured at 12 weeks. (FIG. 59) The pattern of cytokine spikes in Subject 1008 following treatment is shown in FIG. 37. Subjects 1007 and 1009 of the high dose cohort also had stable disease measured at 12 weeks.

Gleason Scores:

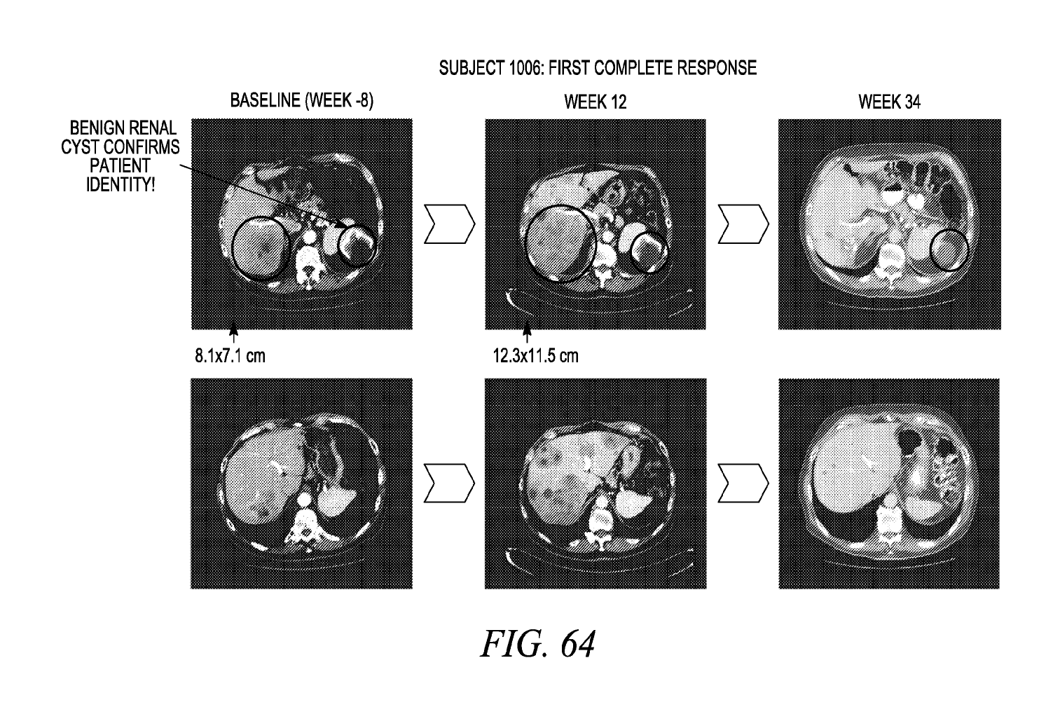

Out of the 12 subjects enrolled in the study to date, 10 had Gleason scores higher than 7. Subject 1003, with a Gleason score of 9, obtained a partial response after treatment with BPX-101. FIG. 40 presents photos showing the tumor shrinkage effect of treatment, as shown for subject 1003. At 26 weeks post treatment, compared to a baseline scan (taken 33 weeks earlier) of an enlarged preaortic lymph node, the node was decreased in size, there was a change from a solid, enhancing mass to a non-enhancing cystic lesion, and the rim of the enhancing tumor tissue was consistent with tumor necrosis. Subject 1006, with a Gleason score of 8, obtained a complete response after combination therapy with BPX-101 followed by a doctaxel-based combination chemotherapy regimen. Subject 1006 presented with a large biopsy-proven prostate cancer metastasis in the liver at baseline. The subject's liver function returned to normal by 15 weeks after vaccine therapy. At 34 weeks, there was no detectable viable tumor, including lung, LN and bone lesions at baseline (FIG. 64). FIG. 53 presents the levels of serum PSA in Subject 1006 over the course of treatment. Subject 1008, with a Gleason score of 10, experienced a complete response in the lung, with near elimination of six separate lung metastases, and stable prostate disease. At enrollment, prostate cancer spread to lungs, lymph nodes and bone. Subject 1008 was treated with 6 doses of BPX-101 (no chemo), starting 6 weeks after baseline scans. Tumors in lungs were eliminated at end of 12 week course of treatment, and the metastases at other sites remained stable. The patient was clinically stable at 20 weeks, with some weight loss, no pain, and bilateral ureteral stents were removed with stable renal function.

Combination Therapy

Subjects 1003, 1004, 1008, 1010, 1011, and 1012 had treatment with Taxotere before participating in the clinical study. Of this group, Subject 1003 obtained a partial response at the end of the study, Subject 1008 obtained a complete response in the lung, and stable prostate disease. Subjects 1011 and 1012 had stable disease at the 12 week point.

Clinical Biomarker Response

Clinical biomarker responses were evident in all three cohorts. Subject 1010 achieved an 85% drop in serum PSA levels after one dose. This patient had been treated with Taxotere before the clinical trial. He received a single dose of BPX-101 before terminating treatment due to clinical progression and a rapidly deteriorating performance status. The Principle Investigator of the trial estimated that the patient had a life expectancy of about one month. However, after a single dose of BPX-101 and no other treatment, the patient has had an improving and now stable course six months later, with an 85% drop in PSA. No scans or other tumor assessments were performed due to the patient's wishes.

A reduction in tumor vasculature was observed in most subjects with measurable soft tissue disease, with subject 1003 obtaining significant tumor shrinkage and an antivascular effect.

An antigen-specific immune response was found in most evaluable patients.

Conclusions:

Summary of clinical observations following a phase I/II clinical trial of BPX-101, a novel drug-activated autologous DC vaccine targeting PSMA. Men with progressive mCRPC following up to one prior chemotherapy regimen were enrolled in a 3+3 dose escalation trial evaluating BPX-101 and CD40 activating agent AP1903. BPX-101 was administered intradermally every 2 weeks for 6 doses, during the induction phase, and for non-progressing patients, every 8 weeks for up to 5 doses during the maintenance phase. AP1903 (0.4 mg/kg) was infused 24 hours after each BPX-101 dose. Radiologic evaluation was performed every 12 weeks. Planned enrollment of 12 subjects has been completed, including 3 each at 4×10⁶ and 12.5×10⁶ cells/dose, and 6 at 25×10⁶ cells/dose. All vaccine products were releasable. Median Halabi-predicted survival was 13.8 months. Two subjects went off protocol prior to the end of induction due to progression, 8 reached end of induction, and 2 are nearing completion of induction. Toxicities (e.g. injection site reactions) were generally mild. One high dose subject experienced a single acute cytokine reaction during infusion of AP1903 at the second vaccination, but continued induction without further drug-related adverse events. Notably, one post-docetaxel subject in the low dose cohort achieved a RECIST PR, and one chemo-naive subject in the mid-dose cohort with extensive visceral, nodal, and bone metastases experienced a RECIST CR with docetaxel-based chemotherapy after induction and maintains an undetectable ultrasensitive PSA (0.009 ng/mL) 10 months after enrollment. A third subject, in the high-dose cohort, experienced near complete elimination of multiple lung metastases with otherwise stable disease by the end of induction. Robust immune responses were seen in all three. BPX-101 can be reliably manufactured and safely administered, followed by AP1903, at doses of at least 25×10⁶ cells. Contrary to the observation that cancer vaccine therapy improves survival without short-term response, BPX-101-treated patients have experienced measurable disease responses, including near elimination of poor-risk visceral disease.

Summary of observations of antigen-specific immunity and tumor inflammation after vaccination with modified antigen-presenting cells, expressing the chimeric protein (BPX-101): Antigen-specific immunity and severe prostate cancer inflammation and necrosis were observed after vaccination in patients enrolled in a Phase 1-2a clinical trial of BPX-101, a drug-activated DC vaccine for mCRPC. Twelve men with progressive, mCRPC were enrolled in a 3+3 dose escalation trial evaluating BPX-101 and activating agent AP1903. BPX-101, which targets Prostate Specific Membrane Antigen (PSMA), was administered intradermally every 2 weeks for 6 doses, followed 24 hours after each dose by infusion of AP1903 (0.4 mg/kg). Injection site skin biopsies were performed after the fourth vaccination. T cells cultured from the skin biopsy ex vivo were stimulated with PSMA protein or control antigens, and were analyzed using Luminex microspheres for 30 inflammatory cytokines/chemokines. One patient (#1007) with an intact prostate developed lower urinary tract bleeding after the fifth vaccination and underwent a transurethral resection of bleeding prostate cancer tissue. Paraffin-embedded blocks were stained for hematoxylin and eosin (H&E). Immunohistochemical stains for CD3, CD4, CD8 and CD34 were also performed. Of 5 subjects with evaluable injection site biopsy results, all exhibited PSMA specific immunity (3 TH1-biased and 2 TH2-biased). Subject 1007's injection site biopsy demonstrated a significant >10-fold increase in IFN-gamma and IL-2 after stimulation by PSMA, compared to stimulation by ovalbumin, consistent with induction of a strong PSMA-specific CTL or TH1-biased immune response. H&E stained resected prostate tissue demonstrated Gleason 8 (4+4) prostate adenocarcinoma exhibiting a severe inflammatory response, consisting of infiltrating plasma cells and CD4+ and CD8+ T cells. Large areas of necrosis were seen adjacent to inflamed prostate cancer tissue. Vaccination with BPX-101 followed by AP1903 can induce a strong, PSMA-specific immune response. Furthermore, evidence of severe prostate cancer-specific inflammation and necrosis, associated with a strong PSMA-specific immune response has been observed after multiple doses of BPX-101.

Summary of observations of the correlation of serum cytokines with clinical responses in patients treated with BPX-101. Men with progressive mCRPC were enrolled in a 3+3 dose escalation trial evaluating BPX-101 and activating agent AP1903. BPX-101 was administered intradermally every 2 weeks for 6 doses, during the induction phase, and for nonprogressing patients, every 8 weeks for up to 5 doses during the maintenance phase. AP1903 (0.4 mg/kg) was infused 24 hours after each BPX-101 dose. Blood samples for immune monitoring were collected weekly during the induction phase, and before and one week after each maintenance dose. GM-CSF, TNF-α, IFN-γ, IP-10, MCP-1, MIP-1α, MIP-1β, and RANTES levels were measured by Luminex microspheres, and IL-6 by ELISA. Planned enrollment of 12 subjects is complete, including 3 each at 4×10⁶ and 12.5×10⁶ cells/dose, and 6 at 25×10⁶ cells/dose. A pattern of spiking levels of serum cytokines one week after each dose, returning to baseline the following week, was observed in subjects with greater disease burden. In one low dose subject who experienced a PR after one year on study, panel cytokines spiked 4-fold on average after each induction phase dose, less than 2-fold after the first two boosters, and between 6-fold and 56-fold after the final three boosters. In a second, high dose subject (#1008), who experienced a near CR of multiple lung metastases with otherwise stable disease, panel cytokines spiked 150-fold on average during the induction phase. In both cases, TNF-α, MIP-1α and MIP-1β spiked the most, including a more than 1.000-fold average spike in TNF-α for subject 1008. Cytokine spikes were not associated with AEs. Conclusions: BPX-101 induces a spiking pattern of cytokine elevations after each dose. In patients who experienced measurable disease reductions, more dramatic spikes in serum inflammatory cytokine levels were seen.

Treatment with BPX-101 and AP1903 elicits both clinical and antigen specific, systemic immune responses. The treatment obtained significant results, either partial or complete responses, or delay of progression, even in subjects with Gleason scores over 7. The treatment, in combination with chemotherapy either before or after BPX-101 and AP1903 treatment, appeared to have a synergistic effect. A reduction in tumor vasculature and tumor size was apparent in certain subjects, as was a reduction in metastatic prostate cancer lesions in lung, liver, bone, and lymph nodes.

### Example 16

### Improving Quality of Life in Cancer Patients

End stage cancer patients usually experience a drastic decrease in their quality of life. Quality of life issues include, for example, cachexia, fatigue, and anemia. By decreasing symptoms of anemia, fatigue, or anemia, the quality of life for patients may be improved.

Cachexia, also known as wasting syndrome, often occurs in patients in end-stage cancer. The term is used to describe the loss of weight, muscle atrophy, fatigue, and weakness seen in cancer patients, and is a positive risk factor for death. Clinical measurements used to determine the level of cachexia in a patient include, for example, hand grip strength, levels of hemoglobin, albumin, C-reactive protein, and fatigue (as measured by, for example, a FACIT-F, a questionnaire that assesses fatigue).

The use of compounds that block IL-6 has been reported to alleviate certain symptoms of cachexia. IL-6 is implicated in many cancers and inflammatory diseases such as, for example, rheumatoid arthritis. The compound ALD-518 is a humanized anti-IL-6 monoclonal antibody that when given in a clinical trial to patients with advanced cancer was reported to reverse fatigue, increase hemoglobin and increase albumin. A trend to increased hand grip strength was also noted. A decrease in C-reactive protein levels was also indicated. Clarke, S. J., et al., 2009, J. Clin. Oncol. 27:15s (suppl.; abstr. 3025). In a larger clinical study of ALD-518 use for cancer-related anemia, cachexia, and fatigue, ALD-518 administration was reported to increase hemoglobin, hematocrit, mean corpuscular hemoglobin, and albumin. M. Schuster, et al., 2010, J. Clin. Oncol. 28-7s (suppl.; abstr. 7631).

Other anti-IL-6 antibodies in clinical trials for rheumatoid arthritis include, for example, elsilimomab and CNTO136. Other methods of blocking IL-6 include blocking the IL-6 receptor (IL-6R). Tocilizumab, also known as atlizumab, is a humanized monoclonal antibody directed against IL6-R. The compound has been indicated for the treatment of inflammatory diseases such as, for example, rheumatoid arthritis.

Administering the transduced or transfected cells, the compounds, or the nucleic acids, and the ligand, of the present methods, may increase the quality of life for cancer patients.

By improving quality of life is meant alleviating at least 1, 2, 3, 4, or 5 symptoms of anemia, cachexia, or fatigue, by at least 10%, 20%, 30%, 40%, 50%, 60%, 70%, 80%, or 90%. For example, where a hemoglobin level in a patient is x, improving the quality of life would include, for example, raising the hemoglobin in the patient after treatment by at least 10%, 20%, 30%, 40%, 50%, 60%. 70%, 80%, or 90%. Alleviating symptoms include, for example, raising hemoglobin, raising hematocrit, increasing weight, raising albumin, decreasing C-reactive protein, decreasing fatigue, and increasing hand grip strength.

By measuring a quality of life indicator symptom is meant measuring or assessing a symptom of anemia, cachexia, or fatigue. For example, the hemoglobin level or a patient, or the hand grip strength of a patient may be measured.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method of treating prostate cancer in a subject, comprising
(a) administering a composition comprising a nucleic acid comprising a promoter operably linked to a nucleotide sequence that encodes a chimeric protein, and a nucleotide sequence encoding a prostate cancer antigen to a subject in need thereof, wherein the chimeric protein comprises a membrane targeting region, a multimeric ligand binding region and a CD40 cytoplasmic polypeptide region lacking the CD40 extracellular domain; and
(b) administering a multimeric ligand that binds to the multimeric ligand binding region;
whereby the composition and ligand are administered in an amount effective to treat the prostate cancer in the subject.

2. A method of treating prostate cancer in a subject, comprising
(a) administering a nucleic acid comprising a promoter operably linked to a nucleotide sequence that encodes a chimeric protein, and a nucleotide sequence encoding a prostate cancer antigen to a subject in need thereof, wherein the chimeric protein comprises a membrane targeting region, a multimeric ligand binding region and a CD40 cytoplasmic polypeptide region lacking the CD40 extracellular domain, and wherein the nucleotide sequence encoding the chimeric protein and the nucleotide sequence encoding a prostate cancer antigen are delivered using an adenovirus vector; and
(b) administering a multimeric ligand that binds to the multimeric ligand binding region;
whereby the nucleotide sequences and ligand are administered in an amount effective to treat the prostate cancer in the subject.

3. The method of claim 1, wherein the membrane targeting region is selected from the group consisting of a myristoylation region, palmitoylation region, prenylation region, and transmembrane sequences of receptors.

4. The method of claim 1, wherein the membrane targeting region is a myristoylation region.

5. The method of claim 1, wherein the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof.

6. The method of claim 1, wherein the multimeric ligand binding region is an FKBP12 region.

7. The method of claim 1, wherein the FKB12 region is an FKB12v₃₆ region.

8. The method of claim 6, wherein the FKBP region is Fv′Fvls.

9. The method of claim 1, wherein the multimeric ligand is an FK506 dimer or a dimeric FK506 analog ligand.

10. The method of claim 1, wherein the ligand is AP1903.

11. The method of claim 1, wherein the CD40 cytoplasmic polypeptide region is encoded by a nucleotide sequence in SEQ ID NO: 1.

12. The composition of claim 1, wherein the nucleic acid is contained within a viral vector.

13. The composition of claim 12, wherein the viral vector is an adenoviral vector.

14. The composition of claim 1, wherein the nucleic acid is contained within a plasmid vector.

15. The method of claim 1, wherein the chimeric protein further comprises a MyD88 polypeptide or a truncated MyD88 polypeptide lacking the TIR domain.

16. The method of claim 15, wherein the truncated MyD88 polypeptide has the peptide sequence of SEQ ID NO: 6, or a fragment thereof, or is encoded by the nucleotide sequence of SEQ ID NO: 5, or a fragment thereof.

17. The method of claim 1, further comprising administering a chemotherapeutic agent, whereby the nucleotide sequences, ligand, and the chemotherapeutic agent are administered in an amount effective to treat the prostate cancer in the subject.

18. The method of claim 17, wherein the chemotherapeutic agent is docetaxel or cabazitaxel.

19. The method of claim 1, wherein the prostate cancer is selected from the group consisting of metastatic, metastatic castration resistant, metastatic castration sensitive, regionally advanced, and localized prostate cancer.

20. The method of claim 1, whereby progression of prostate cancer is prevented or progression of prostate cancer is delayed in the subject.

21. The method of claim 1, wherein the prostate cancer has a Gleason score of 7 or greater.

22. The method of claim 1, wherein the subject has a partial or complete response by 6 months after administration of the multimeric ligand.

23. The method claim 1, wherein the size of the prostate cancer tumor is reduced 20% by 6 months after administration of the multimeric ligand.

24. The method of claim 1, wherein the vascularization of the prostate cancer tumor is reduced 20% by 6 months after administration of the multimeric ligand.

25. The method of claim 1, wherein the prostate cancer antigen is a prostate specific membrane antigen.

26. The method of claims 1, comprising administering a nucleic acid that encodes the chimeric protein and the nucleotide sequence that encodes the prostate cancer antigen.
